(19) United States
(12) Patent Application Publication
(10) Pub. No.: US 2012/0157329 A1
(43) Pub. Date: Jun. 21, 2012

(54) Method and Device for the in Vitro Analysis for MRNA of Genes Involved in Haematological Neoplasias

(76) Inventors: Pilar Giraldo Castellano, Zaragoza (ES); Patricia Alvarez Cabeza, Zaragoza (ES); Miguel Pocovi Mieras, Zaragoza (ES)

(21) Appl. No.: 12/083,824

(22) PCT Filed: May 8, 2006
(86) PCT No.: PCT/ES2006/070054

§ 371 Date: Oct. 2, 2009

(30) Foreign Application Priority Data
Oct. 27, 2005 (ES) 200502618

(51) Int. Cl. C40B 30/04; G06F 19/24; C07H 21/00; C12Q 1/68; C40B 40/06

(52) U.S. Cl. 506/9; 435/6.11; 506/16; 536/24.3; 702/19

(57) ABSTRACT

Method and device for the in vitro analysis of mRNA of genes involved in hematological neoplasias. The device, composed of probes which specifically hybridize with genes involved in hematological neoplasias, designed so that its behaviour in the hybridization is similar, permits the evaluation of the mRNA level in biological samples taken from subjects suspected to be suffering from hematological neoplasia and facilitating the comparison between the different samples and their grouping by similarity in the gene expression patterns, especially when the probes are disposed in the form of microarray. The application of the method of the invention to obtain and process data of gene expression differences from the device of the invention permits the identification of genes significant for distinguishing samples associated to hematological neoplasias, facilitates the diagnosis of neoplasias as CLL and permits making a prognosis of the evolution thereof.

### FIELD OF THE INVENTION

The invention relates to the technical-industrial sector of the extracorporeal in vitro diagnosis of biological samples, by genetic engineering techniques, applied to the diagnosis of specific types of neoplasias from their gene expression patterns and/or to the prognosis of their evolution. More specifically, the invention relates to the identification of neoplasias originating from hematopoietic cells from the evaluation of the levels of messenger RNA of significant genes in biological samples such as peripheral blood samples, preferably by the use of microarrays. With this it is possible to identify samples corresponding to patients suffering from CLL, permitting the diagnosis thereof and, furthermore, it is possible to classify samples from patients suffering from CLL in samples which belong to patients wherein the CLL is going to remain stable or wherein it is going to progress, enabling the prognosis of the future evolution of these patients.

### BACKGROUND OF THE INVENTION

Each day, the human body produces billions of new white and red cells and platelets which replace the hematopoietic cells which are lost as a consequence of a normal process of renewal, disease or trauma. The organized production process of hematopoietic cells and homeostasis is known with the name of hematopoiesis (Weissman I L et al., 2000; Leung A Y H et al., 2005.

In man, hematopoiesis is confined to the bone marrow (B.M.) of the greater part of the bones, and gradually, with age, this is replaced by fat, which in the adult, 70% of the bone marrow is located in the pelvis, vertebra and sternum (Bernard et al., 1976).

All the mature blood cells are generated from a relatively low number of hematopoietic cells known hematopoietic stem cells. The hematopoietic stem cell has two characteristics which are the pluripotentiality or capacity to give rise to different hematopoietic cell strains and the self-renewal or property of self-perpetuation, generating cells the same as its self (Weissman I L et al., 2000). This capacity is essential for the maintenance of hematopoiesis throughout the life which, without self-renewal, would quickly exhaust the reserve of available stem cells. Hematopoietic stem cells are capable of generating different mature hematopoietic cell types through a series of intermediate progenitors and precursors. These progenitors and precursors suffer an ordered sequence of events which transform them into mature cells. This process is known with the name of differentiation (Lee M F et al., 2005). The differentiation of the hematopoietic cells involves changes which affect, among others, the size and form of the cell, gene expression, proteins, response to signals and localization of the cells.

The terminally differentiated cells have lost their capacity for division and suffer apoptosis after a period of time which goes from hours for neutrophils to decades for some lymphocytes. This fact means the B.M. should constantly ensure cell exchange (Datta S R et al., 1999).

The hematopoiesis process comprises a complex interaction between intrinsic genetic events of the hematopoietic cells and environment wherein they are found. This interaction is that which determines if the hematopoietic precursors and progenitors must stay quiescent, proliferate, be differentiated in one or another line or enter into apoptosis (Domen J et al., 1999). All the genetic and environmental mechanisms which govern the production of blood cells operate by altering the relative balance of these fundamental cell processes.

Environmental and genetic factors are critical in hematopoiesis. Thus, for example, the gene expression belongings to the Rb families (Bergh et al., 1999), cyclins (Della Ragione F et al., 1997) or Hox (Magli M C et al., 1997) regulate the proliferation of hematopoietic cells at early stages of differentiation. The genes of the bcl-2 family regulate apoptosis in hematopoietic cells (O'Gorman D M et al., 2001). A great variety of genes among which are found C/EBP (Tenen D G et al., 1997), Pax5 (Nutt S L et al., 1999) and lkaros (Nichogiannopoulou A. et al., 1998) seem to be involved in hematopoietic differentiation and line compromise.

### Hematological Neoplasias

Hematological neoplasias are malignant processes which affect any one of the cell types involved in the hematopoietic system. As a consequence of this transformation, the cell is blocked in a stage of differentiation and starts to accumulate due to uncontrolled proliferation, to a failure of the apoptotic mechanisms or a blocking of its differentiation process.

The malignant transformation of the hematopoietic cells during the different stages they pass through in their differentiation to mature cells originates a great number of different neoplasias (Guttmacher A E et al., 2003). This type of neoplasias is therefore a very heterogeneous group of diseases which only has the hematopoietic origin of the cell type transformed in common.

### Classification of Hematological Neoplasias

Generically, it is possible to establish two groups: lymphoid neoplasias which affect the different cell type and degrees of maturity which form the lymphoid line, both B and T, and the other large group is constituted by the myeloid neoplasis which affect various cell types of the myeloid line. However, this simplistic classification is currently more developed, as detailed below.

From a clinical standpoint, classically, lymphoma leukemias have been differentiated in arbitrary form, indicating the leukemias as those neoplasias which affect the bone marrow and have peripheral expression, i.e. circulation of anomalous cells in blood, and lymphomas as those neoplasias which remain localized in the lymph nodes or other lymphoid tissues and which lack, at least initially, leukemic behaviour. In the case of leukemias, the acute processes of the chronics has initially been differentiated by the morpho-cytological characteristics of the proliferating cells (immature and atypical in the first case and differentiated in the second) and to the clinical manifestations of the disease. At present, the knowledge of the immunological markers and the genetic alterations which affect the hematopoietic cells help to differentiate the different processes more accurately.

Today, it is known that hematological neoplasias, as occurs in other types of cancer, have a multigenic origin. The great technological revolution produced in recent years has made it possible to know the molecular basis of several neoplasias. The use of these techniques makes it possible to identify relevant genes in human cancer, confirm the results obtained in basic research in animal models, establish patters of susceptibility, more accurately classify the neoplasias, improve the diagnosis of the disease, identify new therapeutic targets and improve the therapeutic selection for each patient.

Also, the diversity which exists between individuals is important and has its clinical repercussion, based on the genetic differences: if we are capable of recognising these genetic differences, we will also be capable of advancing in discovering toxicity and differences in response to treatment. (Westbrook C A et al., 2005).

In 1995, the World Health Organization (WHO) in collaboration with the European Hematology Association and pathologists, clinicians and scientists throughout the world, started a project in order to obtain an agreed classification of the hematopoietic tissue and lymphoid organs. This project led to the development of a system for the definition, classification and establishment of agreed diagnostic criteria for myeloid, lymphoid and histiocytic neoplasias (Jaffe E S et al., 2001). The classification criteria of the WHO are the same used in the REAL (Revised European American Lymphoma) classification published by the International Lymphoma Study Group in 1994 (Harris N L et al., 1994). The REAL classification system, unlike other previous classification systems is based on the definition of “real” entities and not morphological subtypes. All available information is used to establish these “real” entities, i.e. morphological, immunophenotypical and biological data are combined with the genetic and clinical characteristics (Harris N L et al., 1999a).

The WHO classification, which was presented in 1997, stratifies the entities in accordance with the cell line affected: myeloid, lymphoid, histiocytic/dentritic and mastocytic. Within each category, the disease is defined in accordance with the morphology, immunophenotype, genetic and clinical data (Harris N L et al., 1999b). In many neoplasias, the stage wherein the accumulated tumour cell is found does not coincide with the stage in which the initial transformer event has occurred. Thus, many hematological neoplasias originate in the initial precursors and the specific genetic alteration may determine which cell continues advancing in its differentiating until stopping and accumulating in more advanced stages of differentiation (Shaffer A L et al., 2002). In contrast, other neoplasias can develop in the more advanced stages of differentiation, as occurs in the cells from the follicular centres wherein the genetic translocations and rearranging produce activation of genes which contribute to tumour development. The classification for each entity reflects the best stimulation for its cell line and stage of differentiation, recognising that the knowledge available at present is imperfect and that changes may occur in the assignment to a cell line and in classification as the available knowledge improves.

The current criteria of diagnosis and classification of these neoplasias are based on a combination of (Braziel R M et al, 2003):
Morphological evaluation of the cell: Observation under the microscope of the cells involved. Information is obtained on the type of cell and degree of its maturity.
Study of the immunophenotype: Recognition of antigens expressed on the surface of the neoplastic cell. These antigens are expressed differently and to different degrees in accordance with the line and of the stage the cell is at. The expression of surface antigens characteristic of the line and stage of differentiation of the cell is known, for example, the expression of CD19 and CD20 is typical of line B cells, whilst the expression of CD3 is typical of line T. The study of CD23 is key when differentiating NHLCM from CLL (Gong J Z et al., 2001).

An attempt has always been made to relate the different types of neoplasias with their corresponding normal cell population through their morphological and immunophenotypical characteristics. Many neoplasias therefore seem “trapped” in determined stages of development as they have morphological and immunophenotypical characteristics similar to those of the hematopoietic cell at that stage of differentiation (Shaffer A L, et al., 2002).
Clinical characteristics: Signs and symptoms of the patient at the time of diagnosis.
Determination of molecular markers: Measurement of some molecules which are associated to concrete entities such as the presence of PMURARA in promyelocytic leukemia or which give a better or worse prognosis, such as, for example, the expression of CD38 in CLL cells marker of bad prognosis (Durig J et al., 2002)
Cytogenetic studies based on the search for genetic alterations in the DNA of tumour cells. In many cases, specific rearranging occur which are characteristic of types of tumour or stages (Mitelman F, et al., 1997). In accordance with the chromosome translocations, it is possible to establish different groups with clinical significance, for example, in LLA-B, where the presence of fusion oncoproteins is frequent, the presence of t(2;21)/TEL1-AML1 and t(1;19)/E2A-PBX1 is associated with a response to the treatment whilst the prognosis for patients with t(9;22)/BCR-ABL and t(4;11)/MLL-AF4 is much worse (Arico M et al., 2000). Searches are also usually made for specific mutations, deletions or insertions in a gene which have been related to more favourable prognosis such as, for example, the myelodysplastic syndromes associated to 5q- (Boultwood J et al., 1994).

As has previously been commented, the WHO establishes four large groups of hematological neoplasias in accordance with the strain involved (myeloid, lymphoid, histiocytic/dentritic and mastocytic lines). Below the neoplasias belonging to the myeloid line and the lymphoid line are described in more detail as they are those which arise with greatest frequency. Those corresponding to the histiocytic/dentritic and mastocytic lines for the moment are very isolated entities.

### 1. Myeloid Neoplasias

They group together all the neoplasias originated in the myeloid line of differentiation, the WHO distinguishes four large groups (Vardiman J W et al., 2002).

1.1 Myeloproliferative Syndromes (MPS)

Myeloproliferative syndromes (MPS) are clonal alterations of the hematopoietic stem cell characterized by effective hematopoiesis which leads to an increase in the blood levels of one or more hematopoietic and hepatosplenomegaly lines. They constitute a group of entities wherein there exists an increase in precursors of the myeloid series or fibrosis of the bone marrow (myelofibrosis); this group also includes systemic mastocytosis. The following can be highlighted:
Chronic myeloproliferative syndromes (CMPS). Clonal alteration of the hematopoeietic stem cell. Characterized by an effective hematopoiesis which produces increase in peripheral blood of one or more cell lines and frequently hepatosplenomegaly, medullary hypercellularity with maturity but without dysplasia.
Chronic myeloid leukemia (CML). It is a clonal process secondary to an acquired genetic alteration of the pluripotent cell. The disease is characterized by the superproduction of neu trophils and of their precursors. It has three phases: the first called chronic phase of undefined duration, followed by the acceleration phase and finally the blastic crisis which is really secondary acute leukemia.

CML has a low incidence of approximately one case per 100,000 inhabitants/year and appears most frequently in the sixth and seventh decades of life. It can be considered a rare disease.

It is the characteristic leukemia par excellence as the term leukaemia was applied to this entity for the first time. 95% of the cases have a genetic marker, the Philadelphia chromosome, originated by the translocation of a fragment of chromosome 22 which adheres to chromosome 9 or t(9;22) (q34;q11). This translocation causes the fusion gene bcr-abl. The protein coded by this chimeric gene, BCR-ABL, has an increased thyrosine-kinase activity compared with the normal abl protein activity as oncogenic growth factor (Pane F et al., 2002), although really the mechanisms which produce the superproduction of myeloid cells are not totally clarified. It is possible that other proto-oncogenes such as p-53 intervene in the process and in the transformation of chronic phase to blastic crisis. The few cases in which the Philadelphia chromosome is detected represent atypical myeloproliferative symptoms and correspond to the variant of MDS known as chronic myelomonocytic leukemia (CMML).

The diagnosis is based on the high cell counts for the blank series, appearance of morphologically normal myeloid cells and in all the stages of differentiation, but with a high number of myelocytes and neutrophils, there are generally basophilia and thrombocytosis. In the acceleration phase an increase in immature cells occurs in the peripheral blood and in the blastic crisis the predominant cell is the myeloblast (65%) or the lymphoblast (35%).
Vaquez's disease (VD). It is the myeloproliferative syndrome characterized by the increase in mass of the red series. Vazquez's disease is a benign haematological disease, whose suffering does not influence shortening of survival. However, it is a clonal disease which may evolve in 15% of patients to myelofibrosis or acute leukemia (5%).
Essential Thrombocythemia (ET). Myeloproliferative syndrome characterized by platelet production 15 times greater than normal. It may be associated to thrombotic or hemorrhagic complications secondary to platelet dysfunction. It appears at around 60 years of age, with equal incidence in both sexes.
Myelofibrosis (MF). It is a neoplastic clonal disorder of the pluripotent stem cell. It is characterized by a great production of abnormal megakaryocytes. These cells release molecules (growth factor derived from platelets, platelet factor 4) which stimulate the proliferation of fibroblasts and build collagen fibres in the bone marrow. The bone marrow is incapable of functioning normally and the hematopoietic precursor cells translate to the liver and spleen, giving rise to extramedullary hematopoiesis. Characterized by fibrosis of B.M and splenomegaly. It appears in people over 50 years of age and has no preference of sex.

Mastocytosis. Group of entities characterized by the proliferation of mastocytic cells in different parts of the body. Systemic mastocytosis (SM), is a rare disease which typically affects adults and has bone alterations in 70% of patients (Chen C C et al., 1994).

1.2. Myelodysplastic/Myeloproliferative Syndromes (MDS/MPS)

The WHO has established a somewhat different classification, separating MDS/MPS as entities differentiated from the other MDS, since they share characteristics with the CMPS that make them different. This group includes three entities: chronic myelomonocytic leukaemia, chronic atypical myeloid, leukeumia, juvenile myelomonocytic leukaemia and non-classifiable MDS/MPS. Myelodysplastic syndromes (MDS) are clonal proliferations of the hematopoeietic stem cell which share at the time of diagnosis, clinical, morphological and analytical data which are superimposed between AML and CMPS. They are characterized by the hypercellularity of bone marrow due to the proliferation of one or more myeloid lines (Heaney M L, 1999). The presence of dysplasia in at least one line (myeloid, erythroid or megakaryocytic-platelet) is a characteristic of MDS. The incidence is variable depending on the variety. An incidence of 3 cases×100,000 inhabitants over 60/year is estimated. The FAB classification establishes 4 diagnostic categories (Bennett J M et al., 1984): simple refractory anemia (RA), refractory anemia with ring sideroblasts (ARS), refractory anemia excess blasts (RAEB) and refractory anemia with excess blasts in transformation (RAEB-T) and chronic myelomonocytic leukemia (CMML).

With regard to the MDS, the WHO establishes five differentiated categories (Harris N L, et al., 1999): refractory anemia, refractory cytopenia with multiline dysplasia, refractory anemia with excess blasts, non-classifiable MDS and MDS associated to an isolated defect in chromosome 5 (of the 5q) or syndrome 5q-.

1.3. Acute Myeloblastic Leukemia (AML)

Clonal proliferation of immature cells of the myeloid line. They may appear de novo or secondary in patients with myelodysplastic syndrome (MDS). The classification prepared by the French-American-British group (FAB) considers eight varieties (M0-M7) based on morphological criteria and on the immunophenotype of the neoplastic cells (Bennett J M, et al., 1976). Despite the fact that this classification has been accepted for many years, the discovery that many genetic alterations have a predictive characteristic and the incorporation of the cytogenetic analysis to the diagnosis of acute leukemias (Bene M C et al., 2001) has made it possible to subclassify the disease and establish the evaluation of the prognosis, as occurs with translocation t(15;17) which characterized promyelocytic variety leukemia which is characterized by the expression of a retinoic acid receptor (RAR), characteristic which makes this type of leukaemia sensitive to treatment with transretinoic acid (TRA) in most cases.

The WHO classifies AML by incorporating morphological, immunophenotypical, genetic and clinical data to be able to define biological homogeneous entities and with clinical relevance. Thus, AML is classified into four large categories: 1.—AML with recurrent genetic anomalies. 2.—AML with multiline dysplasia. 3.—AML related to treatment and 4.—non-classifiable AML (ref WHO). The three first categories recognise the importance of biological factors which predict the evolution of the process. The cytogenic analysis represents the most powerful prognosis factor (Roumier C, et al., 2003). It is used to identify subgroups of AML with different prognosis: low risk with favourable response to treatment (t(8;21), t(15;17) or inv(16)), intermediate risk (normal karyotype or t(9;11) or high risk (inv(3), −5del(5q) or −7del(7q), or more than three alterations). There is molecular heterogeneity within the risk group. In some cases of patients with normal karyotype, the presence of mutations has been found in gene FLT3 (Kottaridis P D, et al., 2001.) and MLL (Dohner K et al., 2002).

The medullary image in the microscopic examination of aspirate is generally that of invasion by cells similar to one another, of immature morphological characteristics which distort the normal cell distribution constituting authentic cell sheets. Medullary hyperproduction conditions which areas of inactive bone marrow come to again present a new focus of hematopoiesis in the adult age, in this case of abnormal cells.

Approximately 80-90% of young patients with AML, achieve complete remission of the disease after chemotherapy. However, the majority relapses, and a cure occurs in 30%. The oncogenic transplant of bone marrow has managed to increase the cure rate to 50%, but it is limited by the availability of identical donor HLA. It is therefore a group of neoplasias with diverse genetic abnormalities and variable response to treatment (Giles F J et al., 2002)

### 2. Lymphoid Neoplasias

The WHO's classification is a refinement of the REAL classification (Harris N L et al. 1994). Three large groups of lymphoid neoplasias: 1.—Lymphoid neoplasias derived from B cells. 2.—Lymphoid neoplasias derived from T and NK cells. 3.—Hodgkin's lymphoma. This classification includes solid neoplasias and lymphoid leukemias, as in many of them their occurs a transformation from one phase to another and the distinction between them, may be artificial. Thus, chronic lymphatic leukemia B and the lymphocytic NHL are originated by the same cell and represent different manifestations of the same neoplasia, the same occurs with lymphoblastic lymphoma and lymphoblastic leukemia

2.1. Neoplasias Derived from B, T and NK Cells

The WHO's classification divides these neoplasias in accordance with the stage of maturity of the cells in neoplasias of precursor cells and neoplasias of mature cells (WHO Classification Tumours of Haematopoietic and lymphoid tissues. In Pathology and genetics of tumours of Haematopoietic and lymphoid tissues. E S Jaffe, N L Harris, H Stein, J W Vardiman. IARC Press. Lyon, 2001). Due to the high number of entities described, the following are highlighted:
Acute lymphoblastic leukemia (ALL): Clonal proliferation of lymphoid precursors. In approximately 80% of the cases, the precursors belong to the lymphoid B line. The molecular analysis of the genetic alterations of the leukemic cells have significantly contributed to the understanding of the pathogenesis and prognosis of ALL (Ferrando A A et al., 2005). Despite the fact that the frequency of genetic subtypes differs in children and in adults, the general mechanisms which lead to ALL are a consequence of the abnormal expression of proto-oncogenes due to chromosome translocations which create fusion genes or a hyperploidy. This initial oncogenic event is probably insufficient to produce leukemia and it is believed that other alterations which cooperate with this first one are necessary to definitively alter the proliferation and survival of the transformed cell. All these alterations contribute to the leukemic transformation of the hematopoietic stem cells or of their progenitors as they affect key regulating processes, maintaining or increasing their capacity for self-renewal, escape from the normal proliferation controls, blocking of differentiation and promoting resistance to apoptotic signals (Hanahan D, et al., 2000).

The overall appearance of the bone marrow is similar to that described for myeloid leukemia. The research of the minimal residual disease is important, a factor which condiciona with su presence the probable relapse of the disease. The FAB classification defines 3 stages in accordance with the morphology (L1-L3).

It is the most frequent leukemia in the childhood, and in the clinical course and the response to treatment depends on the type of genetic alteration, for example, patients with hyperdiploidy have a favourable prognosis when it is treated with treatment schemes which include antimetabolites but, in general terms, children are cured with standard chemotherapy and prophylaxis of the CNS and in adults only 20% have prolonged survival with chemotherapy, the allogenic autologous transplant is useful for cases considered high risk.
Chronic lymphatic leukemia (CLL). CLL is characterized by clonal proliferation and accumulation of lymphocytes with mature appearance and resistant to apoptosis in B.M, blood and lymphoid organs (Galton D A, 1966). When the lymphodenopathy is dominant, the clinical symptoms are called Lymphocytic lymphoma. The lymphocytes affected are line B in 95% of the cases and 5% of the cases involve T lymphocytes.

It is the most frequent leukemia in the Western world. The average age of patients diagnosed is 65 years old, only 10-15% of the cases arise under 50 years (Jemal A et al., 2003). It is the most common cause of leukaemia in adults of the counties of the Western world and involves around 25% of all leukemias. The incidence is 3 cases per each 100,000 inhabitants and year, with a predominance in males, with a male/female proportion of 1.7:1. In recent years, it has increasingly been diagnosed in younger patients. The proportion of cases diagnosed at early stages of the disease (Rai K R, et al., 1975) has increased from 10 to 50%, probably due to an early diagnosis thanks to routine lymphocyte counts. The disease affects more men than women.

The prognosis and clinical course of the disease is extremely variable. Some patients have a rapidly progressive evolution and die in the 2-3 years after the diagnosis, whilst in others, the course is indolent and they live for 10-20 years without problems related to the CLL. Intermediate cases occur in half of patients.

Approximately, 20% of patients are asymptomatic at the time of diagnosis, performing this as a consequence of a routine blood analysis. When symptoms exist, they are not specific and include fatigue, weakness and discomfort.

The Binet classification (Binet J L et al., 1981) defines 3 stages of disease in accordance with the concentration of haemoglobin, number of platelets, number of lymph nodes involved and the presence of visceromegalies. The Rai classification (Rai K R et al., 1975) uses the same indicators but classifies patients in five groups.

This neoplasia is not characterized by a unique and recurrent genomic alteration. There are some markers which give a more unfavourable prognosis such as the presence of deletions in chromosomes 17 and 11 and those patients with absence of mutations in IgVh genes (40% of the cases) and high proportion of cells expressing CD38 is characterized by a more agressive clinical course and a worse response to treatment (Hamblin T J et al., 1999; Durig J et al., 2002). Another recently described marker is ZAP-70, independent prognosis marker whose expression is indirectly related to the mutational state of the gene of the heavy chains of immunoglobulins (Crespo M et al., 2003).
Multiple myeloma: MM). MM is a malignant disease wherein a clone of plasma cells (terminal cells of the B lymphoid line) of the bone marrow suffers uncontrolled proliferation. It involves 10-15% of all the malignant diseases and is characteristic of advanced ages, only 2% of the cases are diagnosed before 40 years of age. For unknown reasons, the incidence of the disease is increasing.

These cells produce and secrete monoclonal immunoglobulin or fragments of immunoglobulins, composed by a heavy and light chain class (kappa or lambda). Occasionally, the myeloma cannot be secreted or the protein is not detectable in serum or urine. The neoplastic plasma cell produces other molecules such as IL6, tumour necrosis factor or osteoclast activator factor which contributes to producing osteolysis, hypercalcemia and renal insufficiency, characteristics alterations of the disease.

The diagnosis can be casual on performing an analysis in patients without symptomology or limited disease (20% of cases). The disease in these patients can remain stable for years and early treatment in the asymptomatic phase does not provide any advantages.

Patients with monoclonal component but which do not meet the MM diagnosis are considered carriers of monoclonal gammapathy of indeterminate meaning (MGIM). Among 10 and 20% of these patients develop MM in 10 years (Kyle R A, 1997; Zhan F et al., 2002). The monoclonal component can also be associated to other diseases such as lymphoma, non-hematological neoplasias and diseases of the connective tissue.
Lymphoplasmocytoid lymphoma and Waldenstrom's macroglobulinemia. It is the clinical expression of a low-degree lymphoproliterative disease, characterized by the infiltration of anomalous lymphoplasmocytic cells in bone marrow, lymph node and spleen, accompanied by monoclonal production of immunoglobin M, which conditions an increase in blood viscosity and the appearance of haemorrhagic vascular manifestations and by difficulty in circulation in the small vessels.
—Non-Hodgkin's lymphoma (NHL). NHL are solid tumours of the lymphoid tissue which are much more heterogeneous than Hodgkin's disease. The complexity and diversity of the NHL as regards morphology, genetics, phenotype and clinical behaviour has given rise to the existence of multiple classifications, none of them completely satisfactory.

It is the most frequent hematological disease and, in terms of years of life lost, it is the fourth most important neoplasia of the Western world and it seems that its incidence is increasing.

It may appear at all ages, but the average appearance is 50 years of age. The cause of the disease is not clear. Specific chromosome translocations have been described associated to certain types of lymphomas, for which reason they are of great use in diagnosis (Montoto S et al., 2003). Most of the Burkitt-type lymphomas present translocation t(8;14), wherein the c-MYC oncogene of chromosome 8 is transferred to the next region in chromosome 14 where the heavy immunoglobins chains are coded. 90% of ollicular lymphomas are characterized by translocation t(14;18), where the bcl-2 gene of the chromosome 18 is transferred to the region of the heavy immunoglobulin chains. It is well known that the overexpression of bcl-2 inhibits apoptosis (programmed cell death). It is easy that this chromosome rearranging requires other stimulation, such as, for example, the coexpression of a second proto-oncogene or an antigenic stimulation to develop the malignant proliferation. An example of combination of multiple combined causes constitute the lymphoma associated to AIDS. The appearance of aggressive extranodal lymphomas is the result of the combination of immunosuppression by HIV, deregulation of a proto-oncogene (c-MYC) and a secondary viral infection (Epstein-Barr's virus), the same occurs in patients subjected to organ transplant (Harris N L et al., 2001).

The clinical presentation of the disease is more irregular than in Hodgkin's disease. It may behave indolently without requiring immediate treatment or, in contrast, behave aggressively which is quickly fatal.

The most frequent nodal condition is cervical. As regards extranodal condition, the signs and symptoms depend on the affected organ. The bone marrow appears infiltrated with greater frequency in the low degree NHL and may cause pancytopenia. The presence of malignant cells in peripheral blood is also frequent in low-degree NHL, but of very bad prognosis in those of high-degree.

The diagnosis is carried out by the histological study of the lymphatic tissue. The additional information is obtained by monoclonal antibodies directed against specific lymphocytic antigens (immunophenotype); this helps to identify the degree of maturity of the malignant cell and determine the T or B origin thereof. The presence of mutation in genes which code Ig in the NHL of strain B are usually used for the identification of some subtypes of NHL (Kuppers R et al., 1999).

2.2. Hodgkin's Lymphoma (LH)

It is an infrequent disease and has predilection for the masculine sex in a proportion of 2/1. It is characterized by the presence of large cells, bi or multi-nucleus called Reed-Sternberg (RS) and other smaller and mononuclear cells which appear in a small quantity in the tumour; the rest of the cells are lymphocytes, granulocytes, fibroblasts and plasma cells. This inflammatory infiltrate probably reflects the immune response of the host with the malignant cells. The nature of the RS and Hodgkin's cells have been greatly studied but continues being disputed. They may be derived from an initial stage of the lymphoid cells.

In some cases, the existence of DNA for Epstein-Barr's virus has been detected in the tumour. One hypothesis is that the bimodal distribution of the disease is due to the infection in young subjects and the other peak would be caused by average environmental causes.

The diagnosis is obtained by biopsy of a lymph node. To plan the treatment, it is necessary to determine the extension of the disease. (Küppers R, 2002; Cossman J, 2001; Devilard E et al., 2002).

### Problems in Classification

The great quantity of hematopoietic cells and the many stages of differentiation through which they pass further complicates the classification of the neoplasis originating from this type of cells. Despite the efforts to establish a classification based on “real” entities, some of the categories are ambiguous and in many cases contain very heterogeneous groups as regards a response to therapy of clinical course. This heterogeneity is that responsible for, on the one hand, the incessant search for markers capable of differentiating some behaviours from others and, on the other hand, that the disputed classification of this type of neoplasis is subjected to continuous revisions.

An ideal classification system should be precise, reproducible, easy to use and should especially have biological and clinical significance (Chan W C, et al., 2005). The current diagnosis systems and the classification of the hematological neoplasias are based on the recognition of histological and morphological, immunophenotypical and cytogenetic characteristics and study of a molecular marker with prognostic value. However, in some of the diagnostic categories defined in this way, the following is observed:
A marked heterogeneous therapy response. Within the same disease there are patients who reach full remission, partial remission, do not respond, which relapse after a certain therapy. The capacity to predict a response is especially important in this type of neoplasis since the transplant of stem cells is an effective but toxic alternative response. The capacity to determine what patients would respond to a conventional therapy before giving it may be beneficial to be able to apply the most effective treatment to each patient.
A variable clinical behaviour. Within this category there are patients whose disease is going to remain stable for long periods of time and which are not going to need therapy and those whose disease is going to progress rapidly requiring aggressive therapy.

These variations point to the existence of molecular heterogeneity within the diagnostic categories, differences which the conventional methods of diagnosis are not capable of determining and hence, the search for new forms of analysis which provide a greater resolution in the characterization of this type of neoplasias.

In this line, the use of expression arrays have demonstrated being effective not only in deciphering the biological and clinical diversity which is found in many tumours, but in understanding the biological and pathological processes which affect many symptoms and, in particular, the hematopoietic system. The expression arrays are ordered arrays of sequences associated to a solid support, complementary to mRNA or to its corresponding cDNA or cRNA, which allow the analysis of the differential expression of hundreds or thousands of genes simultaneously. One of the supports to which they are frequently bound is to rectangular fragments of glass similar to slides, a format which is frequently alluded to by the terms microarray, biochip or, simply, chip. Their use is becoming increasingly frequent for the diagnosis of various diseases or for the evolution of the evaluation of the susceptibility of suffering from them.

### First Works of Arrays and Hematological Neoplasias

In 1999, the Golub group published one of the first articles referring to the role of arrays in the classification of hematological neoplasias (Golub T R et al., 1999). An array with 6817 genes represented was used for the study of expression profiles in AML and ALL. A group of 50 genes was selected with the capacity of predicting the type of leukemia (class predictor) and they were used to classify a group of unknown samples in the correct categories. The study of the expression of these 50 genes is sufficient for the classification of a sample of acute leukemia in AML or ALL. Despite the fact that the distinction between AML and ALL is well established with the current diagnostic methods, the study revealed the existence of specific expression patterns associated with each type of acute leukemia and proved the use of expression profiles in cancer classification.

In 2000, the Alizadeh group published an article in which a specialized array is used, the lymphochip which contains genes expressed preferentially in lymphoid cells or if which an immunological or oncological importance is known with 17,856 sequences (Alizadeh A A et al., 1999). This group used the “lymphochip” for the study of gene expression patterns associated to differences in clinical behaviour in a Diffuse Large B-Cell Lymphoma (DLCL) (Alizadeh A A, et al. 2000). The DLCL is a NHL with a very heterogeneous behaviour and impossible to distinguish using conventional diagnostic methods: 40% of patients respond well to therapy and have prolonged survival whilst 60% die due to the disease. The authors found that the gene expression could be related to the clinical behaviour of the tumours. This was one of the first articles to speak of arrays for the “subclassification” of hematological neoplasias, i.e. the use of expression profiles for the identification of two different groups of DLCL from the transcriptional standpoint, DLCL subtypes with clinical behaviour impossible to predict with conventional diagnostic criteria.

At present there are multiple publications wherein, directly or indirectly appear the arrays applied not only to classification and subclassification, but also to the study, diagnosis, prognosis, identification of new markers in haematological diseases (Greiner TC, 2004; Alizadeh A A et al, 2000; Bea S et al., 2005; Dave S S et al., 2004), as well as patent applications which disclose the use of this type of device for the differentiation between different types of hematological neoplasias. Thus, for example, patent application WO2003/008552 discloses the use with diagnostic purposes of differences in the expression pattern of genes to differentiate between mixed line leukemia (MLL), acute lymphoblastic leukemia (ALL) and acute leukemia myelogenous leukemia (AML), defending the possibility of making this differential diagnosis with the data obtained after the diagnosis of samples from patients afflicted by each one of these types of leukemia by the use of commercial chips from Affymetrix. Although genes are indicated with variations in the expression between the three types of leukemias which would permit the differentiation between them, no specific sequences are mentioned other than those present in the Affymetrix chip which could have been used to detect these genes by devices different from those of said company, nor does it consider the design of devices or methods which would permit the diagnosis of other types of leukemias or, in general, neoplasias derived from hematopoietic cells.

Patent application WO2005/024043, for its part, also relates to the field of gene expression analysis to go into greater detail in the knowledge of differences existing at a molecular level between the different neoplasias derived from hematopoietic cells, specifically centering on the case of lymphomas, to extract data which help in its diagnosis or in the prognosis of its evolution. In particular, it discloses a method to obtain useful functions to predict the evolution of individuals affected by different types of lymphomas evaluating in lymph node biopsies to what extent patterns or genetic prints contribute in each one of them, groups of genes which are expressed in a coordinated manner and which are related to the cell origin of the neoplasia, the different types of non-malignant cells present in the biopsy and the oncogenic mechanisms responsible for cancer. The different patterns or genetic prints are also deduced in this case from the data obtained with commercial chips from Affymetrix. Furthermore, application WO2005/024043 states it provides an alternative microarray, composed of a fewer number of sequences than the Affymetrix microarrays, which would also permit the analysis of differences in gene expression between lymphomas and their application for deducing functions of prediction of survival and for the differentiation between different types of lymphomas. Although it indicates the genes whose analysis would be made possible by that microarray, the specification of application WO2005/024043 does not indicate the sequence of the probes which would compose the microarray, only mentioning that they would be cDNA type and leaving doubts over whether that cDNA would appear complete or the analysis of the corresponding gene expression would be carried out using as probe only one fragment of said cDNA, which would remain tp be determined.

It would be interesting to have compositions and methods which would permit ifferentiation between neoplasias of hematopoietic origin based on their molecular level difference, specifically designed for this group of neoplasias, wherein it would evaluate the expression of a more reduced number of genes than in the commercial microarrays used in the studies described in the aforementioned patient applications and which enabled both the diagnosis of certain neoplasias and the prediction of their future evolution, thus helping in the prescription of a suitable treatment for each patient, a particularly interesting characteristic in those neoplasias, as is the case of CLL, wherein the prognosis of the future evolution of the patient is difficult with the knowledge and tests available to date. Furthermore, it would be particularly convenient that the probes used to evaluate the expression of the expressed genes had been designed specifically so that, in addition to being specific and with a perfectly defined sequence, all had a similar behaviour, which would make them suitable, in general, to use in combination in a same test and, in particular, to form part of the same ordered array associated to a solid support, such as chips or microarrays. The compositions and methods of this invention meet this need.

Instead of commercial microarrays to detect genes significant for distinguishing between neoplasias or creating functions which predict the survival of the individual suffering from it, the invention provides new oligonucleotides, of perfectly defined sequence, capable of specifically detecting genes which have been selected as they are known to be significant for the biology of blood cells or for the pathology of different neoplasias, oligonucleotides which also have the feature of having being designed so that they share common characteristics which have a similar behaviour to those used as probes in hybridization, which makes them suitable to be used in compositions which comprise combinations thereof. Said compositions and in particular those wherein these nucleotides are arranged in ordered form on an easy to handle solid support such as glass similar to slides, are suitable for carrying out tests to detect statistically significant genes or differentiate samples taken from individuals suffering from certain types of neoplasias originating from hematopoietic cells of samples taken from individuals not suffering from said neoplasias, as they are compositions which contain a number of nucleotides less than those commercial microarrays designed with a more general purpose, being specifically designed for the analysis of samples from individuals suffering from neoplasias and composed of a known sequence of probes, perfectly reproducible, which are designed to be used together in the same test as they are of similar behaviour. The additional inclusion in the microarrays of the invention of oligonucleotides of low homology with human genes, but chosen so that the rest of their characteristics are similar to those of the oligonucleotides of the invention designed to act as probes capable of recognizing human genes with high specificity, permits the use of said microarrays for the identification of statistically significant genes in the identification of samples associated to certain neoplasias of hematopoietic origin by the use of tests wherein it is feasible to establish controls in all their phases. As shown in the examples which appear further on, in the present specification the use of these microarrays in combination with various statistical techniques permits the correct classification of different biological samples by a method which is precise, reproducible, easy to use and with biological and clinical significance, as they are based on differences of gene expression with significance for the biological processes which are being analysed. In particular, the use of a microarray of the invention in combination with the method of the invention permits the identification of blood samples in patients suffering from chronic lymphatic leukemia (alteration not considered in applications WO2003/008552 and WO2005/024043 and whose diagnosis has not been described by the use of commercial microarrays), distinguishing those of both samples obtained from healthy individuals and samples related to other types of leukemias, and those corresponding to Jurkat or U937 cells, facilitating the diagnosis of CLL through the analysis of expression levels of statistically significant genes to do this and even permitting the obtainment of functions which enable the mathematical calculation of the probability of a sample belonging to individuals afflicted with stable chronic lymphatic leukemia from samples belonging to individuals afflicted with progressive chronic lymphatic leukemia, a distinction which is now difficult to carry out a priori by the available techniques, which means it is a useful and novel tool for the prognosis of the future evolution of individuals afflicted with this disease, individuals whose diagnosis may also have been carried out by compositions and method of the invention or may have been known thanks to the application of a different method, but for which, on having a tool which makes it possible to make a prognosis on how the CLL they are suffering from is going to later evolve, it would be easier to decide if it is suitable to subject them to an immediate aggressive treatment or simply keep them under observation to check that their gene expression data continue indicating that the disease is going to remain stable for a long period of time.

### SUMMARY OF THE INVENTION

The invention provides compositions which include at least one oligonucleotide from the group composed of:

SG1, SG2, SG3, SG4, SG5, SG6, SG7, SG8, SG9, SG10, SG11, SG12, SG13, SG14, SG15, SG16, SG17, SG18, SG19, SG20, SG21, SG22, SG23, SG24, SG25, SG26, SG27, SG28, SG29, SG30, SG31, SG32, SG33, SG34, SG35, SG36, SG37, SG38, SG39, SG40, SG41, SG42, SG43, SG44, SG45, SG46, SG47, SG48, SG49, SG50, SG51, SG52, SG53, SG54, SG55, SG56, SG57, SG58, SG59, SG60, SG61, SG62, SG63, SG64, SG65, SG66, SG67, SG68, SG69, SG70, SG71, SG72, SG73, SG74, SG75, SG76, SG77, SG78, SG79, SG80, SG81, SG82, SG83, SG84, SG85, SG86, SG87, SG88, SG89, SG90, SG91, SG92, SG93, SG94, SG95, SG96, SG97, SG98, SG99, SG100, SG101, SG102, SG103, SG104, SG105, SG106, SG107, SG108, SG109, SG110, SG111, SG112, SG113, SG114, SG115, SG116, SG117, SG118, SG119, SG120, SG121, SG122, SG123, SG124, SG125, SG126, SG127, SG128, SG129, SG130, SG131, SG132, SG133, SG134, SG135, SG136, SG137, SG138, SG139, SG140, SG141, SG142, SG143, SG144, SG145, SG146, SG147, SG148, SG149, SG150, SG151, SG152, SG153, SG154, SG155, SG156, SG157, SG158, SG159, SG160, SG161, SG162, SG163, SG164, SG165, SG166, SG167, SG168, SG169, SG170, SG171, SG172, SG173, SG174, SG175, SG176, SG177, SG178, SG179, SG180, SG181, SG182, SG183, SG184, SG185, SG186, SG187, SG188, SG189, SG190, SG191, SG192, SG193, SG194, SG195, SG196, SG197, SG198, SG199, SG200, SG201, SG202, SG203, SG204, SG205, SG206, SG207, SG208, SG209, SG210, SG211, SG212, SG213, SG214, SG215, SG216, SG217, SG218, SG219, SG220, SG221, SG222, SG223, SG224, SG225, SG226, SG227, SG228, SG229, SG230, SG231, SG232, SG233, SG234, SG235, SG236, SG237, SG238, SG239, SG240, SG241, SG242, SG243, SG244, SG245, SG246, SG247, SG248, SG249, SG250, SG251, SG252, SG253, SG254, SG255, SG256, SG257, SG258, SG259, SG260, SG261, SG262, SG263, SG264, SG265, SG266, SG267, SG268, SG269, SG270, SG271, SG272, SG273, SG274, SG275, SG276, SG277, SG278, SG279, SG280, SG281, SG282, SG283, SG284, SG285, SG286, SG287, SG288, SG289, SG290, SG291, SG292, SG293, SG294, SG295, SG296, SG297, SG298, SG299, SG300, SG301, SG302, SG303, SG304, SG305, SG306, SG307, SG308, SG309, SG310, SG311, SG312, SG313, SG314, SG315, SG316, SG317, SG318, SG319, SG320, SG321, SG322, SG323, SG324, SG325, SG326, SG327, SG328, SG329, SG330, SG331, SG332, SG333, SG334, SG335, SG336, SG337, SG338, SG339, SG340, SG341, SG342, SG343, SG344, SG345, SG346, SG347, SG348, SG349, SG350, SG351, SG352, SG353, SG354, SG355, SG356, SG357, SG358, SG359, SG360, SG361, SG362, SG363, SG364, SG365, SG366, SG367, SG368, SG369, SG370, SG371, SG372, SG373, SG374, SG375, SG376, SG377, SG378, SG379, SG380, SG381, SG382, SG383, SG384, SG385, SG386, SG387, SG388, SG389, SG390, SG391, SG392, SG393, SG394, SG395, SG396, SG397, SG398, SG399, SG400, SG401, SG402, SG403, SG404, SG405, SG406, SG407, SG408, SG409, SG410, SG411, SG412, SG413, SG414, SG415, SG416, SG417, SG418, SG419, SG420, SG421, SG422, SG423, SG424, SG425, SG426, SG427, SG428, SG429, SG430, SG431, SG432, SG433, SG434, SG435, SG436, SG437, SG438, SG439, SG440, SG441, SG442, SG443, SG444, SG445, SG446, SG447, SG448, SG449, SG450, SG451, SG452, SG453, SG454, SG455, SG456, SG457, SG458, SG459, SG460, SG461, SG462, SG465, SG468, SG469, SG470, SG471, SG472, SG473, SG474, SG475, SG476, SG477, SG478, SG479, SG480, SG481, SG482, SG483, SG484, SG485, SG486, SG487, SG488, SG489, SG490, SG491, SG492, SG493, SG494, SG495, SG496, SG497, SG498, SG499, SG500, SG501, SG502, SG503, SG504, SG505, SG506, SG507, SG508, SG509, SG510, SG511, SG512, SG513, SG514, SG515, SG516, SG517, SG518, SG519, SG520, SG521, SG522, SG523, SG524, SG525, SG526, SG527, SG428, SG529, SG530, SG531, SG532, SG533, SG534, SG535, SG536, SG537, SG538, SG539, SG540, SG541, SG542, SG543, SG544, SG545, SG546, SG547, SG548, SG549, SG550, SG551, SG552, SG553, SG554, SG555, SG556, SG557, SG558, SG559, SG560, SG561, SG562, SG563, or combinations thereof.

Said oligonucleotides have been designed so that, in addition to being specific for the corresponding genes whose expression one wants to evaluate, they have a similar behaviour, as they are of similar lengths and all of them have GC in the range of 40% to 60%, in addition to corresponding to zones situated less than 3000 nucleotides from end 3′ (poly(A)) of the mRNA which one wants to detect and evaluated and of being constituted by sequences which coincide in their sense with those of the corresponding mRNA. Therefore, they are suitable to be used in the same test or form part of a composition which comprises combinations thereof. A particular embodiment of the invention is constituted by the compositions which comprise mixtures of several of said oligonucleotides. Especially preferred are those compositions which comprise mixtures of oligonucleotides which correspond to genes significant for classifying a sample as associated to a certain neoplasia and/or to determine the future evolution thereof. Especially preferred embodiments of the invention are also those compositions which comprise the totality of the oligonucleotides from the group composed of:

SG1, SG2, SG3, SG4, SG5, SG6, SG7, SG8, SG9, SG10, SG11, SG12, SG13, SG14, SG15, SG16, SG17, SG18, SG19, SG20, SG21, SG22, SG23, SG24, SG25, SG26, SG27, SG28, SG29, SG30, SG31, SG32, SG33, SG34, SG35, SG36, SG37, SG38, SG39, SG40, SG41, SG42, SG43, SG44, SG45, SG46, SG47, SG48, SG49, SG50, SG51, SG52, SG53, SG54, SG55, SG56, SG57, SG58, SG59, SG60, SG61, SG62, SG63, SG64, SG65, SG66, SG67, SG68, SG69, SG70, SG71, SG72, SG73, SG74, SG75, SG76, SG77, SG78, SG79, SG80, SG81, SG82, SG83, SG84, SG85, SG86, SG87, SG88, SG89, SG90, SG91, SG92, SG93, SG94, SG95, SG96, SG97, SG98, SG99, SG100, SG101, SG102, SG103, SG104, SG105, SG106, SG107, SG108, SG109, SG110, SG111, SG112, SG113, SG114, SG115, SG116, SG117, SG118, SG119, SG120, SG121, SG122, SG123, SG124, SG125, SG126, SG127, SG128, SG129, SG130, SG131, SG132, SG133, SG134, SG135, SG136, SG137, SG138, SG139, SG140, SG141, SG142, SG143, SG144, SG145, SG146, SG147, SG148, SG149, SG150, SG151, SG152, SG153, SG154, SG155, SG156, SG157, SG158, SG159, SG160, SG161, SG162, SG163, SG164, SG165, SG166, SG167, SG168, SG169, SG170, SG171, SG172, SG173, SG174, SG175, SG176, SG177, SG178, SG179, SG180, SG181, SG182, SG183, SG184, SG185, SG186, SG187, SG188, SG189, SG190, SG191, SG192, SG193, SG194, SG195, SG196, SG197, SG198, SG199, SG200, SG201, SG202, SG203, SG204, SG205, SG206, SG207, SG208, SG209, SG210, SG211, SG212, SG213, SG214, SG215, SG216, SG217, SG218, SG219, SG220, SG221, SG222, SG223, SG224, SG225, SG226, SG227, SG228, SG229, SG230, SG231, SG232, SG233, SG234, SG235, SG236, SG237, SG238, SG239, SG240, SG241, SG242, SG243, SG244, SG245, SG246, SG247, SG248, SG249, SG250, SG251, SG252, SG253, SG254, SG255, SG256, SG257, SG258, SG259, SG260, SG261, SG262, SG263, SG264, SG265, SG266, SG267, SG268, SG269, SG270, SG271, SG272, SG273, SG274, SG275, SG276, SG277, SG278, SG279, SG280, SG281, SG282, SG283, SG284, SG285, SG286, SG287, SG288, SG289, SG290, SG291, SG292, SG293, SG294, SG295, SG296, SG297, SG298, SG299, SG300, SG301, SG302, SG303, SG304, SG305, SG306, SG307, SG308, SG309, SG310, SG311, SG312, SG313, SG314, SG315, SG316, SG317, SG318, SG319, SG320, SG321, SG322, SG323, SG324, SG325, SG326, SG327, SG328, SG329, SG330, SG331, SG332, SG333, SG334, SG335, SG336, SG337, SG338, SG339, SG340, SG341, SG342, SG343, SG344, SG345, SG346, SG347, SG348, SG349, SG350, SG351, SG352, SG353, SG354, SG355, SG356, SG357, SG358, SG359, SG360, SG361, SG362, SG363, SG364, SG365, SG366, SG367, SG368, SG369, SG370, SG371, SG372, SG373, SG374, SG375, SG376, SG377, SG378, SG379, SG380, SG381, SG382, SG383, SG384, SG385, SG386, SG387, SG388, SG389, SG390, SG391, SG392, SG393, SG394, SG395, SG396, SG397, SG398, SG399, SG400, SG401, SG402, SG403, SG404, SG405, SG406, SG407, SG408, SG409, SG410, SG411, SG412, SG413, SG414, SG415, SG416, SG417, SG418, SG419, SG420, SG421, SG422, SG423, SG424, SG425, SG426, SG427, SG428, SG429, SG430, SG431, SG432, SG433, SG434, SG435, SG436, SG437, SG438, SG439, SG440, SG441, SG442, SG443, SG444, SG445, SG446, SG447, SG448, SG449, SG450, SG451, SG452, SG453, SG454, SG455, SG456, SG457, SG458, SG459, SG460, SG461, SG462, SG465, SG468, SG470, SG472, SG473, SG474, SG475, SG476, SG477, SG478, SG479, SG480, SG481, SG482, SG483, SG484, SG485, SG486, SG487, SG488, SG489, SG490, SG491, SG492, SG493, SG494, SG495, SG496, SG497, SG498, SG499, SG500, SG501, SG502, SG503, SG504, SG505, SG506, SG507, SG508, SG509, SG510, SG511, SG512, SG513, SG514, SG515, SG516, SG517, SG518, SG519, SG520, SG521, SG522, SG523, SG524, SG525, SG526, SG527, SG428, SG529, SG530, SG531, SG532, SG533, SG534, SG535, SG536, SG537, SG538, SG539, SG540, SG541, SG542, SG543, SG544, SG545, SG546, SG547, SG548, SG549, SG550, SG551, SG552, SG553, SG554, SG555, SG556, SG557, SG558, SG559, SG560, SG561, SG562, SG563.

Additionally, the invention provides oligonucleotides useful to be used as controls in the method of the invention. On the one hand as integrity controls, the pairs of oligonucleotides SG463 and SG464 (complementary, respectively at ends 5′ and 3′ of the β-actin gene) and SG466 and SG467 (complementary, respectively, to ends 5′ and 3′ of the GAPD gene) are provided. Additionally, oligonucleotides SSPC1, SSPC2, SSPC3, SSPC4, SSPC5, SSPC6 and SSPC7 are provided, which may be used as exogenous internal positive controls of the process quality after adding to the sample which contains the starting mRNA molecules of polyadenylated nucleic acids which contain fragments which correspond in their sequence to those of these oligonucleotides (such as the transcripts corresponding to the genes wherefrom said nucleotides are derived) and which are subjected to the same processing as the starting mRNA, as well as oligonucleotides SCN2, SCN3, SCN6, SCN8, SCN11, SCN12 and SCN13, designed to be used as positive hybridization controls and oligonucleotides SCN1, SCN5, SCN7, SCN10, SC1, SC2, SC3, SC4, SC5, SC6 and SC7, designed to be used as negative controls; they all comply with the conditions of having low homology with human genes, in addition to complying with the same conditions of the oligonucleotides complementary to human genes of being of similar lengths and all of them having GC contents in the range of 40% to 60%, correspond to zones situated at less than 3000 nucleotides from end 3′ (poly(A)) of the non-human mRNA which would be capable of detecting and being constituted by sequences which coincide in their sense with those of the corresponding mRNA. Any composition which contains at least one of oligonucleotides SG463, SG464, SG466, SG467, SSPC1, SSPC2, SSPC3, SSPC4, SSPC5, SSPC6, SSPC7, SCN2, SCN3, SCN6, SCN8, SCN11, SCN12, SCN13, SCN1, SCN5, SCN7, SCN10, SC1, SC2, SC3, SC4, SC5, SC6 and SC7, in combination with at least one of the oligonucleotides complementary to human genes of the invention mentioned above is also a composition included in the scope of the present invention.

It is especially preferred that the oligonucleotides which form part of a composition of the invention are bound to a solid support. In particular, those are preferred of said compositions wherein the distribution of the oligonucleotides on the solid support are of ordered form, whereby the solid support is a rectangular piece of glass similar to a microscope slide and that the oligonucleotides are bound to the glass by covalent bonds; the compositions which meet said characteristics are referred to in the rest of the specification with the words “microarray”, “chip” or “microchip”. Among these compositions in the form of microarray, there is a special preference for those which contain more than one copy of each one of the oligonucleotides which form part thereof, very especially preferring that the number of copies of each one of the nucleotides present is at least 12.

The scope of the invention also includes any diagnostic device which comprises a composition of the invention. The expression “diagnostic device” refers not only to that which serves to determine if the individual suffers from a disease or not but also those which serve to classify the disease an individual is suffering from as belonging to a subtype associated to a determined form of future evolution of said disease and, which therefore, also have a prognostic value of the future evolution of the disease.

The invention also provides a method for diagnosing a neoplasia originating from hematopoietic cells and/or making a prognosis of the evolution thereof which comprises the in vitro detection from a biological sample and the statistical analysis of the expression level of at least one significant gene for classifying the sample as associated or not to said neoplasia, a gene which is selected from the group composed of GABARAP, NPM3, ABCB1, ABCB4, ABCC3, ABCC5, ABCC6, ABHD1, ABL1, ACTN1, AF1q, AKR1A1, ALDH1A1, ALK, ANK2, ANPEP, ANXA6, ANXA7, APAF1, APEX, ARHGEF2, ARS2, ASNS, ATIC, ATM, ATP5O, BAX, BCL10, BCL2, BCL2A1, BCL2L1, BCL2LAA, BCL3, BCL6, BCL7A, BCL7b, BCR, BECN1, BIK, BIRC3, BIRC5, BLMH, BLR1, BLVRB, BMI1, BMP6, BRMS1, BST2, BTG1, BUB1, C21orf33, C5orf13, CA12, CALD1, CANP2, CASC3, CASP1, CASP3, CASP4, CASP5, CASP6, CASP7, CASP8, CASP9, CAST, CATSD, CBFA2T1, CBFB, CCNA1, CCNB1, CCND1, CCND2, CCND3, CCNE1, CCR6, CCR7, CCT6A, CD14, CD19, CD2, CD22, CD24, CD28, CD33, CD34, CD36, CD38, CD3E, CD4, CD44, CD47, CD48, CD5, CD58, CD59, CD6, CD7, CD79A, CD79B, CD8, CD81, CD83, CD86, CD9, CDA, CDC25A, CDC25B, CDK2, CDK4, CDK5R1, CDKN1A, CDKN1B, CDKN1C, CDKN2A, CDKN2B, CDKN2C, CDKN3, CDW52, CEBPA, CEBPB, CEBPD, CFL1, CKMT1, CKS2, CML66, COL3A1, COL4A6, CR2, CREB1, CREBBP, CRYAB, CSF2, CSF3, CSRP2, CTGF, CTSB, CUZD1, CXADR, CXCL9, CXCR3, CXCR4, CYC1, CYP1A1, CYP2A6, DAD-1, DAPK1, DCK, DDX6, DEK, DHFR, DLAD, DNAJA1, DNMT3B, DNTT, DOK1, DPF2, DPP4, DRG1, DRP2, E2F1, EB-1, EBI2, EDF1, EEF1A1, EEF1B2, EEF1D, EEF1G, EFNB1, EGFR, EGR1, EIF2B2, EIF3S2, EIF4B, EIF4E, EIF5A, ELF1, ELF4, ENPP1, EphA3, EPOR, ERBB2, ERBB4, ERCC1, ERCC2, ERCC3, ERCC5, ERCC6, ETS1, ETS2, ETV6, ETV7, EZH2, FABP5, FADD, FAIM3, FAM38A, FARP1, FAT, FCER2, FCGR3A, FCGR3B, FGFR1, FGFR3, FGR, FHIT, FKBP9, FLI1, FLJ22169, FLT3, FN1, FNTB, FOS, FUS, G1P2, GABPB2, GATA1, GATA2, GATA3, GCET2, GDI2, GGA3, GJA1, GLUD1, GNL3, GOT1, GRB2, GRIA3, GRK4, GSTP1, GSTT1, GUSB, GZMA, H2AFX, H3F3A, HCK, HELLS, HIF1A, HIST1H2BN, HLA-A, HLA-DPA1, HLA-DQA1, HLA-DRA, HLA-DRB3, HLF, HMMR, HNRPH3, HNRPL, HOXA10, HOXA9, HOXD8, HOXD9, HRAS, HSD17B1, HSPB1, IBSP, ICAM1, ICAM3, ID2, IER3, IFRD1, IGFBP2, IGFBP3, IGFIR, IGLV6-57, IL10, IL15, IL1B, IL2, IL2RA, IL3, IL32, IL3RA, IL4R, IL6, IL6R, IL8, ILF2, IRF1, IRF2, IRF4, IRF8, ITGA2, ITGA3, ITGA4, ITGA5, ITGA6, ITGAL, ITGAM, ITGAX, ITGB1, ITGB2, JAK1, JAK2, JUNB, KAI1, KIAA0247, KIAA0864, KIT, KLF1, KLF13, KRAS2, KRT18, LADH, LAG3, LASP1, LCK, LCP1, LEPR, LGALS3, LGALS7, LIF, LIMS1, LMO2, LOC285148, LRP, LSP1, LYL1, LYN, LYZ, MAFB, MAFK, MAGEA1, MAL, MAP3K12, MAP4K1, MAPK10, MAZ, MBP1, MCL1, MCM3, MCM7, MDM2, MEIS1, MEN1, MERTK, MKI67, MLF1, MLF2, MLL, MLLT10, MME, MMP2, MMP7, MMP8, MMP9, MNDA, MPL, MPO, MRPL37, MS4A1, MTCP1, MUC-1, MX1, MYB, MYBL1, MYC, MYOD1, NCALD, NCAM1, NCL, NDP52, NDRG1, NDUFA1, NDUFB, NF1, NFATC1, NFIC, NFKB1, NFIB1A, NINJ1, NPM1, NR3C1, NUMA1, NXF1, ODC1, OGGI, OLIG2, OPRD1, p14ARF, P55CDC, PABPC1, PAX5, PAX6, PAX8, PBX1, PBX3, PCA1, PCD, PCNA, PDCD1, PDGFA, PDGFRB, PDHA1, PGF, PGRMC1, PICALM, PLA2G6, PLAU, PLK1, PLP, PLS3, PLZF, PML, PMM1, POLR2c, POU2F2, PPP1CC, PRAME, PRKCI, PRKCQ, PRKDC, PRL, PRTN3, PSMA5, PSMB4, PSMC5, PSMD7, PTEN, PTGS1, PTHLH, PTK2, PTK2B, PTN, PTPRCCD, PYGB, RAD51, RAF1, RAG1, RARA, RARB, RB1, RBBP4, RBBP6, RBBP8, RBP4, RET, RGS1, RGS1, RIS1, RORA, RPL17, RPL23A, RPL24, RPL36A, RPL37A, RPL41, RPS3, RPS5, RPS9, RUNX1, RxRA, S100A2, S100A8, SDC1, SDHD, SELE, SELL, SEPW1, SERPINA9, SERPINB5, SERPNINA9, SFTPB, SIAT4A, SLC7A5, SNRPB, SOSTDC1, SP1, SPI1, SPN, SPRRIA, SREBF1, SSBP1, STAT1, STAT3, STAT5B, SUMO1, TACSTD2, TAGLN2, TAL1, TBP, TCEB1, TCF1, TCF3, TCF7, TCL1A, TCRbeta, TEGT, TERF1, TERT, TFCP2, TFRC, THBS1, THPO, TIA-2, TIAM1, TK1, TLX1, TMEM4, TNF, TNFRSF10C, TNFRSF1A, TNFRSF25, TNFRSF5, TNFRSF6, TNFRSF8, TNFSF10, TNFSF5, TNFSF6, TOP2A, TOPORS, TP73, TRA@, TRADD, TRAF3, TRAP1, TRIB2, TXNRD1, UBE2C, UHRF1, UVRAG, VCAM1, VEGF, VPREB1, WBSCR20C, WNT16, WTI, XBP1, XPO6, XRCC3, XRCC5, ZAP70, ZFPL1, ZNF42, ZNFN1A1, ZYX, 18S rRNA, 28S rRNA, and whose expression level is determined by the evaluation of the concentration of its corresponding mRNA by the use of at least one probe which has a sequence complementary to a fragment of a strand of said gene, a probe which is selected from the group of oligonucleotides composed of: SG1, SG2, SG3, SG4, SG5, SG6, SG7, SG8, SG9, SG10, SG11, SG12, SG13, SG14, SG15, SG16, SG17, SG18, SG19, SG20, SG21, SG22, SG23, SG24, SG25, SG26, SG27, SG28, SG29, SG30, SG31, SG32, SG33, SG34, SG35, SG36, SG37, SG38, SG39, SG40, SG41, SG42, SG43, SG44, SG45, SG46, SG47, SG48, SG49, SG50, SG51, SG52, SG53, SG54, SG55, SG56, SG57, SG58, SG59, SG60, SG61, SG62, SG63, SG64, SG65, SG66, SG67, SG68, SG69, SG70, SG71, SG72, SG73, SG74, SG75, SG76, SG77, SG78, SG79, SG80, SG81, SG82, SG83, SG84, SG85, SG86, SG87, SG88, SG89, SG90, SG91, SG92, SG93, SG94, SG95, SG96, SG97, SG98, SG99, SG100, SG101, SG102, SG103, SG104, SG105, SG106, SG107, SG108, SG109, SG110, SG111, SG112, SG113, SG114, SG115, SG116, SG117, SG118, SG119, SG120, SG121, SG122, SG123, SG124, SG125, SG126, SG127, SG128, SG129, SG130, SG131, SG132, SG133, SG134, SG135, SG136, SG137, SG138, SG139, SG140, SG141, SG142, SG143, SG144, SG145, SG146, SG147, SG148, SG149, SG150, SG151, SG152, SG153, SG154, SG155, SG156, SG157, SG158, SG159, SG160, SG161, SG162, SG163, SG164, SG165, SG166, SG167, SG168, SG169, SG170, SG171, SG172, SG173, SG174, SG175, SG176, SG177, SG178, SG179, SG180, SG181, SG182, SG183, SG184, SG185, SG186, SG187, SG188, SG189, SG190, SG191, SG192, SG193, SG194, SG195, SG196, SG197, SG198, SG199, SG200, SG201, SG202, SG203, SG204, SG205, SG206, SG207, SG208, SG209, SG210, SG211, SG212, SG213, SG214, SG215, SG216, SG217, SG218, SG219, SG220, SG221, SG222, SG223, SG224, SG225, SG226, SG227, SG228, SG229, SG230, SG231, SG232, SG233, SG234, SG235, SG236, SG237, SG238, SG239, SG240, SG241, SG242, SG243, SG244, SG245, SG246, SG247, SG248, SG249, SG250, SG251, SG252, SG253, SG254, SG255, SG256, SG257, SG258, SG259, SG260, SG261, SG262, SG263, SG264, SG265, SG266, SG267, SG268, SG269, SG270, SG271, SG272, SG273, SG274, SG275, SG276, SG277, SG278, SG279, SG280, SG281, SG282, SG283, SG284, SG285, SG286, SG287, SG288, SG289, SG290, SG291, SG292, SG293, SG294, SG295, SG296, SG297, SG298, SG299, SG300, SG301, SG302, SG303, SG304, SG305, SG306, SG307, SG308, SG309, SG310, SG311, SG312, SG313, SG314, SG315, SG316, SG317, SG318, SG319, SG320, SG321, SG322, SG323, SG324, SG325, SG326, SG327, SG328, SG329, SG330, SG331, SG332, SG333, SG334, SG335, SG336, SG337, SG338, SG339, SG340, SG341, SG342, SG343, SG344, SG345, SG346, SG347, SG348, SG349, SG350, SG351, SG352, SG353, SG354, SG355, SG356, SG357, SG358, SG359, SG360, SG361, SG362, SG363, SG364, SG365, SG366, SG367, SG368, SG369, SG370, SG371, SG372, SG373, SG374, SG375, SG376, SG377, SG378, SG379, SG380, SG381, SG382, SG383, SG384, SG385, SG386, SG387, SG388, SG389, SG390, SG391, SG392, SG393, SG394, SG395, SG396, SG397, SG398, SG399, SG400, SG401, SG402, SG403, SG404, SG405, SG406, SG407, SG408, SG409, SG410, SG411, SG412, SG413, SG414, SG415, SG416, SG417, SG418, SG419, SG420, SG421, SG422, SG423, SG424, SG425, SG426, SG427, SG428, SG429, SG430, SG431, SG432, SG433, SG434, SG435, SG436, SG437, SG438, SG439, SG440, SG441, SG442, SG443, SG444, SG445, SG446, SG447, SG448, SG449, SG450, SG451, SG452, SG453, SG454, SG455, SG456, SG457, SG458, SG459, SG460, SG461, SG462, SG465, SG468, SG469, SG470, SG471, SG472, SG473, SG474, SG475, SG476, SG477, SG478, SG479, SG480, SG481, SG482, SG483, SG484, SG485, SG486, SG487, SG488, SG489, SG490, SG491, SG492, SG493, SG494, SG495, SG496, SG497, SG498, SG499, SG500, SG501, SG502, SG503, SG504, SG505, SG506, SG507, SG508, SG509, SG510, SG511, SG512, SG513, SG514, SG515, SG516, SG517, SG518, SG519, SG520, SG521, SG522, SG523, SG524, SG525, SG526, SG527, SG428, SG529, SG530, SG531, SG532, SG533, SG534, SG535, SG536, SG537, SG538, SG539, SG540, SG541, SG542, SG543, SG544, SG545, SG546, SG547, SG548, SG549, SG550, SG551, SG552, SG553, SG554, SG555, SG556, SG557, SG558, SG559, SG560, SG561, SG562, SG563.

The genes which form part of the aforementioned group are human genes. Therefore, whenever the words “subject” or “individual” are used hereinafter, they will make reference to a human being.

A particular case of this method is that which comprises an additional previous step of identification of genes significant for the classification of the biological sample analysed as associated or not to a specific type of neoplasia originating from hematopoietic cells, a classification which includes not only the diagnosis of the existence of said neoplasia in the individual from which the sample has been taken, but which may also consist, in additional or alternative form, of the discrimination between specific subtypes of said neoplasia which correspond to different future forms of evolution of said neoplasia this constituting the classification of one or another subtype of the evolution of the neoplasia considered in the future. In this particular case of the method of the invention which comprises a previous step of identification of genes significant for making the desired classification, said previous step comprises the steps of:
a) deciding the possible categories wherein the sample can be classified;
b) obtaining biological samples from individuals which have previously been assigned by a method different to that claimed to any of the possible classification categories, so that there are samples of each one of the possible categories;
c) obtaining the total mRNA of each one of the samples;
d) obtaining the corresponding total cRNA, labelled by a method which allows its subsequent detection, of at least one aliquot of each one of the samples of mRNA, an aliquot whereto is added before the obtainment of the cRNA at least one sequence of polyadenylated nucleotides of low homology with human genes for which it acts as internal positive control of the process;
e) adding to one of the aliquots of cRNA which are going to be used in step f) at least one oligonucleotide of low homology with human genes different from and not complementary to any possible sequence of nucleotides which have been added in step d), for which it acts as positive hybridization control;
f) hybridizing, in strict conditions, at least one aliquot of total cRNA of each one of the samples with at least one microarray which comprises at least two copies of each one of the oligonucleotides from the group composed of:
   SG1, SG2, SG3, SG4, SG5, SG6, SG7, SG8, SG9, SG10, SG11, SG12, SG13, SG14, SG15, SG16, SG17, SG18, SG19, SG20, SG21, SG22, SG23, SG24, SG25, SG26, SG27, SG28, SG29, SG30, SG31, SG32, SG33, SG34, SG35, SG36, SG37, SG38, SG39, SG40, SG41, SG42, SG43, SG44, SG45, SG46, SG47, SG48, SG49, SG50, SG51, SG52, SG53, SG54, SG55, SG56, SG57, SG58, SG59, SG60, SG61, SG62, SG63, SG64, SG65, SG66, SG67, SG68, SG69, SG70, SG71, SG72, SG73, SG74, SG75, SG76, SG77, SG78, SG79, SG80, SG81, SG82, SG83, SG84, SG85, SG86, SG87, SG88, SG89, SG90, SG91, SG92, SG93, SG94, SG95, SG96, SG97, SG98, SG99, SG100, SG101, SG102, SG103, SG104, SG105, SG106, SG107, SG108, SG109, SG110, SG111, SG112, SG113, SG114, SG115, SG116, SG117, SG118, SG119, SG120, SG121, SG122, SG123, SG124, SG125, SG126, SG127, SG128, SG129, SG130, SG131, SG132, SG133, SG134, SG135, SG136, SG137, SG138, SG139, SG140, SG141, SG142, SG143, SG144, SG145, SG146, SG147, SG148, SG149, SG150, SG151, SG152, SG153, SG154, SG155, SG156, SG157, SG158, SG159, SG160, SG161, SG162, SG163, SG164, SG165, SG166, SG167, SG168, SG169, SG170, SG171, SG172, SG173, SG174, SG175, SG176, SG177, SG178, SG179, SG180, SG181, SG182, SG183, SG184, SG185, SG186, SG187, SG188, SG189, SG190, SG191, SG192, SG193, SG194, SG195, SG196, SG197, SG198, SG199, SG200, SG201, SG202, SG203, SG204, SG205, SG206, SG207, SG208, SG209, SG210, SG211, SG212, SG213, SG214, SG215, SG216, SG217, SG218, SG219, SG220, SG221, SG222, SG223, SG224, SG225, SG226, SG227, SG228, SG229, SG230, SG231, SG232, SG233, SG234, SG235, SG236, SG237, SG238, SG239, SG240, SG241, SG242, SG243, SG244, SG245, SG246, SG247, SG248, SG249, SG250, SG251, SG252, SG253, SG254, SG255, SG256, SG257, SG258, SG259, SG260, SG261, SG262, SG263, SG264, SG265, SG266, SG267, SG268, SG269, SG270, SG271, SG272, SG273, SG274, SG275, SG276, SG277, SG278, SG279, SG280, SG281, SG282, SG283, SG284, SG285, SG286, SG287, SG288, SG289, SG290, SG291, SG292, SG293, SG294, SG295, SG296, SG297, SG298, SG299, SG300, SG301, SG302, SG303, SG304, SG305, SG306, SG307, SG308, SG309, SG310, SG311, SG312, SG313, SG314, SG315, SG316, SG317, SG318, SG319, SG320, SG321, SG322, SG323, SG324, SG325, SG326, SG327, SG328, SG329, SG330, SG331, SG332, SG333, SG334, SG335, SG336, SG337, SG338, SG339, SG340, SG341, SG342, SG343, SG344, SG345, SG346, SG347, SG348, SG349, SG350, SG351, SG352, SG353, SG354, SG355, SG356, SG357, SG358, SG359, SG360, SG361, SG362, SG363, SG364, SG365, SG366, SG367, SG368, SG369, SG370, SG371, SG372, SG373, SG374, SG375, SG376, SG377, SG378, SG379, SG380, SG381, SG382, SG383, SG384, SG385, SG386, SG387, SG388, SG389, SG390, SG391, SG392, SG393, SG394, SG395, SG396, SG397, SG398, SG399, SG400, SG401, SG402, SG403, SG404, SG405, SG406, SG407, SG408, SG409, SG410, SG411, SG412, SG413, SG414, SG415, SG416, SG417, SG418, SG419, SG420, SG421, SG422, SG423, SG424, SG425, SG426, SG427, SG428, SG429, SG430, SG431, SG432, SG433, SG434, SG435, SG436, SG437, SG438, SG439, SG440, SG441, SG442, SG443, SG444, SG445, SG446, SG447, SG448, SG449, SG450, SG451, SG452, SG453, SG454, SG455, SG456, SG457, SG458, SG459, SG460, SG461, SG462, SG465, SG468, SG469, SG470, SG471, SG472, SG473, SG474, SG475, SG476, SG477, SG478, SG479, SG480, SG481, SG482, SG483, SG484, SG485, SG486, SG487, SG488, SG489, SG490, SG491, SG492, SG493, SG494, SG495, SG496, SG497, SG498, SG499, SG500, SG501, SG502, SG503, SG504, SG505, SG506, SG507, SG508, SG509, SG510, SG511, SG512, SG513, SG514, SG515, SG516, SG517, SG518, SG519, SG520, SG521, SG522, SG523, SG524, SG525, SG526, SG527, SG428, SG529, SG530, SG531, SG532, SG533, SG534, SG535, SG536, SG537, SG538, SG539, SG540, SG541, SG542, SG543, SG544, SG545, SG546, SG547, SG548, SG549, SG550, SG551, SG552, SG553, SG554, SG555, SG556, SG557, SG558, SG559, SG560, SG561, SG562, SG563,
   a microarray which additionally comprises:
a. at least two points which correspond to different aliquots of the solvent wherein nucleotides are found at the time of their deposit on the surface of the microarray, for which they serve as blank,
b. at least two copies of at least one oligonucleotide for each one of the polyadenylated sequences added in step d), an oligonucleotide whose sequence will correspond to a fragment, different from the polyadenylation zone, of the sequence of polyadenylated nucleotides whose evolution in the process has to be controlled;
c. for each one of the oligonucleotides added in step e), at least two copies of an oligonucleotide complementary thereto;
d. at least two copies of each member of at least one pair of oligonucleotides wherein the sequence of one of the members corresponds to a sequence of zone 5′ and the sequence of the other corresponds to a sequence of zone 3′ of the mRNA of a gene which is expressed in constitutive form in any cell of hematopoietic origin;
e. at least two copies of at least one oligonucleotide of low homology with human genes different from any of the oligonucleotides defined in section b. and different from any of the synthetic oligonucleotides added optionally in step e);
g) detecting and quantifying the signal of cRNA hybridized with each one of the copies of each one of the oligonucleotides present in the microarray, as well as the signal corresponding to the points of the solvent;
h) calculating the average level of intensity of hybridization of each one of the oligonucleotides of the microarray calculating the average of the intensities of the copies of each one of the oligonucleotides;
i) taking the hybridization as valid if the following conditions are complied with:
   a. the ratio between the average intensity and the average background of all the oligonucleotides of the microarray is greater than 10;
   b. the value of the average coefficient of variation of all the replicas of oligonucleotides should be less than 0.3;
   c. the average value of negative control should be less than 2.5 times the average value of the points corresponding to the solvent;
   d. there is a signal both in the hybridization controls and in the internal positive controls used as process control;
j) normalizing the data;
k) eliminating the oligonucleotides with values of average intensity minus average background noise less than approximately 2 times the average value obtained with the points corresponding to the solvent, as well as the oligonucleotides with an interquartile range of normalized intensity throughout the samples less than 0.3;
l) performing the statistical analysis to find the statistically significant oligonucleotides to differentiate between the different categories and be able to classify a sample which has not been previously assigned to any category, choosing said oligonucleotides among those which have not been eliminated in the previous steps, until obtaining “n” oligonucleotides which either have a value of p less than a limit which is chosen from the open range of 0 to 0.05, preferably using for it a method with capacity to reduce false positives, or that which best defines the category established;
m) checking that the grouping of the samples according to the differences in intensities between the different samples detected for the statistically significant oligonucleotides gives rise to the samples being classified in the same categories as those which had previously been assigned by a different method.

It is preferred that the average value calculated in section h) is the trimmed mean, for which reason it is preferable that the microarray comprises at least four copies of each one of the oligonucleotides present therein.

The normalization can be carried out with different methods. There is preference for the use of functions contained in access packages freely accessed over the Internet designed for the processing, calculation and graphic representation of data, such as the packages designed in R programming language, available to download from CRAN (http://cran.r-project.org/) or Bioconductor (http://www.bioconductor.orq). The “variance stabilization normalization” method available in the “vsn” package in R.

The identification of the statistically significant oligonucleotides to differentiate between the different categories can be carried out using different methods, having preference for those wherein a value p is determined that determines the threshold of probability under which all the genes whose expression difference has a value less than p would be considered significant and, among these, those which have the capacity to carry out corrections on the value of p, such as, among others, Bonferroni's method or Welch's test. The value of p will be chosen from the open range of 0 to 0.05, preferring, when possible, a value of p close to 0.001 and with correction, it being possible to increase said value at maximum to 0.05 (value which is not included among those possible) until which statistically significant oligonucleotides are found to differentiate between the categories among which one wants to classify the samples. A possibility for carrying out these calculations is, again, the use of functions contained in packages freely accessed over the Internet designed for the processing, calculation and graphic representation of data. In particular, the mt.maxT function of the multtest package in R can be used for the identification of the statistically significant oligonucleotides.

Another possibility for the identification of statistically significant oligonucleotides to be able to differentiate between the categories of established samples is the use of the “nearest shrunken centroids” method, a variation of the “nearest centroids” method (Tibshirani et al., 2002), which identifies a group of genes which best characterizes a predefined class and uses this group of genes to predict the class which new samples belong to. To do this, again functions contained in packages freely accessed over the internet may be resorted to, such as the “pam^{a}” package in R, wherein it is possible to find functions to carry out the so-called “Prediction Analysis for Microarrays (PAM)”, which makes use of the “nearest shrunken centroids” method.

After identifying the statistically significant genes to distinguish between categories of samples established from the corresponding oligonucleotides, they can be used for classifying new samples due to similarity between the expression profile of those genes in the sample analysed and those corresponding to each one of the classification categories. Alternatively, when there are only 2 possible classification categories (which will be normal when one wants to diagnose the presence or absence of a certain type of leukaemia in an individual), it is possible to obtain a mathematical function of classification of samples which determine the probability “pᵢ” of a sample “i” belonging to one category or another. To do this, a subunit of the samples is chosen which have been previously assigned to each one of the possible categories by a method different to that of the invention and the value of 0 is arbitrarily associated to each one of the samples of one of the categories “a” (typically, the category of “not” associated to the leukemia one wants to diagnose”) of belonging to the other possible category, whilst each one of the samples of the subunit belonging to the other possible category “b” (typically, the category of “associated” to the leukemia one wants to diagnose”) arbitrarily receives the value “1” for its probability of belonging to its own category. With this, logistical regression is used to calculate, with the aid of the probability values assigned to each one of the samples and the values of normalized trimmed mean intensity obtained for each one of the samples with each one of the “n” oligonucleotides which has been identified as a statistically significant oligonucleotide in the previous step, coefficients for each one of said oligonucleotides which make it possible to obtain a function of probability pᵢ of a sample “i” belonging to category “b”, a function which will be of the type

pᵢ=1/(1+e^{-xi})

and which results from the algebraic transformation of the expression which equals Neperian logarithm of the quotient between the probability of an event occurring and the probability that it does not occur at a function xᵢ which includes as variables each one of the factors which may influence the event, i.e.

$$ln \frac{p}{1 - p} = {x}_{i}$$

function xᵢ which, in the present case, will depend on the intensity values obtained for each one of the statistically significant oligonucleotides and which responds to an expression of the type:

$${x}_{i} = constant + {\sum }_{m = 1}^{n} ( {coeff\_olig}_{m} * {lmn}_{i} {\_olig}_{m} )$$

where
coeff_oligₘ represents the coefficient calculated for a specific oligonucleotide “m”
Imnᵢ_{_{—}}oligₘ represents the average value of normalized intensity obtained in the hybridization of the sample i calculated for the oligonucleotide “m”
“m” varies from 1 to “n”
n is the total number of oligonucleotides considered significant.

The function pᵢ obtained after calculating by logistical regression the coefficient corresponding to each oligonucleotide permits classifying a sample “i” as belonging to one or another category, considering that the values of pᵢ over 0.5 (and which will be less than or equal to 0) indicate that the sample belongs to category “b”, whilst the values of pᵢ less than 0.5 indicate that the sample belongs to category “a”. Said function pᵢ will be considered valid if, on being applied to the samples wherefrom it has been deduced, it is capable of classifying them correctly and, furthermore, as it is applied to the subgroup of samples which have not been taken into account to deduce the function, but whose category is known as it has been previously assigned by a method other than that of the invention, it is also capable of classifying them correctly.

Alternatively, when the identification of the statistically significant genes has been performed using the “Prediction Analysis for Microarrays” method, the classifier can be obtained with the corresponding functions of the “pamr^{a}” package in R, which also starts from the assignment of the value of probability 0 to a subgroup of members of one of the categories and the value of probability 1 to a subgroup of the members of the other category. Again, the calculation of coefficients for statistically significant oligonucleotides permits the calculation of values of probability of belonging to one or another category, also considering that the values over 0.5 indicate belonging to the category whose members are arbitrarily assigned value 1 and the values less than 0.5 indicate belonging to the other category.

A particular case of the method of the invention is that wherein one wants to classify samples as associated or not to a type of leukemia. In that case, blood samples are preferred, especially those of peripheral blood, as biological samples to carry out in vitro the method of the invention.

Once the statistically significant genes have been identified to associate a determined type of neoplasia as originating from hematopoietic cells, the method of the invention can be used for classifying samples according to the expression level of said genes in said samples. The neoplasia can be, for example, a specific type of leukemia. A particular case of this embodiment of the method of the invention is constituted by the association of chronic lymphatic leukemia, thus allowing the diagnosis of this disease by the method of the invention. To do this, significant genes are considered to be those genes whose expression level is analysed on applying the method of the invention at least those of the group of CD79A, FAIM3, HLA-DRA, HLA-DRB3, HLA-DQA1 and the analysis is carried out on blood samples. The method can be additionally applied including the analysis of the expression level of at least genes IRF8 and COL3A1. Preferably, the analysis of the expression level of these genes is carried out by evaluating the level of their corresponding mRNA by hybridization of their corresponding cRNA with oligonucleotides SG117, SG428, SG459, SG507, SG508, SG461 and SG493, which are preferred to be associated to a solid support forming part of a microarray. When the evaluation of the hybridized cRNA with each one of those oligonucleotides is carried out thanks to the prior labelling of the cRNA with biotin, the staining of the microarray hybridized with streptavidin conjugated with a fluorophore and the detection of the signal emitted by said fluorophore, it is preferred that the fluorophore is Cy3, which permits diagnosing the presence of CLL in the subject from which the sample has been taken by the classification of sample “i” analysed as associated to CLL from the calculation of the probability that said sample is associated to CLL from the formula pᵢ=1/(1+e^{-x}^{ᵢ}), wherein xᵢ is calculated by the formula

xᵢ=−719.241486+(2.44756372*Imnᵢ_{_{—}}CD79A)+(7.38657611*Imnᵢ_{_{—}}FAIM3)+(23.1465464*Imnᵢ_{_{—}}HLA-DRA)+(43.6287742*Imnᵢ_{_{—}}IRF8)−(19.3978182*Imnᵢ_{_{—}}COL3A1)−(2.80282646*Imnᵢ_{_{—}}HLA-DRB3)+(49.5345672*Imnᵢ_{_{—}}HLA-DQA1)
formula wherein each one of the values denominated with the abbreviation “Imnᵢ” followed by the abbreviation of a gene makes reference to the average value of normalized intensity obtained after detecting the hybridization signal corresponding to the oligonucleotide which is being used as probe to evaluate the expression of the said gene
   and which permits classifying the subject as subject not suffering from CLL if the value of pᵢ is less than 0.5 and as subject suffering from CLL if the value of pᵢ is greater than 0.5.
   Alternatively, significant genes can be considered as those whose expression level is analysed on applying the method of the invention for the diagnosis of CLL at least those of the group of CD79A, FAIM3, HLA-DRA, HLA-DRB3, HLA-DQA1, additionally including the analysis of the expression level of at least gene CDW52. Preferably, the analysis of the expression level of these genes is carried out by evaluating the level of its corresponding mRNA by hybridization of its corresponding cRNA with oligonucleotides SG117, SG428, SG459, SG507, SG508 and SG237, which it is preferred are associated to a solid support forming part of a microarray.

Another particular case of the application of the method of the invention for classifying samples as associated to a specific type of leukemia according to the expression level in said samples of statistically significant genes constitutes the classification of a sample as associated to a specific subtype of chronic lymphatic leukemia, “stable” CLL or “progressive” CLL, which makes it possible that the method of the invention serves to make a prognosis for the future evolution of subjects which have been diagnosed with CLL. When the samples analysed are of peripheral blood, the genes considered statistically significant to perform the classification of the samples are at least genes PSMB4, FCER2 and POU2F2, it being possible to additionally analyse the expression level of at least one gene selecting the group composed of ODC1, CD79A, CD2, CD3E, CD5, MS4A1, EIF4E, FHIT, NR3C1, LCP1, MAPK10, ABCC5, XRCC3, CML66, PLZF, RBP4 or the totality thereof.

An additional aspect of the invention is the use of devices to evaluate the expression level of at least one of the genes of the group composed of PSMB4, FCER2, POU2F2, ODC1, CD79A, CD2, CD3E, CD5, MS4A1, EIF4E, FHIT, NR3C1, LCP1, MAPK10, ABCC5, XRCC3, CML66, PLZF, RBP4, CD79A, FAIM3, HLA-DRA, HLA-DRB3, HLA-DQA1, IRF8 and COL3A1 with the aim of diagnosing the presence of CLL in an individual and/or making a prognosis of his/her evolution. A particular case of this aspect of the invention is the use of devices of evaluation of the expression level of at least one gene of the group composed of CD79A, FAIM3, HLA-DRA, HLA-DRB3, HLA-DQA1, IRF8 and COL3A1 for the diagnosis of the presence of CLL in an individual, wherein it is preferred that the device evaluates at least the expression level of genes CD79A, FAIM3, HLA-DRA, HLA-DRB3, HLA-DQA1, it being possible for the device to evaluate, additionally, the expression level of at least genes IRF8 and COL3A1 or at least gene CDW52. Another particular case of this aspect of the invention is the use of devices of evaluation of the expression level of at least one gene of the group composed of PSMB4, FCER2, POU2F2, ODC1, CD79A, CD2, CD3E, CD5, MS4A1, EIF4E, FHIT, NR3C1, LCP1, MAPK10, ABCC5, XRCC3, CML66, PLZF, RBP4, CD79A, FAIM3, HLA-DRA, HLA-DRB3, HLA-DQA1, IRF8 and COL3A1 to make a prognosis of the future evolution of CLL in an individual.

### DETAILED DESCRIPTION OF THE INVENTION

### Design of the Microarray Device

### Genes Included in the Microarray

A revision was performed of the scientific literature and genes were selected due to their special involvement in the biology of blood cells or in the pathology of the different neoplasias The genes selected can be included within these 4 large groups:

### a) With an Important Role in the Biology of the Hematopoietic Cells:

Genes whose protein is expressed or repressed in the different steps through which these cells pass in their differentiation to mature forms.
Genes whose protein is specifically expressed in accordance with the line whereto the cell belongs.
Genes which code adhesion molecules

### b) Involved in Different Types of Hematological Neoplasias:

Genes whose expression (a level of mRNA or protein) is altered in different types of neoplasias, or associated to resistance to chemotherapy

### c) Cancer-Related:

Genes which code proteins associated with proliferation, metastasis or genes whose expression is increased in a large number of tumours.

### d) Described in Publications Related to Neoplasias:

Genes which, without having a special ratio with hematological neoplasias or blood cell biology, have appeared in the scientific literature as statistically associated to a type of neoplasia

The characteristics of the genes can be consulted, for example, in: www.ncbi.nlm.nih.qov/Genbank, selecting the “Gene” option in the drop-down menu which appears and entering the corresponding identification number (GenID) in the GenBank. The genes whose expression can be analysed with the microarray, their corresponding identification number in the GenBank, as well as the oligonucleotides present in the microarray to be used as probes to analyse the expression of said genes appear below in Table 1.

Probes of Oligonucleotides which Represent Each Gene.

For each one of the 534 genes related to hematological neoplasias, as well as for the genes corresponding to β-actin, glyceraldehyde-3-phosphate dehydrogenase, 18S rRNA and 28S rRNA, the mrRNA sequence is sought in GenBank (www.ncbi.hlm.nih.gov/Genbank/). An oligonucleotide is designed (probe) from the GenBank sequence, specific for each one of the genes selected. In some genes several oligonucleotides were designed situated in zones 5′ and 3′ of the gene, in order to analyse the integrity of the mRNA.

To ensure specificity in the design of the probes, the following criteria were taken into consideration:
Length of the probe to guarantee that all the probes are going to have a similar behaviour,
GC content of the probe between 40 and 60%. This characteristic is also taken into consideration to ensure that all the probes are going to have a similar behaviour.
Localization in the gene. Probes localized at least 3000 nucleotides from 3′ (poly(A)) of the selected mRNA sequence were localized.
Sense of the probe. A strand was chosen with “sense”, i.e. the sequences of the oligonucleotides coincide with sequences of fragments of the corresponding mRNA, instead of being sequences complementary to said fragments. This decision involves that the labelled genetic material has to be antisense (complementary to sense).
Specificity of the probe. To avoid non-specific hybridization, probes were selected which have a percentage of homology, calculated by the BLAST tool (available on the website http://www.ncbi.nlm.nih.gov/), less than 70%.

The data on the oligonucleotides used as probes, the identification number of its corresponding sequence in the attached list, as well as data (identification number in GenBank, usual abbreviation and name) of the genes for the detection of whose expression said oligonucleotides have been designed, are shown below in the Table 1.

TABLE 1
Oligonucleotides used as probes to detect the expression of human genes

| Oligo- | | | Usual | |
|---|---|---|---|---|
| nucleotide | SEQ ID NO: | GenID | abbreviation | Description |
| SG1 | SEQ ID | 11337 | GABARAP | Protein associated to the GABA receptor |
| | NO: 1 | | | |
| SG2 | SEQ ID | 28778 | IGLV6-57 | Variable lambda immunoglobulin 6-57 |
| | NO: 2 | | | |
| SG3 | SEQ ID | 5092 | PCD | 6-pyruvoyl-tetrahydropterine |
| | NO: 3 | | | synthase/dimerization cofactor of the |
| | | | | nuclear factor of 1 alpha hepatocytes |
| | | | | (TCF1) |
| SG4 | SEQ ID | 83988 | NCALD | delta neurocalcin |
| | NO: 4 | | | |
| SG5 | SEQ ID | 58511 | DLAD | deoxyribonuclease II beta |
| | NO: 5 | | | |
| SG6 | SEQ ID | 25928 | SOSTDC1 | which contains a sclerostin 1 domain |
| | NO: 6 | | | |
| SG7 | SEQ ID | 10630 | TIA-2 | glycoprotein associated to the lung cell |
| | NO: 7 | | | membrane, type I |
| SG8 | SEQ ID | 5834 | PYGB | phosphorylase, glycogen; brain |
| | NO: 8 | | | |
| SG9 | SEQ ID | 3987 | LIMS1 | with domains LIM and similar to the antigen |
| | NO: 9 | | | of senescent cells 1 |
| SG10 | SEQ ID | 25 | ABL1 | homologue to the viral oncogene of Abelson |
| | NO: 10 | | | v-abl 1 murine leukemia |
| SG11 | SEQ ID | 4733 | DRG1 | GTP-binding protein regulated by |
| | NO: 11 | | | development 1 |
| SG12 | SEQ ID | 25855 | BRMS1 | Metastasis suppressor of breast cancer 1 |
| | NO: 12 | | | |
| SG13 | SEQ ID | 84696 | ABHD1 | which contains an abhydrolase 1 domain |
| | NO: 13 | | | |
| SG14 | SEQ ID | 3475 | IFRD1 | Development regulator related to interferon 1 |
| | NO: 14 | | | |
| SG15 | SEQ ID | 6173 | RPL36A | Ribosomal protein L36a |
| | NO: 15 | | | |
| SG16 | SEQ ID | 3485 | IGFBP2 | Binding protein to the growth factor similar |
| | NO: 16 | | | to insulin 2, 36 kDa |
| SG17 | SEQ ID | 10397 | NDRG1 | Gene regulated downstream by N-myc 1 |
| | NO: 17 | | | |
| SG18 | SEQ ID | 11328 | FKBP9 | FK506 9-binding protein, 63 kDa |
| | NO: 18 | | | |
| SG19 | SEQ ID | 10241 | NDP52 | protein of the nuclear domain 10 |
| | NO: 19 | | | |
| SG20 | SEQ ID | 2171 | FABP5 | protein which binds to fatty acids 5 |
| | NO: 20 | | | (associated to psoriasis) |
| SG21 | SEQ ID | 10160 | FARP1 | protein with FERM, RhoGEF (ARHGEF) |
| | NO: 21 | | | and pleckstrin 1 domains (derived from |
| | | | | chrondrocytes) |
| SG22 | SEQ ID | 5228 | PGF | Placental growth factor, protein related to |
| | NO: 22 | | | the endothelial growth factor |
| SG23 | SEQ ID | 2665 | GDI2 | GDP 2 dissociation inhibitor |
| | NO: 23 | | | |
| SG24 | SEQ ID | 8407 | TAGLN2 | transgelin 2 |
| | NO: 24 | | | |
| SG25 | SEQ ID | 645 | BLVRB | biliverdin reductase B (flavin reductase |
| | NO: 25 | | | (NADPH)) |
| SG26 | SEQ ID | 5692 | PSMB4 | subunit of proteasome (prosome, |
| | NO: 26 | | | macropain), beta-type, 4 |
| SG27 | SEQ ID | 4070 | TACSTD2 | transducer of the calcium signal associated |
| | NO: 27 | | | to tumours 2 |
| SG28 | SEQ ID | 6921 | TCEB1 | Elongation factor of transcription B (SIII), |
| | NO: 28 | | | polypeptide 1 (15 kDa, elongin C) |
| SG29 | SEQ ID | 1915 | EEF1A1 | elongation factor in the eukaryotic |
| | NO: 29 | | | translation 1 alpha 1 |
| SG30 | SEQ ID | 3020 | H3F3A | histone H3, family 3A |
| | NO: 30 | | | |
| SG31 | SEQ ID | 4953 | ODC1 | ornithine decarboxylase 1 |
| | NO: 31 | | | |
| SG32 | SEQ ID | 7520 | XRCC5 | Of repair of X rays which complement the |
| | NO: 32 | | | defective repair in Chinese hamster cells 5 |
| | | | | (reconnection of breakages in the double |
| | | | | helix; autoantigen Ku, 80 kDa) |
| SG33 | SEQ ID | 3486 | IGFBP3 | binding protein to growth factor similar to |
| | NO: 33 | | | insulin 3 |
| SG34 | SEQ ID | 4691 | NCL | nucleolin |
| | NO: 34 | | | |
| SG35 | SEQ ID | 6273 | S100A2 | calcium S100 A2-binding protein |
| | NO: 35 | | | |
| SG36 | SEQ ID | 6152 | RPL24 | ribosomal protein L24 |
| | NO: 36 | | | |
| SG37 | SEQ ID | 2697 | GJA1 | Bone filling protein, alpha 1, 43 kDa |
| | NO: 37 | | | (connexin 43) |
| SG38 | SEQ ID | 2990 | GUSB | glucuronidase, beta |
| | NO: 38 | | | |
| SG39 | SEQ ID | 3292 | HSD17B1 | hydroxysteroid (17-beta) dehydrogenase 1 |
| | NO: 39 | | | |
| SG40 | SEQ ID | 6439 | SFTPB | surfactant protein, associated to lung B |
| | NO: 40 | | | |
| SG41 | SEQ ID | 6147 | RPL23A | ribosomal protein L23a |
| | NO: 41 | | | |
| SG42 | SEQ ID | 1466 | CSRP2 | protein rich in cysteine and glycine 2 |
| | NO: 42 | | | |
| SG43 | SEQ ID | 1525 | CXADR | receptor of the coxsackie virus and |
| | NO: 43 | | | adenovirus |
| SG44 | SEQ ID | 1937 | EEF1G | elongation factor of eukartyotic 1 gamma |
| | NO: 44 | | | elongation |
| SG45 | SEQ ID | 1164 | CKS2 | subunit regulating the kinase CDC28 2 |
| | NO: 45 | | | protein |
| SG46 | SEQ ID | 1288 | COL4A6 | collagen, type IV, alpha 6 |
| | NO: 46 | | | |
| SG47 | SEQ ID | 1410 | CRYAB | crystalline, alpha B |
| | NO: 47 | | | |
| SG48 | SEQ ID | 1537 | CYC1 | cytochrome c-1 |
| | NO: 48 | | | |
| SG49 | SEQ ID | 2342 | FNTB | farnesyltransferase, CAAX box, beta |
| | NO: 49 | | | |
| SG50 | SEQ ID | 2805 | GOT1 | glutamic-oxaloacetic transaminase 1, |
| | NO: 50 | | | soluble (aminotransferase 1 aspartate) |
| SG51 | SEQ ID | 3963 | LGALS7 | Lectin, which binds to galactosides, soluble, |
| | NO: 51 | | | 7 (galectin 7) |
| SG52 | SEQ ID | 5268 | SERPINB5 | Serine (or cysteine) inhibitor proteinase, |
| | NO: 52 | | | clade B (ovalbumin, member 5 |
| SG53 | SEQ ID | 5705 | PSMC5 | Subunit of proteasome (prosome, |
| | NO: 53 | | | macropain) 26S, ATPase, 5 |
| SG54 | SEQ ID | 5764 | PTN | pleiotrophin (growth factor of bonding to |
| | NO: 54 | | | heparin 8, growth promoter factor of |
| | | | | neurites 1) |
| SG55 | SEQ ID | 5932 | RBBP8 | Retinoblastoma 8-binding protein |
| | NO: 55 | | | |
| SG56 | SEQ ID | 5996 | RGS1 | Regulator of the signalling by proteins G 1 |
| | NO: 56 | | | |
| SG57 | SEQ ID | 6256 | RXRA | Retinoid X receptor, alpha |
| | NO: 57 | | | |
| SG58 | SEQ ID | 6392 | SDHD | succinate dehydrogenase complex, subunit |
| | NO: 58 | | | D, integral membrane protein |
| SG59 | SEQ ID | 6415 | SEPW1 | selenoprotein W, 1 |
| | NO: 59 | | | |
| SG60 | SEQ ID | 6742 | SSBP1 | binding protein to single-strand DNA 1 |
| | NO: 60 | | | |
| SG61 | SEQ ID | 7009 | TEGT | transcript of gene increased in the testicle |
| | NO: 61 | | | (BAX 1 inhibitor) |
| SG62 | SEQ ID | 8341 | HIST1H2BN | histone 1, H2bn |
| | NO: 62 | | | |
| SG63 | SEQ ID | 8678 | BECN1 | beclin 1 (protein similar to myosin which |
| | NO: 63 | | | interacts with BCL2, of twisted helix) |
| SG64 | SEQ ID | 310 | ANXA7 | annexin A7 |
| | NO: 64 | | | |
| SG65 | SEQ ID | 4694 | NDUFA1 | Alpha subcomplex of NADH |
| | NO: 65 | | | dehydrogenase (ubiquinone) 1, 1, |
| SG66 | SEQ ID | 9181 | ARHGEF2 | guanine rho/rac exchange factor (GEF) 2 |
| | NO: 66 | | | |
| SG67 | SEQ ID | 9315 | C5orfl3 | Open reading frame 13 of chromosome 5 |
| | NO: 67 | | | |
| SG68 | SEQ ID | 7494 | XBP1 | X 1 box-binding protein |
| | NO: 68 | | | |
| SG69 | SEQ ID | 9636 | G1P2 | protein inducible by the alpha interferon |
| | NO: 69 | | | (clone IFI-15K) |
| SG70 | SEQ ID | 2746 | GLUD1 | glutamate dehydrogenase 1 |
| | NO: 70 | | | |
| SG71 | SEQ ID | 6273 | S100A2 | calcium S100 A2-binding protein |
| | NO: 71 | | | |
| SG72 | SEQ ID | 3927 | LASP1 | LIM and SH3 1 protein |
| | NO: 72 | | | |
| SG73 | SEQ ID | 10630 | TIA-2 | glycoprotein associated to the lung cell |
| | NO: 73 | | | membrane type I |
| SG74 | SEQ ID | 10857 | PGRMC1 | Membrane component of the progesterone |
| | NO: 74 | | | 1 receptor |
| SG75 | SEQ ID | 7542 | ZFPL1 | protein similar to that of zinc finger 1 |
| | NO: 75 | | | |
| SG76 | SEQ ID | 11184 | MAP4K1 | Kinase protein activated by mitogens 1 |
| | NO: 76 | | | |
| SG77 | SEQ ID | 6772 | STAT1 | Signal transducer and transcription activator |
| | NO: 77 | | | 1, 91 kDa |
| SG78 | SEQ ID | 3189 | HNRPH3 | Heterogeneous nuclear ribonucleoprotein |
| | NO: 78 | | | H3 (2H9) |
| SG79 | SEQ ID | 10330 | TMEM4 | Transmembrane protein 4 |
| | NO: 79 | | | |
| SG80 | SEQ ID | 9766 | KIAA0247 | KIAA0247 |
| | NO: 80 | | | |
| SG81 | SEQ ID | 25907 | RIS1 | of senescence induced by Ras 1 |
| | NO: 81 | | | |
| SG82 | SEQ ID | 51593 | ARS2 | Arsenate-resistant protein ARS2 |
| | NO: 82 | | | |
| SG83 | SEQ ID | 771 | CA12 | Carbonic anhydrase XII |
| | NO: 83 | | | |
| SG84 | SEQ ID | 1933 | EEF1B2 | elongation factor of the eukaryotic 1 beta 2 |
| | NO: 84 | | | translation |
| SG85 | SEQ ID | 28951 | TRIB2 | homologue to tribbles 2 (Drosophila) |
| | NO: 85 | | | |
| SG86 | SEQ ID | 79065 | FLJ22169 | similar to that of autophagy 9 APG9 1 (S. cerevisiae) |
| | NO: 86 | | | |
| SG87 | SEQ ID | 440 | ASNS | asparagine synthetase |
| | NO: 87 | | | |
| SG88 | SEQ ID | 260294 | WBSCR20C | Chromosome region 20C of Williams |
| | NO: 88 | | | Beuren syndrome |
| SG89 | SEQ ID | 10327 | AKR1A1 | member A1 of the aldo-keto reductase 1 |
| | NO: 89 | | | family (aldehyde reductase) |
| SG90 | SEQ ID | 6698 | SPRR1A | Small proline 1A-rich protein |
| | NO: 90 | | | |
| SG91 | SEQ ID | 1947 | EFNB1 | ephrin-B1 |
| | NO: 91 | | | |
| SG92 | SEQ ID | 6193 | RPS5 | ribosomal protein S5 |
| | NO: 92 | | | |
| SG93 | SEQ ID | 6203 | RPS9 | ribosomal protein S9 |
| | NO: 93 | | | |
| SG94 | SEQ ID | 6139 | RPL17 | ribosomal protein L17 |
| | NO: 94 | | | |
| SG95 | SEQ ID | 2114 | ETS2 | homologue to oncogene E26 of the |
| | NO: 95 | | | erythroblastosis virus v-ets 2 (avian) |
| SG96 | SEQ ID | 1975 | EIF4B | initiation factor of the eukaryotic translation |
| | NO: 96 | | | 4B |
| SG97 | SEQ ID | 7791 | ZYX | Zyxin |
| | NO: 97 | | | |
| SG98 | SEQ ID | 23214 | XPO6 | exportin 6 |
| | NO: 98 | | | |
| SG99 | SEQ ID | 285148 | LOC285148 | Hypothetical protein LOC285148 |
| | NO: 99 | | | |
| SG100 | SEQ ID | 8209 | C21orf33 | open reading frame 33 of chromosome 21 |
| | NO: 100 | | | |
| SG101 | SEQ ID | 1936 | EEF1D | elongation factor of the eukaryotic |
| | NO: 101 | | | translation 1 delta (guanine nucleotide |
| | | | | exchange protein) |
| SG102 | SEQ ID | 26986 | PABPC1 | poly(A)-binding protein, cytoplasmic 1 |
| | NO: 102 | | | |
| SG103 | SEQ ID | 5930 | RBBP6 | retinoblastoma 6-binding protein |
| | NO: 103 | | | |
| SG104 | SEQ ID | 3265 | HRAS | homologue to the viral oncogene of the |
| | NO: 104 | | | Harvey v-Ha-ras rat sarcoma |
| SG105 | SEQ ID | 23163 | GGA3 | ARF-binding protein, which contains |
| | NO: 105 | | | gamma-adaptin, associated to golgi |
| SG106 | SEQ ID | 1072 | CFL1 | cophilin 1 (non-muscular) |
| | NO: 106 | | | |
| SG107 | SEQ ID | 8668 | EIF3S2 | initiation factor of the eukaryotic translation |
| | NO: 107 | | | 3, subunit 2 beta, 36 kDa |
| SG108 | SEQ ID | 3875 | KRT18 | keratine 18 |
| | NO: 108 | | | |
| SG109 | SEQ ID | 3480 | IGFIR | growth factor receptor similar to insulin 1 |
| | NO: 109 | | | |
| SG110 | SEQ ID | 3576 | IL8 | Interleukin 8 |
| | NO: 110 | | | |
| SG111 | SEQ ID | 3659 | IRF1 | Interferon regulating factor 1 |
| | NO: 111 | | | |
| SG112 | SEQ ID | 3660 | IRF2 | Interferon regulating factor 2 |
| | NO: 112 | | | |
| SG113 | SEQ ID | 4067 | LYN | Homologue to the oncogene related to the |
| | NO: 113 | | | viral sarcoma of Yamaguchi V-yes-1 |
| SG114 | SEQ ID | 4069 | LYZ | Lysozyme (renal amiloidosis) |
| | NO: 114 | | | |
| SG115 | SEQ ID | 4792 | NFKB1A | Nuclear factor of the gene enhancer of the |
| | NO: 115 | | | kappa light polypeptide in B L-lymphocytes |
| | | | | (p105) |
| SG116 | SEQ ID | 4150 | MAZ | Zinc finger protein associated to MYC |
| | NO: 116 | | | (transcription factor of binding to purins) |
| SG117 | SEQ ID | 973 | CD79A | Antigen CD79A (associated to alpha |
| | NO: 117 | | | immunoglobulins) |
| SG118 | SEQ ID | 4172 | MCM3 | Deficient maintenance of minichromosomes |
| | NO: 118 | | | (S. cerevisiae) 3 |
| SG119 | SEQ ID | 421 | MEIS1 | Homologue to Meis1 (mouse) |
| | NO: 119 | | | |
| SG120 | SEQ ID | 5657 | PRTN3 | Proteinase 3 (serine proteinase, |
| | NO: 120 | | | autoantigen of Wegener's granulomatosis |
| | | | | of neutrophils) |
| SG121 | SEQ ID | 4313 | MMP2 | Metalloproteinase of matrix 2 (gelatinase A, |
| | NO: 121 | | | 72 kD gelatinase of, 72 kD collagenase type |
| | | | | IV) |
| SG122 | SEQ ID | 4316 | MMP7 | Metalloproteinase of matrix 7 (matrilysin, |
| | NO: 122 | | | uterine) |
| SG123 | SEQ ID | 4317 | MMP8 | Metalloproteinase of matrix 8 (neutrophil |
| | NO: 123 | | | collagenase) |
| SG124 | SEQ ID | 1796 | DOK1 | Adapter protein 1, 62 kD (downstream |
| | NO: 124 | | | respect to thyrosine kinase 1) |
| SG125 | SEQ ID | 5154 | PDGFA | Alpha polypeptide of the platelet-derived |
| | NO: 125 | | | growth factor |
| SG126 | SEQ ID | 5617 | PRL | Prolactin |
| | NO: 126 | | | |
| SG127 | SEQ ID | 5894 | RAF1 | Homologue to the viral oncogene of murine |
| | NO: 127 | | | leukemia V-raf-1 1 |
| SG128 | SEQ ID | 5915 | RARB | Retinoic acid receptor, beta |
| | NO: 128 | | | |
| SG129 | SEQ ID | 4985 | OPRD1 | Opioid receptor, delta 1 |
| | NO: 129 | | | |
| SG130 | SEQ ID | 5979 | RET | proto-oncogene ret (multiple endocrine |
| | NO: 130 | | | neoplasia and medullary thyroid carcinoma |
| | | | | 1, Hirschsprung's disease) |
| SG131 | SEQ ID | 6720 | SREBF1 | Transcription factor of binding to sterol |
| | NO: 131 | | | regulatory elements 1 |
| SG132 | SEQ ID | 7124 | TNF | Tumour necrosing factor (TNF superfamily, |
| | NO: 132 | | | member 2) |
| SG133 | SEQ ID | 7013 | TERF1 | Binding factor to telomeric repetitions |
| | NO: 133 | | | (which interact with NIMA) 1 |
| SG134 | SEQ ID | 7412 | VCAM1 | Molecule of adhesion to vascular cells 1 |
| | NO: 134 | | | |
| SG135 | SEQ ID | 539 | ATP5O | ATP synthase, H+ carrier, mitochondrial F1 |
| | NO: 135 | | | complex, subunit O (protein which gives |
| | | | | sensitivity to oligomycin) |
| SG136 | SEQ ID | 959 | TNFSF5 | Tumour necrosing factor (ligand) |
| | NO: 136 | | | superfamily, member 5 (hyper-IgM |
| | | | | syndrome) |
| SG137 | SEQ ID | 5432 | POLR2C | Polypeptide C (directed at DNA) of the RNA |
| | NO: 137 | | | polymerase II (33 kD) |
| SG138 | SEQ ID | 8398 | PLA2G6 | Phospholipase A2, group VI (cytosolic, |
| | NO: 138 | | | calcium-dependent) |
| SG139 | SEQ ID | 908 | CCT6A | TCP1 which contains chaperonin, subunit |
| | NO: 139 | | | 6A (zeta 1) |
| SG140 | SEQ ID | 5160 | PDHA1 | Pyruvate dehydrogenase (lipoamide) alpha 1 |
| | NO: 140 | | | |
| SG141 | SEQ ID | 3939 | LADH | Lactate dehydrogenase A |
| | NO: 141 | | | |
| SG142 | SEQ ID | 6628 | SNRPB | Polypeptides B and B1 of the small nuclear |
| | NO: 142 | | | ribonucleoprotein |
| SG143 | SEQ ID | 6628 | SNRPB | Polypeptides B and B1 of the small nuclear |
| | NO: 143 | | | ribonucleoprotein |
| SG144 | SEQ ID | 3014 | H2AFX | Histone family H2A, member X |
| | NO: 144 | | | |
| SG145 | SEQ ID | 51253 | MRPL37 | Mitochondrial ribosomal protein L37 |
| | NO: 145 | | | |
| SG146 | SEQ ID | 11065 | UBE2C | Enzyme which conjugates with ubiquin E2C |
| | NO: 146 | | | |
| SG147 | SEQ ID | 6188 | RPS3 | Ribosomal protein S3A |
| | NO: 147 | | | |
| SG148 | SEQ ID | 216 | ALDH1A1 | Aldehyde dehydrogenase 1 family, member |
| | NO: 148 | | | A1 |
| SG149 | SEQ ID | 10962 | AF1q | Lymphoid/myeloid or leukemia or of mixed |
| | NO: 149 | | | line (homologue to trithorax, Drosophila); |
| | | | | translocated to 11 |
| SG150 | SEQ ID | 861 | RUNX1 | Runt 1-related transcription (acute myeloid |
| | NO: 150 | | | leukemia 1; oncogene aml1) |
| SG151 | SEQ ID | 4603 | MYBL1 | Similar to the homologue of the viral |
| | NO: 151 | | | oncogene of avian myeloblastisis V-myb 1 |
| SG152 | SEQ ID | 309 | ANXA6 | Annexin A6 |
| | NO: 152 | | | |
| SG153 | SEQ ID | 238 | ALK | Anaplastic lymphoma kinase (Ki-1) |
| | NO: 153 | | | |
| SG154 | SEQ ID | 4332 | MNDA | Antigen for nuclear differentiation myeloid |
| | NO: 154 | | | cells |
| SG155 | SEQ ID | 317 | APAF1 | Apoptotic protease activator factor |
| | NO: 155 | | | |
| SG156 | SEQ ID | 330 | BIRC3 | which contains IAP repetitions of |
| | NO: 156 | | | baculovirus 3 |
| SG157 | SEQ ID | 368 | ABCC6 | ATP-binding module, subfamily C |
| | NO: 157 | | | (CFTR/MRP), member 6 |
| SG158 | SEQ ID | 471 | ATIC | 5-aminoimidazol-4-carboxamide |
| | NO: 158 | | | ribonucleotide formyltransferase/IMP |
| | | | | cyclohydrolase |
| SG159 | SEQ ID | 472 | ATM | Mutated ataxia-telangiectasia (includes |
| | NO: 159 | | | complementary groups A, C and D) |
| SG160 | SEQ ID | 581 | BAX | protein X associated to BCL2 |
| | NO: 160 | | | |
| SG161 | SEQ ID | 595 | CCND1 | Cyclin D1 (PRAD1: parathyroidal |
| | NO: 161 | | | adenomatosis 1) |
| SG162 | SEQ ID | 596 | BCL2 | CLL/lymphoma of B 2 lymphocytes |
| | NO: 162 | | | |
| SG163 | SEQ ID | 602 | BCL3 | CLL/lymphoma of B 3 lymphocytes |
| | NO: 163 | | | |
| SG164 | SEQ ID | 604 | BCL6 | CLL/lymphoma of B 6 lymphocytes (protein |
| | NO: 164 | | | with zinc fingers 51) |
| SG165 | SEQ ID | 605 | BCL7A | CLL/lymphoma of B 7A lymphocytes |
| | NO: 165 | | | |
| SG166 | SEQ ID | 9275 | BCL7b | CLL/lymphoma of B 7B lymphocytes |
| | NO: 166 | | | |
| SG167 | SEQ ID | 8915 | BCL10 | CLL/lymphoma of B 10 lymphocytes |
| | NO: 167 | | | |
| SG168 | SEQ ID | 598 | BCL2L1 | similar to BCL2 1 |
| | NO: 168 | | | |
| SG169 | SEQ ID | 613 | BCR | Grouping breaking point region |
| | NO: 169 | | | |
| SG170 | SEQ ID | 613 | BCR | Grouping breaking point region |
| | NO: 170 | | | |
| SG171 | SEQ ID | 10215 | OLIG2 | Transcription factor of oligodendrocytes 2 |
| | NO: 171 | | | line |
| SG172 | SEQ ID | 638 | BIK | Mortal factor which interacts with BCL2 |
| | NO: 172 | | | (apoptosis inducer) |
| SG173 | SEQ ID | 10018 | BCL2LAA | Similar to BCL2 (which facilitates |
| | NO: 173 | | | apoptosis) |
| SG174 | SEQ ID | 648 | BMI1 | Homologue to the viral oncogene of murine |
| | NO: 174 | | | leukemia (bmi-1) |
| SG175 | SEQ ID | 642 | BLMH | bleomycin hydrolase |
| | NO: 175 | | | |
| SG176 | SEQ ID | 643 | BLR1 | Burkitt 1 lymphoma receptor, GTP-binding |
| | NO: 176 | | | protein |
| SG177 | SEQ ID | 3381 | IBSP | Integrin-binding sialoprotein (bone |
| | NO: 177 | | | sialoprotein bone sialoprotein II) |
| SG178 | SEQ ID | 694 | BTG1 | Gene of translocation of B lymphocytes, |
| | NO: 178 | | | anti-proliferative |
| SG179 | SEQ ID | 699 | BUB1 | Disinhibited budding by benzimidazoles 1 |
| | NO: 179 | | | (homologue of yeasts) |
| SG180 | SEQ ID | 25 | ABL1 | homologue to the viral oncogene of that of |
| | NO: 180 | | | Abelson's murine leukemia v-abl 1 |
| SG181 | SEQ ID | 834 | CASP1 | caspase 1, apoptosis-related cysteine |
| | NO: 181 | | | protease (interleukin 1, beta, convertase) |
| SG182 | SEQ ID | 836 | CASP3 | caspase 3, apoptosis-related cysteine |
| | NO: 182 | | | protease |
| SG183 | SEQ ID | 837 | CASP4 | caspase 4, apoptosis-related cysteine |
| | NO: 183 | | | protease |
| SG184 | SEQ ID | 838 | CASP5 | caspase 5, apoptosis-related cysteine |
| | NO: 184 | | | protease |
| SG185 | SEQ ID | 839 | CASP6 | caspase 6, apoptosis-related cysteine |
| | NO: 185 | | | protease |
| SG186 | SEQ ID | 840 | CASP7 | caspase 7, apoptosis-related cysteine |
| | NO: 186 | | | protease |
| SG187 | SEQ ID | 841 | CASP8 | caspase 8, apoptosis-related cysteine |
| | NO: 187 | | | protease |
| SG188 | SEQ ID | 842 | CASP9 | caspase 9, apoptosis-related cysteine |
| | NO: 188 | | | protease |
| SG189 | SEQ ID | 865 | CBFB | Nucleus binding factor, beta subunit |
| | NO: 189 | | | |
| SG190 | SEQ ID | 800 | CALD1 | Caldesmon 1 |
| | NO: 190 | | | |
| SG191 | SEQ ID | 831 | CAST | Calpastatin |
| | NO: 191 | | | |
| SG192 | SEQ ID | 993 | CDC25A | Cell division cycle 25A |
| | NO: 192 | | | |
| SG193 | SEQ ID | 994 | CDC25B | Cell division cycle 25B |
| | NO: 193 | | | |
| SG194 | SEQ ID | 914 | CD2 | Antigen CD2 (p50), sheep red blood cell |
| | NO: 194 | | | receptor |
| SG195 | SEQ ID | 916 | CD3E | Antigen CD3E, epilson polypeptide |
| | NO: 195 | | | (complex TiT3) |
| SG196 | SEQ ID | 920 | CD4 | Antigen CD4 (p55) |
| | NO: 196 | | | |
| SG197 | SEQ ID | 921 | CD5 | Antigen CD5 (p56-62) |
| | NO: 197 | | | |
| SG198 | SEQ ID | 923 | CD6 | Antigen CD6 |
| | NO: 198 | | | |
| SG199 | SEQ ID | 924 | CD7 | Antigen CD7 (p41) |
| | NO: 199 | | | |
| SG200 | SEQ ID | 925 | CD8 | Antigen CD8, alpha polypeptide(p32) |
| | NO: 200 | | | |
| SG201 | SEQ ID | 928 | CD9 | Antigen CD9 (p24) |
| | NO: 201 | | | |
| SG202 | SEQ ID | 4311 | MME | Membrane metalloendopeptidase (neutral |
| | NO: 202 | | | endopeptidase, encephalinase, CALLA, |
| | | | | CD10) |
| SG203 | SEQ ID | 3683 | ITGAL | Integrin, alpha L (antigen CD11A (p180), |
| | NO: 203 | | | antigen associated to the function of |
| | | | | lymphocytes 1; alpha polypeptide) |
| SG204 | SEQ ID | 3684 | ITGAM | Integrin, alpha M (complement 3 |
| | NO: 204 | | | component receptor, alpha; also known as |
| | | | | CD11b (p170), polypeptide of the |
| | | | | macrophage alpha antigen) |
| SG205 | SEQ ID | 3687 | ITGAX | Integrin, alpha X (antigen CD11C (p150), |
| | NO: 205 | | | alpha polypeptide) |
| SG206 | SEQ ID | 90 | ANPEP | Alanyl-(membrane)aminopeptidase |
| | NO: 206 | | | (aminopeptidase N, aminopeptidase M, |
| | | | | microsomal aminopeptidase, CD13, p150) |
| SG207 | SEQ ID | 929 | CD14 | Antigen CD14 |
| | NO: 207 | | | |
| SG208 | SEQ ID | 6401 | SELE | Selectin E (endothelial adhesion molecule |
| | NO: 208 | | | 1) |
| SG209 | SEQ ID | 2214 | FCGR3A | Low-affinity receptor IIIa for the Fc fragment |
| | NO: 209 | | | of IgG (CD16) |
| SG210 | SEQ ID | 2215 | FCGR3B | Low-affinity receptor IIIb for the Fc fragment |
| | NO: 210 | | | of IgG (CD16) |
| SG211 | SEQ ID | 3689 | ITGB2 | Integrin, beta 2 (antigen CD18 (p95), |
| | NO: 211 | | | antigen associated to the function of the |
| | | | | lymphocytes 1; beta subunit of the |
| | | | | microphage 1 (mac-1) antigen) |
| SG212 | SEQ ID | 930 | CD19 | Antigen CD19 |
| | NO: 212 | | | |
| SG213 | SEQ ID | 931 | MS4A1 | Of 4 domains which are expanded by the |
| | NO: 213 | | | membrane, subfamily A, member 1 |
| SG214 | SEQ ID | 1380 | CR2 | Complement component receptor |
| | NO: 214 | | | (3d/Epstein Barr's virus) 2 |
| SG215 | SEQ ID | 933 | CD22 | Antigen CD22 |
| | NO: 215 | | | |
| SG216 | SEQ ID | 2208 | FCER2 | Low-affinity receptor II for the Fc fragment |
| | NO: 216 | | | of IgE (CD23A) |
| SG217 | SEQ ID | 934 | CD24 | Antigen CD24 (antigen of carcinoma of |
| | NO: 217 | | | small lung cells of the grouping 4) |
| SG218 | SEQ ID | 3559 | IL2RA | interleukin 2 receptor, alpha |
| | NO: 218 | | | |
| SG219 | SEQ ID | 1803 | DPP4 | Dipeptidyl peptidase IV (CD26, protein |
| | NO: 219 | | | which forms complexes with adenosine |
| | | | | deaminase 2) |
| SG220 | SEQ ID | 3688 | ITGB1 | Integrin, beta 1 (fibronectin receptor, beta |
| | NO: 220 | | | polypeptide, antigen CD29 includes MDF2, |
| | | | | MSK12) |
| SG221 | SEQ ID | 943 | TNFRSF8 | Tumour necrosing factor receptor |
| | NO: 221 | | | superfamily, member 8 |
| SG222 | SEQ ID | 945 | CD33 | Antigen CD33 (gp67) |
| | NO: 222 | | | |
| SG223 | SEQ ID | 947 | CD34 | Antigen CD34 |
| | NO: 223 | | | |
| SG224 | SEQ ID | 948 | CD36 | Antigen CD36 (collagen type I receptor, |
| | NO: 224 | | | thrombospondin receptor) |
| SG225 | SEQ ID | 952 | CD38 | Antigen CD38 (p45) |
| | NO: 225 | | | |
| SG226 | SEQ ID | 958 | TNFRSF5 | Tumour necrosing factor receptor |
| | NO: 226 | | | superfamily, member 5 |
| SG227 | SEQ ID | 6693 | SPN | Sarcospan (Gene associated to the Kras |
| | NO: 227 | | | oncogene) |
| SG228 | SEQ ID | 960 | CD44 | Antigen CD44 (homing function and Indian |
| | NO: 228 | | | blood group function) |
| SG229 | SEQ ID | 960 | CD44v6 | Antigen CD44 (homing function and Indian |
| | NO: 229 | | | blood group function) |
| SG230 | SEQ ID | 5788 | PTPRCCD | Protein thyrosine phosphatase, receptor |
| | NO: 230 | | | type, C |
| SG231 | SEQ ID | 961 | CD47 | Antigen CD47 (Antigen related to Rh, |
| | NO: 231 | | | transducer of the signal associated to |
| | | | | integrins) |
| SG232 | SEQ ID | 3673 | ITGA2 | Integrin, alpha 2 (CD49B, alpha 2 subunit of |
| | NO: 232 | | | receptor VLA-2) |
| SG233 | SEQ ID | 3675 | ITGA3 | Integrin, alpha 3 (Antigen CD49C, alpha |
| | NO: 233 | | | subunit 3 of receptor VLA-3) |
| SG234 | SEQ ID | 3676 | ITGA4 | Integrin, alpha 4 (Antigen CD49D, alpha |
| | NO: 234 | | | subunit 4 of receptor VLA-4) |
| SG235 | SEQ ID | 3678 | ITGA5 | Integrin, alpha 5 (fibronectin receptor, alpha |
| | NO: 235 | | | polypeptide) |
| SG236 | SEQ ID | 3385 | ICAM3 | Intercellular adhesion molecule 3 |
| | NO: 236 | | | |
| SG237 | SEQ ID | 1043 | CDW52 | Antigen CDW52 (antigen CAMPATH-1) |
| | NO: 237 | | | |
| SG238 | SEQ ID | 3383 | ICAM1 | Intercellular adhesion molecule 1 (CD54), |
| | NO: 238 | | | human rhinovirus receptor |
| SG239 | SEQ ID | 4684 | NCAM1 | Neural cell adhesion molecule 1 |
| | NO: 239 | | | |
| SG240 | SEQ ID | 965 | CD58 | Antigen CD58 (antigen associated to the |
| | NO: 240 | | | function of the lymphocytes 3) |
| SG241 | SEQ ID | 966 | CD59 | Antigen CD59 p18-20 (antigen identified by |
| | NO: 241 | | | the monoclonal antibodies 16.3A5, EJ16, |
| | | | | EJ30, EL32 and G344) |
| SG242 | SEQ ID | 6402 | SELL | Selectin L (lymphocyte adhesion molecule |
| | NO: 242 | | | 1) |
| SG243 | SEQ ID | 974 | CD79B | Antigen CD79B (associated to beta |
| | NO: 243 | | | immunoglobulins) |
| SG244 | SEQ ID | 975 | CD81 | Antigen CD81 (target of the antiproliferative |
| | NO: 244 | | | antibody 1) |
| SG245 | SEQ ID | 3732 | KAI1 | Kangai 1 (suppression of tumorigenicity 6, |
| | NO: 245 | | | prostate; antigen CD82 (leukocytes R2 |
| | | | | antigen, antigen detected by the |
| | | | | monoclonal antibody IA4)) |
| SG246 | SEQ ID | 9308 | CD83 | Antigen CD83 (activated B lymphocytes, |
| | NO: 246 | | | immunoglobulins superfamily) |
| SG247 | SEQ ID | 942 | CD86 | Antigen CD86 (ligand 2 of the antigen |
| | NO: 247 | | | CD28, antigen B7-2) |
| SG248 | SEQ ID | 355 | TNFRSF6 | Tumour necrosing factor receptor |
| | NO: 248 | | | superfamily, member 6 |
| SG249 | SEQ ID | 356 | TNFSF6 | Tumour necrosing factor receptor |
| | NO: 249 | | | superfamily (ligand), member 6 |
| SG250 | SEQ ID | 8140 | SLC7A5 | Solute-carrier family 7 (cationic amino acid |
| | NO: 250 | | | carrier, y+ system), member 5 |
| SG251 | SEQ ID | 6382 | SDC1 | Sindecan 1 |
| | NO: 251 | | | |
| SG252 | SEQ ID | 1019 | CDK4 | Cyclin-dependent kinase 4 |
| | NO: 252 | | | |
| SG253 | SEQ ID | 6774 | STAT3 | Signal transducer and transcription activator |
| | NO: 253 | | | 3 (response factor in acute phase) |
| SG254 | SEQ ID | 2268 | FGR | Homologue to Gardner-Rasheed's feline |
| | NO: 254 | | | viral sarcoma oncogene(v-fgr) |
| SG255 | SEQ ID | 2353 | FOS | Homologue to the murine viral |
| | NO: 255 | | | osteosarcoma oncogene V-fos FBJ |
| SG256 | SEQ ID | 898 | CCNE1 | Cyclin E1 |
| | NO: 256 | | | |
| SG257 | SEQ ID | 978 | CDA | Cytidine deaminase |
| | NO: 257 | | | |
| SG258 | SEQ ID | 9935 | MAFB | Homologue to the fibrosarcoma |
| | NO: 258 | | | oncogene (avian) musculoaponeurotic V- |
| | | | | maf |
| SG259 | SEQ ID | 4352 | MPL | Oncogene of myeloproliferative leukemia |
| | NO: 259 | | | virus |
| SG260 | SEQ ID | 4609 | MYC | Homologue to the avian myelocyomatosis |
| | NO: 260 | | | viral oncogene V-myc |
| SG261 | SEQ ID | 4602 | MYB | Homologue to the avian myelocyomatosis |
| | NO: 261 | | | viral oncogene V-myb |
| SG262 | SEQ ID | 1159 | CKMT1 | Creatine kinase, mitochondrial 1 (ubicuous) |
| | NO: 262 | | | |
| SG263 | SEQ ID | 1387 | CREBBP | CREB binding protein (Rubinstein-Taybi's |
| | NO: 263 | | | syndrome) |
| SG264 | SEQ ID | 1490 | CTGF | Connective tissue growth factor |
| | NO: 264 | | | |
| SG265 | SEQ ID | 2833 | CXCR3 | Chemokene receptor 3 (motive C—X—C) |
| | NO: 265 | | | |
| SG266 | SEQ ID | 7852 | CXCR4 | Chemokene receptor 4 (motive C—X—C) |
| | NO: 266 | | | (fusin) |
| SG267 | SEQ ID | 8900 | CCNA1 | Cyclin A1 |
| | NO: 267 | | | |
| SG268 | SEQ ID | 891 | CCNB1 | Cyclin B1 |
| | NO: 268 | | | |
| SG269 | SEQ ID | 894 | CCND2 | Cyclin D2 |
| | NO: 269 | | | |
| SG270 | SEQ ID | 1543 | CYP1A1 | Cytochrome P450, subfamily I (inducible by |
| | NO: 270 | | | aromatic compounds), polypeptide 1 |
| SG271 | SEQ ID | 1565 | CYP2A6 | Cytochrome P450, subfamily IID (of |
| | NO: 271 | | | metabolization of debrisokine, spartin, etc.), |
| | | | | polypeptide 6 |
| SG272 | SEQ ID | 1603 | DAD-1 | Defender against cell death 1 |
| | NO: 272 | | | |
| SG273 | SEQ ID | 8794 | TNFRSF10C | Tumour necrosing factor receptor |
| | NO: 273 | | | superfamily, member 10c, decoy without |
| | | | | intracellular domain |
| SG274 | SEQ ID | 7913 | DEK | Oncogene DEK (which binds to DNA) |
| | NO: 274 | | | |
| SG275 | SEQ ID | 1633 | DCK | Deoxycytidine kinase |
| | NO: 275 | | | |
| SG276 | SEQ ID | 1719 | DHFR | Dihydrofolate reductase |
| | NO: 276 | | | |
| SG277 | SEQ ID | 6929 | TCF3 | Transcription factor 3 (immunoglobulin |
| | NO: 277 | | | enhancer binding factors E2A E12/E47) |
| SG278 | SEQ ID | 1869 | E2F1 | Transcription factor E2F 1 |
| | NO: 278 | | | |
| SG279 | SEQ ID | 6929 | TCF3 | Transcription factor 3 (immunoglobulin |
| | NO: 279 | | | enhancer binding factors E2A E12/E47) |
| SG280 | SEQ ID | 56899 | EB-1 | Protein associated to E2a-Pbx1 |
| | NO: 280 | | | |
| SG281 | SEQ ID | 1236 | CCR7 | Chemokene receptor 7 (motive C-C) |
| | NO: 281 | | | |
| SG282 | SEQ ID | 1880 | EBI2 | Gene induced by Epstein-Barr's 2 disease |
| | NO: 282 | | | (receptor coupled to G proteins specific for |
| | | | | lymphocytes) |
| SG283 | SEQ ID | 4582 | MUC-1 | Mucin 1, transmembrane |
| | NO: 283 | | | |
| SG284 | SEQ ID | 2042 | EphA3 | EPHA3 |
| | NO: 284 | | | |
| SG285 | SEQ ID | 2057 | EPOR | Erythropoietin receptor |
| | NO: 285 | | | |
| SG286 | SEQ ID | 2067 | ERCC1 | Of repair of excision which |
| | NO: 286 | | | intercomplements the deficiency in the |
| | | | | repair of rodents, complementation group 1 |
| | | | | (includes the antisense overlapping |
| | | | | sequence) |
| SG287 | SEQ ID | 2068 | ERCC2 | Of repair of excision which |
| | NO: 287 | | | intercomplements the deficiency in the |
| | | | | repair of rodents, complementation group 2 |
| | | | | (xerodermia pigmentoso 2) |
| SG288 | SEQ ID | 2071 | ERCC3 | Of repair of excision which |
| | NO: 288 | | | intercomplements the deficiency in the |
| | | | | repair of rodents, complementation group 3 |
| | | | | (complements group B of xerodermia |
| | | | | pigmentoso) |
| SG289 | SEQ ID | 2073 | ERCC5 | Of repair of excision which |
| | NO: 289 | | | intercomplements the deficiency in the |
| | | | | repair of rodents, complementation group 5 |
| | | | | (xerodermia pigmentoso, complementation |
| | | | | group G (Cockayne's syndrome)) |
| SG290 | SEQ ID | 2074 | ERCC6 | Of repair of excision which |
| | NO: 290 | | | intercomplements the deficiency in the |
| | | | | repair of rodents, complementation group 6 |
| SG291 | SEQ ID | 50624 | CUZD1 | With CUB domains and similar to the zone |
| | NO: 291 | | | pellucida 1 |
| SG292 | SEQ ID | 2120 | ETV6 | Gene variant of ets 6 (TEL oncogene) |
| | NO: 292 | | | |
| SG293 | SEQ ID | 1977 | EIF4E | Initiation factor of eukaryotic translation 4E |
| | NO: 293 | | | |
| SG294 | SEQ ID | 1984 | EIF5A | Initiation factor of eukaryotic translation 5A |
| | NO: 294 | | | |
| SG295 | SEQ ID | 2146 | EZH2 | Zeste homologue enhancer (Drosophila) 2 |
| | NO: 295 | | | |
| SG296 | SEQ ID | 8772 | FADD | Associated to Fas (TNFRSF6) via |
| | NO: 296 | | | apoptopic domain |
| SG297 | SEQ ID | 5747 | PTK2 | Thyrosine kinase 2 protein |
| | NO: 297 | | | |
| SG298 | SEQ ID | 2195 | FAT | Homologue to the FAT tumour suppressor |
| | NO: 298 | | | (Drosophila) |
| SG299 | SEQ ID | 2260 | FGFR1 | Fibroblast growth factor receptor 1 |
| | NO: 299 | | | (thyrosine kinase related to fms 2, Pfeiffer's |
| | | | | syndrome) |
| SG300 | SEQ ID | 2261 | FGFR3 | Fibroblast growth factor receptor 3 |
| | NO: 300 | | | (achondroplasia, thanatophoric dwarfism) |
| SG301 | SEQ ID | 2272 | FHIT | Fragile histidine triad gene |
| | NO: 301 | | | |
| SG302 | SEQ ID | 2322 | FLT3 | Thyrosine kinase related to Fms 3 |
| | NO: 302 | | | |
| SG303 | SEQ ID | 2892 | GRIA3 | Glutamate receptor, ionotrophic, AMPA 3 |
| | NO: 303 | | | |
| SG304 | SEQ ID | 2521 | FUS | Fusion, derived from the malignant |
| | NO: 304 | | | liposarcoma t(12;16) |
| SG305 | SEQ ID | 1977 | EIF4E | Initiation factor of eukaryotic translation 4E |
| | NO: 305 | | | |
| SG306 | SEQ ID | 6482 | SIAT4A | Sialyltransferase 4A (beta-galactosidase |
| | NO: 306 | | | alpha-2.3-Sialyltransferase) |
| SG307 | SEQ ID | 1440 | CSF3 | Colony stimulating factor 3 (granulocyte) |
| | NO: 307 | | | |
| SG308 | SEQ ID | 1437 | CSF2 | Colony stimulating factor 2 (granulocyte- |
| | NO: 308 | | | microphage) |
| SG309 | SEQ ID | 2908 | NR3C1 | Subfamily of nuclear receptors 3, group C, |
| | NO: 309 | | | member 1 |
| SG310 | SEQ ID | 2952 | GSTT1 | Glutathion S-transferase theta 1 |
| | NO: 310 | | | |
| SG311 | SEQ ID | 3001 | GZMA | Granzime A (granzime 1, serine stearase |
| | NO: 311 | | | associated to cytotoxic T lymphocytes 3) |
| SG312 | SEQ ID | 3301 | DNAJA1 | Homologue to DnaJ (Hsp40), subfamily A, |
| | NO: 312 | | | member 1 |
| SG313 | SEQ ID | 3131 | HLF | Hepatic leukemia factor |
| | NO: 313 | | | |
| SG314 | SEQ ID | 684 | BST2 | Antigen of bone marrow stroma cells 2 |
| | NO: 314 | | | |
| SG315 | SEQ ID | 3205 | HOXA9 | Homeotic box A9 |
| | NO: 315 | | | |
| SG316 | SEQ ID | 3195 | TLX1 | T lymphocyte leukemia, homeotic box 1 |
| | NO: 316 | | | |
| SG317 | SEQ ID | 29128 | UHRF1 | similar to ubiquitine, which contains PHD |
| | NO: 317 | | | domains and RING fingers, 1 |
| SG318 | SEQ ID | 8870 | IER3 | Immediate early response 3 |
| | NO: 318 | | | |
| SG319 | SEQ ID | 3553 | IL1B | Interleukin 1, beta |
| | NO: 319 | | | |
| SG320 | SEQ ID | 3558 | IL2 | Interleukin 2 |
| | NO: 320 | | | |
| SG321 | SEQ ID | 3562 | IL3 | Interleukin 3 (multiple colony stimulating |
| | NO: 321 | | | factor) |
| SG322 | SEQ ID | 3569 | IL6 | Interleukin 6 (interferon, beta 2) |
| | NO: 322 | | | |
| SG323 | SEQ ID | 3570 | IL6R | Interleukin receptor 6 |
| | NO: 323 | | | |
| SG324 | SEQ ID | 3586 | IL10 | Interleukin 10 |
| | NO: 324 | | | |
| SG325 | SEQ ID | 3600 | IL15 | Interleukin 15 |
| | NO: 325 | | | |
| SG326 | SEQ ID | 3662 | IRF4 | Interferon regulating factor 4 |
| | NO: 326 | | | |
| SG327 | SEQ ID | 3716 | JAK1 | Janus 2 kinase (a thyrosine kinase protein) |
| | NO: 327 | | | |
| SG328 | SEQ ID | 3717 | JAK2 | Janus 1 kinase (a thyrosine kinase protein) |
| | NO: 328 | | | |
| SG329 | SEQ ID | 4288 | MKI67 | Antigen identified by monoclonal antibody |
| | NO: 329 | | | Ki-67 |
| SG330 | SEQ ID | 7520 | XRCC5 | Of repair of X rays which complements the |
| | NO: 330 | | | defective repair in Chinese hamster cells 5 |
| | | | | (reconnection of breakages in the double |
| | | | | helix; autoantigen Ku, 80 kDa) |
| SG331 | SEQ ID | 3902 | LAG3 | Lymphocyte activation gene 3 |
| | NO: 331 | | | |
| SG332 | SEQ ID | 3932 | LCK | Lymphocyte specific protein thyrosine |
| | NO: 332 | | | kinase |
| SG333 | SEQ ID | 3936 | LCP1 | Cytosolic protein of lymphocytes 1 (L- |
| | NO: 333 | | | plastin) |
| SG334 | SEQ ID | 3953 | LEPR | Leptin receptor |
| | NO: 334 | | | |
| SG335 | SEQ ID | 4005 | LMO2 | With LIM domains only 2 (similar to |
| | NO: 335 | | | rombotin 1) |
| SG336 | SEQ ID | 3976 | LIF | Leukemia inhibiting factor (cholinergic |
| | NO: 336 | | | differentiation factor) |
| SG337 | SEQ ID | 9961 | LRP | Main leap protein |
| | NO: 337 | | | |
| SG338 | SEQ ID | 4046 | LSP1 | Lymphocyte-specific protein 1 |
| | NO: 338 | | | |
| SG339 | SEQ ID | 4066 | LYL1 | Sequence derived from lymphoblastic |
| | NO: 339 | | | leukemia 1 |
| SG340 | SEQ ID | 4790 | NFKB1 | Nuclear factor of the enhancer of the gene |
| | NO: 340 | | | of the light kappa polypeptide in B-1 |
| | | | | lymphocytes(p105) |
| SG341 | SEQ ID | 4118 | MAL | mal, T lymphocyte differentiation protein |
| | NO: 341 | | | |
| SG342 | SEQ ID | 4100 | MAGEA1 | Melanoma antigen, family A, 1 (directs the |
| | NO: 342 | | | expression of antigen MZ2-E) |
| SG343 | SEQ ID | 5602 | MAPK10 | Protein kinase activated by mitogens 10 |
| | NO: 343 | | | |
| SG344 | SEQ ID | 2023 | MBP1 | Enolase 1, (alpha) |
| | NO: 344 | | | |
| SG345 | SEQ ID | 4170 | MCL1 | Leukemia sequence of myeloid cells 1 |
| | NO: 345 | | | (related to BCL2) |
| SG346 | SEQ ID | 4193 | MDM2 | Human homologue of the double mouse |
| | NO: 346 | | | diminuta 2; p53-binding protein |
| SG347 | SEQ ID | 5243 | ABCB1 | ATP binding module, subfamily B |
| | NO: 347 | | | (MDR/TAP), member 1 |
| SG348 | SEQ ID | 5244 | ABCB4 | ATP binding module, subfamily B |
| | NO: 348 | | | (MDR/TAP), member 4 |
| SG349 | SEQ ID | 4221 | MEN1 | Multiple endocrine neoplasia I |
| | NO: 349 | | | |
| SG350 | SEQ ID | 4283 | CXCL9 | Chemokene ligand 9 (motive C—X—C) |
| | NO: 350 | | | |
| SG351 | SEQ ID | 4291 | MLF1 | Myeloid leukemia factor 1 |
| | NO: 351 | | | |
| SG352 | SEQ ID | 4297 | MLL | Myeloid/lymphoid leukemia or of mixed line |
| | NO: 352 | | | (homologue to trithorax (Drosophila)) |
| SG353 | SEQ ID | 4318 | MMP9 | Metalloproteinase of matrix 9 (gelatinase B, |
| | NO: 353 | | | 92 kD gelatinase, 92 kD collagenase type IV) |
| SG354 | SEQ ID | 4707 | NDUFB | NADH dehydrogenase (ubiquinone) 1 beta |
| | NO: 354 | | | subcomplex, 1 (7 kD, MNLL) |
| SG355 | SEQ ID | 4353 | MPO | Myeloperoxidase |
| | NO: 355 | | | |
| SG356 | SEQ ID | 8714 | ABCC3 | ATP binding module, subfamily C |
| | NO: 356 | | | (CFTR/MRP), member 3 |
| SG357 | SEQ ID | 10057 | ABCC5 | ATP binding module, subfamily C |
| | NO: 357 | | | (CFTR/MRP), member 5 |
| SG358 | SEQ ID | 4515 | MTCP1 | Proliferation of mature T-lymphocytes 1 |
| | NO: 358 | | | |
| SG359 | SEQ ID | 4515 | MTCP1 | Proliferation of mature T-lymphocytes 1 |
| | NO: 359 | | | |
| SG360 | SEQ ID | 4654 | MYOD1 | Myogenic factor 3 |
| | NO: 360 | | | |
| SG361 | SEQ ID | 4599 | MX1 | Resistance to Myxovirus (flu) 1, homologue |
| | NO: 361 | | | to the murine protein (protein inducible by |
| | | | | interferon p78) |
| SG362 | SEQ ID | 4814 | NINJ1 | Ninjurin 1 |
| | NO: 362 | | | |
| SG363 | SEQ ID | 4869 | NPM1 | Nucleophosmin (nucleolar phosphoprotein |
| | NO: 363 | | | B23, numatrin) |
| SG364 | SEQ ID | 9235 | IL32 | Interleukin 32 |
| | NO: 364 | | | |
| SG365 | SEQ ID | 4926 | NUMA1 | Nuclear protein of the myotic apparatus 1 |
| | NO: 365 | | | |
| SG366 | SEQ ID | 5452 | POU2F2 | transcription factor with POU domain, of |
| | NO: 366 | | | class 2, 2 |
| SG367 | SEQ ID | 5452 | POU2F2 | transcription factor with POU domain, of |
| | NO: 367 | | | class 2, 2 |
| SG368 | SEQ ID | 4968 | OGGI | 8-oxoguanine-DNA-glycosilase |
| | NO: 368 | | | |
| SG369 | SEQ ID | 1030 | CDKN2B | Cyclin-dependent kinase inhibitor 2B (p15, |
| | NO: 369 | | | inhibits CDK4) |
| SG370 | SEQ ID | 1029 | CDKN2A | Cyclin-dependent kinase inhibitor 2A |
| | NO: 370 | | | (melanoma, p16, inhibits CDK4) |
| SG371 | SEQ ID | 1031 | CDKN2C | Cyclin-dependent kinase inhibitor 2C (p18, |
| | NO: 371 | | | inhibits CDK4) |
| SG372 | SEQ ID | 1026 | CDKN1A | Cyclin-dependent kinase inhibitor 1A (p21, |
| | NO: 372 | | | Cip1) |
| SG373 | SEQ ID | 1027 | CDKN1B | Cyclin-dependent kinase inhibitor 1B (p27, |
| | NO: 373 | | | Kip1) |
| SG374 | SEQ ID | 8851 | CDK5R1 | Cyclin-dependent kinase 5, regulator |
| | NO: 374 | | | subunit 1 (p35) |
| SG375 | SEQ ID | 10210 | TOPORS | Of binding to topoisomerase I, rich inc |
| | NO: 375 | | | arginine/serine |
| SG376 | SEQ ID | 991 | P55CDC | CDC20 (cell division cycle 20, S. cerevisiae, |
| | NO: 376 | | | homologue) |
| SG377 | SEQ ID | 1028 | CDKN1C | Cyclin-dependent kinase inhibitor 1C (p57, |
| | NO: 377 | | | Kip2) |
| SG378 | SEQ ID | 7161 | TP73 | Tumour protein p73 |
| | NO: 378 | | | |
| SG379 | SEQ ID | 5079 | PAX5 | Paired box gene 5 (specific activating |
| | NO: 379 | | | protein of the B lymphocytes line) |
| SG380 | SEQ ID | 5087 | PBX1 | Transcription factor of B 1 prelymphocytes |
| | NO: 380 | | | leukemia |
| SG381 | SEQ ID | 5090 | PBX3 | Transcription factor of B prelymphocytes |
| | NO: 381 | | | leukemia B 3 |
| SG382 | SEQ ID | 5089 | ENPP1 | Ectonucleotide |
| | NO: 382 | | | pyrophosphatase/phosphodiesterase 1 |
| SG383 | SEQ ID | 5167 | PCA1 | Ectonucleotide |
| | NO: 383 | | | pyrophosphatase/phosphodiesterase 1 |
| SG384 | SEQ ID | 5111 | PCNA | Nuclear antigen of proliferating cells |
| | NO: 384 | | | |
| SG385 | SEQ ID | 5159 | PDGFRB | Platelet-derived growth factor receptor, beta |
| | NO: 385 | | | polypeptide |
| SG386 | SEQ ID | 5588 | PRKCQ | kinase C protein, theta |
| | NO: 386 | | | |
| SG387 | SEQ ID | 5347 | PLK1 | Plasminogen activator, urokinase |
| | NO: 387 | | | |
| SG388 | SEQ ID | 5371 | PML | Promielocytic leukemia |
| | NO: 388 | | | |
| SG389 | SEQ ID | 23532 | PRAME | Antigen expressed preferentially in |
| | NO: 389 | | | melanoma |
| SG390 | SEQ ID | 5584 | PRKCI | protein kinase C, iota |
| | NO: 390 | | | |
| SG391 | SEQ ID | 5728 | PTEN | Phosphatase and homologue to tensin |
| | NO: 391 | | | (mutated in multiple advanced cancers 1) |
| SG392 | SEQ ID | 5742 | PTGS1 | prostaglandin-endoperoxide synthase 1 |
| | NO: 392 | | | (prostaglandin G/H synthase and |
| | | | | cyclooxigenase) |
| SG393 | SEQ ID | 5744 | PTHLH | Hormone similar to the parathyroidal |
| | NO: 393 | | | hormone |
| SG394 | SEQ ID | 6688 | SPI1 | Oncogene of integration of provirus of focus |
| | NO: 394 | | | forming virus in the spleen (SFFV) spi1 |
| SG395 | SEQ ID | 2185 | PTK2B | Thyrosine kinase 2 beta protein |
| | NO: 395 | | | |
| SG396 | SEQ ID | 5889 | RAD51 | Homologue to RAD51 (S. cerevisiae) |
| | NO: 396 | | | (homologue to RecA of E. coli) |
| SG397 | SEQ ID | 5896 | RAG1 | Recombination activator gene 1 |
| | NO: 397 | | | |
| SG398 | SEQ ID | 5914 | RARA | Retinoic acid receptor, alpha |
| | NO: 398 | | | |
| SG399 | SEQ ID | 3845 | KRAS2 | Homologue to the viral oncogene of Kirsten |
| | NO: 399 | | | 2 V-Ki-ras2 rat sarcoma |
| SG400 | SEQ ID | 5925 | RB1 | Retinoblastoma 1 (including osteosarcoma) |
| | NO: 400 | | | |
| SG401 | SEQ ID | 7422 | VEGF | Vascular endothelial growth factor |
| | NO: 401 | | | |
| SG402 | SEQ ID | 7791 | ZYX | Zyxin |
| | NO: 402 | | | |
| SG403 | SEQ ID | 940 | CD28 | Antigen CD28 (Tp44) |
| | NO: 403 | | | |
| SG404 | SEQ ID | 940 | CD28 | Antigen CD28 (Tp44) |
| | NO: 404 | | | |
| SG405 | SEQ ID | 1656 | RBBP4 | retinoblastoma 4 binding protein |
| | NO: 405 | | | |
| SG406 | SEQ ID | 1656 | DDX6 | Polypeptide with DEAD/H box (Asp-Glu- |
| | NO: 406 | | | Ala-Asp/His) 6 (RNA helicase, 54 kD) |
| SG407 | SEQ ID | 5928 | APEX | APEX nuclease (multifunctional DNA repair |
| | NO: 407 | | | enzyme DNA) |
| SG408 | SEQ ID | 5977 | DPF2 | D4, family 2 with zinc fingers and double |
| | NO: 408 | | | PHD |
| SG409 | SEQ ID | 5996 | RGS1 | G protein signalling regulator G 1 |
| | NO: 409 | | | |
| SG410 | SEQ ID | 3161 | HMMR | Motility receptor mediated by hyaluronane |
| | NO: 410 | | | (RHAMM) |
| SG411 | SEQ ID | 6777 | STAT5B | signal transducer and transcription activator |
| | NO: 411 | | | 5B |
| SG412 | SEQ ID | 332 | BIRC5 | Which contains IAP repetitions of |
| | NO: 412 | | | baculovirus 5 (survivin) |
| SG413 | SEQ ID | 6886 | TAL1 | Acute lymphocytic leukemia of T |
| | NO: 413 | | | lymphocytes 1 |
| SG414 | SEQ ID | 10482 | NXF1 | RNA exportation nuclear factor of RNA 1 |
| | NO: 414 | | | |
| SG415 | SEQ ID | 8115 | TCL1A | Leukemia/lymphoma of T 1A lymphocytes |
| | NO: 415 | | | |
| SG416 | SEQ ID | 6955 | TRA@ | T locus alpha lymphocyte receptor |
| | NO: 416 | | | |
| SG417 | SEQ ID | | TCR beta | mRNA of the beta chain of the T |
| | NO: 417 | | | lymphocyte receptor (TCRB) of Homo |
| | | | | sapiens |
| SG418 | SEQ ID | 1791 | DNTT | Deoxynucleotidyl transferase, terminal |
| | NO: 418 | | | |
| SG419 | SEQ ID | 7015 | TERT | Inverse telomerase transcriptase |
| | NO: 419 | | | |
| SG420 | SEQ ID | 2066 | ERBB4 | Similar to the homologue to the viral |
| | NO: 420 | | | oncogene of avian erythroblastic leukemia |
| | | | | V-erb-a 4 |
| SG421 | SEQ ID | 2064 | ERBB2 | Homologue to the to the viral oncogene of |
| | NO: 421 | | | avian erythroblastic leukemia V-erb-b2 2 |
| | | | | (homologue to the oncogene derived from |
| | | | | neuro/glioblastoma) |
| SG422 | SEQ ID | 1956 | EGFR | Epidermal growth factor receptor |
| | NO: 422 | | | (homologue to the viral oncogene of avian |
| | | | | erythroblastic leukemia (v-erb-b)) |
| SG423 | SEQ ID | 7066 | THPO | Thrombopoietin (oncogene ligand of the |
| | NO: 423 | | | myeloproliferative leukemia virus, growth |
| | | | | factor and development of megakaryocytes) |
| SG424 | SEQ ID | 7074 | TIAM1 | Invasion and metastasis of lymphoma of T |
| | NO: 424 | | | lymphocytes 1 |
| SG425 | SEQ ID | 7083 | TK1 | Thymidine kinase 1, soluble |
| | NO: 425 | | | |
| SG426 | SEQ ID | 7132 | TNFRSF1A | Tumour necrosing factor receptor |
| | NO: 426 | | | superfamily, member 1A |
| SG427 | SEQ ID | 7153 | TOP2A | Topoisomerase (DNA) II alpha (170 kD) |
| | NO: 427 | | | |
| SG428 | SEQ ID | 1052 | CEBPD | binding protein to CCAAT/enhancer |
| | NO: 428 | | | (C/EBP), delta |
| SG428 | SEQ ID | 9214 | FAIM3 | Apoptosis inhibitor molecule mediated by |
| | NO: 428 | | | Fas 3 |
| SG429 | SEQ ID | 5358 | PLS3 | Plastin 3 (isoform T) |
| | NO: 429 | | | |
| SG430 | SEQ ID | 8717 | TRADD | associated to TNFRSF1A via the cell death |
| | NO: 430 | | | domain |
| SG431 | SEQ ID | 8743 | TNFSF10 | Tumour necrosing factor superfamily |
| | NO: 431 | | | (ligand) member 10 |
| SG432 | SEQ ID | 10131 | TRAP 1 | Thermal shock protein 75 |
| | NO: 432 | | | |
| SG433 | SEQ ID | 7057 | THBS1 | Thrombospondin 1 |
| | NO: 433 | | | |
| SG434 | SEQ ID | 7341 | SUMO1 | Homologue to the supressor of mif two 3 |
| | NO: 434 | | | SMT3 1 (yeast) |
| SG435 | SEQ ID | 7405 | UVRAG | Gene associated to UV radiation resistance |
| | NO: 435 | | | |
| SG436 | SEQ ID | 7441 | VPREB1 | Gene of B 1 prelymphocytes |
| | NO: 436 | | | |
| SG437 | SEQ ID | 51384 | WNT16 | family of MMTV integration site, Wingless- |
| | NO: 437 | | | type, member 16 |
| SG438 | SEQ ID | 7490 | WT1 | Wilms tumour 1 |
| | NO: 438 | | | |
| SG439 | SEQ ID | 7517 | XRCC3 | Of repair of X rays which complement the |
| | NO: 439 | | | defective repair in Chinese hamster cells 3 |
| SG440 | SEQ ID | 896 | CCND3 | Cyclin D3 |
| | NO: 440 | | | |
| SG441 | SEQ ID | 1017 | CDK2 | Cyclin-dependent kinase 2 |
| | NO: 441 | | | |
| SG442 | SEQ ID | | p14ARF | Gene p14ARF from Homo sapiens, |
| | NO: 442 | | | promoter region, complete sequence |
| SG443 | SEQ ID | 3070 | HELLS | Helicase, specific for lymphoid cells |
| | NO: 443 | | | |
| SG444 | SEQ ID | 2624 | GATA2 | GATA 1-binding protein 2 |
| | NO: 444 | | | |
| SG445 | SEQ ID | 2623 | GATA1 | GATA 1-binding protein (globin transcription |
| | NO: 445 | | | factor 1) |
| SG446 | SEQ ID | 8028 | MLLT10 | Myeloid/lymphoid leukemia or of mixed line |
| | NO: 446 | | | (homologue to trithorax (Drosophila)); |
| | | | | translocated to 10 |
| SG447 | SEQ ID | 8301 | PICALM | Clathrin assembly protein which binds to |
| | NO: 447 | | | phosphatidylinositol |
| SG448 | SEQ ID | 3815 | KIT | Homologue to the viral oncogene of Hardy- |
| | NO: 448 | | | Zuckerman's feline sarcoma 4 V-kit |
| SG449 | SEQ ID | 3563 | IL3RA | Interleukin 3 receptor, alpha (low affinity) |
| | NO: 449 | | | |
| SG450 | SEQ ID | 1050 | CEBPA | binding protein to CCAAT/enhancer |
| | NO: 450 | | | (C/EBP), alpha |
| SG451 | SEQ ID | 3655 | ITGA6 | Integrin, alpha 6 |
| | NO: 451 | | | |
| SG452 | SEQ ID | 84955 | CML66 | Tumour antigen of chronic myelogenous |
| | NO: 452 | | | leukemia 66 |
| SG453 | SEQ ID | 7187 | TRAF3 | Factor associated to the TNF receptor 3 |
| | NO: 453 | | | |
| SG454 | SEQ ID | 1612 | DAPK1 | Kinase protein associated to cell death 1 |
| | NO: 454 | | | |
| SG455 | SEQ ID | 8788 | MAP3K12 | Homologue similar to Delta (Drosophila) |
| | NO: 455 | | | |
| SG456 | SEQ ID | 5591 | PRKDC | Kinase protein, activated by DNA, catalytic |
| | NO: 456 | | | polypeptide |
| SG457 | SEQ ID | 1789 | DNMT3B | (cytosine-5-)-methyltransferase 3 beta of |
| | NO: 457 | | | DNA |
| SG458 | SEQ ID | 2950 | GSTP1 | Glutathion S-transferase pi |
| | NO: 458 | | | |
| SG459 | SEQ ID | 3122 | HLA-DRA | Complex greater than histocompatibility, |
| | NO: 459 | | | class II, DR alpha |
| SG460 | SEQ ID | 3206 | HOXA10 | Homeotic box A10 |
| | NO: 460 | | | |
| SG461 | SEQ ID | 3394 | IRF8 | Binding protein to the agreed sequence of |
| | NO: 461 | | | interferon 1 |
| SG462 | SEQ ID | 3398 | ID2 | DNA binding inhibitor 2, negative dominant |
| | NO: 462 | | | helix-loops-helix protein |
| SG463 | SEQ ID | 60 | ACTB | Actin, beta |
| | NO: 463 | | | |
| SG464 | SEQ ID | 60 | ACTB | Actin, beta |
| | NO: 464 | | | |
| SG465 | SEQ ID | 2868 | GRK4 | Kinase of receptor coupled to a protein G 4 |
| | NO: 465 | | | |
| SG466 | SEQ ID | 2597 | GAPD | Glyceraldehyde-3-phosphate |
| | NO: 466 | | | dehydrogenase |
| SG467 | SEQ ID | 2597 | GAPD | Glyceraldehyde-3-phosphate |
| | NO: 467 | | | dehydrogenase |
| SG468 | SEQ ID | 6772 | STAT1 | Signal transducer and transcription activator |
| | NO: 468 | | | 1, 91 kDa |
| SG469 | SEQ ID | | 18S rRNA | Human rRNA gene 18S |
| | NO: 469 | | | |
| SG470 | SEQ ID | 7037 | TFRC | Transferrin receptor (p90, CD71) |
| | NO: 470 | | | |
| SG471 | SEQ ID | | 28S rRNA | Human ribosomal RNA gene 28S |
| | NO: 471 | | | |
| SG472 | SEQ ID | 6168 | RPL37A | Ribosomal protein L37a |
| | NO: 472 | | | |
| SG473 | SEQ ID | 6171 | RPL41 | Ribosomal protein L41 |
| | NO: 473 | | | |
| SG474 | SEQ ID | 3191 | HNRPL | Heterogeneous nucler ribonucleoprotein L |
| | NO: 474 | | | |
| SG475 | SEQ ID | 3608 | ILF2 | Binding factor to the interleukin-2 enhancer, |
| | NO: 475 | | | 45 kD |
| SG476 | SEQ ID | 8407 | TAGLN2 | Transgelin 2 |
| | NO: 476 | | | |
| SG477 | SEQ ID | 824 | CAPN2 | Calpain 2, (m/II) major subunit |
| | NO: 477 | | | |
| SG478 | SEQ ID | 5686 | PSMA5 | Subunit of proteasome (prosome, |
| | NO: 478 | | | macropain), type alpha, 5 |
| SG479 | SEQ ID | 27254 | PMM1 | Phosphomannomutase 1 |
| | NO: 479 | | | |
| SG480 | SEQ ID | 8079 | MLF2 | Myeloid leukemia factor 2 |
| | NO: 480 | | | |
| SG481 | SEQ ID | 5501 | PPP1CC | Phosphatase protein 1, catalytic subunit, |
| | NO: 481 | | | gamma isoform |
| SG482 | SEQ ID | 22794 | CASC3 | Candidate for susceptibility to cancer 3 |
| | NO: 482 | | | |
| SG483 | SEQ ID | 23164 | KIAA0864 | Protein KIAA0864 |
| | NO: 483 | | | |
| SG484 | SEQ ID | 7296 | TXNRD1 | Thioredoxine reductase 1 |
| | NO: 484 | | | |
| SG485 | SEQ ID | 5713 | PSMD7 | Subunit of proteasome (prosome, |
| | NO: 485 | | | macropain) 26S, no-ATPase, 7 (homologue |
| | | | | to Mov34) |
| SG486 | SEQ ID | 8892 | EIF2B2 | initiation factor of eukaryotic translation 2B, |
| | NO: 486 | | | subunit 2 (beta, 39 kD) |
| SG487 | SEQ ID | 3105 | HLA-A | Complex greater than histocompatibility, |
| | NO: 487 | | | class I, A |
| SG488 | SEQ ID | 4176 | MCM7 | Minichromosome maintenance deficient (S. cerevisiae) 7 |
| | NO: 488 | | | |
| SG489 | SEQ ID | 8718 | TNFRSF25 | Tumour necrosing factor receptor |
| | NO: 489 | | | superfamily, member 25 |
| SG490 | SEQ ID | 3958 | LGALS3 | Lectin, which binds to galactosides, soluble, |
| | NO: 490 | | | 3 (galectin 3) |
| SG491 | SEQ ID | 311 | HLA-DPA1 | Complex greater than histocompatibility, |
| | NO: 491 | | | class II, DP alpha 1 |
| SG492 | SEQ ID | 5328 | PLAU | Plasminogen activator, urokinase |
| | NO: 492 | | | |
| SG493 | SEQ ID | 1281 | COL3A1 | Collagen, type III, alpha 1 (Ehlers-Danlos |
| | NO: 493 | | | type IV syndrome, dominant autosomal) |
| SG494 | SEQ ID | 287 | ANK2 | Ankyrin 2, neuronal |
| | NO: 494 | | | |
| SG495 | SEQ ID | 327657 | SERPINA9 | serine (or cysteine) proteinase inhibitor, |
| | NO: 495 | | | clade A (alpha-1 antiproteinase, |
| | | | | antitrypsin), member 9 |
| SG496 | SEQ ID | 10360 | NPM3 | Nucleophosmin/nucleoplasmin 3 |
| | NO: 496 | | | |
| SG497 | SEQ ID | 1235 | CCR6 | Receptor 6 of chemokenes (motive C-C) |
| | NO: 497 | | | |
| SG498 | SEQ ID | 3055 | HCK | Hematopoietic cell kinase |
| | NO: 498 | | | |
| SG499 | SEQ ID | 26354 | GNL3 | similar to guanine 3 nucleotide binding |
| | NO: 499 | | | protein (nucleolar) |
| SG500 | SEQ ID | 2885 | GRB2 | Protein bound to growth factor receptor 2 |
| | NO: 500 | | | |
| SG501 | SEQ ID | 597 | BCL2A1 | protein related to BCL2 A1 |
| | NO: 501 | | | |
| SG502 | SEQ ID | 1997 | ELF1 | Factor similar to E74 1 (transcription factor |
| | NO: 502 | | | with ets domain) |
| SG503 | SEQ ID | 1508 | CTSB | Catepsin B |
| | NO: 503 | | | |
| SG504 | SEQ ID | 257144 | GCET2 | transcript expressed in the budding centre 2 |
| | NO: 504 | | | |
| SG505 | SEQ ID | 2335 | FN1 | Fibronectin 1 |
| | NO: 505 | | | |
| SG506 | SEQ ID | 5133 | PDCD1 | Programme cell death 1 |
| | NO: 506 | | | |
| SG507 | SEQ ID | 3125 | HLA-DRB3 | Complex greater than histocompatibility, |
| | NO: 507 | | | class II, DR beta 3 |
| SG508 | SEQ ID | 3117 | HLA-DQA1 | Complex greater than histocompatibility, |
| | NO: 508 | | | class II, DQ alpha 1 |
| SG509 | SEQ ID | 257144 | GCET2 | transcript expressed in the budding centre |
| | NO: 509 | | | germinal 2 |
| SG510 | SEQ ID | 327657 | SERPINA9 | serine (or cysteine) proteinase inhibitor, |
| | NO: 510 | | | clade A (alpha-1 antiproteinase, |
| | | | | antitrypsin), member 9 |
| SG511 | SEQ ID | 1033 | CDKN3 | Cyclin dependent kinases 3 (phosphatase |
| | NO: 511 | | | of dual specificity associated CDK2) |
| SG512 | SEQ ID | 1997 | ELF1 | Factor similar to E74 1 (transcription factor |
| | NO: 512 | | | with ets domain) |
| SG513 | SEQ ID | 1509 | CATSD | Catepsin D (liposomal aspartylprotease) |
| | NO: 513 | | | |
| SG514 | SEQ ID | 3315 | HSPB1 | Thermal shock protein of 27 kD 1 |
| | NO: 514 | | | |
| SG515 | SEQ ID | 87 | ACTN1 | Actinin, alpha 1 |
| | NO: 515 | | | |
| SG516 | SEQ ID | 654 | BMP6 | Morphogenetic bone protein 6 |
| | NO: 516 | | | |
| SG517 | SEQ ID | 9780 | FAM38A | family with similarity of sequence 38, member A |
| | NO: 517 | | | |
| SG518 | SEQ ID | 962 | CD48 | Antigen CD48 (membrane protein of B |
| | NO: 518 | | | lymphocytes) |
| SG519 | SEQ ID | 3566 | IL4R | Interleukin 4 receptor |
| | NO: 519 | | | |
| SG520 | SEQ ID | 1821 | DRP2 | Dystrophin related protein 2 |
| | NO: 520 | | | |
| SG521 | SEQ ID | 3726 | JUNB | Jun B Proto-oncogene |
| | NO: 521 | | | |
| SG522 | SEQ ID | 6279 | S100A8 | Calcium-binding protein S100 A8 |
| | NO: 522 | | | (calgranuline A) |
| SG523 | SEQ ID | 10320 | ZNFN1A1 | Protein with zinc fingers, subfamily 1A, 1 |
| | NO: 523 | | | (lkaros) |
| SG524 | SEQ ID | 10461 | MERTK | Thyrosine kinase of the C-mer proto- |
| | NO: 524 | | | oncogene |
| SG525 | SEQ ID | 51621 | KLF13 | Factor similar to that of Kruppel 13 |
| | NO: 525 | | | |
| SG526 | SEQ ID | 865 | CBFB | Nucleus-binding factor, beta subunit |
| | NO: 526 | | | |
| SG527 | SEQ ID | 1051 | CEBPB | binding protein to CCAAT/enhancer |
| | NO: 527 | | | (C/EBP), beta |
| SG529 | SEQ ID | 7024 | TFCP2 | Transcription factor CP2 |
| | NO: 529 | | | |
| SG530 | SEQ ID | 1385 | CREB1 | CAMP response element binding protein |
| | NO: 530 | | | |
| SG531 | SEQ ID | 4782 | NFIC | I/C nuclear factor (CCAAT binding factor |
| | NO: 531 | | | transcription factor) |
| SG532 | SEQ ID | 2553 | GABPB2 | Transcription factor of the protein which |
| | NO: 532 | | | binds to GA, beta 2 subunit (47 kD) |
| SG533 | SEQ ID | 1958 | EGR1 | Early growth response 1 |
| | NO: 533 | | | |
| SG534 | SEQ ID | 10661 | KLF1 | Factor similar to that of Kruppel 1 |
| | NO: 534 | | | (erythroid) |
| SG535 | SEQ ID | 1997 | ELF1 | Factor similar to E74 1 (transcription factor |
| | NO: 535 | | | with ets domain) |
| SG536 | SEQ ID | 2113 | ETS1 | homologue to the E26 oncogene of |
| | NO: 536 | | | erythroblastosis virus v-ets 1 (avian) |
| SG537 | SEQ ID | 2114 | ETS2 | homologue to the E26 oncogene of |
| | NO: 537 | | | erythroblastosis virus v-ets 2 (avian) |
| SG538 | SEQ ID | 2313 | FLI1 | Integration of Friend 1 leukemia virus |
| | NO: 538 | | | |
| SG539 | SEQ ID | 2625 | GATA3 | GATA 3 binding protein |
| | NO: 539 | | | |
| SG540 | SEQ ID | 862 | CBFA2T1 | Nucleus binding factor, runt domain, alpha |
| | NO: 540 | | | 2 subunit; translocated to 1; related to cyclin D |
| SG541 | SEQ ID | 3091 | HIF1A | Factor inducible by hypoxia 1, alpha subunit |
| | NO: 541 | | | (basic transcription factor of helix-loops- |
| | | | | helix) |
| SG542 | SEQ ID | 6927 | TCF1 | Transcription factor 1, hepatic; LF-B1, |
| | NO: 542 | | | hepatic nuclear factor (HNF1), proximal |
| | | | | factor of albumin |
| SG543 | SEQ ID | 3234 | HOXD8 | Homeotic box D8 |
| | NO: 543 | | | |
| SG544 | SEQ ID | 3235 | HOXD9 | Homeotic box D9 |
| | NO: 544 | | | |
| SG545 | SEQ ID | 9935 | MAFB | Family of the oncogene of |
| | NO: 545 | | | musculoaponeurotic fibrosarcoma (avian) |
| | | | | V-maf |
| SG546 | SEQ ID | 7975 | MAFK | Family of the oncogene of |
| | NO: 546 | | | musculoaponeurotic fibrosarcoma (avian) |
| | | | | V-maf, protein K |
| SG547 | SEQ ID | 8721 | EDF1 | factor related to edothelialdifferentiation 1 |
| | NO: 547 | | | |
| SG548 | SEQ ID | 2000 | ELF4 | Factor similar to E74 4 (transcription factor |
| | NO: 548 | | | with ets domain) |
| SG549 | SEQ ID | 7593 | ZNF42 | Protein with zinc fingers 142 (clone pHZ-49) |
| | NO: 549 | | | |
| SG550 | SEQ ID | 4763 | NF1 | neurofibromin 1 |
| | NO: 550 | | | |
| SG551 | SEQ ID | 4772 | NFATC1 | Nuclear factor of activated T-lymphocytes, |
| | NO: 551 | | | cytoplasmic, calcineurin 1-dependent |
| SG552 | SEQ ID | 5080 | PAX6 | Paired box gene 6 (aniridia, keratitis) |
| | NO: 552 | | | |
| SG553 | SEQ ID | 7849 | PAX8 | Paired box gene 8 |
| | NO: 553 | | | |
| SG554 | SEQ ID | 57026 | PLP | Pyridoxal (pyridoxine, vitamin B6) |
| | NO: 554 | | | phosphatase |
| SG555 | SEQ ID | 2274 | PLZF | With four LIM domains and average 2 |
| | NO: 555 | | | |
| SG556 | SEQ ID | 5950 | RBP4 | Retinol 4-binding protein, plasma |
| | NO: 556 | | | |
| SG557 | SEQ ID | 6095 | RORA | Orphan receptor related to RAR A |
| | NO: 557 | | | |
| SG558 | SEQ ID | 6667 | SP1 | Transcription factor Sp1 |
| | NO: 558 | | | |
| SG559 | SEQ ID | 6772 | STAT1 | Signal transducer and transcription activator |
| | NO: 559 | | | 1, 91 kDa |
| SG560 | SEQ ID | 6908 | TBP | TATA box-binding protein |
| | NO: 560 | | | |
| SG561 | SEQ ID | 6932 | TCF7 | Transcription factor 7 (T lymphocyte |
| | NO: 561 | | | specific, box HMG) |
| SG562 | SEQ ID | 51513 | ETV7 | Gene variant of ets 7 (oncogene TEL2) |
| | NO: 562 | | | |
| SG563 | SEQ ID | 7535 | ZAP70 | Zeta chain associated kinase protein (TCR) |
| | NO: 563 | | | (70 kD) |

From among these genes, four of them (ACTB, GAPD, 18S rRNA and 28S rRNA), do not have a special relation with neoplasias and were initially included in the microarray because, for a long time, it was believed that their expression remained constant and they were used when normalizing the microarray data: they are the type of genes alluded to when we speak of “constitutive” genes at other points in the specification. At present, it is not thought that there is a gene whose expression remains constant in any circumstance, for which reason, in the present study, the genes ACTB, GAPD, 18S rRNA and 28S rRNA have received the same treatment as the other genes of the microarray, except for the fact that the first two of them have been used as integrity controls, as described further on.

In Table 1 it can be observed that there are genes which are represented by more than one oligonucleotide. This is the case because the existence of two or more probes per gene can be used to measure the integrity of the synthesized cRNA. The genes for which more than one oligonucleotide have been designed to act as probe, each one of which hybridizes with a different sequence, are indicated below in Table 2.

TABLE 2
Genes represented by more than one oligonucleotide as probe

| Usual abbreviation | | | |
|---|---|---|---|
| of the gene | Probe1 | Probe2 | Probe3 |
| ABL1 | SG10 | SG180 | |
| BCR | SG169 | SG170 | |
| CBFB | SG189 | SG526 | |
| CD28 | SG403 | SG404 | |
| EIF4E | SG293 | SG305 | |
| ELF1 | SG512 | SG535 | SG502 |
| ETS2 | SG95 | SG537 | |
| GCET2 | SG504 | SG509 | |
| MAFB | SG258 | SG545 | |
| MTCP1 | SG358 | SG359 | |
| POU2F2 | SG366 | SG367 | |
| RGS1 | SG56 | SG409 | |
| S100A2 | SG35 | SG71 | |
| SNRPB | SG142 | SG143 | |
| STAT1 | SG77 | SG559 | SG468 |
| TIA-2 | SG7 | SG73 | |
| TAGLN2 | SG24 | SG476 | |
| TCF3 | SG277 | SG279 | |
| XRCC5 | SG32 | SG330 | |
| ZYX | SG97 | SG402 | |
| CD44 | SG228 | SG229 | |
| ACTB | SG463 | SG464 | |
| GAPD | SG464 | SG467 | |

### Establishment of Control Probes

To decrease the variability, a large number of controls were included in each microarray. These controls suppose an objective measurement on the process quality, and therefore, of the quality of the data obtained. They are of several types and origins:

a) Probes Used as Integrity Controls

These probes were 2 pairs of oligonucleotides complementary to ends 5′ and 3′ of the β-actin genes (probes code SG463 and SG464) and glyceraldehyde-3-phosphate dehydrogenase (probes code SG466 and SG467). The ratio between the intensities of the probe located at end 3′ and 5′ makes it possible to check the quality of the starting RNA and the functioning of the labelling reaction. The details on these oligonucleotides appear in Table 3.

TABLE 3
Oligonucleotides used as integrity controls

| Oligo- | | | Gene | GenID |
|---|---|---|---|---|
| nucleotide | SEQ ID NO: | Source gene | Abbreviation | No. |
| SG463 | SEQ ID | β-actin | ACTB | 60 |
| | NO: 463 | | | |
| SG464 | SEQ ID | β-actin | ACTB | 60 |
| | NO: 464 | | | |
| SG466 | SEQ ID | Glyceraldehyde-3- | GAPD | 2597 |
| | | phosphate | | |
| | NO: 466 | dehydrogenase | | |
| SG467 | SEQ ID | Glyceraldehyde-3- | GAPD | 2597 |
| | | phosphate | | |
| | NO: 467 | dehydrogenase | | |

### b) Probes Used as Negative Controls

These probes are largely formed by a group of oligonucleotides of 50 nucleotides (50-mer) which are not complementary to any known human sequence. For them, the BLAST tool was applied to these probes and it was observed that they did not hybridize with any human sequence. They are identified with codes SC1 (SEQ ID NO:564), SC2 (SEQ ID NO:565), SC3 (SEQ ID NO:566), SC4 (SEQ ID NO:567), SC5 (SEQ ID NO:568), SC6 (SEQ ID NO:569) and SC7 (SEQ ID NO:570) and oligonucleotides SCN1 (SEQ ID NO:571), SCN5 (SEQ ID NO:575), SCN7 (SEQ ID NO:577) and SCN10 (SEQ ID NO:580) are also used as negative controls. They are used to determine the optimum conditions of hybridization, washing and developing of the chips or microarrays. The appearance of a signal associated to them indicates the existence of non-specific hybridization.

c) Exogenous Probes Used as Internal Positive Controls: “Spiked Controls”

“Spiked controls” are synthetic oligonucleotides whose sequence coincides with a fragment of a transcript of a non-human gene or of any other sequence of nucleotides of low homology with transcripts of human genes which is polyadenylated at 3′, which is used as positive control, in the determination of the process quality, in the normalization of data and for the establishment of the linear range of the process (Benes V et al., 2003). To do this, the transcripts or corresponding polyadenylated sequences are added to the total starting RNA before starting the labelling process, and therefore, they suffer the same reactions (labelling, hybridization and developing) as the total

RNA of the samples.

7 “Spiked controls” are used. To ensure low homology with human genes 5 transcripts of Bacillus subtilis genes (dap, thr, trp, phe and lys) and 2 transcripts of genes of the Sharkav virus are used, frequently referred to as “Plum poxvirus” (Sppv), which is a plant virus. The details on these oligonucleotides are shown below in Table 4. The ATCC (American Type Culture Collection) numbers which appear after the name of the source genes refer to the identification number in the ATCC of E. coli strains containing recombinant plasmids which contain the sequence of the genes from which the transcripts added to the RNA are obtained and which were also used for the design of the sequences of the corresponding oligonucleotides bound to the microarray.

TABLE 4
Oligonucleotides used as “Spiked Controls”

| | | | | | Concentration |
|---|---|---|---|---|---|
| | | | | Transcript | (pM) in the |
| Oligo- | | | Gene Bank | size | “spiked controls” |
| nucleotides | SEQ ID NO: | Source gene | code | (nt) | solution |
| SSPC1 | SEQ ID | Dap | L38424 | 1820 | 2000 |
| | NO: 584 | (ATCC no. 87486) | | | |
| SSPC2 | SEQ ID | Lys | X17013 | 1000 | 1250 |
| | NO: 585 | (ATCC no. 87482) | | | |
| SSPC3 | SEQ ID | Thr | X04603 | 1980 | 5 |
| | NO: 586 | (ATCC no. 87484) | | | |
| SSPC4 | SEQ ID | Plum pox virus, | AF401296 | | 100 |
| | NO: 587 | isolated PENN2 (Sppv1) | | | |
| SSPC5 | SEQ ID | Plum pox potyvirus, | X57975 | | 750 |
| | NO: 588 | mRNA coated protein (Sppv2) | | | |
| SSPC6 | SEQ ID | Phe | M24537 | 1320 | 1000 |
| | NO: 589 | (ATCC no. 87483) | | | |
| SSPC7 | SEQ ID | Trp | K01391 | 2500 | 500 |
| | NO: 590 | (ATCC no. 87485) | | | |

c. 1.: Preparation of the 5 “Spiked controls” of Bacillus subtilis

The E. coli bacteria with the recombinant plasmids were acquired from ATCC (Rockville, Md. USA) The plasmids (pBluescript II-KS) contained the cloned cDNA of a Bacillus subtilis gene, with cut-off sites for the NotI enzymes at end 5′ and BamHI at end 3′ and a poly extension (dA) prior to the cut-off site for BamHI.

After reconstituting and allowing the cells to grow during the night at 37° C. in LB+Ampicillin medium, the plasmid was obtained with the Midipreps kit (Jetstar) following the manufacturer's recommendations. 10 μg of each one of the plasmids was linearized by digestion with 30 U of NotI restriction enzyme, in the presence of 1XNE3 and 1XBSA buffer during 3 hours at 37° C. The linearized plasmids were subjected to extraction with phenol:chloroform:isoamilic alcohol (Ambion), precipitation with 0.1 vol of 3M sodium acetate (Sigma) and 2.5 vol of 100% Ethanol and elimination of salts with 80% Ethanol, following the aforementioned protocol. The DNA obtained was resuspended in 10 μl of RNase-free water.

Next, the transcripts with sense were synthesized with an in vitro transcription reaction (I.V.T) from 1 μg of plasmid linearized using the MegaScript T3 kit (Ambion) and following the manufacturer's recommendations. The plasmids obtained were purified with the RNeasy Total RNA Isolation Kit (QIAGEN), following the manufacturer's recommendations.

The quantification, determination of the purity, quality and size of the transcripts obtained were performed following the same methods which are described below for the total RNA.

c.2. Preparation of the 2 “Spiked Controls” which Represent SPPV Genes

The recombinant plasmids (Progenika Biopharma) contained the cloned

cDNA of the two sppvl and sspv2 genes inserted between two PvuII and PstI restriction sites. End 3′ of each insert contains a polyadenylation extension.

JM109 cells were transformed with the plasmids which contained the transcripts. The cells were left to grow in plates with LB+Ampicillin medium at 37° C., the colonies with the transferred cells were selected and they were grown in LB+AMP liquid medium.

The recovery of the plasmids was performed with the Midipreps Plasmid Purification kit (Qiagen), following the manufacturer's recommendations. 10 μg of each plasmid was linearized with 30 U of the PvuII restriction enzyme. The insert was extracted with phenol:chloroform:isoamilic alcohol (Ambion), precipitation with 0.1 volumes of 7.5 M sodium acetate and 2.5 volumes of 100% ethanol. The salts were eliminated by two washings with 80% ethanol. The DNA obtained was resuspended in 10 μl of Rnase-free water.

Next, the transcripts with sense were synthesized with 1 μg of plasmid linearized using the T7 MegaScript kit (Ambion) and following the manufacturer's recommendations. The product of the reaction was cleaned with the RNeasy Total RNA Isolation Kit (Qiagen).

The quantification, measuring of the purity of the transcripts obtained and verification of their size were then performed

A solution of “Spiked controls” was prepared from the transcripts obtained with different concentrations of each one of those “spiked” (see Table 3), so that they covered the whole range of intensities of the “scanner” reader system (values of intensity which go from 0 to 65,535 in arbitrary units). This solution was added in the same quantity to 5□ μg of total starting RNA from each sample before starting the process.

c.3. Design of Probes Representative of Each One of the Transcripts:

So that the behaviour of the probes was as similar as possible to the probes designed for the genes to be studied, with the Oligo 6.0 programme (M.B.I), those sequences were selected for each “Spiked control” which complied with the same requirements established for the probes of the genes represented (length, GC content, “sense” strand and distance to end 3′) and which did not form stable loops (energy less than −7 Kcal/mol). The BLAST tool was applied to the sequences which complied with those requirements and that with less homology with human sequences was chosen.

After depositing and immobilizing the probes corresponding to the “Spiked controls” on the glass, it was verified: a) that the probes did not hybridrize in non-specific manner with the samples to analyse, b) that all the probes had similar hybridization characteristics, and c) that the signal of intensity obtained from each one of them can be related to the quantity of transcript added to the RNA.

d) Hybridization Controls

Snthetic oligonucleotides of DNA with 70 nucleotides (70-mer) were used As hybridization controls, modified at one end with a biotin molecule. These molecules are added in the same quantity to the sample just before hybridization, so that their value only depends on the processes of hybridization, developing and capture of images of the microarray. For each one of these 70-mer oligonucleotides, on the microarray there are several copies of an oligonucleotide with 50 nucleotides in length (50-mer), complementary to the corresponding 70-mer oligonucleotide with which it must hybridize. The 50-mer oligonucleotides which form part of the microarray and which are complementary to 70-mer oligonucleotides which are added to the cRNA before hybridizing are of codes SCN2, SCN3, SCN6, SCN8, SCN11, SCN12 and

SCN13. To ensure low homology with human sequences, the sequences of these oligonucleotides were obtained from sequences of Arabidopsis thaliana and Tripanosoma brucei. Their characteristics appear in Table 5

TABLE 5
Oligonucleotides used in the microarray as positive hybridization controls

| 50-mer | | | | |
|---|---|---|---|---|
| oligonucleotide | | | | Complementary |
| present in | | | GenBank | 70-mer |
| the microarray | SEQ ID NO: | Source gene | code | oligonucleotide |
| SCN2 | SEQ ID NO: 572 | Alpha-1.4- | AY026941 | C2 |
| | | fucosyltransferase (FT4-M) | | |
| | | from Arabidopsis thaliana | | |
| SCN3 | SEQ ID NO: 573 | mRNA of the | AJ239128 | C3 |
| | | thioredoxine of | | |
| | | Tripanosoma brucei | | |
| SCN6 | SEQ ID NO: 576 | mRNA from a supposed | AY051079 | C6 |
| | | expression protein of the | | |
| | | RBP (complete CDS) | | |
| | | from Arabidopsis thaliana | | |
| SCN8 | SEQ ID NO: 578 | mRNA from a supposed | AY045879 | C8 |
| | | transfer protein of lipids | | |
| | | (At1g48750) (complete | | |
| | | CDS) from Arabidopsis thaliana | | |
| SCN11 | SEQ ID NO: 581 | mRNA from a supposed | AY045879 | C11 |
| | | transfer protein of lipids | | |
| | | (At1g48750) (complete | | |
| | | CDS) from Arabidopsis thaliana | | |
| SCN12 | SEQ ID NO: 582 | mRNA from a supposed | AY045879 | C12 |
| | | transfer protein of lipids | | |
| | | (At1g48750) (complete | | |
| | | CDS) from Arabidopsis thaliana | | |
| SCN13 | SEQ ID NO: 583 | mRNA of the papain- | AF191028 | C13 |
| | | type cysteine endopeptidase | | |
| | | XCP2 (complete CDS) | | |
| | | from Arabidopsis thaliana | | |

For the design of the 50-mer oligonucleotides it was verified, in a manner similar to that previously described for the “Spiked controls”, that the oligonucleotides to be used did not hybridize in non-specific form with the samples to be analysed, that all the probes had similar hybridization characteristics and that the signal of intensity obtained from each one of them could be related to the quantity of the corresponding 70-mer oligonucleotide added to the cRNA. This made it possible to take as valid the oligonucleotides indicated in Table 5. The SCN4 (SEQ ID NO:574) and SCN9 (SEQ ID NO:579) oligonucleotides, designed in principle to act as hybridization controls, were seen to produce specific hybridization when human cRNA hybridized, for which reason they also appear in the microarray, as if they were probes which represent a human gene, but they are not taken into account as positive hybridization controls. For their part, oligonucleotides SCN1 (SEQ ID NO:571), SCN5 (SEQ ID NO:575), SCN7 (SEQ ID NO:577) and SCN10 (SEQ ID NO:580), which did not hybridize either in non-specific form with the samples, are also present in the microarray as negative hybridization controls, as no oligonucleotide complementary thereto were added to the cRNA.

For its part, the hybridization controls solution, which contained the 70-mer oligonucleotides complementary to the 50-mer oligonucleotides present in the microarray as positive hybridization controls, was prepared from the corresponding biotinylated 70-mer sequences using a different concentration for each one of them, as shown in Table 6:

TABLE 6
Composition of the positive hybridization controls solution

| 70-mer | | | |
|---|---|---|---|
| Oligonucleotide | | Complementary | Concentration (pM) |
| added to the | | 50-mer | in the hybridization |
| cRNA | SEQ ID NO: | oligonucleotide | control solution |
| C2 | SEQ ID NO: 591 | SCN2 | 750 |
| C3 | SEQ ID NO: 592 | SCN3 | 250 |
| C6 | SEQ ID NO: 593 | SCN6 | 1500 |
| C8 | SEQ ID NO: 594 | SCN8 | 1250 |
| C11 | SEQ ID NO: 595 | SCN11 | 2000 |
| C12 | SEQ ID NO. 596 | SCN12 | 4500 |
| C13 | SEQ ID NO: 597 | SCN13 | 2500 |

### Blanks

Dimethyl sulfoxide (DMSO) without any probe was used, as this is the solvent wherein the oligonucleotides are found at the time of being deposited on the surface of the microarray.

### Description of the Microarray Device

Twelve replicas of each probe were deposited in different localizations on the surface of a solid support (glass in similar form to a microscope slide) using Microgrid II Spoter (Biorobotics). The 12 replicas of each probe were distributed on the support at random: 6 in the upper area and 6 in the lower area. Aminosylanized glass (Corning) was used as solid support. The moisture and the temperature were controlled throughout the printing process.

The covalent binding of the probes to the solid supports was carried out by cross-linking by ultraviolet radiation using the “Stratalinker” apparatus (Stratagene).

The quality control of the production process of the microarrays was the following: a) In each production run a microarray was stained with ethydium bromide which made it possible to analyze the size and form of the points printed. b) Another array of each run was hybridized with an already hybridized cRNA, analysing the hybridization signal, the background noise and the reproducibility of the replicas.

The characteristics of the array are shown below in Table 7:

TABLE 7
Characteristics of the microarray

| | |
|---|---|
| Number of genes represented | 538 |
| Length of the oligonucleotides | 25-55 mer |
| Strand analysed | Sense |
| Number of oligonucleotides per gene | 1 (except 21 genes which |
| | are represented by 2 or 3 |
| | different oligonucleotides) |
| Number of replicas of each oligonucleotide | 12 |
| Blank | DMSO |
| Integrity controls | 4 |
| Spiked controls (internal positive controls) | 7 |
| Positive hybridization controls | 9 |
| Negative controls | 11 |
| Total number of points | 8192 (32 areas × 16 × 16) |
| Size of the microarray | 25 × 75 mm |
| Area spotted | 16.38 × 17.82 mm |
| Distance between points | x- y- axis 360 μm |

### Treatment of the Samples

### Cell Cultures

Cultures of Jurkat cells (cell line from Leukemia T) and U937 (cell line from promonocytic leukemia) were centrifuged for 10 minutes at 1200 rpm and, after decanting the supernatant, the precipitate was resuspended in RNAlater (Ambion Inc) and it was stored at −80° C. at the time of extraction of the RNA. The RNA was extracted with TRIzol (Gibco-BRL Carlbad, Calif., USA) following the manufacturer's recommendations.

### Blood Samples

The blood samples were directly collected in PAXgene Blood RNA Tubes-PreAnalytix (Qiagen) tubes. 2.5 ml of blood were extracted in each tube and two tubes per individual. The tubes were inverted several times to allow the blood to mix with the stabilizing liquid which the tube contains, and they were stored at −20° C. until the night before RNA extraction.

### Extraction of the Total RNA

The tubes with the sample were incubated at ambient temperature during the night previous to the RNA extraction. The PAXgene Blood RNA kit (Qiagen) was used for the extraction following the manufacturer's recommendations, including the intermediate step of treatment with DNase (RNase-Free DNase Set, Quiagen) in column. The RNA of each extraction tube was eluted in 80 μl of BR5 buffer. The RNA of the two tubes which correspond to each patient was gathered in a single tube.

### Purification of the Total RNA

To ensure that the RNA obtained is free from free from contaminants that can interfere in later labelling reactions, it was purified in the following way: 16 μl (0.1 vol) of 7.5 M sodium acetate (Sigma) and 400 μl (2.5 vol) of 100% ethanol were added to 160 μl of total RNA solution. The solution was mixed in a “vortex” stirrer and it was incubated for 1 hour at −20° C. After 20 minutes of centrifugation at 12,000×g at 4° C., the precipitate was washed twice with 500 μl of 80% ethanol and it was resuspended in 35 μl of Rnase-free water. The RNAs obtained were stored at −80° C. until their later use.

### Quantification of the Total RNA

The quantification of the total RNA was carried out by the measurement of the absorbance at 260 nm in a spectrophotometer (DU 65, Beckman Coulter). 2 μl of the total RNA solution were diluted in 98 μl of 1 mM Tris-HCl pH 7.5 and the concentration was estimated (μg/ml) taking into account that 1 Unit of Optical Density at 260 nm corresponds to a RNA concentration of 44 μg/ml.

### Determination of the Purity and Quality of the RNA

The degree of purity was established from the absorbance ratio A260/A280 (nucleic acid/proteins), considering that the RNA is suitable, of “good quality”, when the A260/A280 ratio is between 1.9 and 2.1.

The quality of the total RNA was determined by viewing the RNA after electrophoresis. 500 ng of total RNA were subjected to electrophoresis in 1% agarose gel (FMC) in TAE 1× buffer with BrEt (0.5 mg/ml), under a potential differenceof 100V for 25 minutes in AC electrophoresis cuvettes (BioRad). As marker of molecular weights, phage φ29 digested with the BamH I restriction enzyme was used. The gels were viewed in a Gel Doc (BioRad) ultraviolet light transiluminator.

### Sample Labelling

The choice of the strand with sense as probe limited the labelling strategy at those approximations which yield an antisense labelled product (complementary to the probe immobilized on the solid support).

cRNA Labelling

This type of labelling was performed during the course of an amplification process which consists of the use for the synthesis of single-strand cDNA, of an oligo(dT) primer which contains a promoter for the polymerase RNA enzyme of the T7 phage, an enzyme which will be used in the sample amplifications step.

a.—cDNA synthesis: step wherein DNA (cDNA) complementary to the starting mRNA was synthesized. 5 μg of total RNA was incubated with 2 μl of the “Spiked controls” solution and 100 pmol of T7-(dT)24 (Genset Corp) primer in final volume of 12 μl during 10 minutes at 70° C. in a thermoblock, the mixture was cooled on ice and 4 μl of 5× First Strand Buffer (Gibco BRL Life Technologies), 0.1M 2 μl DTT (Gibco BRL Life Technologies), 1 μl dNTP mix 10 mM (Gibco BRL Life Technologies) and 1 μl of SuperScript II RNase H RT (200 OR/μl) (Gibco BRL Life Technologies) were added. After 1 hour of incubation in a bath equipped with a thermostat (Selecta) at 42° C., the reaction was cooled on ice.

b.—Double chain DNA synthesis (dsDNA): a double chain of DNA was synthesized from the cDNA synthesized in the previous step. To 20 μl of previous reaction were added 91 μl of Rnase-free water, 30 μl of “Second Strand Reaction buffer” (Gibco BRL Life Technologies), 3 μl 10 mM dNTPs (Gibco BRL Life Technologies), 10 U E. coli DNA Ligase (Gibco BRL Life Technologies), 40 O E. coli DNA polymerase I (Gibco BRL Life Technologies), 2 U E. coli RNase H (Gibco BRL Life Technologies) in a final volume of 150 μl. The reaction was incubated in a thermoblock at 16° C. for 2 hours. Next, 10 U of T4 DNA Polymerase (Gibco BRL Life Technologies) were added and the mixture was incubated at 16° C. for 5 minutes. To stop the reaction, 10 μl of 0.5 M EDTA were added.

c.—Purification of the dsDNA: To eliminate possible remains of reaction products which may interfere in later labelling reactions, the DNA obtained through phenol/chloroform extraction and later precipitation was purified. To 162 μl of previous reaction 162 μl of phenol: chloroform: isoamilic alcohol solution (25:24:1) (Ambion) were added. It was centrifuged for 2 min at 12,000×g in a centrifuge at ambient temperature, the upper aqueous phase was collected. To this upper phase 0.5 volumes of 7.5M ammonium acetate (Sigma Chemical) and 2.5 volumes of 100% ethanol cooled to −20° C.) were added. After stirring with “vortex” to mix well the components and centrifugation for 20 minutes at 12000×g at ambient temperature, the supernatant was eliminated and the precipitate was washed twice with 80% ethanol. The DNA obtained was resuspended in 10 μl of RNase-free water and it was concentrated in a “Speed-Vac” concentrator to a volume of 2 μl. This DNase was stored at −20° C. until its later use.

d.—Synthesis and labelling of the cRNA: This reaction was carried out in a volume of 20 μl and using the T7 Megascript kit (Ambion), following the manufacturer's instructions and incorporating nucleotides modified with biotin, Bio-11-CTP and Bio-11 UTP (Perkin Elmer) in non-modified nucleotide/modified nucleotide ratio of 1:3. The reaction was incubated during 5 h and 15 minutes in a bath with thermostat (Selecta) at 37° C., stirring the reaction every 45 minutes. After this incubation, 1 μl of DNase was added and it was incubated for 30 min at 37° C.

e.—Purification of the biotinylated cRNA: The biotinylated cRNA was purified with the RNeasy Total RNA Isolation Kit (Qiagen) following the manufacturer's instructions. The biotinylated cRNAs obtained were eluted in a volume of 80 μl and they were stored at −80° C. until its later use.

The quantity, purity and quality of the cRNA obtained were determined following the same methods described for the total RNA.

The cRNA was stored at −80° C. until its later use.

Fragmentation of the Biotinylated cRNA

10 μg of biotinylated cRNA were fragmented in the presence of 5× (200 mM Tris-acetate, pH 8.1, 500 mM HOAC, 150 mM MgOAc) fragmentation buffer during 35 minutes at 94° C. in a thermoblock. It was verified that the fragmentation reaction had been carried out by viewing 1 μl of fragmentation solution in electrophoresis on 1% agarose gel.

Hybridization of the cRNA Labelled with the Probes of the Microarray

In this step the labelled genetic material were placed in contact with the probes immobilized on the solid support.

10 μl of the hybridization control solution were added to the biotinylated and fragmented cRNA solution and the mixture was incubated for 3 min at 95° C. to denature the possible secondary structures. After incubation, the mixture was immediately taken to ice to prevent the possible renaturing of the sample.

The hybridization was carried out for 6 hours at 42° C. in the Ventana Discovery automatic hybridization station (Ventana Medical Systems). The hybridization and washing buffers were supplied by Ventana Medical System. The microarrays were automatically stained in the hybridization station with streptavidin conjugated with Cy3 (Amersham Biosciences) using the manufacturer's recommendations.

### Capture of Images and Quantification of the Microarrays

After the hybridization and developing, the images of the microarrays were identified and analysed by the ScanArray 4000 confocal fluorescent scanner (Perkin Elmer) equipped with a laser for the green (543 nm to excite the fluorophore Cy3). The “software” used was ScanArray 3.1. The use of the computer programme QuantArray 3.0 (Perkin Elmer) provided the absolute values of the intensity of hybridization and background noise in accordance with the light emitted by the Cy3 in each probe in an Excel format.

### Data Analysis: Preliminary Processing

In first place, the value of the background noise were subtracted from the values of absolute intensity of all the oligonucleotides. To do this, the values of absolute intensity and the values of background noise, which the programme used to convert the signals of the fluorophore returns, automatically, were used for each one of the microarray points: the corresponding in tensity value is obtained from the zone which has been defined as point and the value of the background noise is obtained from the zone situated around the point.

Next, the average level of hybridization intensity of each one of the oligonucleotides of the microarray was calculated from the trimmed mean of the intensities of the 12 replicas of each one of the oligonucleotides. To do this, before calculating the average, the upper and lower values of the distribution points of hybridization signals obtained with each one of the replicas of the same oligonucleotide have to be eliminated. The calculation was performed using the Excel programme from Microsoft and, specifically, the TRIMMEAN function thereof, wherein the “percentage” parameter was set at 0.2, which supposes fixing the percentage of values eliminated in 20% of the upper values and 20% of the lower values; the function rounds up the number of data points excluded to the closest multiple of 2.

In last place, and to be able to determined the validity of the hybridization, it is necessary that a series of established criteria are met: 1) the ratio between the average intensity and the aver age background of all the oligonucleotides of the chip is greater than 10; 2) the value of the average coefficient of variation (standard deviation of the replicas compared with the average of the replicas) of all the replicas of oligonucleotides of the chip should be less than 0.3; 3) the average value of the negative control should be less than 2.5 times the value of the DMSO medium; 4) a signal should be obtained both in the hybridization controls and in the exogenous internal positive controls (Spiked controls).

The data analysis was performed in R, version 1.9.1. R is a programming language wherein both classical and modern statistical techniques can be applied (R Developmental Core Team, 2004; http://www.R-project.org), which has a series of functions stored in packages for the handling, calculation and graphic representation of data (Venables et al., 2004). There are hundreds of packages written by different authors for R, with special statistical functions or which permit the access and handling of data and are available for downloading from the websites of CRAN (http://cran.r-project.org/) or Bioconductor (http://www.bioconductor.org). In some specific cases, the SPSS commercial statistical analysis software was used (Chicago, USA).

### EXAMPLES

### Example 1

### Results Obtained on Using the Microarray Device with Samples of U937 Vs Jurkat Cells

In order to know if the device permits differentiating two cells lines hybridized in 10 microchips: 5 samples of biotinylated cRNAs synthesized following the optimized working protocol, obtained from RNA of U937 cells (cell line from promonocytic leukemia) and 5 samples of biotinylated cRNAs obtained from RNA of Jurkat cells (cell line from T Leukemia).

The initial steps of preliminary processing of the data and validation of the hybridization mentioned previously in the “Data analysis: Preliminary processing” section were carried out and then the data was normalized and filtered:
Data normalization. The “variance stabilization normalization” method was used, available in the “vsn” package in R. There are different packages available on the Internet for R, with special statistical functions or which permit the access and processing of data and are available for downloading from CRAN (http://cran.r-project.orq/) or Bioconductor (http://www.bioconductor.orq)
ata filtering. Two filtering operations have been carried out with the “Filterfun” function of the of the “Genefilter” filter in R. The genes which did not pass any of the two filters were not used in the data analysis. The filters carried out were:
   Filtering to exclude genes with an intensity value close to the DMSO. This filter made it possible to work with genes with an intensity value minus average background noise greater than 550 arbitrary units (approximately 2 times the value of the DMSO).
   Filtering to exclude genes with minimum intensity variation throughout the samples. Genes were worked with an interquartile range of normalized intensity throughout samples greater than 0.3.

The data filtering left 83 probes which constituted the working list. With them a grouping was made of the non-supervised samples, which are those groupings wherein the structure of the data is not previously known, the system learning how the data are distributed among classes based on a distance function. A tree or hierarchical group was obtained with the grouping, wherein the samples are grouped in accordance with their similarity in the expression of certain genes, those corresponding to the oligonucleotides of the working list, so that the closest samples are those which have a similar expression profile. The grouping was performed with the hclust function of the stats package in R. The non-supervised analysis of the 10 samples produced their separation in two groups or main branches in accordance with the cell type whereto the samples belong: a group contains the 5 hybridizations carried out from U937 cells and the other group contains the 5 hybridizations carried out from Jurkat cells. The resulting tree of this non-supervised grouping is shown in part A of FIG. 1.

Next, to find out if there were statistically significant differences between the two groups of samples, the “Step-down maxT multiple testing methods” method (maxT) was used, which is an application of the mt.maxT function of the multtest package of the software in R from Bioconductor, which applies a statistical test and carries out a strong control over the rate of false positives. To this function, the following should be provided:

a) Values on which one wants to apply the statistical tests, in this case, on the normalized values of the 83 oligonucleotides which passed the filters

b) Groups of which one wants to seek differences, in this case the 5 samples of Jurkat cells against the 5 samples of cells U937

c) Number of permutations one wants to perform. In this case, 100,000 permutations are carried out.

d) By default, Welch's test was chosen to specify the statistical tool to be used to test the hypothesis of non-association between the variables and the class labels.

The application of this analysis with a value of p<0.001 provided a list of 69 statistically significant probes between the two groups, which are the following:
SG12, SG20, SG23, SG24, SG38, SG39, SG45, SG49, SG53, SG59, SG60, SG62, SG76, SG78, SG89, SG92, SG94, SG102, SG474, SG478, SG487, SG114, SG120, SG140, SG142, SG145, SG150, SG154, SG158, SG174, SG175, SG194, SG195, SG211, SG230, SG231, SG235, SG260, SG264,
SG266, SG268, SG270, SG272, SG282, SG294, SG308, SG311, SG330, SG332,
SG333, SG339, SG344, SG364, SG403, SG423, SG434, SG456, SG506, SG513, SG514, SG515, SG524, SG533, SG538, SG541, SG559

Once the statistically significant genes to distinguish between the two groups of samples are known (which would be the genes corresponding to the probes identified as statistically significant) the supervised grouping was carried out of the samples in accordance with the intensity of the signal of the 69 statistically significant probes obtained. The term “supervised”, applied to a grouping, makes reference to the fact that the data structure is previously known, which makes it possible to use the prior information; with this, after a training process which allows the system to learn to distinguish between classes, it is possible to use the network to assign new members to the predefined classes. In this case, the supervised grouping of the samples in accordance with the intensity of the signal obtained with the 69 statistically significant probes obtained, is again a tree which is divided in two main branches in accordance with the cell type to which the samples belong. The tree obtained with the supervised grouping is shown in part B of FIG. 1.

### Example 2

### Results Obtained on Using the “Array” Device with Samples from Healthy Subjects Vs U937 and Jurkat Cells

The expression of 5 samples of U937 cells and 5 samples of Jurkat cells was compared with the expression of 10 samples from total blood from healthy subjects. In a manner similar to that carried out in Example 1, the initial data processing steps, validation of the hybridizations, normalization and filtering were carried out. A total of 180 genes passed the filtering processes. The non-supervised grouping of the samples (carried out with the hclust function of the stats package of R applying Pearson's correlation) in accordance with the expression of the 180 genes, provided a tree with two main branches: one branch contains all the samples from cell cultures and the other branch contains all the samples from total blood from healthy subjects, which demonstrates that the tool is capable of finding expression differences. The tree obtained after making this non-supervised grouping is shown in part A of FIG. 2.

The maxT test (p<0.001) to find genes with statistically significant differences between the samples from U937 and Jurkart cell cultures and the 10 samples from total blood of healthy subjects was performed. The statistical analysis provided a list of 131 probes with statistically significant differences between both groups of samples. They are the following:

SG1, SG4, SG7, SG8, SG10, SG13, SG15, SG16, SG17, SG18, SG19, SG20, SG26, SG29, SG30, SG34, SG36, SG39, SG42, SG44, SG49, SG51, SG52, SG58, SG64, SG65, SG67, SG76, SG77, SG80, SG84, SG86, SG89, SG92, SG93, SG94, SG98, SG99, SG101, SG102, SG107, SG463, SG464, SG474, SG475, SG485, SG487, SG466, SG467, SG471, SG472, SG473, SG120, SG129, SG138, SG141, SG144, SG145, SG147, SG158, SG163, SG164, SG176, SG185, SG186, SG197, SG207, SG208, SG217, SG227, SG231, SG265, SG266, SG277, SG278, SG283, SG285, SG299, SG307, SG308, SG311, SG313, SG318, SG319, SG328, SG333, SG336, SG342, SG344, SG357, SG361, SG376, SG384, SG389, SG395, SG398, SG403, SG404, SG407, SG416, SG420, SG423, SG430, SG436, SG446, SG455, SG461, SG489, SG491, SG492, SG493, SG498, SG500, SG504, SG505, SG506, SG514, SG516, SG517, SG520, SG526, SG530, SG533, SG538, SG545, SG547, SG554, SG555, SG558.

The grouping of the 20 samples, in accordance with the expression of the statistically significant probes found, gave rise again to a tree with two main branches, one corresponding to the samples from cell cultures and another corresponding to the samples from healthy individuals. Said grouping appears in part B of FIG. 2.

### Example 3

### Results Obtained with Samples from Patients with Chronic Lymphatic Leukemia (CLL) Vs U937 and Jurkat Cells

The expression profiles were compared of samples from U937 and Jurkats cell cultures with 26 samples from total blood of subjects with CLL.

The samples underwent preliminary processing of the data, they were normalized and filtered in a manner analogous to those used in Examples 1 and 2 and a total of 236 probes passed through the filters. The non-supervised grouping of the samples in accordance with the expression of the probes which passed through the filters showed a tree with two main branches: one which contained the samples of cell cultures and the other the CLL samples. Said tree is shown in part A of FIG. 3.

The maxT test (p<0.001) to find genes with statistically significant differences between the two groups of samples was carried out. This analysis provided a list of 120 probes. They are the following: SG2, SG4, SG8, SG10, SG13, SG15, SG16, SG19, SG20, SG23, SG26, SG28, SG31, SG34, SG36, SG39, SG48, SG58, SG60, SG65, SG76, SG77, SG84, SG89, SG94, SG9, SG97, SG99, SG102, SG106, SG107, SG463, SG464, SG474, SG475, SG481, SG465, SG485, SG487, SG466, SG467, SG471, SG473, SG115, SG116, SG117, SG120, SG129, SG134, SG135, SG138, SG139, SG141, SG145, SG158, SG161, SG163, SG176, SG178, SG185, SG207, SG208, SG210, SG217, SG227, SG231, SG237, SG264, SG272, SG277, SG281, SG283, SG286, SG294, SG298, SG299, SG307, SG308, SG319, SG328, SG330, SG333, SG336, SG342, SG344, SG345, SG347, SG361, SG384, SG389, SG395, SG404, SG407, SG416, SG423, SG428, SG430, SG432, SG434, SG444, SG446, SG453, SG458, SG459, SG491, SG498, SG507, SG508, SG511, SG517, SG518, SG522, SG526, SG530, SG533, SG538, SG541, SG554, SG558, SG561.

The grouping of the 30 samples in accordance with the expression of the 120 statistically significant probes found again gave rise to a tree with two main branches, one corresponding to the samples from cell cultures and another corresponding to the samples from healthy individuals. Said grouping appears in part B of FIG. 3.

### Example 4

### Results Obtained with Samples from Healthy Subjects Vs Patients with Chronic Lymphatic Leukemia (CLL)

68 hybridizations which met the quality criteria from 68 samples of different healthy subjects and with clinical diagnosis of CLL were divided in 2 groups: Training Group used to obtain the functions of the classifier and Test Group, used to test the classifier obtained. The Training group was composed of 30 samples (10 from healthy subjects and 20 from CLL subjects) and the Test Group was composed of 38 samples (5 samples from healthy subjects and 33 samples from subjects with CLL).

To obtain the classification function, the results obtained from the hybridizations of the Training group were worked with. The steps carried out to obtain the classification function were:
Data normalization. The “variance stabilization normalization” method, available in the “vsn” package in R, was used.
Data filtering. Two filtering operations have been carried out with the “Filterfun” function of thee “Genefilter” package in R. The genes which did not pass any of the two filters were not used in the data analysis. From the 588 oligonucleotides of the chip, 224 passed through the 2 filters and constituted the working list.
2. Filtering to exclude genes with an intensity value close to the DMSO. This filter made it possible to work with genes with an intensity value minus average background noise greater than 550 arbitrary units (approximately 2 times the value of the DMSO) in more than 25% of the samples (7 samples) which compose the Training group.
3. Filtering to exclude genes with minimum intensity variation throughout the samples. Genes were worked with which had an interquartile range of normalized intensity throughout samples greater than 0.3.

Two classification systems are used:

4.1.—Construction of a Classification System with PAM.

To identify groups of genes which best characterize each type of sample and verify the classification rate of these groups of genes Prediction Analysis for Microarrays (PAM) was used, available as “pamr^{a}” package in R. It is a statistical technique which identifies a group of genes which best characterizes a predefined class and uses this group of genes to predict the class whereto new samples belong. PAM uses a modified version of the “nearest centroids” classification method (Tibshirani et al., 2002) called “Nearest Shrunken Centroids”. A validation called “10 fold cross validation” was performed, which consists of constructing the model with 90% of the samples and an attempt is made to predict the class of 10% of the samples which have not intervened in the construction of the model. This method is repeated 10 times and the classification error of 10% of the samples is added to calculate the overall error. This error reflects the number of badly classified samples (Bullinger et al., 2005).

4.1.1. Construction of the model. From the filtered and normalized data of the 30 samples which compose the Training group, attributing in an arbitrary form the Healthy Group to group 0 and the CLL Group to group 1, performing the 10 cross-validations and with a threshold value of Delta 3.1. The model obtained was formed by the following oligonucleotides: SG459, SG428, SG507, SG508, SG117, SG237. The coefficients of the classifier corresponding to each one of these oligonucleotides are shown below in Table 8:

TABLE 8
Coefficients of the PAM classifier

| id | Value 0 | Value 1 |
|---|---|---|
| [1,] SG459 | −0.4344 | 0.2172 |
| [2,] SG428 | −0.146 | 0.073 |
| [3,] SG507 | −0.1111 | 0.0555 |
| [4,] SG508 | −0.1044 | 0.0522 |
| [5,] SG117 | −0.1003 | 0.0502 |
| [6,] SG237 | −0.0539 | 0.027 |

4.1.2. Validation of the PAM classifier. The cross-validation of the samples which compose the Training group correctly classified 28 of the 30 samples.

From the filtered and normalized data of the 38 samples which compose the Test Group, probability values p were obtained belonging to group 0 (healthy group) or group 1 (CLL group). The greater the value of p, the greater the probability of belonging to that group. It has been considered that the values greater than 0.5 indicate belonging to that group. The values of p obtained for each sample are indicated in Table 9.

TABLE 9
Probability values obtained with the PAM classifier for the Test Group

| Sample | p (Group 0) | p (Group 1) | |
|---|---|---|---|
| S229 | 0.8031905 | 0.1968095 | |
| S231 | 0.7403173 | 0.2596827 | |
| S232 | 0.8810574 | 0.1189426 | |
| S233 | 0.7973159 | 0.2026841 | |
| S251 | 0.8714224 | 0.1285776 | |
| CLL166 | 0.3764637 | 0.6235363 | |
| CLL184 | 0.1278230 | 0.8721770 | |
| CLL132 | 0.2081423 | 0.7918577 | |
| CLL210 | 0.3248082 | 0.6751918 | |
| CLL213 | 0.3536033 | 0.6463967 | |
| CLL214 | 0.2705277 | 0.7294723 | |
| CLL221 | 0.3650277 | 0.6349723 | |
| CLL208 | 0.2323872 | 0.7676128 | |
| CLL225 | 0.4034316 | 0.5965684 | |
| CLL236 | 0.4893545 | 0.5106455 | |
| CLL240 | 0.3807527 | 0.6192473 | |
| CLL168 | 0.1616066 | 0.8383934 | |
| CLL172 | 0.2002317 | 0.7997683 | |
| CLL174 | 0.1601147 | 0.8398853 | |
| CLL175 | 0.6009558 | 0.3990442: | →Only badly classified sample |
| CLL177 | 0.1634185 | 0.8365815 | |
| CLL179 | 0.2300440 | 0.7699560 | |
| CLL181 | 0.2177406 | 0.7822594 | |
| CLL182 | 0.3450880 | 0.6549120 | |
| CLL164 | 0.2590083 | 0.7409917 | |
| CLL159 | 0.3688586 | 0.6311414 | |
| CLL142R | 0.2111712 | 0.7888288 | |
| CLL105 | 0.2962797 | 0.7037203 | |
| CLL107 | 0.3764637 | 0.6235363 | |
| CLL109 | 0.3525788 | 0.6474212 | |
| CLL112 | 0.2059187 | 0.7940813 | |
| CLL151 | 0.2951067 | 0.7048933 | |
| CLL158 | 0.1932882 | 0.8067118 | |
| CLL169 | 0.3525937 | 0.6474063 | |
| CLL171 | 0.1495153 | 0.8504847 | |
| CLL178 | 0.2260191 | 0.7739809 | |
| CLL111 | 0.2951168 | 0.7048832 | |
| CLL155 | 0.2832151 | 0.7167849 | |

With this model 37 of the 38 samples of the Test Group are correctly classified: all the samples corresponding to healthy individuals (those whose name is headed by the letter “S”) have a probability greater than 0.5 of belonging to group 0, whilst all the samples corresponding to individuals suffering from CLL (which are the samples whose name starts with letters “CLL”) minus one have a probability greater than 0.5 of belonging to group 1.

4.2.—Construction of a Classification System with Logistical Regression.

4.2.1.—Selection of genes with statistically significant differences among healthy and CLL (Training group). From the filtered and normalized data as has been previously described, the “Step-down maxT multiple testing methods” method (maxT) was used for the selection of genes with significant differences, which is an application of the mt.maxT function of the multtest package of the software in R from Bioconductor, which applies a statistical test and carries out a strong control over the rate of false positives. The application of this statistical test, with a value of p<0.001, to the 224 oligonucleotides which passed through the filters, produced a list of 7 oligonucleotides: SG117, SG428, SG459, SG461, SG493, SG507, SG508.

The steps used to obtain the list of 7 significant genes among healthy and CLL were:
Method which makes permutations and adjusts the values of p resT<-mt.maxT(exprs(224 oligonucleotides which have passed through the filters and normalized of the training group, Types of samples in the training group, test=“t”, B=100000): mt.maxT function which through permutations adjusts the probability values (signification) which entails a strong control of the rate of false positives.

To this function the following should be provided:

1. Values on which one wants to apply the statistical tests, in this case, on the normalized values of the 83 oligonucleotides which passed through the filters
2. Groups of which one wants to seek differences, in this case the 5 samples of Jurkat cells against the 5 samples of cells U937
3. Number of permutations one wants to perform. In this case, 100,000 permutations are carried out.
4. By default, Welch's test was chosen as statistical test.

The statistically significant genes at a level of p<0.001 were selected by this test and a number of 7 was obtained.

4.2.2.—Obtainment of the classification function with SPSS. By logistical regression from the normalized values of the 7 statistically significant oligonucleotides obtained from the 30 samples which compose the Training group and assigning in arbitrary manner group 0 to the healthy samples and group 1 to the CLL samples, the values of the classification function were obtained. The coefficients corresponding to each oligonucleotide were those which are shown below in Table 10:

TABLE 10
Coefficients of the classification function
Calculated by logistical regression

| Oligonucleotide | Coefficients (Coeff) |
|---|---|
| SG117 | 2.44756372 |
| SG428 | 7.38657611 |
| SG459 | 23.1465464 |
| SG461 | 43.6287742 |
| SG493 | −19.3978182 |
| SG507 | −2.80282646 |
| SG508 | 49.5345672 |
| Constant | −719.241486 |

From these coefficients, for each sample i a value xᵢ is calculated as follows:

xᵢ=Constant+(Coeff ohle0009*ImnᵢSG117)+(Coeff SG428*ImnᵢSG428)+(Coeff SG459*Imnᵢ SG459)+(Coeff SG461*ImnᵢSG461)+(Coeff SG493*Imnᵢ SG493)+(Coeff SG507*ImnᵢSG507)+(Coeff SG508*ImnᵢSG508).

where Imnᵢ is the average value of normalized intensity of the sample i.

From the value xᵢ a value of probability (pᵢ) is calculated. The closer the value of p is to 0, the greater the probability of belonging to the group of healthy subjects (assigned as group 0) and the closer the value of p is to 1, the greater the probability there is of the sample belonging to the group of CLL subjects (assigned as group 1). The formula used to determine the value of p is:

pᵢ=1/(1+e^{-xi}).

As is shown in Table 11, the function obtained correctly classified the 30 samples belonging to the training group. The closer to 0, the greater the probability that it is healthy and the closer to 1 the greater probability of CLL.

TABLE 11
Classification table^{a}

| | | | Prognosis | | |
|---|---|---|---|---|---|
| | | | EVOL | | Correct |
| | Observed | | 0 | 1 | percentage |
| Step 1 | EVOL | 0 | 10 | 0 | 100.0 |
| | | 1 | 0 | 20 | 100.0 |
| | Overall percentage | | | | 100.0 |

^{a}The cut-off value is ,500

4.2.3. Validation of the system classifier.—From the filtered and normalized filters as detailed above, the Imnᵢ values were obtained of the 7 oligonucleotides which compose the classifier of each one of the 38 samples which compose the Test Group.

Results of the validation of the system classifier. Below, tables are shown wherein the Imnᵢ value is obtained of each one of the 7 oligonucleotides included in the classifier and the values of xᵢ and pᵢ calculated according to the formulas previously described, obtained for each one of the 38 samples of the Test Group. The samples which begin with S correspond to healthy subjects and the samples which start with CLL are from CLL subjects. 37 out of 38 samples are correctly classified. Only sample CLL175, for which a value of pi=0 is obtained is incorrectly classified.

TABLE 12
Results obtained with the Test Group by the classification function obtained by logistical regression

| lmnᵢ | S229 | S231 | S232 | S233 | S251 | CLL166 | CLL184 | CLL132 | CLL210 | CLL213 | CLL214 |
|---|---|---|---|---|---|---|---|---|---|---|---|
| SG117 | 4.89 | 5.17 | 5.14 | 5.33 | 5.37 | 6.17 | 7.45 | 7.05 | 7.06 | 6.88 | 6.86 |
| SG428 | 4.82 | 4.80 | 4.52 | 4.69 | 4.52 | 5.74 | 6.97 | 6.46 | 6.66 | 6.08 | 6.49 |
| SG459 | 6.50 | 6.95 | 6.10 | 6.59 | 5.96 | 8.11 | 9.04 | 8.71 | 8.13 | 8.15 | 8.40 |
| SG461 | 6.47 | 6.41 | 6.44 | 6.43 | 6.05 | 6.35 | 7.22 | 6.99 | 6.75 | 6.75 | 7.04 |
| SG493 | 7.20 | 7.31 | 7.02 | 7.16 | 6.83 | 7.53 | 7.69 | 7.35 | 7.43 | 7.35 | 7.57 |
| SG507 | 8.32 | 7.82 | 7.26 | 7.77 | 7.47 | 9.11 | 9.82 | 9.32 | 9.19 | 8.49 | 9.18 |
| SG508 | 6.67 | 6.82 | 6.34 | 6.82 | 6.81 | 7.79 | 8.71 | 7.80 | 7.55 | 7.66 | 7.68 |
| xᵢ | −71.35 | −56.83 | −93.68 | −61.26 | −86.57 | 17.23 | 129.51 | 70.11 | 33.93 | 38.26 | 55.19 |
| pᵢ | 0.00 | 0.00 | 0.00 | 0.00 | 0.00 | 1.00 | 1.00 | 1.00 | 1.00 | 1.00 | 1.00 |

| lmnᵢ | CLL221 | CLL208 | CLL225 | CLL236 | CLL240 | CLL168 | CLL172 | CLL174 | CLL175 | CLL177 |
|---|---|---|---|---|---|---|---|---|---|---|
| SG117 | 7.00 | 7.42 | 6.91 | 6.40 | 6.76 | 7.34 | 7.32 | 7.40 | 5.32 | 7.72 |
| SG428 | 6.40 | 6.98 | 6.00 | 6.24 | 6.09 | 6.77 | 6.16 | 7.49 | 4.85 | 6.38 |
| SG459 | 7.93 | 8.54 | 8.01 | 7.55 | 8.19 | 8.94 | 8.86 | 8.69 | 7.72 | 8.78 |
| SG461 | 6.77 | 7.18 | 6.89 | 6.79 | 6.54 | 6.72 | 7.02 | 7.18 | 5.98 | 6.86 |
| SG493 | 7.14 | 7.92 | 7.72 | 7.47 | 6.73 | 7.97 | 7.96 | 8.45 | 7.16 | 8.05 |
| SG507 | 9.00 | 8.98 | 8.41 | 8.74 | 8.80 | 9.53 | 9.67 | 10.09 | 8.76 | 9.76 |
| SG508 | 7.50 | 7.43 | 7.22 | 7.36 | 6.90 | 8.08 | 7.38 | 7.81 | 6.43 | 7.94 |
| xᵢ | 31.80 | 50.30 | 12.52 | 8.94 | 3.77 | 67.59 | 39.95 | 63.19 | −75.92 | 59.42 |
| pᵢ | 1.00 | 1.00 | 1.00 | 1.00 | 0.98 | 1.00 | 1.00 | 1.00 | 0.00 | 1.00 |

| lmnᵢ | CLL179 | CLL181 | CLL182 | CLL164 | CLL159 | CLL142R | CLL105 | CLL107 | CLL109 | CLL112 |
|---|---|---|---|---|---|---|---|---|---|---|
| SG117 | 6.89 | 6.92 | 6.14 | 8.03 | 6.98 | 6.80 | 6.52 | 6.17 | 6.79 | 7.31 |
| SG428 | 6.32 | 6.22 | 5.64 | 5.40 | 5.55 | 5.94 | 6.05 | 5.74 | 5.81 | 6.52 |
| SG459 | 8.52 | 8.71 | 8.35 | 8.54 | 8.00 | 8.87 | 8.32 | 8.11 | 8.20 | 8.77 |
| SG461 | 6.83 | 6.95 | 6.87 | 6.96 | 6.63 | 7.07 | 6.72 | 6.35 | 6.66 | 6.80 |
| SG493 | 7.99 | 7.92 | 7.71 | 7.73 | 7.49 | 7.96 | 7.79 | 7.53 | 7.70 | 7.99 |
| SG507 | 9.40 | 9.53 | 9.37 | 8.86 | 9.34 | 9.49 | 9.49 | 9.11 | 8.89 | 9.41 |
| SG508 | 8.17 | 8.18 | 7.37 | 7.80 | 7.81 | 7.97 | 7.75 | 7.79 | 7.46 | 7.69 |
| xᵢ | 62.78 | 73.52 | 19.76 | 53.03 | 28.34 | 68.92 | 33.36 | 17.23 | 16.00 | 45.86 |
| pᵢ | 1.00 | 1.00 | 1.00 | 1.00 | 1.00 | 1.00 | 1.00 | 1.00 | 1.00 | 1.00 |

| lmnᵢ | CLL151 | CLL158 | CLL169 | CLL171 | CLL178 | CLL111 | CLL155 |
|---|---|---|---|---|---|---|---|
| SG117 | 6.51 | 7.26 | 6.79 | 7.76 | 6.62 | 6.51 | 6.90 |
| SG428 | 6.14 | 6.17 | 5.81 | 5.96 | 5.97 | 6.14 | 5.44 |
| SG459 | 8.40 | 8.81 | 8.20 | 9.18 | 8.77 | 8.40 | 8.44 |
| SG461 | 6.64 | 7.01 | 6.66 | 7.10 | 6.79 | 6.64 | 6.71 |
| SG493 | 8.00 | 8.12 | 7.70 | 8.03 | 8.05 | 8.00 | 7.91 |
| SG507 | 9.11 | 9.36 | 8.89 | 9.52 | 9.36 | 9.11 | 9.35 |
| SG508 | 7.87 | 8.29 | 7.46 | 7.99 | 8.06 | 7.87 | 8.23 |
| xᵢ | 35.32 | 81.12 | 16.00 | 79.33 | 57.33 | 35.32 | 53.92 |
| pᵢ | 1.00 | 1.00 | 1.00 | 1.00 | 1.00 | 1.00 | 1.00 |

A third group of 40 samples was formed. To do this, replicas of hybridization or of labelling were used (the samples whose name begins with S and Strans are samples from people considered healthy and those which start with CLL are samples from patients with chronic lymphatic leukemia). This group of samples was used to validate the classification system. The data were normalized as has been previously described. The results of the classification are shown in the Table 13. 40 out of the 40 samples are correctly classified.

TABLE 13
Results obtained in the validation of the classification function obtained by logistical regression

| lmnᵢ | S120.7 | S120.14 | Strans.3 | Strans.4 | S150.2 | S228.6 | S229.7 | CLL142.8 | CLL147.9 | S120.7 |
|---|---|---|---|---|---|---|---|---|---|---|
| SG117 | 5.22 | 5.40 | 5.41 | 4.95 | 5.47 | 6.05 | 5.34 | 7.06 | 5.39 | 5.22 |
| SG428 | 4.95 | 4.64 | 4.29 | 4.20 | 5.01 | 3.89 | 5.07 | 6.31 | 5.59 | 4.95 |
| SG459 | 6.39 | 6.14 | 5.46 | 4.98 | 7.14 | 5.72 | 6.23 | 9.01 | 8.56 | 6.39 |
| SG461 | 5.73 | 6.16 | 6.23 | 6.38 | 6.72 | 6.01 | 6.39 | 7.02 | 7.05 | 5.73 |
| SG493 | 6.78 | 7.05 | 5.03 | 5.15 | 6.95 | 6.37 | 6.22 | 7.87 | 8.07 | 6.78 |
| SG507 | 7.83 | 7.62 | 5.83 | 5.78 | 8.17 | 7.26 | 7.85 | 9.74 | 8.82 | 7.83 |
| SG508 | 6.42 | 6.46 | 6.90 | 4.56 | 6.62 | 6.70 | 6.71 | 8.19 | 8.17 | 6.42 |
| xᵢ | −107.44 | −99.01 | −48.21 | −172.85 | −40.21 | −93.50 | −56.20 | 85.27 | 64.43 | −107.44 |
| pᵢ | 0.00 | 0.00 | 0.00 | 0.00 | 0.00 | 0.00 | 0.00 | 1.00 | 1.00 | 0.00 |

| lmnᵢ | CLL148b.10 | CLL148c.11 | CLL111.12 | CLL163.13 | CLL108.15 | CLL160.1 | CLL160.2 | CL L187.5 |
|---|---|---|---|---|---|---|---|---|
| SG117 | 6.98 | 6.83 | 6.54 | 6.16 | 6.87 | 7.74 | 7.71 | 7.58 |
| SG428 | 6.64 | 6.63 | 6.17 | 5.61 | 6.56 | 5.92 | 5.69 | 7.24 |
| SG459 | 9.13 | 9.29 | 8.44 | 8.33 | 8.55 | 8.29 | 8.25 | 9.01 |
| SG461 | 7.16 | 7.48 | 6.67 | 6.61 | 6.86 | 7.21 | 7.25 | 7.35 |
| SG493 | 7.70 | 7.89 | 8.05 | 7.92 | 7.67 | 7.66 | 7.58 | 8.05 |
| SG507 | 9.90 | 10.02 | 9.16 | 9.16 | 9.66 | 8.88 | 8.93 | 9.99 |
| SG508 | 8.27 | 8.45 | 7.92 | 7.86 | 7.89 | 7.57 | 7.51 | 8.30 |
| xᵢ | 102.70 | 125.01 | 39.43 | 28.30 | 58.29 | 51.63 | 48.76 | 109.23 |
| pᵢ | 1.00 | 1.00 | 1.00 | 1.00 | 1.00 | 1.00 | 1.00 | 1.00 |

| lmnᵢ | CLL197.14 | CLL198.15 | CLL199.16 | CLL200.17 | CLL201.18 | CLL20_LE | CLL208.1 | CLL210.2 |
|---|---|---|---|---|---|---|---|---|
| SG117 | 6.66 | 6.13 | 7.14 | 7.58 | 7.97 | 6.67 | 7.40 | 7.11 |
| SG428 | 5.98 | 5.10 | 6.71 | 6.72 | 7.54 | 5.97 | 6.77 | 6.71 |
| SG459 | 8.35 | 7.97 | 8.34 | 8.99 | 9.30 | 8.26 | 8.34 | 8.19 |
| SG461 | 6.76 | 6.36 | 7.02 | 7.26 | 7.35 | 6.83 | 6.99 | 6.79 |
| SG493 | 7.80 | 7.27 | 7.96 | 8.09 | 8.39 | 7.53 | 7.44 | 7.48 |
| SG507 | 9.38 | 8.71 | 9.58 | 9.87 | 10.23 | 9.04 | 8.65 | 9.26 |
| SG508 | 8.00 | 7.48 | 7.72 | 8.64 | 8.76 | 7.17 | 7.09 | 7.61 |
| xᵢ | 48.40 | 0.40 | 48.33 | 116.75 | 134.69 | 14.31 | 29.64 | 39.38 |
| pᵢ | 1.00 | 0.60 | 1.00 | 1.00 | 1.00 | 1.00 | 1.00 | 1.00 |

| lmnᵢ | CLL225.6 | CLL236.7 | CLL240.8 | CLL184b.9 | CLL184C.10 | CLL208.1 | CLL213.5 | CLL214.6 |
|---|---|---|---|---|---|---|---|---|
| SG117 | 7.35 | 6.54 | 6.80 | 7.12 | 7.06 | 7.24 | 6.97 | 7.06 |
| SG428 | 6.46 | 6.43 | 6.11 | 6.97 | 6.36 | 6.34 | 5.80 | 6.45 |
| SG459 | 8.20 | 7.60 | 8.24 | 8.37 | 8.37 | 8.07 | 8.17 | 8.35 |
| SG461 | 6.90 | 6.85 | 6.58 | 7.05 | 6.73 | 6.53 | 6.43 | 6.79 |
| SG493 | 7.28 | 6.90 | 6.77 | 7.40 | 7.17 | 7.59 | 7.47 | 7.63 |
| SG507 | 8.45 | 8.55 | 8.85 | 9.22 | 8.88 | 8.08 | 8.61 | 9.13 |
| SG508 | 7.13 | 7.29 | 6.93 | 8.21 | 8.01 | 7.14 | 7.77 | 7.72 |
| xᵢ | 25.36 | 22.47 | 7.52 | 88.13 | 65.29 | 0.80 | 26.04 | 44.00 |
| pᵢ | 1.00 | 1.00 | 1.00 | 1.00 | 1.00 | 0.69 | 1.00 | 1.00 |

| lmnᵢ | CLL221.7 | CLL193.1 | CLL193.2 | CLL197.1 | CLL197.2 |
|---|---|---|---|---|---|
| SG117 | 6.97 | 6.76 | 6.73 | 6.22 | 6.00 |
| SG428 | 6.47 | 7.26 | 7.22 | 5.81 | 5.71 |
| SG459 | 7.98 | 8.28 | 8.27 | 8.26 | 8.07 |
| SG461 | 6.60 | 7.18 | 7.17 | 6.78 | 6.62 |
| SG493 | 7.66 | 7.95 | 7.93 | 7.79 | 7.85 |
| SG507 | 9.23 | 9.01 | 9.04 | 8.86 | 8.72 |
| SG508 | 7.74 | 7.68 | 7.77 | 7.55 | 7.64 |
| xᵢ | 27.46 | 56.71 | 60.37 | 23.63 | 14.87 |
| pᵢ | 1.00 | 1.00 | 1.00 | 1.00 | 1.00 |

### Example 5

### Results Obtained with “Stable” CLL Samples Compared with “Progressive” CLL Samples

“CLL-stable type” (S) samples are considered those of patients who have had stable CLL for over 5 years and “CLL-progressive type” (P) samples are considered the samples of patients classified as stable at the time of diagnosis and whose disease has progressed in less than one year.

In total 6 S samples and 6 P samples were analysed. The 12 samples were collected at the time of diagnosis, without clinical differences between them, but after one year, 6 of those patients had progressed. The 12 hybridizations have passed the aforementioned quality criteria.

Stable samples: E142R, E148, E156, E163, E164, E193

Progressive samples: P111, P105, P177, P158, P157 and P197.

All the data analysis was performed in R version 1.9.1.

Data normalization. In this case, and to avoid the significant genes obtained are due to a real difference between samples and not to the effect of normalization, the data were normalized in two different forms (“variance stabilization normalization” (vsn) and by robust quantiles) and the same statistical analysis was performed with each one of the normalizations.
Statistical analysis with normalized data by “variance stabilization normalization”. The list of statistically significant genes was obtained from a Welch's test with the mt.maxT function of the multtest package in R, with a value of p<0.05 without adjusting, i.e. without performing any control on the false positives and produced a list of 29 genes with stat istically significant differences between the CLL-stable type and CLL-progressive type groups.

The statistically significant oligonucleotides obtained were:

SG26, SG31, SG70, SG98, SG177, SG194, SG195, SG208, SG213, SG216, SG272, SG293, SG301, SG309, SG321, SG333, SG343, SG352, SG357, SG366, SG368, SG405, SG426, SG439, SG447, SG452, SG521, SG555, SG556.

The samples were grouped, which was performed with the hclust function of the stats package in R applying Pearson correlations. The tree obtained is shown in part A of FIG. 4.

The hierarchical grouping of the 12 samples in accordance with the expression of the 29 statistically significant genes obtained grouped the samples correctly: the tree contains two large branches, of which the right branch contains the 6 stable samples and the left branch contains the 6 progressive samples.
Statistical analysis with normalized data by robust quantiles The list of statistically significant genes was obtained from a Welch's test with the mt.maxT function of the multtest package in R with the values of p without adjusting i.e. without exerting any control over the rate of false positives, with a value of p<0.05, and produced a list of 19 genes with statistically significant differences between the CLL-stable type and CLL-progressive type groups:
SG26, SG31, SG177, SG194, SG195, SG197, SG213, SG216, SG293, SG301, SG309, SG333, SG343, SG357, SG366, SG439, SG452, SG555, SG556.

The supervised grouping of the 12 samples in accordance with the expression of the 19 statistically significant genes obtained gave rise to the tree which appears in part B of FIG. 4, wherein the samples also appear correctly grouped.

18 oligonucleotides common to both lists of statistically significant genes were selected and the average intensity of each one of them in the group of stable samples and in the group of progressive samples was calculated, as well as the variation in average intensity between the stable and progressive groups.

The values obtained are shown in Table 14.

TABLE 14
Values corresponding to the intensity of 18 significant oligonucleotides
to distinguish between CLL-stable and CLL-progressive

| | | Stable CLL | | Progressive CLL | | Change |
|---|---|---|---|---|---|---|
| | | group | | group | | stable/ |
| | Significance | Average | | Average | | progres- |
| Probe | (p data vsn) | Intensity | SD | Intensity | SD | sive |
| SG177 | 0.001 | 14 | 1.71 | 21 | 4.84 | 0.7 |
| SG366 | 0.001 | 18 | 2.33 | 14 | 1.80 | 1.3 |
| SG309 | 0.004 | 20 | 2.76 | 15 | 3.58 | 1.4 |
| SG26 | 0.005 | 97 | 19.20 | 70, | 13.24 | 1.4 |
| SG452 | 0.010 | 16 | 2.19 | 12 | 3.08 | 1.3 |
| SG216 | 0.012 | 46 | 14.64 | 31 | 7.13 | 1.5 |
| SG333 | 0.013 | 36 | 7.28 | 53 | 16.25 | 0.7 |
| SG357 | 0.014 | 134 | 6.50 | 175 | 38.67 | 0.8 |
| SG213 | 0.014 | 26 | 5.51 | 41 | 17.20 | 0.6 |
| SG31 | 0.014 | 69 | 32.50 | 30 | 10.57 | 1.8 |
| SG301 | 0.014 | 21 | 5.02 | 16 | 3.10 | 1.4 |
| SG194 | 0.019 | 37 | 9.52 | 50 | 9.95 | 0.7 |
| SG456 | 0.022 | 11 | 2.06 | 14 | 2.08 | 0.8 |
| SG293 | 0.029 | 17 | 1.88 | 21 | 3.72 | 0.8 |
| SG343 | 0.033 | 27 | 7.43 | 21 | 1.61 | 1.3 |
| SG439 | 0.038 | 18 | 2.00 | 20 | 1.74 | 0.9 |
| SG195 | 0.041 | 21 | 3.56 | 25, | 4.60 | 0.8 |
| SG555 | 0.049 | 163 | 23.55 | 137 | 20.69 | 1.2 |

To validate the results obtained with the microarray, 5 of the common statistically significant probes were selected obtained on comparing expression data from stable CLL subjects compared to progressive CLL subjects and the expression was studied with RT-PCR of the genes represented by those probes. The criteria used to select the 5 probes were: hybridization intensity, change of intensity between groups of stable and progressive and value of statistical significance. In this way, 5 probes were selected which represent genes PSMB4, CD23A, LCP1, ABCC5 and POU2F2. The expression of these 5 genes was determined in 11 of the 12 CLL type samples, as there was no total RNA of sample 105. With the expression value of the genes in each sample, the rate of change was determined between the group of stable and progressive and the value of significance of that variation and it was compared with the results obtained with the microarrays.

The technique used for the validation was RT-PCR or PCR in real time using a LightCycler. This technique is the technique of choice to validate data chips and as with the microarrays, measures mRNA level.

Primers were designed for each one of the 5 genes whose representative oligonucleotide was selected. The details thereof are shown below in Table 15.

TABLE 15
Primers and amplification products of the genes selected
for their validation by RT-PCR

| | | | Amplified | |
|---|---|---|---|---|
| | | | product | |
| Gene | Primer sequences (5′-3′) | SEQ ID NO: | size | Tₘ |
| PSMB4 | Direct: PSMB4_F | SEQ ID NO: 598 | 95 pb | 81° C. |
| | TTCTGGGAGATGGACACAGCTATA | | | |
| | Inverse: PSMB4_R | SEQ ID NO: 599 | | |
| | CCACAAAGGGTTCATCTTCGA | | | |
| CD23A | Direct: CD23A_F | SEQ ID NO: 600 | 97 pb | 82° C. |
| | TGCCCTGAAAAGTGGATCAAT | | | |
| | Reverse: CD23A_R | SEQ ID NO: 601 | | |
| | CCATGTCGTCACAGGCATACC | | | |
| LCP1 | Direct: LCP1_F | SEQ ID NO: 602 | 126 pb | 77° C. |
| | CCAGGTACCCTTCTCGCTTTT | | | |
| | Reverse: LCP1_R | SEQ ID NO: 603 | | |
| | CTCCTGGCCCTCATCTTGAA | | | |
| ABCC5 | Direct: ABCC5_F | SEQ ID NO: 604 | 119 pb | 82° C. |
| | CCCTCAAAGTCTGCAACTTTAAGC | | | |
| | Reverse: ABCC5_R | SEQ ID NO: 605 | | |
| | ACACACCAAACCACACAGCAA | | | |
| POU2F2 | Direct: POU2F2_F | SEQ ID NO: 606 | 136 pb | 82° C. |
| | GAGGACCAGCATCGAGACAAA | | | |
| | Reverse: POU2F2_R | SEQ ID NO: 607 | | |
| | AACCAGACGCGGATCACTTC | | | |

FIG. 5 shows the distribution of the expression data obtained by RT-PCR (left graphic) and by the microarray (right graphic). Part A corresponds to gene PSMB4, part B to gene CD23A and part C to gene POU2F2.

Below, in Table 16, the results obtained with the microarray and with RT-PCR are obtained of the change values of the 5 genes selected in thr group of stable samples compared with the group of progressive samples obtained as significance of the change. In 3 of the 5 genes selected (PSMB4, CD23A and POU2F2) the values of change, the direction of the change and the significance values obtained with RT-PCR agree with those obtained with the microarray, for which reason those 3 genes are considered valid, i.e. the results obtained for those 3 genes with the microarray coincide with the results obtained by another techniques which also measures mRNA level.

TABLE 16
Values of change and significance of the change obtained with
the microarray and by RT-PCR

| | | Stable/progressive | | | |
|---|---|---|---|---|---|
| | | change | | Significance of the change | |
| Probes | Genes | Array | RT-PCR | Array | RT-PCR |
| SG26 | PSBM4 | 1.3 | 1.5 | 0.04 | 0.15 |
| SG216 | CD23A | 1.5 | 3.2 | 0.04 | 0.03 |
| SG333 | LCP1 | 0.7 | 1 | 0.05 | 0.97 |
| SG357 | ABCC5 | 0.8 | 1.3 | 0.10 | 0.28 |
| SG366 | POU2F2 | 1.3 | 2.3 | 0.01 | 0.05 |

### BIBLIOGRAPHIC REFERENCES

Alizadeh A, Eisen M, Davis R E, et al The lymphochip: a specialized cDNA microarray for the genomic-scale analysis of gene expression in normal and malignant lymphocytes. Cold Spring Harb Symp Quant Biol. 1999; 64:71-8.
Alizadeh A A, Eisen M B, Davis R E, et al Distinct types of diffuse large B-cell lymphoma identified by gene expression profiling. Nature. 2000; 403:503-11.
Arico M, Valsecchi M G, Camitta B, Schrappe M, Chessells J, Baruchel A, Gaynon P, Silverman L, Janka-Schaub G, Kamps W, Pui C H, Masera G. Outcome of treatment in children with Philadelphia chromosome-positive acute lymphoblastic leukemia N Engl J. Med. 2000; 342:998-1006.).
Bea S, Zettl A, Wright G, et al. Diffuse Large B-Cell Lymphoma Subgroups Have Distinct Genetic Profiles that Influence Tumor Biology and Improve Gene Expression-Based Survival Prediction. Blood. 2005 04:-1399
Bene M C, Bernier M, Casasnovas R O, et al: Acute myeloid leukaemia M O: Haematological, immunophenotypic and cytogenetic characteristics and their prognostic significance: An analysis in 241 patients. Br J Haemat 113:737, 2001.

Benes V, Muckenthaler M. Standardization of protocols in cDNA microarray analysis. Trends Biochem Sci. 2003; 28:244-9
Bennett J M, Catovsky D, Daniel M T, et al. Myelodysplastic syndromes: is another classification necessary? Br J. Haematol. 1984; 56:515-7.
Bennett J M, Catovsky D, Daniel M T, et al. Proposals for the classification of the acute leukaemias. French-American-British (FAB) co-operative group. Br J. Haematol. 1976; 33:451-8.
Bergh G, Ehinger M, Olsson I, Jacobsen S E, Gullberg U. Involvement of the retinoblastoma protein in monocytic and neutrophilic lineage commitment of human bone marrow progenitor cells Blood. 1999; 94:1971-8
Bernard J, Levy J P, Varet B. Hematologie. Collection Medico Chirurgicale. Paris: Flammarion, 1976; 5-22
Binet J L, Auquier A, Dighiero G, et al. A new prognostic classification of chronic lymphocytic leukemia derived from a multivariate survival analysis. Cancer. 1981; 48:198-206
Boultwood J, Lewis S, Wainscoat J S The 5q-syndrome. Blood. 1994; 84:3253-60
Braziel R M, Shipp M A, Feldman A L, et al. Molecular diagnostics. Hematology (Am Soc Hematol Educ Program). 2003; 279-93
Bullinger, Milner et al. Use of gene-expression profiling to identify prognostic subclasses in adult acute myeloid leukemia N Engl Med 2005; 350:1605-16
Chan W C. Gene expression profiling in lymphoma diagnosis and research. Croat Med J. 2005; 46:349-59
Chen C C, Andrich M P, Metcalfe D D: A retrospective analysis of bone scan abnormalities in mastocytosis: Correlation with disease category and prognosis. J Nucl Med 35:1471, 1994
Cossman J: Gene expression analysis of single neoplastic cells and the pathogenesis of Hodgkin's lymphoma. J Histochem Cytochem 2001; 49:799-800
Crespo M, Bosch F, Villamor N, et al. ZAP-70 expression as a surrogate for immunoglobulin-variable-region mutations in chronic lymphocytic leukemia. N Engl J. Med. 2003; 348:1764-75
Datta S R, Brunet A, Greenberg M E. Cellular survival: A play in three acts. Genes Dev 1999; 13: 2905-2927
Devilard E, Bertucci F, Tremat P, et al: Gene expression profiling defines molecular subtypes of classical Hodgkin's lymphoma. Oncogene 2002; 21:3095-3102
Dohner K, Tobis K, Ulrich R, Frohling S, Benner A, Schlenk R F, Dohner H. Prognostic significance of partial tandem duplications of the MLL gene in adult patients 16 to 60 years old with acute myeloid leukemia and normal cytogenetics: a study of the Acute Myeloid Leukemia Study Group Ulm. J Clin Oncol. 2002; 20:3254-61
Domen J, Weissman I L. Self-renewal, differentiation or death: regulation and manipulation of hematopoietic stem cell fate. Mol Med. Today. 1999; 5:201-8
Durig J, Naschar M, Schmucker U, Renzing-Kohler K, Holter T, Huttmann A, Duhrsen U. CD38 expression is an important prognostic marker in chronic lymphocytic leukaemia. Leukemia. 2002; 16:30-5
Ferrando A A, Look A T Pathobiology of acute lymphoblastic leukemia. In Hoffman R Hematology Basic Principles and practice. 4 ed. Churchill Livingstone New York 2005; 1135-1147
Galton D A. The pathogenesis of chronic lymphocytic leukemia Can Med Assoc J. 1966; 94:1005-10
Giles F J, Keating A, Goldstone A H, Avivi I, Willman C L, Kantarjian H M Acute myeloid leukemia. Hematology (Am Soc Hematol Educ Program). 2002; 73-110.).
Golub T R, Slonim D K, Tamayo P, et al. Molecular classification of cancer: class discovery and class prediction by gene expression monitoring. Science. 1999; 286:531-7
Gong J Z, Lagoo A S, Peters D, Horvatinovich J, Benz P, Buckley P J. Value of CD23 determination by flow cytometry in differentiating mantle cell lymphoma from chronic lymphocytic leukemia/small lymphocytic lymphoma Am J Clin Pathol. 2001; 116:893-7
Greiner TC.mRNA microarray analysis in lymphoma and leukemia. Cancer Treat Res. 2004; 121:1-12
Guttmacher A E, Collins F S. Welcome to the genomic era N Engl J. Med. 2003; 349:996-8
Hamblin T J, Davis Z, Gardiner A, Oscier D G, Stevenson F K Unmutated Ig V(H) genes are associated with a more aggressive form of chronic lymphocytic leukemia. Blood. 1999; 94:1848-54
Hanahan D, Weinberg R A: The hallmarks of cancer. Cell 100:57-70, 2000
Harris N L, Jaffe E S, Diebold J, et al. The World Health Organization classification of neoplastic diseases of the hematopoietic and lymphoid tissues. Report of the Clinical Advisory Committee meeting, Airlie House, Virginia, November, 1997 Ann Oncol. 1999; 10:1419-32
Harris N L, Jaffe E S, Diebold J, et al. World Health Organization classification of neoplastic diseases of the hematopoietic and lymphoid tissues: report of the Clinical Advisory Committee meeting, Airlie House, Virginia, November 1997 J Clin Oncol. 1999; 17:3835-49
Harris N L, Jaffe E S, Stein H, et al A revised European-American classification of lymphoid neoplasms: a proposal from the International Lymphoma Study Group Blood. 1994; 84:1361-92
Harris N L, Stein H, Coupland S E, Hummel M, Favera R D, Pasqualucci L, Chan W C New approaches to lymphoma diagnosis. Hematology (Am Soc Hematol Educ Program). 2001; 194-220.
Heaney M L, Golde D W Myelodysplasia. N Engl J. Med. 1999; 340:1649-60
Jaffe E S, Harris N L, Stein H, Vardiman J W. WHO Classification Tumours of Heamotopoietic and lymphoid tissues. En Pathology and Genetics of tumours of Haematopoietic and lymphoid tissues. IARC Press. Lyon, 2001.
Jemal A, Murray T, Samuels A, et al: Cancer statistics, 2003. CA Cancer J Clin 53:5, 2003
Kottaridis P D, Gale R E, Frew M E, et al. The presence of a FLT3 internal tandem duplication in patients with acute myeloid leukemia (AML) adds important prognostic information to cytogenetic risk group and response to the first cycle of chemotherapy: analysis of 854 patients from the United Kingdom Medical Research Council AML 10 and 12 trials. Blood. 2001; 98:1752-9
Kuppers R, Klein U, Hansmann M L, Rajewsky K. Cellular origin of human B-cell lymphomas. N Engl J. Med. 1999; 341:1520-9
Küppers R: Molecular biology of Hodgkin's lymphoma. Adv Cancer Res 2002; 84:277-312
Kyle R A: Monoclonal gammopathy of undetermined significance and solitary plasmacytoma: Implications for progression to overt multiple myeloma. Hematol Oncol Clin North Am 11:71, 1997
Lee M F, Dang C V Control of cell division. In Hoffman R Hematology Basic Principles and practice. 4 ed. Churchill Livingstone New York 2005; 69-81
Leung AYH, Verfaillie C M Stem cell model of hematopoyesis. In Hoffman R Hematology Basic Principles and practice. 4 ed. Churchill Livingstone New York 2005; 200-214
Magli M C, Largman C, Lawrence H J Effects of HOX homeobox genes in blood cell differentiation J Cell Physiol. 1997; 173:168-77
Mitelman F, Mertens F, Johansson B A breakpoint map of recurrent chromosomal rearrangements in human neoplasia. Nat. Genet. 1997; 15 Spec No:417-74
Montoto S, Lopez-Guillermo A, Colomer D, et al. Incidence and clinical significance of bcl-2/IgH rearrangements in follicular lymphoma. Leuk Lymphoma. 2003; 44:71-6.
Nichogiannopoulou A, Trevisan M, Friedrich C, Georgopoulos K. Ikaros in hemopoietic lineage determination and homeostasis Semin Immunol. 1998; 10:119-25.
Nutt S L, Heavey B, Rolink A G, Busslinger M. Commitment to the B-lymphoid lineage depends on the transcription factor Pax5. Nature. 1999; 401:556-62
O'Gorman D M, Cotter T G Molecular signals in anti-apoptotic survival pathways. Leukemia. 2001; 15:21-34
Pane F, Intrieri M, Quintarelli C, Izzo B, Muccioli G C, Salvatore F BCR/ABL genes and leukemic phenotype: from molecular mechanisms to clinical correlations. Oncogene. 2002; 21:8652-67.
R Development Core Team. R: A language and environment for statistical computing. R Foundation for Statistical Computing, Vienna, Austria, 2004
Rai K R, Sawitsky A, Cronkite E P, Chanana A D, Levy R N, Pasternack B S. Clinical staging of chronic lymphocytic leukemia. Blood. 1975; 46:219-34
Roumier C, Eclache V, Imbert M, et al. M O AML, clinical and biologic features of the disease, including AML1 gene mutations: A report of 59 cases by the Groupe Francais d'Hematologie Cellulaire (GFHC) and the Groupe Francais de Bytogenetique Hematologique (GFCH). Blood 101:1277, 2003
Shaffer A L, Rosenwald A, Staudt L M Lymphoid malignancies: the dark side of B-cell differentiation Nat Rev Immunol. 2002; 2:920-32
Tenen D G, Hromas R, Licht J D, Zhang D E Transcription factors, normal myeloid development, and leucemia. Blood. 1997; 90:489-519
Tibshirani R, Hastie T, Narasimhan B, Chu G. Diagnosis of multiple cancer types by shrunken centroids of gene expression. Proc. Natl. Acad. Sci. 2002; 99:6567-72
Vardiman J W, Harris N L, Brunning R D The World Health Organization (WHO) classification of the myeloid neoplasms. Blood. 2002; 100:2292-302
Venables, Smith et al. An Introduction to R, Notes on R: A programming Environment for Data Analysis. 2004
Weissman I L Stem cells: Units of regeneration and units in evolution. Cell, 2000; 100:157-168.
Westbrook C A. The molecular basis of neoplasia. In Hoffman R Hematology Basic Principles and practice. 4 ed. Churchill Livingstone New York 2005; 941-945
Zhan F, Hardin J, Kordsmeier B, et al: Global gene expression profiling of multiple myeloma, monoclonal gammopathy of undetermined significance, and normal bone marrow plasma cells. Blood 99:1745, 2002

### BRIEF DESCRIPTION OF THE FIGURES

FIG. 1 shows the grouping of samples of cells U937 compared with Jurkat cells in accordance with differences in the gene expression between the samples. Part A corresponds to the non-supervised grouping; part B corresponds to the supervised grouping.

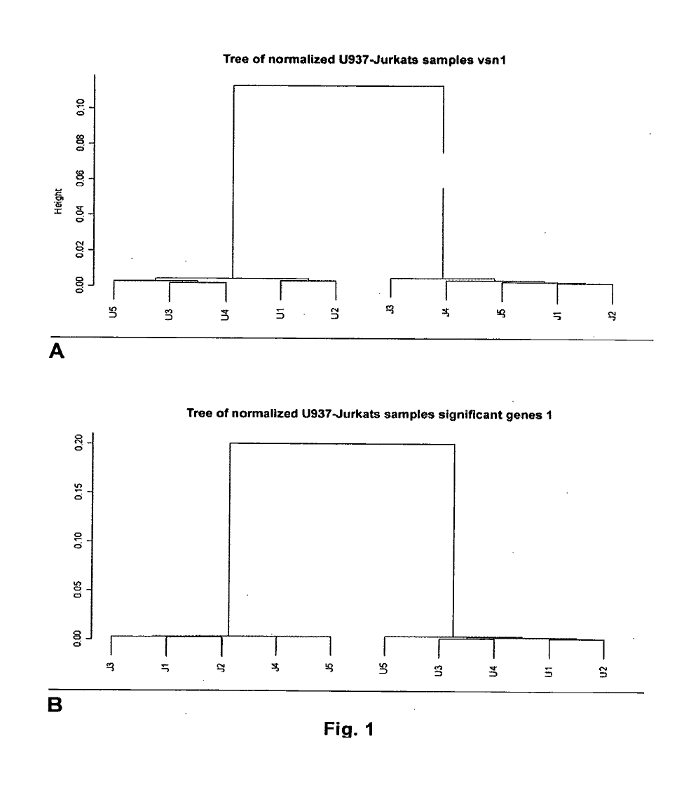

FIG. 2 shows the grouping of samples of healthy subjects compared with U937 and Jurkat cells in accordance with differences in the gene expression between the samples. Part A corresponds to the non-supervised grouping; part B corresponds to the supervised grouping.

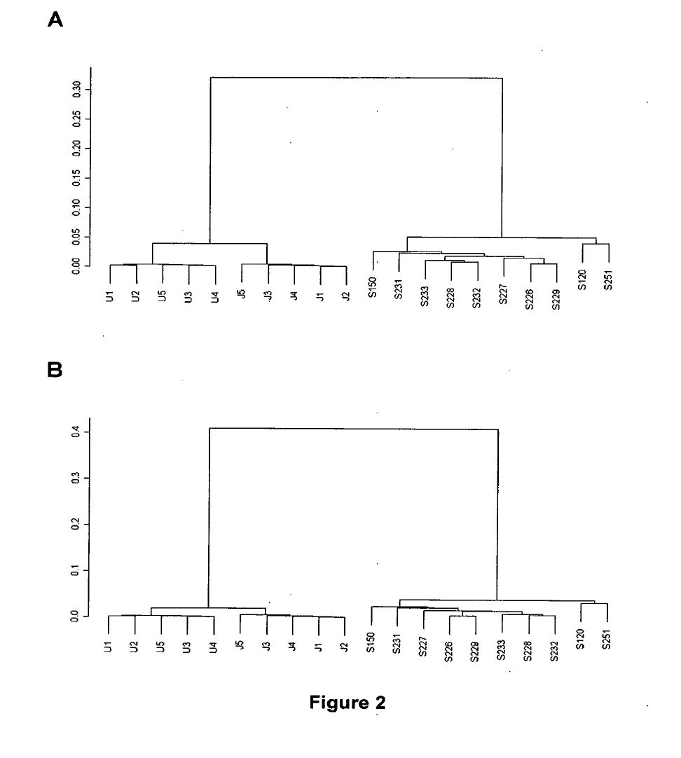

FIG. 3 shows the grouping of samples of patients with chronic lymphatic leukemia compared with U937 and Jurkat cells in accordance with differences in the gene expression between the samples. Part A corresponds to the non-supervised grouping; part B corresponds to the supervised grouping.

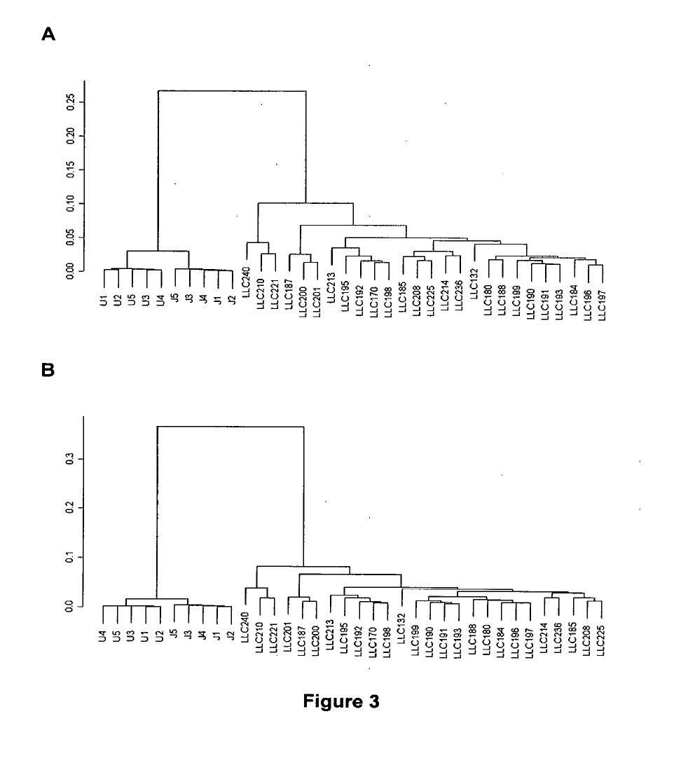

FIG. 4 shows the grouping of samples of patients with “stable” chronic lymphatic leukemia compared with samples of patients with “progressive” chronic lymphatic leukemia in accordance with differences in gene expression. Part A corresponds to the grouping in accordance with the genes identified as significant after normalization with “vsn” and use of the mt.maxT function in R; part B corresponds to the grouping in accordance with the genes identified as significant after normalization by robust quartiles and use of the mt.maxT function in R.

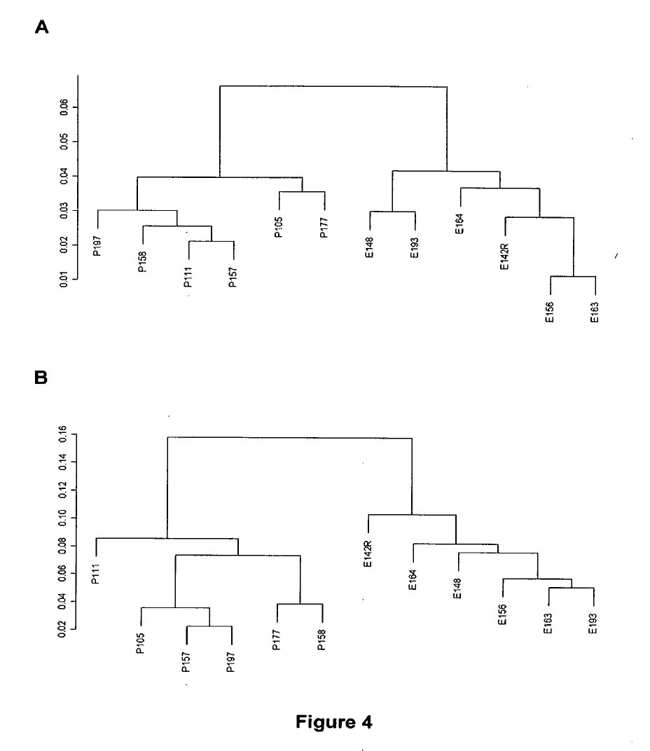

FIG. 5 shows the distribution of the expression data obtained by RT-PCR (left-hand graphic) and from the intensity values obtained from the microarray (right-hand graphic) for the PSMB4 genes (part A: upper graphic), CD23A (part B: intermediate graphic) and POU2F2 (part C: lower graphics) in samples of patients with “stable” chronic lymphatic leukemia (bars marked with “E”) and in samples of patients with “progressive” chronic lymphatic leukemia (bars marked with “P”).

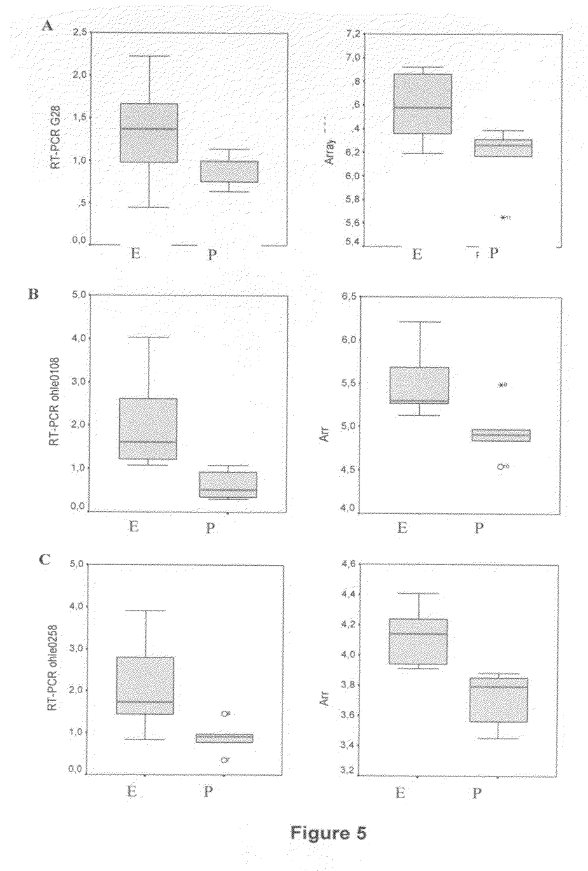

1. A composition which comprises at least one oligonucleotide from the group composed of:
SG1, SG2, SG3, SG4, SG5, SG6, SG7, SG8, SG9, SG10, SG11, SG12, SG13, SG14, SG15, SG16, SG17, SG18, SG19, SG20, SG21, SG22, SG23, SG24, SG25, SG26, SG27, SG28, SG29, SG30, SG31, SG32, SG33, SG34, SG35, SG36, SG37, SG38, SG39, SG40, SG41, SG42, SG43, SG44, SG45, SG46, SG47, SG48, SG49, SG50, SG51, SG52, SG53, SG54, SG55, SG56, SG57, SG58, SG59, SG60, SG61, SG62, SG63, SG64, SG65, SG66, SG67, SG68, SG69, SG70, SG71, SG72, SG73, SG74, SG75, SG76, SG77, SG78, SG79, SG80, SG81, SG82, SG83, SG84, SG85, SG86, SG87, SG88, SG89, SG90, SG91, SG92, SG93, SG94, SG95, SG96, SG97, SG98, SG99, SG100, SG101, SG102, SG103, SG104, SG105, SG106, SG107, SG108, SG109, SG110, SG111, SG112, SG113, SG114, SG115, SG116, SG117, SG118, SG119, SG120, SG121, SG122, SG123, SG124, SG125, SG126, SG127, SG128, SG129, SG130, SG131, SG132, SG133, SG134, SG135, SG136, SG137, SG138, SG139, SG140, SG141, SG142, SG143, SG144, SG145, SG146, SG147, SG148, SG149, SG150, SG151, SG152, SG153, SG154, SG155, SG156, SG157, SG158, SG159, SG160, SG161, SG162, SG163, SG164, SG165, SG166, SG167, SG168, SG169, SG170, SG171, SG172, SG173, SG174, SG175, SG176, SG177, SG178, SG179, SG180, SG181, SG182, SG183, SG184, SG185, SG186, SG187, SG188, SG189, SG190, SG191, SG192, SG193, SG194, SG195, SG196, SG197, SG198, SG199, SG200, SG201, SG202, SG203, SG204, SG205, SG206, SG207, SG208, SG209, SG210, SG211, SG212, SG213, SG214, SG215, SG216, SG217, SG218, SG219, SG220, SG221, SG222, SG223, SG224, SG225, SG226, SG227, SG228, SG229, SG230, SG231, SG232, SG233, SG234, SG235, SG236, SG237, SG238, SG239, SG240, SG241, SG242, SG243, SG244, SG245, SG246, SG247, SG248, SG249, SG250, SG251, SG252, SG253, SG254, SG255, SG256, SG257, SG258, SG259, SG260, SG261, SG262, SG263, SG264, SG265, SG266, SG267, SG268, SG269, SG270, SG271, SG272, SG273, SG274, SG275, SG276, SG277, SG278, SG279, SG280, SG281, SG282, SG283, SG284, SG285, SG286, SG287, SG288, SG289, SG290, SG291, SG292, SG293, SG294, SG295, SG296, SG297, SG298, SG299, SG300, SG301, SG302, SG303, SG304, SG305, SG306, SG307, SG308, SG309, SG310, SG311, SG312, SG313, SG314, SG315, SG316, SG317, SG318, SG319, SG320, SG321, SG322, SG323, SG324, SG325, SG326, SG327, SG328, SG329, SG330, SG331, SG332, SG333, SG334, SG335, SG336, SG337, SG338, SG339, SG340, SG341, SG342, SG343, SG344, SG345, SG346, SG347, SG348, SG349, SG350, SG351, SG352, SG353, SG354, SG355, SG356, SG357, SG358, SG359, SG360, SG361, SG362, SG363, SG364, SG365, SG366, SG367, SG368, SG369, SG370, SG371, SG372, SG373, SG374, SG375, SG376, SG377, SG378, SG379, SG380, SG381, SG382, SG383, SG384, SG385, SG386, SG387, SG388, SG389, SG390, SG391, SG392, SG393, SG394, SG395, SG396, SG397, SG398, SG399, SG400, SG401, SG402, SG403, SG404, SG405, SG406, SG407, SG408, SG409, SG410, SG411, SG412, SG413, SG414, SG415, SG416, SG417, SG418, SG419, SG420, SG421, SG422, SG423, SG424, SG425, SG426, SG427, SG428, SG429, SG430, SG431, SG432, SG433, SG434, SG435, SG436, SG437, SG438, SG439, SG440, SG441, SG442, SG443, SG444, SG445, SG446, SG447, SG448, SG449, SG450, SG451, SG452, SG453, SG454, SG455, SG456, SG457, SG458, SG459, SG460, SG461, SG462, SG465, SG468, SG469, SG470, SG471, SG472, SG473, SG474, SG475, SG476, SG477, SG478, SG479, SG480, SG481, SG482, SG483, SG484, SG485, SG486, SG487, SG488, SG489, SG490, SG491, SG492, SG493, SG494, SG495, SG496, SG497, SG498, SG499, SG500, SG501, SG502, SG503, SG504, SG505, SG506, SG507, SG508, SG509, SG510, SG511, SG512, SG513, SG514, SG515, SG516, SG517, SG518, SG519, SG520, SG521, SG522, SG523, SG524, SG525, SG526, SG527, SG428, SG529, SG530, SG531, SG532, SG533, SG534, SG535, SG536, SG537, SG538, SG539, SG540, SG541, SG542, SG543, SG544, SG545, SG546, SG547, SG548, SG549, SG550, SG551, SG552, SG553, SG554, SG555, SG556, SG557, SG558, SG559, SG560, SG561, SG562, SG563,
or combinations thereof, to be used as probe in the determination of the expression level of a gene which possesses a sequence complementary to said oligonucleotide by the evaluation of the mRNA level corresponding to that gene, of application in the in vitro diagnosis of neoplasias originating from hematopoietic cells and/or in the in vitro prognosis of the evolution of said disease.

2. A composition according to claim 1, which comprises at least the oligonucleotides SG117, SG428, SG459, SG507, SG508.

3. A composition according to claim 2, which additionally comprises at least oligonucleotides SG461 and SG493.

4. A composition according to claim 2, which additionally comprises at least the oligonucleotide SG237.

5. A composition according to claim 4, which additionally comprises at least one oligonucleotide selected from the group of SG2, SG4, SG8, SG10, SG13, SG15, SG16, SG19, SG20, SG23, SG26, SG28, SG31, SG34, SG36, SG39, SG48, SG58, SG60, SG65, SG76, SG77, SG84, SG89, SG94, SG9, SG97, SG99, SG102, SG106, SG107, SG463, SG115, SG116, SG120, SG129, SG134, SG135, SG138, SG139, SG141, SG145, SG158, SG161, SG163, SG176, SG178, SG185, SG207, SG208, SG210, SG217, SG227, SG231, SG237, SG264, SG272, SG277, SG281, SG283, SG286, SG294, SG298, SG299, SG307, SG308, SG319, SG328, SG330, SG333, SG336, SG342, SG344, SG345, SG347, SG361, SG384, SG389, SG395, SG404, SG407, SG416, SG423, SG430, SG432, SG434, SG444, SG446, SG453, SG458, SG464, SG465, SG466, SG467, SG471, SG473, SG474, SG475, SG481, SG485, SG487, SG491, SG498, SG511, SG517, SG518, SG522, SG526, SG530, SG533, SG538, SG541, SG554, SG558, SG561 or combinations thereof.

6. A composition according to claim 2, to be used in the in vitro diagnosis of chronic lymphatic leukemia.

7. A composition according to claim 1, which comprises at least oligonucleotides SG26, SG216, SG366.

8. A composition according to claim 7, which additionally comprises at least one oligonucleotide selected from the group of SG31, SG177, SG194, SG195, SG197, SG213, SG293, SG301, SG309, SG333, SG343, SG357, SG439, SG452, SG555, SG556.

9. A composition according to claim 7, to be used in the in vitro prognosis of the future evolution of the disease in a patient suffering from chronic lymphatic leukemia.

10. A composition according to claim 1, which comprises the totality of the nucleotides of the group composed of:
SG1, SG2, SG3, SG4, SG5, SG6, SG7, SG8, SG9, SG10, SG11, SG12, SG13, SG14, SG15, SG16, SG17, SG18, SG19, SG20, SG21, SG22, SG23, SG24, SG25, SG26, SG27, SG28, SG29, SG30, SG31, SG32, SG33, SG34, SG35, SG36, SG37, SG38, SG39, SG40, SG41, SG42, SG43, SG44, SG45, SG46, SG47, SG48, SG49, SG50, SG51, SG52, SG53, SG54, SG55, SG56, SG57, SG58, SG59, SG60, SG61, SG62, SG63, SG64, SG65, SG66, SG67, SG68, SG69, SG70, SG71, SG72, SG73, SG74, SG75, SG76, SG77, SG78, SG79, SG80, SG81, SG82, SG83, SG84, SG85, SG86, SG87, SG88, SG89, SG90, SG91, SG92, SG93, SG94, SG95, SG96, SG97, SG98, SG99, SG100, SG101, SG102, SG103, SG104, SG105, SG106, SG107, SG108, SG109, SG110, SG111, SG112, SG113, SG114, SG115, SG116, SG117, SG118, SG119, SG120, SG121, SG122, SG123, SG124, SG125, SG126, SG127, SG128, SG129, SG130, SG131, SG132, SG133, SG134, SG135, SG136, SG137, SG138, SG139, SG140, SG141, SG142, SG143, SG144, SG145, SG146, SG147, SG148, SG149, SG150, SG151, SG152, SG153, SG154, SG155, SG156, SG157, SG158, SG159, SG160, SG161, SG162, SG163, SG164, SG165, SG166, SG167, SG168, SG169, SG170, SG171, SG172, SG173, SG174, SG175, SG176, SG177, SG178, SG179, SG180, SG181, SG182, SG183, SG184, SG185, SG186, SG187, SG188, SG189, SG190, SG191, SG192, SG193, SG194, SG195, SG196, SG197, SG198, SG199, SG200, SG201, SG202, SG203, SG204, SG205, SG206, SG207, SG208, SG209, SG210, SG211, SG212, SG213, SG214, SG215, SG216, SG217, SG218, SG219, SG220, SG221, SG222, SG223, SG224, SG225, SG226, SG227, SG228, SG229, SG230, SG231, SG232, SG233, SG234, SG235, SG236, SG237, SG238, SG239, SG240, SG241, SG242, SG243, SG244, SG245, SG246, SG247, SG248, SG249, SG250, SG251, SG252, SG253, SG254, SG255, SG256, SG257, SG258, SG259, SG260, SG261, SG262, SG263, SG264, SG265, SG266, SG267, SG268, SG269, SG270, SG271, SG272, SG273, SG274, SG275, SG276, SG277, SG278, SG279, SG280, SG281, SG282, SG283, SG284, SG285, SG286, SG287, SG288, SG289, SG290, SG291, SG292, SG293, SG294, SG295, SG296, SG297, SG298, SG299, SG300, SG301, SG302, SG303, SG304, SG305, SG306, SG307, SG308, SG309, SG310, SG311, SG312, SG313, SG314, SG315, SG316, SG317, SG318, SG319, SG320, SG321, SG322, SG323, SG324, SG325, SG326, SG327, SG328, SG329, SG330, SG331, SG332, SG333, SG334, SG335, SG336, SG337, SG338, SG339, SG340, SG341, SG342, SG343, SG344, SG345, SG346, SG347, SG348, SG349, SG350, SG351, SG352, SG353, SG354, SG355, SG356, SG357, SG358, SG359, SG360, SG361, SG362, SG363, SG364, SG365, SG366, SG367, SG368, SG369, SG370, SG371, SG372, SG373, SG374, SG375, SG376, SG377, SG378, SG379, SG380, SG381, SG382, SG383, SG384, SG385, SG386, SG387, SG388, SG389, SG390, SG391, SG392, SG393, SG394, SG395, SG396, SG397, SG398, SG399, SG400, SG401, SG402, SG403, SG404, SG405, SG406, SG407, SG408, SG409, SG410, SG411, SG412, SG413, SG414, SG415, SG416, SG417, SG418, SG419, SG420, SG421, SG422, SG423, SG424, SG425, SG426, SG427, SG428, SG429, SG430, SG431, SG432, SG433, SG434, SG435, SG436, SG437, SG438; SG439, SG440, SG441, SG442, SG443, SG444, SG445, SG446, SG447, SG448, SG449, SG450, SG451, SG452, SG453, SG454, SG455, SG456, SG457, SG458, SG459, SG460, SG461, SG462, SG465, SG468, SG469, SG470, SG471, SG472, SG473, SG474, SG475, SG476, SG477, SG478, SG479, SG480, SG481, SG482, SG483, SG484, SG485, SG486, SG487, SG488, SG489, SG490, SG491, SG492, SG493, SG494, SG495, SG496, SG497, SG498, SG499, SG500, SG501, SG502, SG503, SG504, SG505, SG506, SG507, SG508, SG509, SG510, SG511, SG512, SG513, SG514, SG515, SG516, SG517, SG518, SG519, SG520, SG521, SG522, SG523, SG524, SG525, SG526, SG527, SG428, SG529, SG530, SG531, SG532, SG533, SG534, SG535, SG536, SG537, SG538, SG539, SG540, SG541, SG542, SG543, SG544, SG545, SG546, SG547, SG548, SG549, SG550, SG551, SG552, SG553, SG554, SG555, SG556, SG557, SG558, SG559, SG560, SG561, SG562, SG563.

11. A composition according to claim 1, characterized in that it additionally comprises at least one oligonucleotide selected from the group composed of SG463, SG464, SG466, SG467, SSPC1, SSPC2, SSPC3, SSPC4, SSPC5, SSPC6, SSPC7, SCN1, SCN2, SCN3, SCN5, SCN6, SCN7, SCN8, SCN10, SCN11, SCN12, SCN13, SC1, SC2, SC3, SC4, SC5, SC6 and SC7.

12. A composition according to claim 11, which comprises all the oligonucleotides from the group composed of SG463, SG464, SG466, SG467, SSPC1, SSPC2, SSPC3, SSPC4, SSPC5, SSPC6, SSPC7, SCN1, SCN2, SCN3, SCN5, SCN6, SCN7, SCN8, SCN10, SCN11, SCN12, SCN13, SC1, SC2, SC3, SC4, SC5, SC6 and SC7.

13. A composition according to claim 1, wherein the oligonucleotides are disposed on a solid support.

14. A composition according to claim 13, wherein the oligonucleotides are disposed in an ordered fashion on a solid support which is glass similar to a slide whereto the oligonucleotides are bound by covalent bonds, forming a microarray.

15. A composition in the form of microarray according to claim 14, which comprises the totality of the oligonucleotides from the group composed of
SG1, SG2, SG3, SG4, SG5, SG6, SG7, SG8, SG9, SG10, SG11, SG12, SG13, SG14, SG15, SG16, SG17, SG18, SG19, SG20, SG21, SG22, SG23, SG24, SG25, SG26, SG27, SG28, SG29, SG30, SG31, SG32, SG33, SG34, SG35, SG36, SG37, SG38, SG39, SG40, SG41, SG42, SG43, SG44, SG45, SG46, SG47, SG48, SG49, SG50, SG51, SG52, SG53, SG54, SG55, SG56, SG57, SG58, SG59, SG60, SG61, SG62, SG63, SG64, SG65, SG66, SG67, SG68, SG69, SG70, SG71, SG72, SG73, SG74, SG75, SG76, SG77, SG78, SG79, SG80, SG81, SG82, SG83, SG84, SG85, SG86, SG87, SG88, SG89, SG90, SG91, SG92, SG93, SG94, SG95, SG96, SG97, SG98, SG99, SG100, SG101, SG102, SG103, SG104, SG105, SG106, SG107, SG108, SG109, SG110, SG111, SG112, SG113, SG114, SG115, SG116, SG117, SG118, SG119, SG120, SG121, SG122, SG123, SG124, SG125, SG126, SG127, SG128, SG129, SG130, SG131, SG132, SG133, SG134, SG135, SG136, SG137, SG138, SG139, SG140, SG141, SG142, SG143, SG144, SG145, SG146, SG147, SG148, SG149, SG150, SG151, SG152, SG153, SG154, SG155, SG156, SG157, SG158, SG159, SG160, SG161, SG162, SG163, SG164, SG165, SG166, SG167, SG168, SG169, SG170, SG171, SG172, SG173, SG174, SG175, SG176, SG177, SG178, SG179, SG180, SG181, SG182, SG183, SG184, SG185, SG186, SG187, SG188, SG189, SG190, SG191, SG192, SG193, SG194, SG195, SG196, SG197, SG198, SG199, SG200, SG201, SG202, SG203, SG204, SG205, SG206, SG207, SG208, SG209, SG210, SG211, SG212, SG213, SG214, SG215, SG216, SG217, SG218, SG219, SG220, SG221, SG222, SG223, SG224, SG225, SG226, SG227, SG228, SG229, SG230, SG231, SG232, SG233, SG234, SG235, SG236, SG237, SG238, SG239, SG240, SG241, SG242, SG243, SG244, SG245, SG246, SG247, SG248, SG249, SG250, SG251, SG252, SG253, SG254, SG255, SG256, SG257, SG258, SG259, SG260, SG261, SG262, SG263, SG264, SG265, SG266, SG267, SG268, SG269, SG270, SG271, SG272, SG273, SG274, SG275, SG276, SG277, SG278, SG279, SG280, SG281, SG282, SG283, SG284, SG285, SG286, SG287, SG288, SG289, SG290, SG291, SG292, SG293, SG294, SG295, SG296, SG297, SG298, SG299, SG300, SG301, SG302, SG303, SG304, SG305, SG306, SG307, SG308, SG309, SG310, SG311, SG312, SG313, SG314, SG315, SG316, SG317, SG318, SG319, SG320, SG321, SG322, SG323, SG324, SG325, SG326, SG327, SG328, SG329, SG330, SG331, SG332, SG333, SG334, SG335, SG336, SG337, SG338, SG339, SG340, SG341, SG342, SG343, SG344, SG345, SG346, SG347, SG348, SG349, SG350, SG351, SG352, SG353, SG354, SG355, SG356, SG357, SG358, SG359, SG360, SG361, SG362, SG363, SG364, SG365, SG366, SG367, SG368, SG369, SG370, SG371, SG372, SG373, SG374, SG375, SG376, SG377, SG378, SG379, SG380, SG381, SG382, SG383, SG384, SG385, SG386, SG387, SG388, SG389, SG390, SG391, SG392, SG393, SG394, SG395, SG396, SG397, SG398, SG399, SG400, SG401, SG402, SG403, SG404, SG405, SG406, SG407, SG408, SG409, SG410, SG411, SG412, SG413, SG414, SG415, SG416, SG417, SG418, SG419, SG420, SG421, SG422, SG423, SG424, SG425, SG426, SG427, SG428, SG429, SG430, SG431, SG432, SG433, SG434, SG435, SG436, SG437, SG438, SG439, SG440, SG441, SG442, SG443, SG444, SG445, SG446, SG447, SG448, SG449, SG450, SG451, SG452, SG453, SG454, SG455, SG456, SG457, SG458, SG459, SG460, SG461, SG462, SG465, SG468, SG469, SG470, SG471, SG472, SG473, SG474, SG475, SG476, SG477, SG478, SG479, SG480, SG481, SG482, SG483, SG484, SG485, SG486, SG487, SG488, SG489, SG490, SG491, SG492, SG493, SG494, SG495, SG496, SG497, SG498, SG499, SG500, SG501, SG502, SG503, SG504, SG505, SG506, SG507, SG508, SG509, SG510, SG511, SG512, SG513, SG514, SG515, SG516, SG517, SG518, SG519, SG520, SG521, SG522, SG523, SG524, SG525, SG526, SG527, SG428, SG529, SG530, SG531, SG532, SG533, SG534, SG535, SG536, SG537, SG538, SG539, SG540, SG541, SG542, SG543, SG544, SG545, SG546, SG547, SG548, SG549, SG550, SG551, SG552, SG553, SG554, SG555, SG556, SG557, SG558, SG559, SG560, SG561, SG562, SG563.

16. A composition in the form of microarray according to claim 15, which additionally comprises at least one pair of oligonucleotides selected from that composed of the oligonucleotides SG463 and SG464 and that composed of the oligonucleotides SG466 and SG467, at least one oligonucleotide from the group composed of SSPC1, SSPC2, SSPC3, SSPC4, SSPC5, SSPC6 and SSPC7, at least one oligonucleotide from the group composed of SCN2, SCN3, SCN6, SCN8, SCN11, SCN12 and SCN13 and at least one oligonucleotide from the group composed of SC1, SC2, SC3, SC4, SC5, SC6, SC7, SCN1, SCN5, SCN7 and SCN10.

17. A composition in the form of microarray according to claim 16, which comprises the totality of the oligonucleotides from the group composed of SG463, SG464, SG466, SG467, SSPC1, SSPC2, SSPC3, SSPC4, SSPC5, SSPC6, SSPC7, SCN2, SCN3, SCN6, SCN8, SCN11, SCN12, SCN13, SC1, SC2, SC3, SC4, SC5, SC6, SC7, SCN1, SCN5, SCN7 and SCN10.

18. A composition in the form of microarray according to claim 17, which additionally comprises points lacking oligonucleotides wherein the solvent wherein the oligonucleotides are found on being deposited on said glass is bound to the glass.

19. A composition in the form of microarray according to claim 18, which comprises at least twelve copies of each one of the different oligonucleotides present therein, as well as at least twelve points lacking oligonucleotides wherein the solvent wherein the oligonucleotides are found on being deposited on said glass is bound to the glass.

20. A composition in the form of microarray according to claim 18, wherein in the points lacking oligonucleotides the DMSO solvent is bound to the glass.

21. A composition in the form of microarray according to claim 15 to be used in the in vitro diagnosis of chronic lymphatic leukemia and/or for the in vitro prognosis of the evolution of said disease.

22. A device for the in vitro diagnosis of a neoplasia originating from hematopoietic cells and/or for the in vitro prognosis of the evolution thereof, which comprises a composition according to claim 1.

23. A combination comprising the device for the diagnosis of a neoplasia originating from hematopoietic cells and/or for the in vitro prognosis of the evolution thereof according to claim 22, and a composition in the form of microarray wherein the oligonucleotides are disposed in an ordered fashion on a solid support which is glass similar to a slide whereto the oligonucleotides are bound by covalent bonds, forming a microarray.

24. A combination comprising the device for the in vitro diagnosis of a neoplasia originating from hematopoietic cells and/or for the in vitro prognosis of the evolution thereof according to claim 22, and a composition in the form of microarray.

25. A device for the in vitro diagnosis of a neoplasia originating from hematopoietic cells and/or for the in vitro prognosis of the evolution thereof according to claim 23, wherein the neoplasia which is diagnosed or a whose evolution a prognosis is made of is chronic lymphatic leukemia.

26. A method for the in vitro diagnosis of a neoplasia originating from hematopoietic cells and/or in vitro prognosis of the evolution thereof which comprises the in vitro detection from a biological sample and the statistical analysis of the expression level of at least one significant gene for classifying the sample associated or not to said neoplasia, a gene which is selected from the group composed of GABARAP, NPM3, ABCB1, ABCB4, ABCC3, ABCC5, ABCC6, ABHD1, ABL1, ACTN1, AF1q, AKR1A1, ALDH1A1, ALK, ANK2, ANPEP, ANXA6, ANXA7, APAF1, APEX, ARHGEF2, ARS2, ASNS, ATIC, ATM, ATP50, BAX, BCL10, BCL2, BCL2A1, BCL2L1, BCL2LAA, BCL3, BCL6, BCL7A, BCL7b, BCR, BECN1, BIK, BIRC3, BIRC5, BLMH, BLR1, BLVRB, BMI1, BMP6, BRMS1, BST2, BTG1, BUB1, C21orf33, C5orfl3, CA12, CALD1, CANP2, CASC3, CASP1, CASP3, CASP4, CASP5, CASP6, CASP7, CASP8, CASP9, CAST, CATSD, CBFA2T1, CBFB, CCNA1, CCNB1, CCND1, CCND2, CCND3, CCNE1, CCR6, CCR7, CCT6A, CD14, CD19, CD2, CD22, CD24, CD28, CD33, CD34, CD36, CD38, CD3E, CD4, CD44, CD47, CD48, CD5, CD58, CD59, CD6, CD7, CD79A, CD79B, CD8, CD81, CD83, CD86, CD9, CDA, CDC25A, CDC25B, CDK2, CDK4, CDK5R1, CDKN1A, CDKN1B, CDKN1C, CDKN2A, CDKN2B, CDKN2C, CDKN3, CDW52, CEBPA, CEBPB, CEBPD, CFL1, CKMT1, CKS2, CML66, COL3A1, COL4A6, CR2, CREB1, CREBBP, CRYAB, CSF2, CSF3, CSRP2, CTGF, CTSB, CUZD1, CXADR, CXCL9, CXCR3, CXCR4, CYC1, CYP1A1, CYP2A6, DAD-1, DAPK1, DCK, DDX6, DEK, DHFR, DLAD, DNAJA1, DNMT3B, DNTT, DOK1, DPF2, DPP4, DRG1, DRP2, E2F1, EB-1, EBI2, EDF1, EEF1A1, EEF1B2, EEF1D, EEF1G, EFNB1, EGFR, EGR1, EIF2B2, EIF3S2, EIF4B, EIF4E, EIF5A, ELF1, ELF4, ENPP1, EphA3, EPOR, ERBB2, ERBB4, ERCC1, ERCC2, ERCC3, ERCC5, ERCC6, ETS1, ETS2, ETV6, ETV7, EZH2, FABP5, FADD, FAIM3, FAM38A, FARP1, FAT, FCER2, FCGR3A, FCGR3B, FGFR1, FGFR3, FGR, FHIT, FKBP9, FLI1, FLJ22169, FLT3, FN1, FNTB, FOS, FUS, G1P2, GABPB2, GATA1, GATA2, GATA3, GCET2, GDI2, GGA3, GJA1, GLUD1, GNL3, GOT1, GRB2, GRIA3, GRK4, GSTP1, GSTT1, GUSB, GZMA, H2AFX, H3F₃A, HCK, HELLS, HIF1A, HIST1H₂BN, HLA-A, HLA-DPA1, HLA-DQA1, HLA-DRA, HLA-DRB3, HLF, HMMR, HNRPH3, HNRPL, HOXA10, HOXA9, HOXD8, HOXD9, HRAS, HSD17B1, HSPB1, IBSP, ICAM1, ICAM3, ID2, IER3, IFRD1, IGFBP2, IGFBP3, IGFIR, IGLV6-57, IL10, IL15, IL1B, IL2, IL2RA, IL3, IL32, IL3RA, IL4R, IL6, IL6R, IL8, ILF2, IRF1, IRF2, IRF4, IRF8, ITGA2, ITGA3, ITGA4, ITGA5, ITGA6, ITGAL, ITGAM, ITGAX, ITGB1, ITGB2, JAK1, JAK2, JUNB, KAI1, KIAA0247, KIAA0864, KIT, KLF1, KLF13, KRAS2, KRT18, LADH, LAG3, LASP1, LCK, LCP1, LEPR, LGALS3, LGALS7, LIF, LIMS1, LMO2, LOC285148, LRP, LSP1, LYL1, LYN, LYZ, MAFB, MAFK, MAGEA1, MAL, MAP3K12, MAP4K1, MAPK10, MAZ, MBP1, MCL1, MCM3, MCM7, MDM2, MEIS1, MEN1, MERTK, MKI67, MLF1, MLF2, MLL, MLLT10, MME, MMP2, MMP7, MMP8, MMP9, MNDA, MPL, MPO, MRPL37, MS4A1, MTCP1, MUC-1, MX1, MYB, MYBL1, MYC, MYOD1, NCALD, NCAM1, NCL, NDP52, NDRG1, NDUFA1, NDUFB, NF1, NFATC1, NFIC, NFKB1, NFKB1A, NINJ1, NPM1, NR3C1, NUMA1, NXF1, ODC1, OGGI, OLIG2, OPRD1, p14ARF, P55CDC, PABPC1, PAX5, PAX6, PAX8, PBX1, PBX3, PCA1, PCD, PCNA, PDCD1, PDGFA, PDGFRB, PDHA1, PGF, PGRMC1, PICALM, PLA2G6, PLAU, PLK1, PLP, PLS3, PLZF, PML, PMM1, POLR2c, POU2F2, PPP1CC, PRAME, PRKCl, PRKCQ, PRKDC, PRL, PRTN3, PSMA5, PSMB4, PSMC5, PSMD7, PTEN, PTGS1, PTHLH, PTK2, PTK2B, PTN, PTPRCCD, PYGB, RAD51, RAF1, RAG1, RARA, RARB, RB1, RBBP4, RBBP6, RBBP8, RBP4, RET, RGS1, RGS1, RIS1, RORA, RPL17, RPL23A, RPL24, RPL36A, RPL37A, RPL41, RPS3, RPS5, RPS9, RUNX1, RxRA, S100A2, S100A8, SDC1, SDHD, SELE, SELL, SEPW1, SERPINA9, SERPINB5, SERPNINA9, SFTPB, SIAT4A, SLC7A5, SNRPB, SOSTDC1, SP1, SPI1, SPN, SPRR1A, SREBF1, SSBP1, STAT1, STAT3, STAT5B, SUMO1, TACSTD2, TAGLN2, TAL1, TBP, TCEB1, TCF1, TCF3, TCF7, TCL1A, TCRbeta, TEGT, TERF1, TERT, TFCP2, TFRC, THBS1, THPO, TIA-2, TIAM1, TK1, TLX1, TMEM4, TNF, TNFRSF10C, TNFRSF1A, TNFRSF25, TNFRSF5, TNFRSF6, TNFRSF8, TNFSF10, TNFSF5, TNFSF6, TOP2A, TOPORS, TP73, TRA@, TRADD, TRAF3, TRAP1, TRIB2, TXNRD1, UBE2C, UHRF1, UVRAG, VCAM1, VEGF, VPREB1, WBSCR20C, WNT16, WT1, XBP1, XPO6, XRCC3, XRCC5, ZAP70, ZFPL1, ZNF42, ZNFN1A1, ZYX, 18S rRNA, 28S rRNA and whose expression level is determined by the evaluation of the concentration of its corresponding mRNA by the use of at least one probe which has a sequence complementary to a fragment of a strand of said gene, a probe which is selected from the group of oligonucleotides composed of:
SG1, SG2, SG3, SG4, SG5, SG6, SG7, SG8, SG9, SG10, SG11, SG12, SG13, SG14, SG15, SG16, SG17, SG18, SG19, SG20, SG21, SG22, SG23, SG24, SG25, SG26, SG27, SG28, SG29, SG30, SG31, SG32, SG33, SG34, SG35, SG36, SG37, SG38, SG39, SG40, SG41, SG42, SG43, SG44, SG45, SG46, SG47, SG48, SG49, SG50, SG51, SG52, SG53, SG54, SG55, SG56, SG57, SG58, SG59, SG60, SG61, SG62, SG63, SG64, SG65, SG66, SG67, SG68, SG69, SG70, SG71, SG72, SG73, SG74, SG75, SG76, SG77, SG78, SG79, SG80, SG81, SG82, SG83, SG84, SG85, SG86, SG87, SG88, SG89, SG90, SG91, SG92, SG93, SG94, SG95, SG96, SG97, SG98, SG99, SG100, SG101, SG102, SG103, SG104, SG105, SG106, SG107, SG108, SG109, SG110, SG111, SG112, SG113, SG114, SG115, SG116, SG117, SG118, SG119, SG120, SG121, SG122, SG123, SG124, SG125, SG126, SG127, SG128, SG129, SG130, SG131, SG132, SG133, SG134, SG135, SG136, SG137, SG138, SG139, SG140, SG141, SG142, SG143, SG144, SG145, SG146, SG147, SG148, SG149, SG150, SG151, SG152, SG153, SG154, SG155, SG156, SG157, SG158, SG159, SG160, SG161, SG162, SG163, SG164, SG165, SG166, SG167, SG168, SG169, SG170, SG171, SG172, SG173, SG174, SG175, SG176, SG177, SG178, SG179, SG180, SG181, SG182, SG183, SG184, SG185, SG186, SG187, SG188, SG189, SG190, SG191, SG192, SG193, SG194, SG195, SG196, SG197, SG198, SG199, SG200, SG201, SG202, SG203, SG204, SG205, SG206, SG207, SG208, SG209, SG210, SG211, SG212, SG213, SG214, SG215, SG216, SG217, SG218, SG219, SG220, SG221, SG222, SG223, SG224, SG225, SG226, SG227, SG228, SG229, SG230, SG231, SG232, SG233, SG234, SG235, SG236, SG237, SG238, SG239, SG240, SG241, SG242, SG243, SG244, SG245, SG246, SG247, SG248, SG249, SG250, SG251, SG252, SG253, SG254, SG255, SG256, SG257, SG258, SG259, SG260, SG261, SG262, SG263, SG264, SG265, SG266, SG267, SG268, SG269, SG270, SG271, SG272, SG273, SG274, SG275, SG276, SG277, SG278, SG279, SG280, SG281, SG282, SG283, SG284, SG285, SG286, SG287, SG288, SG289, SG290, SG291, SG292, SG293, SG294, SG295, SG296, SG297, SG298, SG299, SG300, SG301, SG302, SG303, SG304, SG305, SG306, SG307, SG308, SG309, SG310, SG311, SG312, SG313, SG314, SG315, SG316, SG317, SG318, SG319, SG320, SG321, SG322, SG323, SG324, SG325, SG326, SG327, SG328, SG329, SG330, SG331, SG332, SG333, SG334, SG335, SG336, SG337, SG338, SG339, SG340, SG341, SG342, SG343, SG344, SG345, SG346, SG347, SG348, SG349, SG350, SG351, SG352, SG353, SG354, SG355, SG356, SG357, SG358, SG359, SG360, SG361, SG362, SG363, SG364, SG365, SG366, SG367, SG368, SG369, SG370, SG371, SG372, SG373, SG374, SG375, SG376, SG377, SG378, SG379, SG380, SG381, SG382, SG383, SG384, SG385, SG386, SG387, SG388, SG389, SG390, SG391, SG392, SG393, SG394, SG395, SG396, SG397, SG398, SG399, SG400, SG401, SG402, SG403, SG404, SG405, SG406, SG407, SG408, SG409, SG410, SG411, SG412, SG413, SG414, SG415, SG416, SG417, SG418, SG419, SG420, SG421, SG422, SG423, SG424, SG425, SG426, SG427, SG428, SG429, SG430, SG431, SG432, SG433, SG434, SG435, SG436, SG437, SG438, SG439, SG440, SG441, SG442, SG443, SG444, SG445, SG446, SG447, SG448, SG449, SG450, SG451, SG452, SG453, SG454, SG455, SG456, SG457, SG458, SG459, SG460, SG461, SG462, SG465, SG468, SG469, SG470, SG471, SG472, SG473, SG474, SG475, SG476, SG477, SG478, SG479, SG480, SG481, SG482, SG483, SG484, SG485, SG486, SG487, SG488, SG489, SG490, SG491, SG492, SG493, SG494, SG495, SG496, SG497, SG498, SG499, SG500, SG501, SG502, SG503, SG504, SG505, SG506, SG507, SG508, SG509, SG510, SG511, SG512, SG513, SG514, SG515, SG516, SG517, SG518, SG519, SG520, SG521, SG522, SG523, SG524, SG525, SG526, SG527, SG428, SG529, SG530, SG531, SG532, SG533, SG534, SG535, SG536, SG537, SG538, SG539, SG540, SG541, SG542, SG543, SG544, SG545, SG546, SG547, SG548, SG549, SG550, SG551, SG552, SG553, SG554, SG555, SG556, SG557, SG558, SG559, SG560, SG561, SG562, SG563, or combinations thereof.

27. Method for the in vitro diagnosis of a neoplasia originating from hematopoietic cells and/or the in vitro prognosis of the evolution thereof according to claim 26, which additionally comprises a previous optional step of identification of genes significant for the classification of a sample as associated or not to a specific type of neoplasia originating from hematopoietic cells, a previous step which comprises the substeps of:
a) deciding the possible categories wherein the sample can be classified;
b) obtaining biological samples from individuals which have previously been assigned by a method different to that claimed to any of the possible classification categories, so that there are samples of each one of the possible categories;
c) obtaining the total mRNA of each one of the samples;
d) obtaining the corresponding total cRNA, labelled by a method which allows its subsequent detection, of at least one aliquot of each one of the samples of mRNA, an aliquot whereto is added before the obtainment of the cRNA at least one sequence of polyadenylated nucleotides of low homology with human genes for which it acts as internal positive control of the process;
e) adding to one of the aliquots of cRNA which are going to be used in step f) at least one oligonucleotide of low homology with human genes different from and not complementary to any possible sequence of nucleotides which have been added in step d), for which it acts as positive hybridization control;
f) hybridizing, in strict conditions, at least one aliquot of total cRNA of each one of the samples with at least one microarray which comprises at least two copies of each one of the oligonucleotides from the group composed of:
SG1, SG2, SG3, SG4, SG5, SG6, SG7, SG8, SG9, SG10, SG11, SG12, SG13, SG14, SG15, SG16, SG17, SG18, SG19, SG20, SG21, SG22, SG23, SG24, SG25, SG26, SG27, SG28, SG29, SG30, SG31, SG32, SG33, SG34, SG35, SG36, SG37, SG38, SG39, SG40, SG41, SG42, SG43, SG44, SG45, SG46, SG47, SG48, SG49, SG50, SG51, SG52, SG53, SG54, SG55, SG56, SG57, SG58, SG59, SG60, SG61, SG62, SG63, SG64, SG65, SG66, SG67, SG68, SG69, SG70, SG71, SG72, SG73, SG74, SG75, SG76, SG77, SG78, SG79, SG80, SG81, SG82, SG83, SG84, SG85, SG86, SG87, SG88, SG89, SG90, SG91, SG92, SG93, SG94, SG95, SG96, SG97, SG98, SG99, SG100, SG101, SG102, SG103, SG104, SG105, SG106, SG107, SG108, SG109, SG110, SG111, SG112, SG113, SG114, SG115, SG116, SG117, SG118, SG119, SG120, SG121, SG122, SG123, SG124, SG125, SG126, SG127, SG128, SG129, SG130, SG131, SG132, SG133, SG134, SG135, SG136, SG137, SG138, SG139, SG140, SG141, SG142, SG143, SG144, SG145, SG146, SG147, SG148, SG149, SG150, SG151, SG152, SG153, SG154, SG155, SG156, SG157, SG158, SG159, SG160, SG161, SG162, SG163, SG164, SG165, SG166, SG167, SG168, SG169, SG170, SG171, SG172, SG173, SG174, SG175, SG176, SG177, SG178, SG179, SG180, SG181, SG182, SG183, SG184, SG185, SG186, SG187, SG188, SG189, SG190, SG191, SG192, SG193, SG194, SG195, SG196, SG197, SG198, SG199, SG200, SG201, SG202, SG203, SG204, SG205, SG206, SG207, SG208, SG209, SG210, SG211, SG212, SG213, SG214, SG215, SG216, SG217, SG218, SG219, SG220, SG221, SG222, SG223, SG224, SG225, SG226, SG227, SG228, SG229, SG230, SG231, SG232, SG233, SG234, SG235, SG236, SG237, SG238, SG239, SG240, SG241, SG242, SG243, SG244, SG245, SG246, SG247, SG248, SG249, SG250, SG251, SG252, SG253, SG254, SG255, SG256, SG257, SG258, SG259, SG260, SG261, SG262, SG263, SG264, SG265, SG266, SG267, SG268, SG269, SG270, SG271, SG272, SG273, SG274, SG275, SG276, SG277, SG278, SG279, SG280, SG281, SG282, SG283, SG284, SG285, SG286, SG287, SG288, SG289, SG290, SG291, SG292, SG293, SG294, SG295, SG296, SG297, SG298, SG299, SG300, SG301, SG302, SG303, SG304, SG305, SG306, SG307, SG308, SG309, SG310, SG311, SG312, SG313, SG314, SG315, SG316, SG317, SG318, SG319, SG320, SG321, SG322, SG323, SG324, SG325, SG326, SG327, SG328, SG329, SG330, SG331, SG332, SG333, SG334, SG335, SG336, SG337, SG338, SG339, SG340, SG341, SG342, SG343, SG344, SG345, SG346, SG347, SG348, SG349, SG350, SG351, SG352, SG353, SG354, SG355, SG356, SG357, SG358, SG359, SG360, SG361, SG362, SG363, SG364, SG365, SG366, SG367, SG368, SG369, SG370, SG371, SG372, SG373, SG374, SG375, SG376, SG377, SG378, SG379, SG380, SG381, SG382, SG383, SG384, SG385, SG386, SG387, SG388, SG389, SG390, SG391, SG392, SG393, SG394, SG395, SG396, SG397, SG398, SG399, SG400, SG401, SG402, SG403, SG404, SG405, SG406, SG407, SG408, SG409, SG410, SG411, SG412, SG413, SG414, SG415, SG416, SG417, SG418, SG419, SG420, SG421, SG422, SG423, SG424, SG425, SG426, SG427, SG428, SG429, SG430, SG431, SG432, SG433, SG434, SG435, SG436, SG437, SG438, SG439, SG440, SG441, SG442, SG443, SG444, SG445, SG446, SG447, SG448, SG449, SG450, SG451, SG452, SG453, SG454, SG455, SG456, SG457, SG458, SG459, SG460, SG461, SG462, SG465, SG468, SG469, SG470, SG471, SG472, SG473, SG474, SG475, SG476, SG477, SG478, SG479, SG480, SG481, SG482, SG483, SG484, SG485, SG486, SG487, SG488, SG489, SG490, SG491, SG492, SG493, SG494, SG495, SG496, SG497, SG498, SG499, SG500, SG501, SG502, SG503, SG504, SG505, SG506, SG507, SG508, SG509, SG510, SG511, SG512, SG513, SG514, SG515, SG516, SG517, SG518, SG519, SG520, SG521, SG522, SG523, SG524, SG525, SG526, SG527, SG428, SG529, SG530, SG531, SG532, SG533, SG534, SG535, SG536, SG537, SG538, SG539, SG540, SG541, SG542, SG543, SG544, SG545, SG546, SG547, SG548, SG549, SG550, SG551, SG552, SG553, SG554, SG555, SG556, SG557, SG558, SG559, SG560, SG561, SG562, SG563,
a microarray which additionally comprises:
a. at least two points which correspond to different aliquots of the solvent wherein nucleotides are found at the time of their deposit on the surface of the microarray, for which they serve as blank,
b. at least two copies of at least one oligonucleotide for each one of the polyadenylated sequences added in step d), an oligonucleotide whose sequence will correspond to a fragment, different from the polyadenylation zone, of the sequence of polyadenylated nucleotides whose evolution in the process has to be controlled;
c. for each one of the oligonucleotides added in step e), at least two copies of an oligonucleotide complementary thereto;
d. at least two copies of each member of at least one pair of oligonucleotides wherein the sequence of one of the members corresponds to a sequence of zone 5′ and the sequence of the other corresponds to a sequence of zone 3′ of the mRNA of a gene which is expressed in constitutive form in any cell of hematopoietic origin;
e. at least two copies of at least one oligonucleotide of low homology with human genes different from any of the oligonucleotides defined in section b. and different from any of the synthetic oligonucleotides added optionally in step e);
g) detecting and quantifying the signal of cRNA hybridized with each one of the copies of each one of the oligonucleotides present in the microarray, as well as the signal corresponding to the points of the solvent;
h) calculating the average level of intensity of hybridization of each one of the oligonucleotides of the microarray calculating the average of the intensities of the copies of each one of the oligonucleotides;
i) taking the hybridization as valid if the following conditions are complied with:
a. the ratio between the average intensity and the average background of all the oligonucleotides of the microarray is greater than 10;
b. the value of the average coefficient of variation of all the replicas of oligonucleotides should be less than 0.3;
c. the average value of negative control should be less than 2.5 times the average value of the points corresponding to the solvent;
d. there is a signal both in the hybridization controls and in the internal positive controls used as process control;
j) normalizing the data;
k) eliminating the oligonucleotides with values of average intensity minus average background noise less than approximately 2 times the average value obtained with the points corresponding to the solvent, as well as the oligonucleotides with an interquartile range of normalized intensity throughout the samples less than 0.3;
l) performing the statistical analysis to find the statistically significant oligonucleotides to differentiate between the different categories and be able to classify a sample which has not been previously assigned to any category, choosing said oligonucleotides among those which have not been eliminated in the previous steps, until obtaining “n” oligonucleotides which either have a value of p less than a limit which is chosen from the open range of 0 to 0.05, preferably using for it a method with capacity to reduce false positives, or that which best defines the category established;
m) checking that the grouping of the samples according to the differences in intensities between the different samples detected for the statistically significant oligonucleotides gives rise to the samples being classified in the same categories as those which had previously been assigned by a different method.

28. Method according to claim 27, wherein the microarray comprises at least four copies of each one of the oligonucleotides present in it and the average of the intensities of the copies of each one of the oligonucleotides which is calculated in h) is a trimmed mean.

29. Method according to claim 28, wherein the normalization is carried out using the “variance stabilization normalization” method available in the “vsn” package in R.

30. Method according to claim 27, wherein the statistical analysis to find the statistically significant oligonucleotides to differentiate between the different categories is carried out using the mt.maxT function of the multtest package in R.

31. Method according to claim 27, wherein the diagnosis is done with a diagnostic device which comprises a composition containing at least one oligonucleotide from the group composed of:
SG1, SG2, SG3, SG4, SG5, SG6, SG7, SG8, SG9, SG10, SG11, SG12, SG13, SG14, SG15, SG16, SG17, SG18, SG19, SG20, SG21, SG22, SG23, SG24, SG25, SG26, SG27, SG28, SG29, SG30, SG31, SG32, SG33, SG34, SG35, SG36, SG37, SG38, SG39, SG40, SG41, SG42, SG43, SG44, SG45, SG46, SG47, SG48, SG49, SG50, SG51, SG52, SG53, SG54, SG55, SG56, SG57, SG58, SG59, SG60, SG61, SG62, SG63, SG64, SG65, SG66, SG67, SG68, SG69, SG70, SG71, SG72, SG73, SG74, SG75, SG76, SG77, SG78, SG79, SG80, SG81, SG82, SG83, SG84, SG85, SG86, SG87, SG88, SG89, SG90, SG91, SG92, SG93, SG94, SG95, SG96, SG97, SG98, SG99, SG100, SG101, SG102, SG103, SG104, SG105, SG106, SG107, SG108, SG109, SG110, SG111, SG112, SG113, SG114, SG115, SG116, SG117, SG118, SG119, SG120, SG121, SG122, SG123, SG124, SG125, SG126, SG127, SG128, SG129, SG130, SG131, SG132, SG133, SG134, SG135, SG136, SG137, SG138, SG139, SG140, SG141, SG142, SG143, SG144, SG145, SG146, SG147, SG148, SG149, SG150, SG151, SG152, SG153, SG154, SG155, SG156, SG157, SG158, SG159, SG160, SG161, SG162, SG163, SG164, SG165, SG166, SG167, SG168, SG169, SG170, SG171, SG172, SG173, SG174, SG175, SG176, SG177, SG178, SG179, SG180, SG181, SG182, SG183, SG184, SG185, SG186, SG187, SG188, SG189, SG190, SG191, SG192, SG193, SG194, SG195, SG196, SG197, SG198, SG199, SG200, SG201, SG202, SG203, SG204, SG205, SG206, SG207, SG208, SG209, SG210, SG211, SG212, SG213, SG214, SG215, SG216, SG217, SG218, SG219, SG220, SG221, SG222, SG223, SG224, SG225, SG226, SG227, SG228, SG229, SG230, SG231, SG232, SG233, SG234, SG235, SG236, SG237, SG238, SG239, SG240, SG241, SG242, SG243, SG244, SG245, SG246, SG247, SG248, SG249, SG250, SG251, SG252, SG253, SG254, SG255, SG256, SG257, SG258, SG259, SG260, SG261, SG262, SG263, SG264, SG265, SG266, SG267, SG268, SG269, SG270, SG271, SG272, SG273, SG274, SG275, SG276, SG277, SG278, SG279, SG280, SG281, SG282, SG283, SG284, SG285, SG286, SG287, SG288, SG289, SG290, SG291, SG292, SG293, SG294, SG295, SG296, SG297, SG298, SG299, SG300, SG301, SG302, SG303, SG304, SG305, SG306, SG307, SG308, SG309, SG310, SG311, SG312, SG313, SG314, SG315, SG316, SG317, SG318, SG319, SG320, SG321, SG322, SG323, SG324, SG325, SG326, SG327, SG328, SG329, SG330, SG331, SG332, SG333, SG334, SG335, SG336, SG337, SG338, SG339, SG340, SG341, SG342, SG343, SG344, SG345, SG346, SG347, SG348, SG349, SG350, SG351, SG352, SG353, SG354, SG355, SG356, SG357, SG358, SG359, SG360, SG361, SG362, SG363, SG364, SG365, SG366, SG367, SG368, SG369, SG370, SG371, SG372, SG373, SG374, SG375, SG376, SG377, SG378, SG379, SG380, SG381, SG382, SG383, SG384, SG385, SG386, SG387, SG388, SG389, SG390, SG391, SG392, SG393, SG394, SG395, SG396, SG397, SG398, SG399, SG400, SG401, SG402, SG403, SG404, SG405, SG406, SG407, SG408, SG409, SG410, SG411, SG412, SG413, SG414, SG415, SG416, SG417, SG418, SG419, SG420, SG421, SG422, SG423, SG424, SG425, SG426, SG427, SG428, SG429, SG430, SG431, SG432, SG433, SG434, SG435, SG436, SG437, SG438, SG439, SG440, SG441, SG442, SG443, SG444, SG445, SG446, SG447, SG448, SG449, SG450, SG451, SG452, SG453, SG454, SG455, SG456, SG457, SG458, SG459, SG460, SG461, SG462, SG465, SG468, SG469, SG470, SG471, SG472, SG473, SG474, SG475, SG476, SG477, SG478, SG479, SG480, SG481, SG482, SG483, SG484, SG485, SG486, SG487, SG488, SG489, SG490, SG491, SG492, SG493, SG494, SG495, SG496, SG497, SG498, SG499, SG500, SG501, SG502, SG503, SG504, SG505, SG506, SG507, SG508, SG509, SG510, SG511, SG512, SG513, SG514, SG515, SG516, SG517, SG518, SG519, SG520, SG521, SG522, SG523, SG524, SG525, SG526, SG527, SG428, SG529, SG530, SG531, SG532, SG533, SG534, SG535, SG536, SG537, SG538, SG539, SG540, SG541, SG542, SG543, SG544, SG545, SG546, SG547, SG548, SG549, SG550, SG551, SG552, SG553, SG554, SG555, SG556, SG557, SG558, SG559, SG560, SG561, SG562, SG563,
or combinations thereof, to be used as probe in the determination of the expression level of a gene which possesses a sequence complementary to said oligonucleotide by the evaluation of the mRNA level corresponding to that gene, of application in the in vitro diagnosis of neoplasias originating from hematopoietic cells and/or in the in vitro prognosis of the evolution of said disease.

32. Method according to claim 27, which comprises an optional step of obtainment of a classification function for each sample by the arbitrary assignment of the value of 0 to one of the possible categories “a” and of the value 1 to the other possible category “b” wherein it is possible to classify the sample and the obtainment by logistical regression of a coefficient for each one of the oligonucleotides which make it possible to calculate a value xᵢ for each sample by a function of the type:
n
xᵢ=constant+Σ(coeff_oligₘ*Imnᵢ_{_{—}}oligₘ)
m=1
where
coeff_oligₘ represents the coefficient calculated for a specific oligonucleotide
Imnᵢ_{_{—}}oligₘ represents the average value of normalized intensity obtained in the hybridization of the sample i calculated for the oligonucleotide
“m” varies from 1 to “n”
n is the total number of oligonucleotides considered significant
value “xᵢ” wherefrom the probability “pᵢ” that a sample “i” belongs to one or another category is calculated using the formula pᵢ=1/(1+e^{-xi) and classifying the sample as belonging to category “a” or “b” according to its corresponding value p}ᵢ is closer to 0 or 1, respectively.

33. Method according to claim 27, wherein the statistical analysis to find the significant oligonucleotides to differentiate between the different categories is carried out using the “Nearest Shrunken Centroids” method.

34. Method according to claim 27, wherein the biological samples analysed in vitro are samples of peripheral blood.

35. Method according to claim 34, wherein a leukemia is diagnosed in vitro or a prognosis is made of the evolution thereof.

36. Method according to claim 35, wherein it is diagnosed in vitro if an individual suffers from chronic lymphatic leukemia.

37. Method according to claim 35, wherein an in vitro prognosis is made of the evolution of the chronic lymphatic leukemia in a subject classifying a sample of blood extracted therefrom as “associated to stable chronic lymphatic leukemia” or as “associated to progressive chronic lymphatic leukemia”.

38. Method to make an in vitro diagnosis of a neoplasia originating from hematopoietic cells and/or an in vitro prognosis of the evolution thereof which comprises the in vitro detection and the statistical analysis of the expression level of at least one significant gene for classifying the sample as belonging to a healthy individual or associating it to a type of neoplasia originating from hematopoietic cells according to claim 26, wherein the neoplasia which is diagnosed and/or whose evolution a prognosis is made of is a leukemia.

39. Method according to claim 38, wherein a diagnosis/or prognosis is made of the evolution of the chronic lymphatic leukemia.

40. Method to make an in vitro diagnosis of chronic lymphatic leukemia and/or make an in vitro prognosis of its evolution according to claim 39, wherein the in vitro detection of the expression level of at least one significant gene is carried out from samples of peripheral blood.

41. Method to make an in vitro diagnosis of chronic lymphatic leukemia according to claim 40, wherein the subjects wherefrom the corresponding blood samples have been taken are classified in the category of subject not suffering from CLL or in the category of subject suffering from CLL.

42. Method to make an in vitro diagnosis of chronic lymphatic leukemia according to claim 41, wherein the classification of the subjects is carried out after the in vitro detection and the statistical analysis of the expression level in the corresponding blood samples of at least genes CD79A, FAIM3, HLA-DRA, HLA-DRB3, HLA-DQA1.

43. Method to make an in vitro diagnosis of chronic lymphatic leukemia according to claim 42, wherein the classification of the subjects is carried out after the in vitro detection and the statistical analysis of the expression level in the corresponding blood samples additionally of genes IRF8 and COL3A1.

44. Method to make an in vitro diagnosis of chronic lymphatic leukemia according to claim 43, wherein the in vitro detection and the statistical analysis of the expression level of genes CD79A, FAIM3, HLA-DRA, HLA-DRB3, HLA-DQA1, IRF8 and COL3A1 is carried out by the evaluation of the corresponding mRNA by hybridization of its corresponding cRNA using as probes the oligonucleotides SG117, SG428, SG459, SG507, SG508, SG461 and SG493.

45. Method to make an in vitro diagnosis of chronic lymphatic leukemia according to claim 44, wherein the oligonucleotides form part of a composition in the form of microarray.

46. Method to make an in vitro diagnosis of chronic lymphatic leukemia according to claim 45, wherein the evaluation of the hybridized cRNA is carried out thanks to the prior labelling of cRNA with biotin, the staining of the hybridized microarray with streptavidin conjugated with a fluorophore and the detection of the signal emitted by said fluorophore.

47. Method to make an in vitro diagnosis of chronic lymphatic leukemia according to claim 46, wherein the fluorophore is Cy3.

48. Method to make an in vitro diagnosis of chronic lymphatic leukemia according to claim 47, wherein the classification of a subject from which the sample I has been taken analysed in the category of subject not suffering from CLL or in the category of subject suffering from CLL is carried out by calculating for said subject a value of probability p, =1/(1+e^{-xi}) after obtaining its corresponding value of xᵢ by the formula
xᵢ=−719.241486+(2.44756372*Imnᵢ_{_{—}}CD79A)+(7.38657611*Imnᵢ_{_{—}}FAIM3)+(23.1465464*Imnᵢ_{_{—}}HLA-DRA)+(43.6287742*Imnᵢ_{_{—}}IRF8)−(19.3978182*Imnᵢ_{_{—}}COL3A1)−(2.80282646*Imnᵢ_{_{—}}HLA-DRB3)+(49.5345672*Imnᵢ_{_{—}}HLA-DQA1)
formula wherein each one of the values called abbreviation “Imnᵢ” followed by the abbreviation of a gene makes reference to the average value of normalized intensity obtained after detecting the hybridization signal corresponding to the oligonucleotide which is being used as probe to evaluate the expression of the said gene
and classifying the subject as subject not suffering from CLL if the value of pᵢ is less than 0.5 and as subject suffering from CLL if the value of pᵢ is greater than 0.5.

49. Method to make an in vitro prognosis of the evolution of the disease in a subject suffering from chronic lymphatic leukemia according to claim 40, wherein the subjects from which the corresponding blood samples have been taken are classified in the category of subject with stable CLL or in the category of subject with progressive CLL.

50. Method to make an in vitro prognosis of the evolution of the disease in a subject suffering from chronic lymphatic leukemia according to claim 49, wherein the classification of the subjects is carried out after the in vitro detection and the statistical analysis of the expression level in the corresponding blood samples of at least genes PSMB4, FCER2 and POU2F2.

51. Method to make an in vitro prognosis of the evolution of the disease in a subject suffering from chronic lymphatic leukemia according to claim 50, wherein the classification of the subjects is carried out after the in vitro detection and the statistical analysis of the expression level in the corresponding blood samples additionally of at least one gene selected from the group composed of ODC1, CD79A, CD2, CD3E, CD5, MS4A1, EIF4E, FHIT, NR3C1, LCP1, MAPK10, ABCC5, XRCC3, CML66, PLZF, RBP4.

52. Method to make an in vitro prognosis of the evolution of the disease in a subject suffering from chronic lymphatic leukemia according to claim 51, wherein the classification of the subjects is carried out after the in vitro detection and the statistical analysis of the expression level in the corresponding blood samples of at least the genes of the group composed of PSMB4, FCER2, POU2F2, ODC1, CD79A, CD2, CD3E, CD5, MS4A1, EIF4E, FHIT, NR3C1, LCP1, MAPK10, ABCC5, XRCC3, CML66, PLZF, RBP4.

53. Method to make an in vitro prognosis of the evolution of the disease in a subject suffering from chronic lymphatic leukemia according to either of claims 51, wherein the in vitro detection and the statistical analysis of the expression level of the genes examined is carried out by the evaluation of the corresponding mRNA by hybridization of its corresponding cRNA using as probes the corresponding oligonucleotides selected from the group composed of SG26, SG216, SG366, SG31, SG177, SG194, SG195, SG197, SG213, SG293, SG301, SG309, SG33, SG343, SG357, SG439, SG452, SG555, SG556.

54. Method to make an in vitro prognosis of the evolution of the disease in a subject suffering from chronic lymphatic leukemia according to claim 53, wherein the oligonucleotides form part of a composition in the form of microarray.

55. Method to make an in vitro prognosis of the evolution of the disease in a subject suffering from chronic lymphatic leukemia according to claim 54, wherein the evaluation of the corresponding mRNA of the sample analysed by the detection of the corresponding hybridized cRNA to the corresponding oligonucleotide is carried out thanks to the previous labelling of the cRNA with biotin, the staining of the microarray hybridized with streptavidin conjugated with a fluorophore and the detection of the signal emitted by said fluorophore.

56. Method to make an in vitro prognosis of the evolution of the disease in an individual suffering from chronic lymphatic leukemia according to claim 55, wherein the fluorophore is Cy3.

57. A method comprising use of a device for evaluation of the expression level of at least one gene of the group composed of PSMB4, FCER2, POU2F2, ODC1, CD79A, CD2, CD3E, CD5, MS4A1, EIF4E, FHIT, NR3C1, LCP1, MAPK10, ABCC5, XRCC3, CML66, PLZF, RBP4, CD79A, FAIM3, HLA-DRA, HLA-DRB3, HLA-DQA1, IRF8 and COL3A1 for the in vitro diagnosis of the existence of chronic lymphatic leukemia in a subject and/or for the in vitro prognosis of the evolution of the chronic lymphatic leukemia in a subject.

58. A method comprising use of a device for evaluation of the expression level of genes according to claim 57, wherein the expression level of at least one gene of the group composed of CD79A, FAIM3, HLA-DRA, HLA-DRB3, HLA-DQA1, IRF8 and COL3A1 is evaluated for the in vitro diagnosis of the existence of chronic lymphatic leukemia in a subject.

59. A method comprising use of a device for evaluation of the expression level of genes according to claim 57, wherein the expression level of at least genes CD79A, FAIM3, HLA-DRA, HLA-DRB3, HLA-DQA1 is evaluated for the in vitro diagnosis of the existence of chronic lymphatic leukemia in a subject.

60. A method comprising use of a device for evaluation of the expression level of genes according to claim 59, wherein additionally the expression level of at least genes IRF8 and COL3A1 are evaluated for the in vitro diagnosis of the existence of chronic lymphatic leukemia in a subject.

61. A method comprising use of a device for evaluation of the expression level of genes according to claim 57, wherein the expression level of at least one gene of the group composed of PSMB4, FCER2, POU2F2, ODC1, CD79A, CD2, CD3E, CD5, MS4A1, EIF4E, FHIT, NR3C1, LCP1, MAPK10, ABCC5, XRCC3, CML66, PLZF, RBP4 is evaluated, to make an in vitro prognosis of the evolution of the disease in a subject suffering from chronic lymphatic leukemia.


SEQUENCE LISTING (607 sequences)

SEQ ID NO: 1 - DNA, 50 bp, Artificial Sequence
  Probe SG1 designed to hybridize with cRNA of the human gene GABARAP
tccagtgattgttttggtttctgttccctttctgactgcccaaggggctc

SEQ ID NO: 2 - DNA, 50 bp, Artificial Sequence
  Probe SG2 designed to hybridize with cRNA of the human gene IGLV6-57
ttatgatagcacctatcatgtgatattcggcggagggaccaagctgaccg

SEQ ID NO: 3 - DNA, 55 bp, Artificial Sequence
  Probe SG3 designed to hybridize with cRNA of the human gene PCD
gccgtctcttgctccctaattcatttcccaggaagctgtgatacagggtgaaata

SEQ ID NO: 4 - DNA, 53 bp, Artificial Sequence
  Probe SG4 designed to hybridize with cRNA of the human gene NCALD
ccactgaccctgtctgtattttctcggaggttgtttttctccttctccttccc

SEQ ID NO: 5 - DNA, 55 bp, Artificial Sequence
  Probe SG5 designed to hybridize with cRNA of the human gene DLAD
gagtccacaccaagccttcagaagtggaggattcatttgtacccagaattggcaa

SEQ ID NO: 6 - DNA, 55 bp, Artificial Sequence
  Probe SG6 designed to hybridize with cRNA of the human gene SOSTDC1
cgcttggaatggaatgccagaaatgcatggcagcagctaataagtaaagctgatt

SEQ ID NO: 7 - DNA, 55 bp, Artificial Sequence
  Probe SG7 designed to hybridize with cRNA of the human gene T1A-2
ggaaagaccgttcaccagacttggctcctctaaacatttgctgttcaaacatgtt

SEQ ID NO: 8 - DNA, 55 bp, Artificial Sequence
  Probe SG8 designed to hybridize with cRNA of the human gene PYGB
ccctgaccaccactgtgcccctcattgttactgccttgtgagataaaaactgatt

SEQ ID NO: 9 - DNA, 51 bp, Artificial Sequence
  Probe SG9 designed to hybridize with cRNA of the human gene LIMS1
atgggcaagcagtggcatgtggagcattttgtttgtgccaagtgtgagaaa

SEQ ID NO: 10 - DNA, 51 bp, Artificial Sequence
  Probe SG10 designed to hybridize with cRNA of the human gene ABL1
atccaaatctgtcctcgtcctccagctgttatctggaagaagcccttcagc

SEQ ID NO: 11 - DNA, 50 bp, Artificial Sequence
  Probe SG11 designed to hybridize with cRNA of the human gene DRG1
attgtgaagaagtgaaacctttcccttttcccatctgccggacgaaccac

SEQ ID NO: 12 - DNA, 50 bp, Artificial Sequence
  Probe SG12 designed to hybridize with cRNA of the human gene BRMS1
agaagacagccgaagtcaaagccacatcctcttgctgatgttggatgcag

SEQ ID NO: 13 - DNA, 55 bp, Artificial Sequence
  Probe SG13 designed to hybridize with cRNA of the human gene ABHD1
gaggacagaaacagctgacaagagtaccatttggggtctcagttcactctttcct

SEQ ID NO: 14 - DNA, 55 bp, Artificial Sequence
  Probe SG14 designed to hybridize with cRNAof the human gene IFRD1
tgaattccttcgaaatgtatttgaacttggacccccagtgatgcttgatgctgca

SEQ ID NO: 15 - DNA, 55 bp, Artificial Sequence
  Probe SG15 designed to hybridize with cRNA of the human gene RPL36A
cgttgagcccaactgcagatctaagagaatgctggctattaaaagatgcaagcat

SEQ ID NO: 16 - DNA, 50 bp, Artificial Sequence
  Probe SG16 designed to hybridize with cRNA of the human gene IGFBP2
agttctgacacacgtatttatatttggaaagagaccagcaccgagctcgg

SEQ ID NO: 17 - DNA, 50 bp, Artificial Sequence
  Probe SG17 of designed to hybridize with cRNA the human gene NDRG1
gactttccttccctctcctgcttcctcttttcctgctccctaacctttcg

SEQ ID NO: 18 - DNA, 55 bp, Artificial Sequence
  Probe SG18 designed to hybridize with cRNA of the human gene FKBP9
cgtgactcaccatgcccagccacttagttttttcttattcccacctttctatccc

SEQ ID NO: 19 - DNA, 50 bp, Artificial Sequence
  Probe SG19 designed to hybridize with cRNA of the human gene NDP52
ttttttttttgaaacagtctctctctgtcacccaggctggcgtgcaatgg

SEQ ID NO: 20 - DNA, 55 bp, Artificial Sequence
  Probe SG20 designed to hybridize with cRNA of the human gene FABP5
ggtgcattggttcagcatcaggagtgggatgggaaggaaagcacaataacaagaa

SEQ ID NO: 21 - DNA, 55 bp, Artificial Sequence
  Probe SG21 designed to hybridize with cRNA of the human gene FARP1
acggtttccagtactaacaaagggaataaaaatacctcacgccacaatccagcat

SEQ ID NO: 22 - DNA, 46 bp, Artificial Sequence
  Probe SG22 designed to hybridize with cRNA of the human gene PGF
ggaagaggaggagagagaagcagagacccacagactgccacctgtg

SEQ ID NO: 23 - DNA, 55 bp, Artificial Sequence
  Probe SG23 designed to hybridize with cRNA of the human gene GDI2
gcctgagaaggaaatcagaccagctttggagctcttggaaccaattgaacagaaa

SEQ ID NO: 24 - DNA, 55 bp, Artificial Sequence
  Probe SG24 designed to hybridize with cRNA of the human gene TAGLN2
gagggctgcctaccatggtctggggcttgaggaagatgagtttgttgatttaaat

SEQ ID NO: 25 - DNA, 55 bp, Artificial Sequence
  Probe SG25 designed to hybridize with cRNA of the human gene BLVRB
ggacatgaggagcaaaggaagggggcaataaatgttgagccaagagcttcaaatt

SEQ ID NO: 26 - DNA, 50 bp, Artificial Sequence
  Probe SG26 designed to hybridize with cRNA of the human gene PSMB4
ttacaaccggtttcaaaccgccactgtcaccgaaaaaggtgttgaaatag

SEQ ID NO: 27 - DNA, 50 bp, Artificial Sequence
  Probe SG27 designed to hybridize with cRNA of the human gene TACSTD2
gaccacatatgcttgtcactgggaaagaagcctgtttcagctgcctgaac

SEQ ID NO: 28 - DNA, 50 bp, Artificial Sequence
  Probe SG28 designed to hybridize with cRNA of the human gene TCEB1
acaaggttcgctacactaacagctccaccgagattcctgaattcccaatt

SEQ ID NO: 29 - DNA, 55 bp, Artificial Sequence
  Probe SG29 designed to hybridize with cRNA of the human gene EEF1A1
gagaatgttttgtggacacgttggttttcttttttgcgtgtggcagttttagtta

SEQ ID NO: 30 - DNA, 55 bp, Artificial Sequence
  Probe SG30 designed to hybridize with cRNA of the human gene H3F3A
ccgcatacgtggagaacgtgcttaagaatccactatgatgggaaacatttcattc

SEQ ID NO: 31 - DNA, 55 bp, Artificial Sequence
  Probe SG31 designed to hybridize with cRNA of the human gene ODC1
gcatttgtagcttgtacaatggcagaatgggccaaaagcttagtgttgtgacctg

SEQ ID NO: 32 - DNA, 55 bp, Artificial Sequence
  Probe SG32 designed to hybridize with cRNA of the human gene XRCC5
cccagtcacctctgtcttcagcaccctcataagtcgtcactaatacacagttttg

SEQ ID NO: 33 - DNA, 50 bp, Artificial Sequence
  Probe SG33 designed to hybridize with cRNA of the human gene IGFBP3
ttcacttctctgtcttctgtttctctcccacagtgtcgcccttccaaagg

SEQ ID NO: 34 - DNA, 51 bp, Artificial Sequence
  Probe SG34 designed to hybridize with cRNA of the human gene NCL
aggaggaggaggtgaccacaagccacaaggaaagaagacgaagtttgaata

SEQ ID NO: 35 - DNA, 55 bp, Artificial Sequence
  Probe SG35 cRNA designed to hybridize with cRNA of the human gene S100A2
ctgccatggatctcttgggcccaggactgttgatgcctttgagttttgtattcaa

SEQ ID NO: 36 - DNA, 53 bp, Artificial Sequence
  Probe SG36 designed to hybridize with cRNA of the human gene RPL24
tcagctccccgagttggtggaaaacgctaaactggcagattagatttttaaat

SEQ ID NO: 37 - DNA, 55 bp, Artificial Sequence
  Probe SG37 designed to hybridize with cRNA of the human gene GJA1
cacccctaagaatggttctgtgtatgtgaatgagcgggtggtaattgtggctaaa

SEQ ID NO: 38 - DNA, 50 bp, Artificial Sequence
  Probe SG38 designed to hybridize with cRNA of the human gene GUSB
aatgtttggaaaacagcccgtttacttgagcaagactgataccacctgcg

SEQ ID NO: 39 - DNA, 55 bp, Artificial Sequence
  Probe SG39 designed to hybridize with cRNA of the human gene HSD17B1
gttggagaccagccagagcaacacagtgagacccccatctctacaaaaataaaga

SEQ ID NO: 40 - DNA, 50 bp, Artificial Sequence
  Probe SG40 designed to hybridize with cRNA of the human gene SFTPB
ttttcaaaaaatcagcaattccccagcgtagtcaagggtggacactgcac

SEQ ID NO: 41 - DNA, 50 bp, Artificial Sequence
  Probe SG41 designed to hybridize with cRNA of the human gene RPL23A
attcggcctgatggagagaagaaggcatatgttcgactggctcctgatta

SEQ ID NO: 42 - DNA, 55 bp, Artificial Sequence
  Probe SG42 designed to hybridize with cRNA of the human gene CSRP2
ggctaactcgtaggaagagagcactgtatggtatccttttgctttattcaccagc

SEQ ID NO: 43 - DNA, 55 bp, Artificial Sequence
  Probe SG43 designed to hybridize with cRNA of the human gene CXADR
tgctgttgtgtgatcaaacatgtctctgtgtagttccagcaaatcaagctgagct

SEQ ID NO: 44 - DNA, 50 bp, Artificial Sequence
  Probe SG49 designed to hybridize with cRNA of the human gene EEF1G
tttggaaccaacaatagcagctccatttctggagtctgggtcttccgagg

SEQ ID NO: 45 - DNA, 54 bp, Artificial Sequence
  Probe SG45 designed to hybridize with cRNA of the human gene CKS2
tgcatgagctgtattcttcacagcaacagagctcagttaaatgcaactgcaagt

SEQ ID NO: 46 - DNA, 51 bp, Artificial Sequence
  Probe SG46 designed to hybridize with cRNA of the human gene COL4A6
attaatggcatatggtcctagacaaaccatctcctccttgccggctccccc

SEQ ID NO: 47 - DNA, 55 bp, Artificial Sequence
  Probe SG47 designed to hybridize with cRNA of the human gene CRYAB
gctgtcaccgcagcccccaagaaatagatgccctttcttgaattgcattttttaa

SEQ ID NO: 48 - DNA, 55 bp, Artificial Sequence
  Probe SG48 designed to hybridize with cRNA of the human gene CYC1
gctctagctctgggccctccttcagcccccatcatgggaataaattaattttctc

SEQ ID NO: 49 - DNA, 50 bp, Artificial Sequence
  Probe SG49 designed to hybridize with cRNA of the human gene FNTB
tcactcctccctatcatgtaccgtgaaaaccccctctgatggcctcaagg

SEQ ID NO: 50 - DNA, 50 bp, Artificial Sequence
  Probe SG50 designed to hybridize with cRNA of the human gene GOT1
aaagatcaagttgtctgaaggagccaaagtgtgaatgtgggtgtcggctg

SEQ ID NO: 51 - DNA, 46 bp, Artificial Sequence
  Probe SG51 designed to hybridize with cRNA of the human gene LGALS7
cttggttcctcccaatgccagcaggttccatgtaaacctgctgtgc

SEQ ID NO: 52 - DNA, 50 bp, Artificial Sequence
  Probe SG52 designed to hybridize with cRNA of the human gene SERPINB5
ttgatagctgtcccatctggtcatgtggttggcactagactggtggcagg

SEQ ID NO: 53 - DNA, 50 bp, Artificial Sequence
  Probe SG53 designed to hybridize with cRNA of the human gene PSMC5
agaaaaattgctgagctcatgccaggagcatcaggggctgaagtgaaggg

SEQ ID NO: 54 - DNA, 55 bp, Artificial Sequence
  Probe SG54 designed to hybridize with cRNA of the human gene PTN
ccctgtggcaaactgaccaagcccaaacctcaagcagaatctaagaagaagaaaa

SEQ ID NO: 55 - DNA, 55 bp, Artificial Sequence
  Probe SG55 designed to hybridize with cRNA of the human gene RBBP8
ggcaaggagcagaagacatagacgttgaaacagaaacagaaggatgaaggacagt

SEQ ID NO: 56 - DNA, 55 bp, Artificial Sequence
  Probe SG56 designed to hybridize with cRNA of the human gene RGS1
gcccctcagaactgggaaggccaggtaactctagttacacagaaactgtgactaa

SEQ ID NO: 57 - DNA, 50 bp, Artificial Sequence
  Probe SG57 designed to hybridize with cRNA of the human gene RXRA
tttcgagtaatttttaaagccttgctctgttgtgtcctgttgccggctct

SEQ ID NO: 58 - DNA, 54 bp, Artificial Sequence
  Probe SG58 designed to hybridize with cRNA of the human gene SDHD
gcttggagtgcttctgaatatacagaagttccatttaagggcaagtttccccgt

SEQ ID NO: 59 - DNA, 50 bp, Artificial Sequence
  Probe SG59 designed to hybridize with cRNA of the human gene SEPW1
tctcaaggggaagccaagagaggcatcaggatgggtgggtttctgattgt

SEQ ID NO: 60 - DNA, 55 bp, Artificial Sequence
  Probe SG60 designed to hybridize with cRNA of the human gene SSBP1
gccaggcctcagagacgtggcatatcaatatgtgaaaaaggggtctcgaatttat

SEQ ID NO: 61 - DNA, 55 bp, Artificial Sequence
  Probe SG61 designed to hybridize with cRNA of the human gene TEGT
acgaaatcatttgtttctaagttgtgtttattcctggagtgacatgccaccccga

SEQ ID NO: 62 - DNA, 50 bp, Artificial Sequence
  Probe SG62 designed to hybridize with cRNA of the human gene HIST1H2BN
tctcgtccaaggccatgggcatcatgaactccttcgtcaatgacatcttc

SEQ ID NO: 63 - DNA, 55 bp, Artificial Sequence
  Probe SG63 designed to hybridize with cRNA of the human gene BECN1
ccagccctggtcagttttgattcttaaccccatggactcctttccctttcttctc

SEQ ID NO: 64 - DNA, 55 bp, Artificial Sequence
  Probe SG64 designed to hybridize with cRNA of the human gene ANXA7
gtcaaggaccgtatcagggtaatgtgcttggtttgcacatgttgttattgcctta

SEQ ID NO: 65 - DNA, 55 bp, Artificial Sequence
  Probe SG65 designed to hybridize with cRNA of the human gene NDUFA1
gcgcatctctggagttgatcgttactatgtgtcaaagggtttggagaacattgat

SEQ ID NO: 66 - DNA, 50 bp, Artificial Sequence
  Probe SG66 designed to hybridize with cRNA of the human gene ARHGEF2
ctcaacctcttactgctgttctccctttctccgtccttcatggaagccct

SEQ ID NO: 67 - DNA, 55 bp, Artificial Sequence
  Probe SG67 designed to hybridize with cRNA of the human gene C5orfl3
ccattggcctgagtttcttgtgcattactcctctccctccttcgttagaataggt

SEQ ID NO: 68 - DNA, 52 bp, Artificial Sequence
  Probe SG68 designed to hybridize with cRNA of the human gene XBP1
gcatcctggcttgcctccagttttaggtcctttagtttgcttctgtaagcaa

SEQ ID NO: 69 - DNA, 50 bp, Artificial Sequence
  Probe SG69 designed to hybridize with cRNA of the human gene G1P2
agcatccgagcaggatcaagggccggaaataaaggctgttgtaagagaat

SEQ ID NO: 70 - DNA, 53 bp, Artificial Sequence
  Probe SG70 designed to hybridize with cRNA of the human gene GLUD1
gcccctactcacctgactttgtatcctctccttttagaggctttgcattctgc

SEQ ID NO: 71 - DNA, 55 bp, Artificial Sequence; identical to SEQ ID NO: 35
  Probe SG71 designed to hybridize with cRNA of the human gene S100A2

SEQ ID NO: 72 - DNA, 50 bp, Artificial Sequence
  Probe SG72 designed to hybridize with cRNA of the human gene LASP1
tactaagaataacaagaagcccagtggtgaggaaagtgcgttctcccagc

SEQ ID NO: 73 - DNA, 50 bp, Artificial Sequence
  Probe SG73 designed to hybridize with cRNA of the human gene TIA-2
tgtgaaaaataacagtccaccccaagtcatacactggacccagtgcctgc

SEQ ID NO: 74 - DNA, 55 bp, Artificial Sequence
  Probe SG74 designed to hybridize with cRNA of the human gene PGRMC1
cctgaaccacgaggaaacagtacagtcgctagtcaagtggtttttaaagtaaagt

SEQ ID NO: 75 - DNA, 50 bp, Artificial Sequence
  Probe SG75 designed to hybridize with cRNA of the human gene ZFPL1
caagcctaagacactaagaccccagacccaaagccaagtccaccagagtg

SEQ ID NO: 76 - DNA, 50 bp, Artificial Sequence
  Probe SG76 designed to hybridize with cRNA of the human gene MAP4K1
atatggtgatggtgttgatggatggctctgtgaagctggtgaccccggag

SEQ ID NO: 77 - DNA, 55 bp, Artificial Sequence
  Probe SG77 designed to hybridize with cRNA of the human gene STAT1
gtggcactgcatacaatctgaggcctcctctctcagtttttatatagatggcgag

SEQ ID NO: 78 - DNA, 54 bp, Artificial Sequence
  Probe SG78 designed to hybridize with cRNA of the human gene HNRPH3
gctgaattctagccctagattttggagtaaaaccccttcagcacttgaccgaaa

SEQ ID NO: 79 - DNA, 53 bp, Artificial Sequence
  Probe SG79 designed to hybridize with cRNA of the human gene TMEM4
tcaagtttgcgtgtgagagcattgtggaggaatacgaggatgaactcattgaa

SEQ ID NO: 80 - DNA, 51 bp, Artificial Sequence
  Probe SG80 designed to hybridize with cRNA of the human gene KIAA0247
gtttggtctgaatggtgtagttgctggttccctagagaggaaaaggtggca

SEQ ID NO: 81 - DNA, 55 bp, Artificial Sequence
  Probe SG81 designed to hybridize with cRNA of the human gene RIS1
gtggcctggtgggcgtttcttcttgtacttatgtggttttttggcttttaataca

SEQ ID NO: 82 - DNA, 53 bp, Artificial Sequence
  Probe SG82 designed to hybridize with cRNA of the human gene ARS2
tcttcaacaagcatgcagagaaaattgaggaagtgaaaaaggaagtcgcgttt

SEQ ID NO: 83 - DNA, 50 bp, Artificial Sequence
  Probe SG83 designed to hybridize with cRNA of the human gene CA12
catccactggctgaatgatcagggaggcatagcagtgagagccataggtc

SEQ ID NO: 84 - DNA, 55 bp, Artificial Sequence
  Probe SG84 designed to hybridize with cRNA of the human gene EEF1B2
actgcttttgaggactatgtgcagtccatggatgtggctgctttcaacaagatct

SEQ ID NO: 85 - DNA, 55 bp, Artificial Sequence
  Probe SG85 designed to hybridize with cRNA of the human gene TRIB2
gcctgtatatgttttgtgaaatggtcctgtttttgggtaggtgacacgtggactc

SEQ ID NO: 86 - DNA, 55 bp, Artificial Sequence
  Probe SG86 designed to hybridize with cRNA of the human gene FLJ22169
agcgtccagctcccctcaacgctatattttgacactaaaaaagaaggtttctaaa

SEQ ID NO: 87 - DNA, 50 bp, Artificial Sequence
  Probe SG87 designed to hybridize with cRNA of the human gene ASNS
aaagaaggatattactaccgtcaagtctttgaacgccattacccaggccg

SEQ ID NO: 88 - DNA, 55 bp, Artificial Sequence
  Probe SG88 designed to hybridize with cRNA of the human gene WBSCR20C
ggtgagcaaaagtgttgcctgcagaaataaaatgcagaacgtactctacgataaa

SEQ ID NO: 89 - DNA, 51 bp, Artificial Sequence
  Probe SG89 designed to hybridize with cRNA of the human gene AKR1A1
atcctctgtacccctttaatgacccgtactgagaccacagcttcttggcct

SEQ ID NO: 90 - DNA, 55 bp, Artificial Sequence
  Probe SG90 designed to hybridize with cRNA of the human gene SPRR1A
cgggtgcatttgaggatggatttggggagggatcaagtgaaccatccctagtctt

SEQ ID NO: 91 - DNA, 50 bp, Artificial Sequence
  Probe SG91 designed to hybridize with cRNA of the human gene EFNB1
attaatgtcttaattggctgttgcctggggaacaggagagctgctgcagg

SEQ ID NO: 92 - DNA, 50 bp, Artificial Sequence
  Probe SG92 designed to hybridize with cRNA of the human gene RPS5
tcaatgctgccaagggctcctcgaactcctatgccattaagaagaaggac

SEQ ID NO: 93 - DNA, 51 bp, Artificial Sequence
  Probe SG93 designed to hybridize with cRNA of the human gene RPS9
tgctggattgtctcgttttcctgccaaataaacaggatcagcgctttaaaa

SEQ ID NO: 94 - DNA, 55 bp, Artificial Sequence
  Probe SG94 designed to hybridize with cRNA of the human gene RPL17
acggaaaaggaacagattgttcctaaaccagaagaggaggttgcccagaagaaaa

SEQ ID NO: 95 - DNA, 55 bp, Artificial Sequence
  Probe SG95 designed to hybridize with cRNA of the human gene ETS2
cgctgcctcaatacccacaaaagaccattcccagtatacataagcacaggatgtt

SEQ ID NO: 96 - DNA, 55 bp, Artificial Sequence
  Probe SG96 designed to hybridize with cRNA of the human gene EIF4B
tccttgcctgctcctgatgcttggaccccttttattgatcagagtgctctagaat

SEQ ID NO: 97 - DNA, 50 bp, Artificial Sequence
  Probe SG97 designed to hybridize with cRNA of the human gene ZYX
tttgatgtctagcccctcccatttccaacccctccctagcatcccaggtg

SEQ ID NO: 98 - DNA, 55 bp, Artificial Sequence
  Probe SG98 designed to hybridize with cRNA of the human gene XPO6
cggacagtcctttctccagcccgacatccacctttttaaacaaaatctcttctac

SEQ ID NO: 99 - DNA, 55 bp, Artificial Sequence
  Probe SG99 designed to hybridize with cRNA of the human gene LOC285148
gtcaatacgtagaggtacgcttttttcctcaggcttaaacctttgccactgatat

SEQ ID NO: 100 - DNA, 50 bp, Artificial Sequence
  Probe SG100 designed to hybridize with cRNA of the human gene C21orf33
aataaacgccttcccaaaatggcaacttcccacagccacatttcagacct

SEQ ID NO: 101 - DNA, 50 bp, Artificial Sequence
  Probe SG101 designed to hybridize with cRNA of the human gene EEF1D
agtgtcgatatcgcagctttcaacaagatctgaagcctgagtgtgtgtac

SEQ ID NO: 102 - DNA, 51 bp, Artificial Sequence
  Probe SG102 designed to hybridize with cRNA of the human gene PABPC1
tactacaagcccaccaagctaaagaggctgcccagaaagcagttaacagtg

SEQ ID NO: 103 - DNA, 55 bp, Artificial Sequence
  Probe SG103 designed to hybridize with cRNA of the human gene RBBP6
gccagctcagcagaaagtcaggacagcaagaagaagaagaaaaagaaggaaaaga

SEQ ID NO: 104 - DNA, 50 bp, Artificial Sequence
  Probe SG104 designed to hybridize with cRNA of the human gene HRAS
tttgaggacatccaccagtacagggagcagatcaaacgggtgaaggactc

SEQ ID NO: 105 - DNA, 50 bp, Artificial Sequence
  Probe SG105 designed to hybridize with cRNA of the human gene GGA3
tactccaagcctccgtcacaaaaaaaaaatcaccagctgccatagacacg

SEQ ID NO: 106 - DNA, 54 bp, Artificial Sequence
  Probe SG106 designed to hybridize with cRNA of the human gene CFL1
cctgtattttttttaacaacatccccattccccacctggtcctcccccttccca

SEQ ID NO: 107 - DNA, 55 bp, Artificial Sequence
  Probe SG107 designed to hybridize with cRNA of the human gene EIF3S2
gtcattgcactccatcctgagccacaagagcaaaactccgtctcaaaaaaaaaaa

SEQ ID NO: 108 - DNA, 55 bp, Artificial Sequence
  Probe SG108 designed to hybridize with cRNA of the human gene KRT18
gcagggtaccctttggggagcaggaggccaataaaaagttcagagttcaaaaaaa

SEQ ID NO: 109 - DNA, 50 bp, Artificial Sequence
  Probe SG109 designed to hybridize with cRNA of the human gene IGFIR
gaaatggagaataatccagtcctagcacctccaagcctgagcaagatgat

SEQ ID NO: 110 - DNA, 50 bp, Artificial Sequence
  Probe SG110 designed to hybridize with cRNA of the human gene IL8
agtgattgagagtggaccacactgcgccaacacagaaattattgtaaagc

SEQ ID NO: 111 - DNA, 50 bp, Artificial Sequence
  Probe SG111 designed to hybridize with cRNA of the human gene IRF1
ccggacagcaccagtgatctgtacaacttccaggtgtcacccatgccctc

SEQ ID NO: 112 - DNA, 50 bp, Artificial Sequence
  Probbe SG112 designed to hybridize with cRNA of the human gene IRF2
tcatcaagaaaacatcggatatcacccaggcccgcgtcaagagctgttaa

SEQ ID NO: 113 - DNA, 50 bp, Artificial Sequence
  Probe SG113 designed to hybridize with cRNA of the human gene LYN
tatgtgagagatccaacgtccaataaacagcaaaggccagttccagaatc

SEQ ID NO: 114 - DNA, 50 bp, Artificial Sequence
  Probe SG114 designed to hybridize with cRNA of the human gene LYZ
gttgtcaaaacagagatgtccgtcagtatgttcaaggttgtggagtgtaa

SEQ ID NO: 115 - DNA, 50 bp, Artificial Sequence
  Probe SG115 designed to hybridize with cRNA of the human gene NFKB1A
gaggacgagctgccctatgatgactgtgtgtttggaggccagcgtctgac

SEQ ID NO: 116 - DNA, 50 bp, Artificial Sequence
  Probe SG116 designed to hybridize with cRNA of the human gene MAZ
ccagccctggtcttgtcttttcatccctcttccccacgacagaagaagtt

SEQ ID NO: 117 - DNA, 50 bp, Artificial Sequence
  Probe SG117 designed to hybridize with cRNA of the human gene CD79A
caccaagaaccgaatcatcacagccgaggggatcatcctcctgttctgcg

SEQ ID NO: 118 - DNA, 50 bp, Artificial Sequence
  Probe SG118 designed to hybridize with cRNA of the human gene MCM3
gaggaaatgcctcaagtacacactccaaagacggcagactcacaggagac

SEQ ID NO: 119 - DNA, 50 bp, Artificial Sequence
  Probe SG119 designed to hybridize with cRNA of the human gene MEIS1
gtttcgtaatggacggtcagcaacatatgggaattagagcaccaggacct

SEQ ID NO: 120 - DNA, 50 bp, Artificial Sequence
  Probe SG120 designed to hybridize with cRNA of the human gene PRTN3
cttcgtgatctggggatgtgccacccgccttttccctgacttcttcacgc

SEQ ID NO: 121 - DNA, 50 bp, Artificial Sequence
  Probe SG121 designed to hybridize with cRNA of the human gene MMP2
gaaccaaagtctgaagagcgtgaagtttggaagcatcaaatccgactggc

SEQ ID NO: 122 - DNA, 50 bp, Artificial Sequence
  Probe SG122 designed to hybridize with cRNA of the human gene MMP7
gtagcagtctagggattaacttcctgtatgctgcaactcatgaacttggc

SEQ ID NO: 123 - DNA, 50 bp, Artificial Sequence
  Probe SG123 designed to hybridize with cRNA of the human gene MMP8
ttaccagagttgcaagaggcaataaatggcttaactgtagatatggctga

SEQ ID NO: 124 - DNA, 50 bp, Artificial Sequence
  Probe SG124 designed to hybridize with cRNA of the human gene DOK1
cctggtactgcaactggcagtggcatcaaaagccacaactcagccctgta

SEQ ID NO: 125 - DNA, 50 bp, Artificial Sequence
  Probe SG125 designed to hybridize with cRNA of the human gene PDGFA
ccgctgtctgcaagaccaggacggtcatttacgagattcctcggagtcag

SEQ ID NO: 126 - DNA, 50 bp, Artificial Sequence
  Probe SG126 designed to hybridize with cRNA of the human gene PRL
attatctcaagctcctgaagtgccgaatcatccacaacaacaactgctaa

SEQ ID NO: 127 - DNA, 50 bp, Artificial Sequence
  Probe SG127 designed to hybridize with cRNA of the human gene RAF1
ctattggtgatagtggagtcccagcactaccttctttgactatgcgtcgt

SEQ ID NO: 128 - DNA, 50 bp, Artificial Sequence
  Probe SG128 designed to hybridize with cRNA of the human gene RARB
tcagtggattgacccaaaccgaatggcagcatcggcacactgctcaatca

SEQ ID NO: 129 - DNA, 50 bp, Artificial Sequence
  Probe SG129 designed to hybridize with cRNA of the human gene OPRD1
acggtgaccaagatctgcgtgttcctcttcgccttcgtggtgcccatcct

SEQ ID NO: 130 - DNA, 50 bp, Artificial Sequence
  Probe SG130 designed to hybridize with cRNA of the human gene RET
caggcaggtgtgagtggaggcaaggagatggcaaagggatcaccaggaac

SEQ ID NO: 131 - DNA, 50 bp, Artificial Sequence
  Probe SG131 designed to hybridize with cRNA of the human gene SREBF1
ttggtgcttctctttgtctacggtgagccagtcacacggccccactcagg

SEQ ID NO: 132 - DNA, 50 bp, Artificial Sequence
  Probe SG132 designed to hybridize with cRNA of the human gene TNF
agccctctggcccaggcagtcagatcatcttctcgaaccccgagtgacaa

SEQ ID NO: 133 - DNA, 50 bp, Artificial Sequence
  Probe SG133 designed to hybridize with cRNA of the human gene TERF1
cctgggtgacaggatcatacgtaccccaaacctcaacatcacacagtata

SEQ ID NO: 134 - DNA, 50 bp, Artificial Sequence
  Probe SG134 designed to hybridize with cRNA of the human gene VCAM1
tctcccctggaccccggattgctgctcagattggagactcagtcatgttg

SEQ ID NO: 135 - DNA, 50 bp, Artificial Sequence
  Probe SG135 designed to hybridize with cRNA of the human gene ATP5O
ccaagtattgaaattggaggctaagactgatccgtcaatcttgggtggaa

SEQ ID NO: 136 - DNA, 50 bp, Artificial Sequence
  Probe SG136 designed to hybridize with cRNA of the human gene TNFSF5
caggtgcttcggtgtttgtcaatgtgactgatccaagccaagtgagccat

SEQ ID NO: 137 - DNA, 50 bp, Artificial Sequence
  Probe SG137 designed to hybridize with cRNA of the human gene POLR2C
gaagaaactgagtgatttacaaactcaattaagccacgagatccagagtg

SEQ ID NO: 138 - DNA, 50 bp, Artificial Sequence
  Probe SG138 designed to hybridize with cRNA of the human gene PLA2G6
agctcagcccccaccgatcagcctaaacaacctagaactacaggatctca

SEQ ID NO: 139 - DNA, 50 bp, Artificial Sequence
  Probe SG139 designed to hybridize with cRNA of the human gene CCT6A
tcttggttgatgagatcatgcgagctggaatgtcttctctgaaaggttga

SEQ ID NO: 140 - DNA, 50 bp, Artificial Sequence
  Probe SG140 designed to hybridize with cRNA of the human gene PDHA1
ccacagaccatctcatcacagcctaccgggctcacggctttactttcacc

SEQ ID NO: 141 - DNA, 50 bp, Artificial Sequence
  Probe SG141 designed to hybridize with cRNA of the human gene LADH
cggaataaaggatgatgtcttccttagtgttccttgcattttgggacaga

SEQ ID NO: 142 - DNA, 50 bp, Artificial Sequence
  Probe SG142 designed to hybridize with cRNA of the human gene SNRPB
gaactccaaacaagcagaaagggaagagaagcgagtcctcggtctggtgc

SEQ ID NO: 143 - DNA, 50 bp, Artificial Sequence
  Probe SG143 designed to hybridize with cRNA of the human gene SNRPB
tacaaacccgaggaccctcttagggaagaagccctacatttgcagtgatt

SEQ ID NO: 144 - DNA, 50 bp, Artificial Sequence
  Probe SG144 designed to hybridize with cRNA of the human gene H2AFX
aggcgccctcgggcggcaagaaggccacccaggcctcccaggagtactaa

SEQ ID NO: 145 - DNA, 50 bp, Artificial Sequence
  Probe SG145 designed to hybridize with cRNA of the human gene MRPL37
ggctatccttacccctatccccataccctgtacttactggacaaagccaa

SEQ ID NO: 146 - DNA, 50 bp, Artificial Sequence
  Probe SG146 designed to hybridize with cRNA of the human gene UBE2C
cctgctatcaccccaacgtggacacccagggtaacatatgcctggacatc

SEQ ID NO: 147 - DNA, 50 bp, Artificial Sequence
  Probe SG147 designed to hybridize with cRNA of the human gene RPS3
aagaaagcattcatgggaccactgaagaaagaccgaattgcaaaggaaga

SEQ ID NO: 148 - DNA, 50 bp, Artificial Sequence
  Probe SG148 designed to hybridize with cRNA of the human gene ALDH1A1
actgggagagtacggtttccatgaatatacagaggtcaaaacagtcacag

SEQ ID NO: 149 - DNA, 50 bp, Artificial Sequence
  Probe SG149 designed to hybridize with cRNA of the human gene AF1q
gcagaccaggagaaaaaccctgaaggtgatggcctccttgagtacagcac

SEQ ID NO: 150 - DNA, 50 bp, Artificial Sequence
  Probe SG150 designed to hybridize with cRNA of the human gene RUNX1
ctccctgaaccactccactgcctttaaccctcagcctcagagtcagatgc

SEQ ID NO: 151 - DNA, 50 bp, Artificial Sequence
  Probe SG151 designed to hybridize with cRNA of the human gene MYBL1
atgggaagacagaagaccaacttattatgactgaacaagcaagaagatat

SEQ ID NO: 152 - DNA, 50 bp, Artificial Sequence
  Probe SG152 designed to hybridize with cRNA of the human gene ANXA6
tgagaaatatgacaagtctctccaccaagccattgagggtgacacctccg

SEQ ID NO: 153 - DNA, 50 bp, Artificial Sequence
  Probe SG153 designed to hybridize with cRNA of the human gene ALK
gtcattacgaggataccattctgaaaagcaagaatagcatgaaccagcct

SEQ ID NO: 154 - DNA, 50 bp, Artificial Sequence
  Probe SG154 designed to hybridize with cRNA of the human gene MNDA
gtgtgagaaaggagataaacttcgactcttctgccttcaactgagaacag

SEQ ID NO: 155 - DNA, 50 bp, Artificial Sequence
  Probe SG155 designed to hybridize with cRNA of the human gene APAF1
ggtggaacgttgtcactggggaatcctcacagaccttctacacaaatgga

SEQ ID NO: 156 - DNA, 50 bp, Artificial Sequence
  Probe SG156 designed to hybridize with cRNA of the human gene BIRC3
aggcgacactttcctaattgcttctttgttttgggccggaatcttaatat

SEQ ID NO: 157 - DNA, 50 bp, Artificial Sequence
  Probe SG157 designed to hybridize with cRNA of the human gene ABCC6
tgtccttcaagatccacgcaggagagaaggtgggcatcgttggcaggacc

SEQ ID NO: 158 - DNA, 50 bp, Artificial Sequence
  Probe SG158 designed to hybridize with cRNA of the human gene ATIC
gaactgggaatcatcctcgctcatacgaaccttcggctcttccaccactg

SEQ ID NO: 159 - DNA, 50 bp, Artificial Sequence
  Probe SG159 designed to hybridize with cRNA of the human gene ATM
tgaggaattagatgaacgtgagcgaaactctcgtccttctagtaaaaatc

SEQ ID NO: 160 - DNA, 50 bp, Artificial Sequence
  Probe SG160 designed to hybridize with cRNA of the human gene BAX
ttgtcgcccttttctactttgccagcaaactggtgctcaaggccctgtgc

SEQ ID NO: 161 - DNA, 50 bp, Artificial Sequence
  Probe SG161 designed to hybridize with cRNA of the human gene CCND1
cttcctgtcctactaccgcctcacacgcttcctctccagagtgatcaagt

SEQ ID NO: 162 - DNA, 50 bp, Artificial Sequence
  Probe SG162 designed to hybridize with cRNA of the human gene BCL2
ttgctgggactgtacccaagaattaaaccaagacaaaggagcttggcctg

SEQ ID NO: 163 - DNA, 50 bp, Artificial Sequence
  Probe SG163 designed to hybridize with cRNA of the human gene BCL3
tttcctctggtgaacctgcctacacccctataccccatgatgtgccccat

SEQ ID NO: 164 - DNA, 50 bp, Artificial Sequence
  Probe SG164 designed to hybridize with cRNA of the human gene BCL6
tcaataacatcgttaacaggtccatgacgggctctccccgcagcagcagc

SEQ ID NO: 165 - DNA, 50 bp, Artificial Sequence
  Probe SG165 designed to hybridize with cRNA of the human gene BCL7A
gttgtcgggggatctggaaggagtgccaccctctaaaaagatgaaactgg

SEQ ID NO: 166 - DNA, 50 bp, Artificial Sequence
  Probe SG166 designed to hybridize with cRNA of the human gene BCL7b
caaggaagaaccagttccactagagacacaggtcgttgaggaagaggaag

SEQ ID NO: 167 - DNA, 50 bp, Artificial Sequence
  Probe SG167 designed to hybridize with cRNA of the human gene BCL10
gcacgacgccctttttttctactaattcttctctgaatttgcctgttcta

SEQ ID NO: 168 - DNA, 50 bp, Artificial Sequence
  Probe SG168 designed to hybridize with cRNA of the human gene BCL2L1
gtagacaaggagatgcaggtattggtgagtcggatcgcagcttggatggc

SEQ ID NO: 169 - DNA, 50 bp, Artificial Sequence
  Probe SG169 designed to hybridize with cRNA of the human gene BCR
gcagggagttcagcttgaagaggatgccgtcccgaaaacagacaggggtc

SEQ ID NO: 170 - DNA, 50 bp, Artificial Sequence
  Probe SG170 designed to hybridize with cRNA of the human gene BCR
aatgtctgaggaagcatcggagactgggtcccgccatgcctgtgtcatct

SEQ ID NO: 171 - DNA, 50 bp, Artificial Sequence
  Probe SG171 designed to hybridize with cRNA of the human gene OLIG2
caactacatcctcatgctcaccaactcgctggaggagatgaagcgactgg

SEQ ID NO: 172 - DNA, 50 bp, Artificial Sequence
  Probe SG172 designed to hybridize with cRNA of the human gene BIK
caccacacttaaggagaacataatgaggttctggagatccccgaaccccg

SEQ ID NO: 173 - DNA, 50 bp, Artificial Sequence
  Probe SG173 designed to hybridize with cRNA of the human gene BCL2LAA
cagagccacaaggtaatcctgaaggcaatcacggaggtgaaggggacagc

SEQ ID NO: 174 - DNA, 50 bp, Artificial Sequence
  Probe SG174 designed to hybridize with cRNA of the human gene BMI1
caatagacctcgaaaatcatcagtaaatgggtcatcagcaacttcttctg

SEQ ID NO: 175 - DNA, 50 bp, Artificial Sequence
  Probe SG175 designed to hybridize with cRNA of the human gene BLMH
ctaggccccagcacaagtacaacaaactttacacagtggaatacttaagc

SEQ ID NO: 176 - DNA, 50 bp, Artificial Sequence
  Probe SG176 designed to hybridize with cRNA of the human gene BLR1
ttctgggaactggacagattggacaactataacgacacctccctggtgga

SEQ ID NO: 177 - DNA, 50 bp, Artificial Sequence
  Probe SG177 designed to hybridize with cRNA of the human gene IBSP
cgtcaatgaatacgacaatggatatgaaatctatgaaagtgagaacgggg

SEQ ID NO: 178 - DNA, 50 bp, Artificial Sequence
  Probe SG178 designed to hybridize with cRNA of the human gene BTG1
gtagcactcaaaacagcaccaacgtgcaaatggtagacagccgaatcagc

SEQ ID NO: 179 - DNA, 50 bp, Artificial Sequence
  Probe SG179 designed to hybridize with cRNA of the human gene BUB1
gaaatacaatcaacggagaaagcatgagcaatgggtaaatgaagacagac

SEQ ID NO: 180 - DNA, 50 bp, Artificial Sequence
  Probe SG180 designed to hybridize with cRNA of the human gene ABL1
gcaagctcctcagttcggtgaaggaaatcagtgacatagtgcagaggtag

SEQ ID NO: 181 - DNA, 50 bp, Artificial Sequence
  Probe SG171 designed to hybridize with cRNA of the human gene CASP1
ggactctcagcagatcaaacatctggaaattaccttaatatgcaagactc

SEQ ID NO: 182 - DNA, 50 bp, Artificial Sequence
  Probe SG182 designed to hybridize with cRNA of the human gene CASP3
cctggttattattcttggcgaaattcaaaggatggctcctggttcatcca

SEQ ID NO: 183 - DNA, 50 bp, Artificial Sequence
  Probe SG183 designed to hybridize with cRNA of the human gene CASP4
aaatatcccccaataaaaaagctcatccgaatatggaggctggaccacct

SEQ ID NO: 184 - DNA, 50 bp, Artificial Sequence
  Probe SG184 designed to hybridize with cRNA of the human gene CASP5
ctggacaaacatctatccagaccctagtacctaatacggatcaaaagtcg

SEQ ID NO: 185 - DNA, 50 bp, Artificial Sequence
  Probe SG185 designed to hybridize with cRNA of the human gene CASP6
ggttccctgttttgcctcaatgctaactaaaaagctgcatttctttccaa

SEQ ID NO: 186 - DNA, 50 bp, Artificial Sequence
  Probe SG186 designed to hybridize with cRNA of the human gene CASP7
tcccctgtgtggtctccatgctcaccaaggaactctacttcagtcaatag

SEQ ID NO: 187 - DNA, 50 bp, Artificial Sequence
  Probe SG187 designed to hybridize with cRNA of the human gene CASP8
gcagcagccttgaaggaagtcctgatgaattttcaaatgactttggacaa

SEQ ID NO: 188 - DNA, 50 bp, Artificial Sequence
  Probe SG188 designed to hybridize with cRNA of the human gene CASP9
gtccctcctgcttagggtcgctaatgctgtttcggtgaaagggatttata

SEQ ID NO: 189 - DNA, 50 bp, Artificial Sequence
  Probe SG189 designed to hybridize with cRNA of the human gene CBFB
gctcccatgattctgaatggagtctgtgttatctggaaaggctggattga

SEQ ID NO: 190 - DNA, 50 bp, Artificial Sequence
  Probe S90 designed to hybridize with cRNA of the human gene CALD1
caaaagagggaggagatgcgactcgaagcagaaagaatcgcctaccagag

SEQ ID NO: 191 - DNA, 50 bp, Artificial Sequence
  Probe SG191 designed to hybridize with cRNA of the human gene CAST
agcaggcatcagatacaggaagtaacgatgctcacaataaaaaagcagtt

SEQ ID NO: 192 - DNA, 50 bp, Artificial Sequence
  Probe SG192 designed to hybridize with cRNA of the human gene CDC25A
caggggagaagagcaagagggagatgtacagtcgtctgaagaagctctga

SEQ ID NO: 193 - DNA, 50 bp, Artificial Sequence
  Probe SG193 designed to hybridize with cRNA of the human gene CDC25B
ttccctcagcacccgaacttctgtgaaccccaggactaccggcccatgaa

SEQ ID NO: 194 - DNA, 50 bp, Artificial Sequence
  Probe SG194 designed to hybridize with cRNA of the human gene CD2
aaattccagcttcaacccctcagaatccagcaacttcccaacatcctcct

SEQ ID NO: 195 - DNA, 50 bp, Artificial Sequence
  Probe SG195 designed to hybridize with cRNA of the human gene CD3E
aagatgcgaacttttatctctacctgagggcaagagtgtgtgagaactgc

SEQ ID NO: 196 - DNA, 50 bp, Artificial Sequence
  Probe SG196 designed to hybridize with cRNA of the human gene CD4
ctccaaccagataaagattctgggaaatcagggctccttcttaactaaag

SEQ ID NO: 197 - DNA, 50 bp, Artificial Sequence
  Probe SG197 designed to hybridize with cRNA of the human gene CD5
ggaaaatatgccctccccgtaatggtgaaccaccagcacctacccaccac

SEQ ID NO: 198 - DNA, 50 bp, Artificial Sequence
  Probe SG198 designed to hybridize with cRNA of the human gene CD6
tcatctaaagacaccttcctttccactggctgtcaagccacagggcacca

SEQ ID NO: 199 - DNA, 50 bp, Artificial Sequence
  Probe SG199 designed to hybridize with cRNA of the human gene CD7
gtgctcgtggcgggataagaattcggcggcatgtgtggtgtacgaggaca

SEQ ID NO: 200 - DNA, 50 bp, Artificial Sequence
  Probe SG200 designed to hybridize with cRNA of the human gene CD8
ttcattctcaatctcacaagcgtgaagccggaagacagtggcatctactt

SEQ ID NO: 201 - DNA, 50 bp, Artificial Sequence
  Probe SG201 designed to hybridize with cRNA of the human gene CD9
gtgaagtcctgtcctgatgccatcaaagaggtcttcgacaataaattcca

SEQ ID NO: 202 - DNA, 50 bp, Artificial Sequence
  Probe SG202 designed to hybridize with cRNA of the human gene MME
tcagaagcctttcactgccgcaagaattcatacatgaatccagaaaagaa

SEQ ID NO: 203 - DNA, 50 bp, Artificial Sequence
  Probe SG203 designed to hybridize with cRNA of the human gene ITGAL
tgcctgaagcccctccatgagaaggactctgagagtggtggtggcaagga

SEQ ID NO: 204 - DNA, 50 bp, Artificial Sequence
  Probe SG204 Designed to hybridize with cRNA of the human gene ITGAM
tttgtgaggtcccagacggagaccaaagtggagccgttcgaggtccccaa

SEQ ID NO: 205 - DNA, 50 bp, Artificial Sequence
  Probe SG205 Designed to hybridize with cRNA of the human gene ITGAX
aggaggcaaatggacaaattgccccagaaaacgggacacagacccccagc

SEQ ID NO: 206 - DNA, 50 bp, Artificial Sequence
  Probe SG206 Designed to hybridize with cRNA of the human gene ANPEP
agccctggagaagacgaaagccaacatcaagtgggtgaaggagaacaagg

SEQ ID NO: 207 - DNA, 50 bp, Artificial Sequence
  Probe SG207 Designed to hybridize with cRNA of the human gene CD14
tggataacctgacactggacgggaatcccttcctggtccctggaactgcc

SEQ ID NO: 208 - DNA, 50 bp, Artificial Sequence
  Probe SG208 Designed to hybridize with cRNA of the human gene SELE
aattgggctcctgcacacagtggcccgctacaagttctacctggctttcg

SEQ ID NO: 209 - DNA, 50 bp, Artificial Sequence
  Probe SG209 Designed to hybridize with cRNA of the human gene FCGR3A
gtgaagacaaacattcgaagctcaacaagagactggaaggaccataaatt

SEQ ID NO: 210 - DNA, 50 bp, Artificial Sequence
  Probe SG210 Designed to hybridize with cRNA of the human gene FCGR3B
gcttggtgatggtactcctttttgcagtggacacaggactatatttctct

SEQ ID NO: 211 - DNA, 50 bp, Artificial Sequence
  Probe SG211 Designed to hybridize with cRNA of the human gene ITGB2
gataatccccttttcaagagcgccaccacgacggtcatgaaccccaagtt

SEQ ID NO: 212 - DNA, 50 bp, Artificial Sequence
  Probe SG212 Designed to hybridize with cRNA of the human gene CD19
actccattcggggccagcctggacccaatcatgaggaagatgcagactct

SEQ ID NO: 213 - DNA, 50 bp, Artificial Sequence
  Probe SG213 Designed to hybridize with cRNA of the human gene MS4A1
ctccccaagatcaggaatcctcaccaatagaaaatgacagctctccttaa

SEQ ID NO: 214 - DNA, 50 bp, Artificial Sequence
  Probe SG214 Designed to hybridize with cRNA of the human gene CR2
catttagaagcacgagaagtatattctgttgatccatacaacccagccag

SEQ ID NO: 215 - DNA, 50 bp, Artificial Sequence
  Probe SG215 Designed to hybridize with cRNA of the human gene CD22
acgtcattccagattttccagaagatgaggggattcattactcagagctg

SEQ ID NO: 216 - DNA, 50 bp, Artificial Sequence
  Probe SG216 Designed to hybridize with cRNA of the human gene FCER2
actccgggaggaggtgacaaagctaaggatggagttgcaggtgtccagcg

SEQ ID NO: 217 - DNA, 50 bp, Artificial Sequence
  Probe SG217 Designed to hybridize with cRNA of the human gene CD24
cagccagtctcttcgtggtctcactctctcttctgcatctctactcttaa

SEQ ID NO: 218 - DNA, 50 bp, Artificial Sequence
  Probe SG218 Designed to hybridize with cRNA of the human gene IL2RA
ctgctgagagcgtctgcaaaatgacccacgggaagacaaggtggacccag

SEQ ID NO: 219 - DNA, 50 bp, Artificial Sequence
  Probe SG219 Designed to hybridize with cRNA of the human gene DPP4
gacaaccttgaccattacagaaattcaacagtcatgagcagagctgaaaa

SEQ ID NO: 220 - DNA, 50 bp, Artificial Sequence
  Probe SG220 Designed to hybridize with cRNA of the human gene ITGB1
gagaaaatgaatgccaaatgggacacgcaagaaaatccgatttacaagag

SEQ ID NO: 221 - DNA, 50 bp, Artificial Sequence
  Probe SG221 Designed to hybridize with cRNA of the human gene TNFRSF8
agacggtcaccaagccccaggatatggctgagaaggacaccacctttgag

SEQ ID NO: 222 - DNA, 50 bp, Artificial Sequence
  Probe SG222 Designed to hybridize with cRNA of the human gene CD33
ttacatggccccactgaaacctcaagctgttcaggtgccgcccctactgt

SEQ ID NO: 223 - DNA, 50 bp, Artificial Sequence
  Probe SG223 Designed to hybridize with cRNA of the human gene CD34
cctgctggctgtcttgggcatcactggctatttcctgatgaatcgccgca

SEQ ID NO: 224 - DNA, 50 bp, Artificial Sequence
  Probe SG224 Designed to hybridize with cRNA of the human gene CD36
ggcctttgcctctccagttgaaaacccagacaactattgtttctgcacag

SEQ ID NO: 225 - DNA, 50 bp, Artificial Sequence
  Probe SG225 Designed to hybridize with cRNA of the human gene CD38
agtgtggaagtccataatttgcaaccagagaaggttcagacactagaggc

SEQ ID NO: 226 - DNA, 50 bp, Artificial Sequence
  Probe SG226 Designed to hybridize with cRNA of the human gene TNFRSF5
acccaggaggatggcaaagagagtcgcatctcagtgcaggagagacagtg

SEQ ID NO: 227 - DNA, 50 bp, Artificial Sequence; identical to SEQ ID NO: 226
  Probe SG227 Designed to hybridize with cRNA of the human gene SPN

SEQ ID NO: 228 - DNA, 50 bp, Artificial Sequence
  Probe SG228 Designed to hybridize with cRNA of the human gene CD44
gagctgtggaggacagaaagccaagtggactcaacggagaggccagcaag

SEQ ID NO: 229 - DNA, 50 bp, Artificial Sequence
  Probe SG229 Designed to hybridize with cRNA of the human gene CD44v6
ctgggattggttttcatggttgtttctaccatcagagtcaaagaatcatc

SEQ ID NO: 230 - DNA, 50 bp, Artificial Sequence
  Probe SG230 Designed to hybridize with cRNA of the human gene PTPRCCD
aacattctgtcaatggtcctgcaagtccagccttaaatcaaggttcatag

SEQ ID NO: 231 - DNA, 50 bp, Artificial Sequence
  Probe SG231 Designed to hybridize with cRNA of the human gene CD47
tcaggtgatagcctatatcctcgctgtggttggactgagtctctgtattg

SEQ ID NO: 232 - DNA, 50 bp, Artificial Sequence
  Probe SG232 Designed to hybridize with cRNA of the human gene ITGA2
agccgaagtaccaacaggagttataataggaagtataattgctggaatcc

SEQ ID NO: 233 - DNA, 50 bp, Artificial Sequence
  Probe SG233 Designed to hybridize with cRNA of the human gene ITGA3
ctaagaggcagaaggcggagatgaagagccagccgtcagagacagagagg

SEQ ID NO: 234 - DNA, 50 bp, Artificial Sequence
  Probe SG234 Designed to hybridize with cRNA of the human gene ITGA4
cttggacttattgtacttctgttgatctcatatgttatgtggaaggctgg

SEQ ID NO: 235 - DNA, 50 bp, Artificial Sequence
  Probe SG235 Designed to hybridize with cRNA of the human gene ITGA5
tcaactgcaccaccaatcaccccattaacccaaagggcctggagttggat

SEQ ID NO: 236 - DNA, 50 bp, Artificial Sequence
  Probe SG236 Designed to hybridize with cRNA of the human gene ICAM3
ctatgcagccgacagaagcaatgggggaagaaccgtccagagctgagtga

SEQ ID NO: 237 - DNA, 50 bp, Artificial Sequence
  Probe SG237 Designed to hybridize with cRNA of the human gene CDW52
ccctcagcatccagcagcatgagcggaggcattttccttttcttcgtggc

SEQ ID NO: 238 - DNA, 50 bp, Artificial Sequence
  Probe SG238 Designed to hybridize with cRNA of the human gene ICAM1
gaacaaccggaaggtgtatgaactgagcaatgtgcaagaagatagccaac

SEQ ID NO: 239 - DNA, 50 bp, Artificial Sequence
  Probe SG239 Designed to hybridize with cRNA of the human gene NCAM1
gaggtgaccccagactctgagaatgattttgggaactacaactgtactgc

SEQ ID NO: 240 - DNA, 50 bp, Artificial Sequence
  Probe SG240 Designed to hybridize with cRNA of the human gene CD58
catcaatcattttgacaacctgtatcccaagcagcggtcattcaagacac

SEQ ID NO: 241 - DNA, 50 bp, Artificial Sequence
  Probe SG241 Designed to hybridize with cRNA of the human gene CD59
cgaacagcttgaaaatggtgggacatccttatcagagaaaacagttcttc

SEQ ID NO: 242 - DNA, 50 bp, Artificial Sequence
  Probe SG242 Designed to hybridize with cRNA of the human gene SELL
taggaacatatggagtttttacaaacgctgcatttgacccgagtccttaa

SEQ ID NO: 243 - DNA, 50 bp, Artificial Sequence
  Probe SG243 Designed to hybridize with cRNA of the human gene CD79B
gccacctatgaggacatagtgacgctgcggacaggggaagtgaagtggtc

SEQ ID NO: 244 - DNA, 50 bp, Artificial Sequence
  Probe SG244 Designed to hybridize with cRNA of the human gene CD81
cttcaaggaggactgccaccagaagatcgatgacctcttctccgggaagc

SEQ ID NO: 245 - DNA, 50 bp, Artificial Sequence
  Probe SG245 Designed to hybridize with cRNA of the human gene KAI1
cctgaggtcacctacccctgttcctgcgaagtcaagggggaagaggacaa

SEQ ID NO: 246 - DNA, 50 bp, Artificial Sequence
  Probe SG246 Designed to hybridize with cRNA of the human gene CD83
caaataagcatttagggctagtgactcctcacaagacagaactggtatga

SEQ ID NO: 247 - DNA, 50 bp, Artificial Sequence
  Probe SG247 Designed to hybridize with cRNA of the human gene CD86
caactcttataaatgtggaaccaacacaatggagagggaagagagtgaac

SEQ ID NO: 248 - DNA, 50 bp, Artificial Sequence
  Probe SG248 Designed to hybridize with cRNA of the human gene TNFRSF6
cttccaaatgcagaagatgtagattgtgtgatgaaggacatgatgtgaac

SEQ ID NO: 249 - DNA, 50 bp, Artificial Sequence
  Probe SG249 Designed to hybridize with cRNA of the human gene TNFSF6
tgaggaactctaagtatccccaggatctggtgatgatggaggggaagatg

SEQ ID NO: 250 - DNA, 50 bp, Artificial Sequence
  Probe SG250 Designed to hybridize with cRNA of the human gene SLC7A5
tgacatcccaggggctgtaagtgccaacatgactgtgaagggccagagtg

SEQ ID NO: 251 - DNA, 50 bp, Artificial Sequence
  Probe SG251 Designed to hybridize with cRNA of the human gene SDC1
aaacaagccaacggcggggcctaccagaagcccaccaaacaggaggaatt

SEQ ID NO: 252 - DNA, 50 bp, Artificial Sequence
  Probe SG252 Designed to hybridize with cRNA of the human gene CDK4
ctggaaatgctgacttttaacccacacaagcgaatctctgcctttcgagc

SEQ ID NO: 253 - DNA, 50 bp, Artificial Sequence
  Probe SG253 Designed to hybridize with cRNA of the human gene STAT3
agggcagtttgagtccctcacctttgacatggagttgacctcggagtgcg

SEQ ID NO: 254 - DNA, 50 bp, Artificial Sequence
  Probe SG254 Designed to hybridize with cRNA of the human gene FGR
gactatgaggctcgaactgaggatgacctcaccttcaccaagggcgagaa

SEQ ID NO: 255 - DNA, 50 bp, Artificial Sequence
  Probe SG255 Designed to hybridize with cRNA of the human gene FOS
gagcgcagagcattggcaggaggggcaaggtggaacagttatctccagaa

SEQ ID NO: 256 - DNA, 50 bp, Artificial Sequence
  Probe SG256 Designed to hybridize with cRNA of the human gene CCNE1
tgtaccaagtggagcaggtggttgcgggcaagcgttgtgcagagcccata

SEQ ID NO: 257 - DNA, 50 bp, Artificial Sequence
  Probe SG257 Designed to hybridize with cRNA of the human gene CDA
ccgctatccagaaggccgtctcagaagggtacaaggatttcagggcaatt

SEQ ID NO: 258 - DNA, 50 bp, Artificial Sequence
  Probe SG258 Designed to hybridize with cRNA of the human gene MAFB
gcaaggaggaggtgatccggctgaagcagaagaggcggaccctgaaaaac

SEQ ID NO: 259 - DNA, 50 bp, Artificial Sequence
  Probe SG259 Designed to hybridize with cRNA of the human gene MPL
tgctgtaccacccacattgccaaccattcctacctaccactaagctattg

SEQ ID NO: 260 - DNA, 50 bp, Artificial Sequence
  Probe SG260 Designed to hybridize with cRNA of the human gene MYC
gttgcggaaacgacgagaacagttgaaacacaaacttgaacagctacgga

SEQ ID NO: 261 - DNA, 50 bp, Artificial Sequence
  Probe SG261 Designed to hybridize with cRNA of the human gene MYB
agctaaaaggacagcaggtgctaccagtaagactgtcatcatgtgcttga

SEQ ID NO: 262 - DNA, 50 bp, Artificial Sequence
  Probe SG262 Designed to hybridize with cRNA of the human gene CKMT1
gctggtgcagatggtggtggacggagtgaagctgctcatcgagatggaac

SEQ ID NO: 263 - DNA, 50 bp, Artificial Sequence
  Probe SG263 Designed to hybridize with cRNA of the human gene CREBBP
cacgtctcaccccagactggttccccccaccccggactcgcagtcaccat

SEQ ID NO: 264 - DNA, 50 bp, Artificial Sequence
  Probe SG264 Designed to hybridize with cRNA of the human gene CTGF
catcaagacctgtgcctgccattacaactgtcccggagacaatgacatct

SEQ ID NO: 265 - DNA, 50 bp, Artificial Sequence
  Probe SG265 Designed to hybridize with cRNA of the human gene CXCR3
agctttgaccgctacctgaacatagttcatgccacccagctctaccgccg

SEQ ID NO: 266 - DNA, 50 bp, Artificial Sequence
  Probe SG266 Designed to hybridize with cRNA of the human gene CXCR4
attcatctgtttccactgagtctgagtcttcaagttttcactccagctaa

SEQ ID NO: 267 - DNA, 50 bp, Artificial Sequence
  Probe SG267 Designed to hybridize with cRNA of the human gene CCNA1
gcaatcatgtaccctggatcttttattgggggctggggagaagagtatct

SEQ ID NO: 268 - DNA, 50 bp, Artificial Sequence
  Probe SG268 Designed to hybridize with cRNA of the human gene CCNB1
ggacttacaaagcacatgactgtcaagaacaagtatgccacatcgaagca

SEQ ID NO: 269 - DNA, 50 bp, Artificial Sequence
  Probe SG269 Designed to hybridize with cRNA of the human gene CCMD2
aactggaccaagccagcacccctacagacgtgcgggatatcgacctgtga

SEQ ID NO: 270 - DNA, 50 bp, Artificial Sequence
  Probe SG270 Designed to hybridize with cRNA of the human gene CYP1A1
accaagaactgcttagcctagtcaacctgaataataatttcggggaggtg

SEQ ID NO: 271 - DNA, 50 bp, Artificial Sequence
  Probe SG271 Designed to hybridize with cRNA of the human gene CYP2A6
gcgcatgatgctaggaatcttccagttcacgtcaacctccacggggcagc

SEQ ID NO: 272 - DNA, 50 bp, Artificial Sequence
  Probe SG272 Designed to hybridize with cRNA of the human gene DAD-
cggtttgcctgagaatacagatcaacccacagaacaaagcggatttccaa

SEQ ID NO: 273 - DNA, 50 bp, Artificial Sequence
  Probe SG273 Designed to hybridize with cRNA of the human gene TNFRSF10C
tcatgcaccatcgtagggatcatagttctaattgtgcttctgattgtgtt

SEQ ID NO: 274 - DNA, 50 bp, Artificial Sequence
  Probe SG274 Designed to hybridize with cRNA of the human gene DEK
acagaaagctacttctaaaagtaaaaaatctgtgaaaagtgccaatgtta

SEQ ID NO: 275 - DNA, 50 bp, Artificial Sequence
  Probe SG275 Designed to hybridize with cRNA of the human gene DCK
gctggctcctgcataggacactgaaaaccaacttcgattatcttcaagag

SEQ ID NO: 276 - DNA, 50 bp, Artificial Sequence
  Probe SG276 Designed to hybridize with cRNA of the human gene DHFR
ccaggtgttctctctgatgtccaggaggagaaaggcattaagtacaaatt

SEQ ID NO: 277 - DNA, 50 bp, Artificial Sequence
  Designed to hybridize with cRNA of the human gene TCF3
tcaggtgtggttggagacccccagatggtgctttcagctccccacccagg

SEQ ID NO: 278 - DNA, 50 bp, Artificial Sequence
  Probe SG278 Designed to hybridize with cRNA of the human gene E2F1
tcagagacctcttcgactgtgactttggggacctcacccccctggatttc

SEQ ID NO: 279 - DNA, 50 bp, Artificial Sequence
  Probe SG279 Designed to hybridize with cRNA of the human gene TCF3
tgccagatgcacctcaagtcggacaaagcgcagaccaagctgctcatcct

SEQ ID NO: 280 - DNA, 50 bp, Artificial Sequence
  Probe SG280 Designed to hybridize with cRNA of the human gene EB-1
tactttgatgatattccccgatcaaaactggagaggcagatggctcagac

SEQ ID NO: 281 - DNA, 50 bp, Artificial Sequence
  Probe SG281 Designed to hybridize with cRNA of the human gene CCR7
gccttcatcggcgtcaagttccgcaacgatctcttcaagctcttcaagga

SEQ ID NO: 282 - DNA, 50 bp, Artificial Sequence
  Probe SG282 Designed to hybridize with cRNA of the human gene EBI2
gtcagtgtatcgatttctagtgctgtgaagtcagcccctgaagaaaattc

SEQ ID NO: 283 - DNA, 50 bp, Artificial Sequence
  Probe SG283 Designed to hybridize with cRNA of the human gene MUC-1
tacctcctctcacctcctccaatcacagcacttctccccagttgtctact

SEQ ID NO: 284 - DNA, 50 bp, Artificial Sequence
  Probe SG284 Designed to hybridize with cRNA of the human gene EphA3
atgtgggcattcagctccgacggcccttgttccagaaggtaaccacggtg

SEQ ID NO: 285 - DNA, 50 bp, Artificial Sequence
  Probe SG285 Designed to hybridize with cRNA of the human gene EPOR
tcagaagcatcctcctgctcatctgctttggctaccttccagcataacta

SEQ ID NO: 286 - DNA, 50 bp, Artificial Sequence
  Probe SG286 Designed to hybridize with cRNA of the human gene ERCC1
aaacggacagtcagaccctcctgaccacatttggatctctggaacagctc

SEQ ID NO: 287 - DNA, 50 bp, Artificial Sequence
  Probe SG287 Designed to hybridize with cRNA of the human gene ERCC2
tcaggggcaagacggactacggcctcatggtctttgccgacaagcggttt

SEQ ID NO: 288 - DNA, 50 bp, Artificial Sequence
  Probe SG288 Designed to hybridize with cRNA of the human gene ERCC3
cggcgctttggcaccatgagttctatgtctggggccgacgacactgtgta

SEQ ID NO: 289 - DNA, 50 bp, Artificial Sequence
  Probe SG289 Designed to hybridize with cRNA of the human gene ERCC5
tagtgatgacgatggagggaaagagaagatggtcctcgtgaccgccagat

SEQ ID NO: 290 - DNA, 50 bp, Artificial Sequence
  Probe SG290 Designed to hybridize with cRNA of the human gene ERCC6
tgcactttccatagaacttctggtggtgaaggaatttggaaactcaagcc

SEQ ID NO: 291 - DNA, 50 bp, Artificial Sequence
  Probe SG291 Designed to hybridize with cRNA of the human gene CUZD1
cacagtgaggcattttgtaaatcaacgggcagactacaaataccagaagc

SEQ ID NO: 292 - DNA, 50 bp, Artificial Sequence
  Probe SG292 Designed to hybridize with cRNA of the human gene ETV6
aatcatgagtggccgaacagaccgtctggagcacctagagtcccaggagc

SEQ ID NO: 293 - DNA, 50 bp, Artificial Sequence
  Probe SG293 Designed to hybridize with cRNA of the human gene EIF4E
attatggaatacaaaactcacaccgacagcatcaaaatgccaggcaggct

SEQ ID NO: 294 - DNA, 50 bp, Artificial Sequence
  Probe SG294 Designed to hybridize with cRNA of the human gene EIF5A
ccttggcaaggagattgagcagaagtacgactgtggagaagagatcctga

SEQ ID NO: 295 - DNA, 50 bp, Artificial Sequence
  Probe SG295 Designed to hybridize with cRNA of the human gene EZH2
tccacggcagccttgtgacagttcgtgcccttgtgtgatagcacaaaatt

SEQ ID NO: 296 - DNA, 50 bp, Artificial Sequence
  Probe SG296 Designed to hybridize with cRNA of the human gene FADD
atgtccccgatgtcatggaactcagacgcatctacctccgaagcgtcctg

SEQ ID NO: 297 - DNA, 50 bp, Artificial Sequence
  Probe SG297 Designed to hybridize with cRNA of the human gene PTK2
gcacccaccgagagattgagatggcacagaagctattgaactctgacctg

SEQ ID NO: 298 - DNA, 50 bp, Artificial Sequence
  Probe SG298 Designed to hybridize with cRNA of the human gene FAT
ttgtagagaaccccatgccccttacccgccagggtatcaaagacacttcg

SEQ ID NO: 299 - DNA, 50 bp, Artificial Sequence
  Probe SG299 Designed to hybridize with cRNA of the human gene FGFR1
ttcttctggttcggccatcacggctctcctccagtgggactcccatgcta

SEQ ID NO: 300 - DNA, 50 bp, Artificial Sequence
  Probe SG300 Designed to hybridize with cRNA of the human gene FGFR3
aggagctagaggttctctccttgcacaacgtcacctttgaggacgccggg

SEQ ID NO: 301 - DNA, 50 bp, Artificial Sequence
  Probe SG301 Designed to hybridize with cRNA of the human gene FHIT
tcctgcctcttggagatcagaggaggaaatggcagcagaagccgcagctc

SEQ ID NO: 302 - DNA, 50 bp, Artificial Sequence
  Probe SG302 Designed to hybridize with cRNA of the human gene FLT3
tggagcgcgtgaacacggagatacactttgtcaccaaatgtgcctttcag

SEQ ID NO: 303 - DNA, 50 bp, Artificial Sequence
  Probe SG303 Designed to hybridize with cRNA of the human gene GRIA3
gcgatacttgattgactgcgaagtcgaaaggattaacacaattttggaac

SEQ ID NO: 304 - DNA, 50 bp, Artificial Sequence
  Probe SG304 Designed to hybridize with cRNA of the human gene FUS
gggacagcccatgattaatttgtacacagacagggaaactggcaagctga

SEQ ID NO: 305 - DNA, 50 bp, Artificial Sequence
  Probe SG305 Designed to hybridize with cRNA of the human gene EIF4E
cccacgcagacacagctactaagagcggctccaccactaaaaataggttt

SEQ ID NO: 306 - DNA, 50 bp, Artificial Sequence
  Probe SG306 Designed to hybridize with cRNA of the human gene SIAT4A
agactttgagtctaacgtgacggccaccttggcctccatcaataaaatcc

SEQ ID NO: 307 - DNA, 50 bp, Artificial Sequence
  Probe SG307 Designed to hybridize with cRNA of the human gene CSF3
gcttcctggaggtgtcgtaccgcgttctacgccaccttgcccagccctga

SEQ ID NO: 308 - DNA, 50 bp, Artificial Sequence
  Probe SG308 Designed to hybridize with cRNA of the human gene CSF2
ccctccaaccccggaaacttcctgtgcaacccagattatcacctttgaaa

SEQ ID NO: 309 - DNA, 50 bp, Artificial Sequence
  Probe SG309 Designed to hybridize with cRNA of the human gene NR3C1
cagccagaactggcagcggttttatcaactgacaaaactcttggattcta

SEQ ID NO: 310 - DNA, 50 bp, Artificial Sequence
  Probe SG310 Designed to hybridize with cRNA of the human gene GSTT1
ataaggtgatgttccctgtgttcctgggtgggccagtatctccccagaca

SEQ ID NO: 311 - DNA, 50 bp, Artificial Sequence
  Probe SG311 Designed to hybridize with cRNA of the human gene GZMA
ggcctggtgtctatattcttctctcaaagaaacacctcaactggataatt

SEQ ID NO: 312 - DNA, 50 bp, Artificial Sequence
  Probe SG312 Designed to hybridize with cRNA of the human gene DNAJA1
agatcttctgccatctagaccggaagttcctaacataattggagaaacag

SEQ ID NO: 313 - DNA, 50 bp, Artificial Sequence
  Probe SG313 Designed to hybridize with cRNA of the human gene HLF
gatcctgacaccatccaggtcccagtgggttatgagccagacccagcaga

SEQ ID NO: 314 - DNA, 50 bp, Artificial Sequence
  Probe SG314 Designed to hybridize with cRNA of the human gene BST2
agaagagaaaaccaggtcttaagcgtgagaatcgcggacaagaagtacta

SEQ ID NO: 315 - DNA, 50 bp, Artificial Sequence
  Probe SG315 Designed to hybridize with cRNA of the human gene HOXA9
ggggtgactgtcccacgcttgacactcacactttgtccctgactgactat

SEQ ID NO: 316 - DNA, 50 bp, Artificial Sequence
  Probe SG316 Designed to hybridize with cRNA of the human gene TLX1
gacaggttcacaggtcacccctatcagaaccggacgccccccaagaagaa

SEQ ID NO: 317 - DNA, 50 bp, Artificial Sequence
  Probe SG317 Designed to hybridize with cRNA of the human gene UHRF1
cgctacgatggcatctacaaggttgtgaaatactggcccgagaaggggaa

SEQ ID NO: 318 - DNA, 50 bp, Artificial Sequence
  Probe SG318 Designed to hybridize with cRNA of the human gene IER3
aaaaggcttctctttctgctgctcaccatcgtcttctgccagatcctgat

SEQ ID NO: 319 - DNA, 50 bp, Artificial Sequence
  Probe SG319 Designed to hybridize with cRNA of the human gene IL1B
cagaccttccaggagaatgacctgagcaccttctttcccttcatctttga

SEQ ID NO: 320 - DNA, 50 bp, Artificial Sequence
  Probe SG320 Designed to hybridize with cRNA of the human gene IL2
agcaaccattgtagaatttctgaacagatggattaccttttgtcaaagca

SEQ ID NO: 321 - DNA, 50 bp, Artificial Sequence
  Probe SG321 Designed to hybridize with cRNA of the human gene IL3
gcacccacgcgacatccaatccatatcaaggacggtgactggaatgaatt

SEQ ID NO: 322 - DNA, 50 bp, Artificial Sequence
  Probe SG322 Designed to hybridize with cRNA of the human gene IL6
gaacaagccagagctgtccagatgagtacaaaagtcctgatccagttcct

SEQ ID NO: 323 - DNA, 50 bp, Artificial Sequence
  Probe SG323 Designed to hybridize with cRNA of the human gene IL6R
cacggagcccttatgacatcagcaatacagactacttcttccccagatag

SEQ ID NO: 324 - DNA, 50 bp, Artificial Sequence
  Probe SG324 Designed to hybridize with cRNA of the human gene IL10
ccaagagaaaggcatctacaaagccatgagtgagtttgacatcttcatca

SEQ ID NO: 325 - DNA, 50 bp, Artificial Sequence
  Probe SG325 Designed to hybridize with cRNA of the human gene IL15
atacggaaagtgatgttcaccccagttgcaaagtaacagcaatgaagtgc

SEQ ID NO: 326 - DNA, 50 bp, Artificial Sequence
  Probe SG326 Designed to hybridize with cRNA of the human gene IRF4
atccagaagattaccacagatctatccgccattcctctattcaagaatga

SEQ ID NO: 327 - DNA, 50 bp, Artificial Sequence
  Probe SG327 Designed to hybridize with cRNA of the human gene JAK1
aacgcctgccgtgcccacctaactgtccagatgaggtttatcagcttatg

SEQ ID NO: 328 - DNA, 50 bp, Artificial Sequence
  Probe SG328 Designed to hybridize with cRNA of the human gene JAK2
aatcaacgcccctcctttagggatctagctcttcgagtggatcaaataag

SEQ ID NO: 329 - DNA, 50 bp, Artificial Sequence
  Probe SG329 Designed to hybridize with cRNA of the human gene MKI67
tgtgtcaagaaaataacaaccagaagtcatagggacagtgaagatatttg

SEQ ID NO: 330 - DNA, 50 bp, Artificial Sequence
  Probe SG330 Designed to hybridize with cRNA of the human gene XRCC5
tgggaaattgttgtccaggatggaattactctgatcaccaaagaggaagc

SEQ ID NO: 331 - DNA, 50 bp, Artificial Sequence
  Probe SG331 Designed to hybridize with cRNA of the human gene LAG3
agcaagggattcaccctcgccaggctcagacgcaagatagaggagctgga

SEQ ID NO: 332 - DNA, 50 bp, Artificial Sequence
  Probe SG332 Designed to hybridize with cRNA of the human gene LCK
cccttcaggcatcaagttgaccatcaacaaactcctggacatggcagccc

SEQ ID NO: 333 - DNA, 50 bp, Artificial Sequence
  Probe SG333 Designed to hybridize with cRNA of the human gene LCP1
tttcaaggacccgaagattagtacaagtctgcctgttctggacctcatcg

SEQ ID NO: 334 - DNA, 50 bp, Artificial Sequence
  Probe SG334 Designed to hybridize with cRNA of the human gene LEPR
agaattgttcctgggcacaaggacttaattttcagaagagaacggacatt

SEQ ID NO: 335 - DNA, 50 bp, Artificial Sequence
  Probe SG335 Designed to hybridize with cRNA of the human gene LMO2
tgtgcgaacaggacatctacgagtggactaagatcaatgggatgatatag

SEQ ID NO: 336 - DNA, 50 bp, Artificial Sequence
  Probe SG336 Designed to hybridize with cRNA of the human gene LIF
cgtgacggacttcccgcccttccacgccaacggcacggagaaggccaagc

SEQ ID NO: 337 - DNA, 50 bp, Artificial Sequence
  Probe SG337 Designed to hybridize with cRNA of the human gene LRP
ctggaccagtcagaagccgagaaagctcgcaaggaacttttggagctgga

SEQ ID NO: 338 - DNA, 50 bp, Artificial Sequence
  Probe SG338 Designed to hybridize with cRNA of the human gene LSP1
ggaccgagtccctaaaccgctccatagagaagagtaacagtgtgaagaaa

SEQ ID NO: 339 - DNA, 50 bp, Artificial Sequence
  Probe SG339 Designed to hybridize with cRNA of the human gene LYL1
agtgtctacattgggccagcaggaccttttagcatcttccctagcagccg

SEQ ID NO: 340 - DNA, 50 bp, Artificial Sequence
  Probe SG340 Designed to hybridize with cRNA of the human gene NFKB1
catccttccgcaaactcagctttaccgagtctctgaccagtggtgcctca

SEQ ID NO: 341 - DNA, 50 bp, Artificial Sequence
  Probe SG341 Designed to hybridize with cRNA of the human gene MAL
caccttgatcatcctgtacataattggagcccacggtggagagacttcct

SEQ ID NO: 342 - DNA, 50 bp, Artificial Sequence
  Probe SG342 Designed to hybridize with cRNA of the human gene MAGEA1
ctcctcccctcctctcctctcagctagtccatgtttctccaacacaagtt

SEQ ID NO: 343 - DNA, 50 bp, Artificial Sequence
  Probe SG343 Designed to hybridize with cRNA of the human gene MAPK10
agactaaaaatggtgtagtaaaaggacagccttctccttcagcacaggtg

SEQ ID NO: 344 - DNA, 50 bp, Artificial Sequence
  Probe SG344 Designed to hybridize with cRNA of the human gene MBP1
ccatgcagagtctgtcctcccagtcggtgcagcaaactcagggaagccat

SEQ ID NO: 345 - DNA, 50 bp, Artificial Sequence
  Probe SG345 Designed to hybridize with cRNA of the human gene MCL1
ggctcccagtgactactttttgacttctgtttgtcttacgcttctctcag

SEQ ID NO: 346 - DNA, 50 bp, Artificial Sequence
  Probe SG346 Designed to hybridize with cRNA of the human gene MDM2
tgaatctctcgactcagaagattatagccttagtgaagaaggacaagaac

SEQ ID NO: 347 - DNA, 50 bp, Artificial Sequence
  Probe SG347 Designed to hybridize with cRNA of the human gene ABCB1
tagctcgtgcccttgttagacagcctcatattttgcttttggatgaagcc

SEQ ID NO: 348 - DNA, 50 bp, Artificial Sequence
  Probe SG348 Designed to hybridize with cRNA of the human gene ABCB4
tagcgatttttggaccaggcgatgatgcagtgaagcagcagaagtgcaac

SEQ ID NO: 349 - DNA, 50 bp, Artificial Sequence
  Probe SG349 Designed to hybridize with cRNA of the human gene MEN1
attgcctcagccaagacctactatcgggatgaacacatctacccctacat

SEQ ID NO: 350 - DNA, 50 bp, Artificial Sequence
  Probe SG350 Designed to hybridize with cRNA of the human gene CXCL9
gaaggaactgattaaaaagtgggagaaacaggtcagccaaaagaaaaagc

SEQ ID NO: 351 - DNA, 50 bp, Artificial Sequence
  Probe SG351 Designed to hybridize with cRNA of the human gene MLF1
ggggacaaactccacatcaaaggctcatctgtgaaaagcaacaaaaaata

SEQ ID NO: 352 - DNA, 50 bp, Artificial Sequence
  Probe SG352 Designed to hybridize with cRNA of the human gene MLL
gaaaacaggaccattgcccatagtccaacatcttttacagaaagttcatc

SEQ ID NO: 353 - DNA, 50 bp, Artificial Sequence
  Probe SG353 Designed to hybridize with cRNA of the human gene MMP9
agtgagttgaaccaggtggaccaagtgggctacgtgacctatgacatcct

SEQ ID NO: 354 - DNA, 50 bp, Artificial Sequence
  Probe SG354 Designed to hybridize with cRNA of the human gene NDUFB
ccggaacaagagtatgttatttaaaagggaattgcaacccagtgaagaag

SEQ ID NO: 355 - DNA, 50 bp, Artificial Sequence
  Probe SG355 Designed to hybridize with cRNA of the human gene MPO
atcaccaccgtgtctaagaacaacatcttcatgtccaactcatatccccg

SEQ ID NO: 356 - DNA, 50 bp, Artificial Sequence
  Probe SG356 Designed to hybridize with cRNA of the human gene ABCC3
ccaggtaaagcaaatgaaattgaaggactcgcgcatcaagctgatgagtg

SEQ ID NO: 357 - DNA, 50 bp, Artificial Sequence
  Probe SG357 Designed to hybridize with cRNA of the human gene ABCC5
catcggtccttctgtccaacgacagttcccgattctatgccatgtttgct

SEQ ID NO: 358 - DNA, 50 bp, Artificial Sequence
  Probe SG358 Designed to hybridize with cRNA of the human gene MTCP1
cagatacagcatcatttaatggtcaggggagtacaggagctgttgcttaa

SEQ ID NO: 359 - DNA, 50 bp, Artificial Sequence
  Probe SG359 Designed to hybridize with cRNA of the human gene MTCP1
gtgttgtgctcagtatcccaagggaagatctgtcgtctgttcaggatttg

SEQ ID NO: 360 - DNA, 50 bp, Artificial Sequence
  Probe SG360 Designed to hybridize with cRNA of the human gene MYOD1
ttgccacaacggacgacttctatgacgacccgtgtttcgactccccggac

SEQ ID NO: 361 - DNA, 50 bp, Artificial Sequence
  Probe SG361 Designed to hybridize with cRNA of the human gene MX1
cgtcctgtcggagccctgtctctctctctgtaataaactcatttctagca

SEQ ID NO: 362 - DNA, 50 bp, Artificial Sequence
  Probe SG362 Designed to hybridize with cRNA of the human gene NINJ1
agtacgaccttaacaacccggacaagcacgccaagctggacttcctcaac

SEQ ID NO: 363 - DNA, 50 bp, Artificial Sequence
  Probe SG363 Designed to hybridize with cRNA of the human gene NPM1
tagagtgatggattgatacctagcagcctgtgaaaaggaatcagtgaatt

SEQ ID NO: 364 - DNA, 50 bp, Artificial Sequence
  Probe SG364 Designed to hybridize with cRNA of the human gene IL32
aggaggagctgacaccccagaagtgctctgaaccccaatcctcaaaatga

SEQ ID NO: 365 - DNA, 50 bp, Artificial Sequence
  Probe SG365 Designed to hybridize with cRNA of the human gene NUMA1
ccccaacactcgcagtggaacccgccgttctccgcgcattgccaccacca

SEQ ID NO: 366 - DNA, 50 bp, Artificial Sequence
  Probe SG366 Designed to hybridize with cRNA of the human gene POU2F2
ccaactggccccagtacaaagatcaaggctgaagaccccagtggcgattc

SEQ ID NO: 367 - DNA, 50 bp, Artificial Sequence
  Probe SG367 Designed to hybridize with cRNA of the human gene POU2F2
tcccaagcttccagcagtctgagcacaacagttactaccttatcctcagc

SEQ ID NO: 368 - DNA, 50 bp, Artificial Sequence
  Probe SG368 Designed to hybridize with cRNA of the human gene OGGI
gaccccatcgaatgccttttctcttttatctgttcctccaacaacaacat

SEQ ID NO: 369 - DNA, 50 bp, Artificial Sequence
  Probe SG369 Designed to hybridize with cRNA of the human gene CDKN2B
gcggatcccaacggagtcaaccgtttcgggaggcgcgcgatccaggtcat

SEQ ID NO: 370 - DNA, 50 bp, Artificial Sequence
  Probe SG370 Designed to hybridize with cRNA of the human gene CDKN2A
tgaggagccagcgtctagggcagcagccgcttcctagaagaccaggtcat

SEQ ID NO: 371 - DNA, 50 bp, Artificial Sequence
  Probe SG371 Designed to hybridize with cRNA of the human gene CDKN2C
ttagcctgatgcaggcaaacggggctgggggagccacaaatcttcaataa

SEQ ID NO: 372 - DNA, 50 bp, Artificial Sequence
  Probe SG372 Designed to hybridize with cRNA of the human gene CDKN1A
gaggcccgtgagcgatggaacttcgactttgtcaccgagacaccactgga

SEQ ID NO: 373 - DNA, 50 bp, Artificial Sequence
  Probe SG373 Designed to hybridize with cRNA of the human gene CDKN1B
cctgcaaccgacgattcttctactcaaaacaaaagagccaacagaacaga

SEQ ID NO: 374 - DNA, 50 bp, Artificial Sequence
  Probe SG374 Designed to hybridize with cRNA of the human gene CDK5R1
agaacctgaagaagtcgctgtcgtgcgccaacctgtccacattcgcccag

SEQ ID NO: 375 - DNA, 50 bp, Artificial Sequence
  Probe SG375 Designed to hybridize with cRNA of the human gene TOPORS
aaccagcccagtaacattgtgtctcttcaaactgagccatcaaggcaatt

SEQ ID NO: 376 - DNA, 50 bp, Artificial Sequence
  Probe SG376 Designed to hybridize with cRNA of the human gene P55CDC
ggatgcccattcccaggtgtgctccatcctctggtctccccattacaagg

SEQ ID NO: 377 - DNA, 50 bp, Artificial Sequence
  Probe SG377 Designed to hybridize with cRNA of the human gene CDKN1C
cgcctgagaagtcgtcgggcgatgtccccgcgccgtgtccctctccaagc

SEQ ID NO: 378 - DNA, 50 bp, Artificial Sequence
  Probe SG378 Designed to hybridize with cRNA of the human gene TP73
ctgcatcgagtatttcacctcccaagggttacagagcatttaccacctgc

SEQ ID NO: 379 - DNA, 50 bp, Artificial Sequence
  Probe SGD379 esigned to hybridize with cRNA of the human gene PAX5
cccccccgctggacagggcagctactcagcaccgacgctgacagggatgg

SEQ ID NO: 380 - DNA, 50 bp, Artificial Sequence
  Probe SG380 Designed to hybridize with cRNA of the human gene PBX1
acagtccgcagggcatcagtgctaatggaggttggcaggatgctactacc

SEQ ID NO: 381 - DNA, 50 bp, Artificial Sequence
  Probe SG381 Designed to hybridize with cRNA of the human gene PBX3
ccaaattccggttcttctggttcttttaacctcccaaattctggggacat

SEQ ID NO: 382 - DNA, 50 bp, Artificial Sequence
  Probe SG382 Designed to hybridize with cRNA of the human gene ENPP1
aaacatctatgctgtcaagaccgccgtgtcagtcacccaggggggccaca

SEQ ID NO: 383 - DNA, 50 bp, Artificial Sequence
  Probe SG383 Designed to hybridize with cRNA of the human gene PCA1
tcactggactcagcttctatcaacaaagaaaagagccagtttcagacatt

SEQ ID NO: 384 - DNA, 50 bp, Artificial Sequence
  Probe SG384 Designed to hybridize with cRNA of the human gene PCNA
gctcctgaagccgaaaccagctagactttcctccttcccgcctgcctgta

SEQ ID NO: 385 - DNA, 50 bp, Artificial Sequence
  Probe SG385 Designed to hybridize with cRNA of the human gene PDGFRB
acgactatatcatccccctgcctgaccccaaacccgaggttgctgacgag

SEQ ID NO: 386 - DNA, 50 bp, Artificial Sequence
  Probe SG386 Designed to hybridize with cRNA of the human gene PRKCQ
ggctgtcatttgccgacagagcactgatcaacagcatggaccagaatatg

SEQ ID NO: 387 - DNA, 50 bp, Artificial Sequence
  Probe SG387 Designed to hybridize with cRNA of the human gene PLK1
cctcagcaacggcagcgtgcagatcaacttcttccaggatcacaccaagc

SEQ ID NO: 388 - DNA, 50 bp, Artificial Sequence
  Probe SG388 Designed to hybridize with cRNA of the human gene PML
actcaagaacctggcccagacctacctggcgagaaacatgagcgagcgca

SEQ ID NO: 389 - DNA, 50 bp, Artificial Sequence
  Probe SG389 Designed to hybridize with cRNA of the human gene PRAME
tttgatgagtgtgggatcacggatgatcagctccttgccctcctgccttc

SEQ ID NO: 390 - DNA, 50 bp, Artificial Sequence
  Probe SG390 Designed to hybridize with cRNA of the human gene PRKCI
tctcagtttactaatgaacctgtccagctcactccagatgacgatgacat

SEQ ID NO: 391 - DNA, 50 bp, Artificial Sequence
  Probe SG391 Designed to hybridize with cRNA of the human gene PTEN
atattctgacaccactgactctgatccagagaatgaaccttttgatgaag

SEQ ID NO: 392 - DNA, 50 bp, Artificial Sequence
  Probe SG392 Designed to hybridize with cRNA of the human gene PTGS1
gtacagctacgagcagttcttgttcaacacctccatgttggtggactatg

SEQ ID NO: 393 - DNA, 50 bp, Artificial Sequence
  Probe SG393 Designed to hybridize with cRNA of the human gene PTHLH
agtgactgggagtgggctagaaggggaccacctgtctgacacctccacaa

SEQ ID NO: 394 - DNA, 50 bp, Artificial Sequence
  Probe SG394 Designed to hybridize with cRNA of the human gene SPI1
gatctataccaacgccaaacgcacgagtattacccctatctcagcagtga

SEQ ID NO: 395 - DNA, 50 bp, Artificial Sequence
  Probe SG395 Designed to hybridize with cRNA of the human gene PTK2B
ggagcgtggatgatctcctgccttccttgccgtcatcttcacggacagag

SEQ ID NO: 396 - DNA, 50 bp, Artificial Sequence
  Probe SG396 Designed to hybridize with cRNA of the human gene RAD51
ggttccttgagcacccggaaacggtcacgagacccagaggaagaattata

SEQ ID NO: 397 - DNA, 50 bp, Artificial Sequence
  Probe SG397 Designed to hybridize with cRNA of the human gene RAG1
ggcaagcttaggggacccattaggcatagaggactctctggaaagccaag

SEQ ID NO: 398 - DNA, 50 bp, Artificial Sequence
  Probe SG398 Designed to hybridize with cRNA of the human gene RARA
cagcttccagttagtggatatagcacaccatccccagccaccattgagac

SEQ ID NO: 399 - DNA, 50 bp, Artificial Sequence
  Probe SG399 Designed to hybridize with cRNA of the human gene KRAS2
gttgatgatgccttctatacattagttcgagaaattcgaaaacataaaga

SEQ ID NO: 400 - DNA, 50 bp, Artificial Sequence
  Probe SG400 Designed to hybridize with cRNA of the human gene RB1
aactggcagaaatgacttctactcgaacacgaatgcaaaagcagaaaatg

SEQ ID NO: 401 - DNA, 50 bp, Artificial Sequence
  Probe SG401 Designed to hybridize with cRNA of the human gene VEGF
aaatgttcctgcaaaaacacacactcgcgttgcaaggcgaggcagcttga

SEQ ID NO: 402 - DNA, 50 bp, Artificial Sequence
  Probe SG402 Designed to hybridize with cRNA of the human gene ZYX
gaagaagttcggccctgtggtggccccaaagcccaaagtgaatcccttcc

SEQ ID NO: 403 - DNA, 50 bp, Artificial Sequence
  Probe SG403 Designed to hybridize with cRNA of the human gene CD28
cccctatttcccggaccttctaagcccttttgggtgctggtggtggttgg

SEQ ID NO: 404 - DNA, 50 bp, Artificial Sequence
  Probe SG404 Designed to hybridize with cRNA of the human gene CD28
caaaattgaagttatgtatcctcctccttacctagacaatgagaagagca

SEQ ID NO: 405 - DNA, 50 bp, Artificial Sequence
  Probe SG405 Designed to hybridize with cRNA of the human gene RBBP4
ttctgtatcagaagacaatatcatgcaagtgtggcaaatggcagagaaca

SEQ ID NO: 406 - DNA, 50 bp, Artificial Sequence
  Probe SG406 Designed to hybridize with cRNA of the human gene DDX6
tgtatgtggcagaataccacagcgagcctgtagaagatgagaaaccttaa

SEQ ID NO: 407 - DNA, 50 bp, Artificial Sequence
  Probe SG407 Designed to hybridize with cRNA of the human gene APEX
gcgccttgattactttttgttgtcccactctctgttacctgcattgtgtg

SEQ ID NO: 408 - DNA, 50 bp, Artificial Sequence
  Probe SG408 Designed to hybridize with cRNA of the human gene DPF2
cagggcatccatcttgcctccaatttacccccgtgatgatggcggcagtg

SEQ ID NO: 409 - DNA, 50 bp, Artificial Sequence
  Probe SG409 Designed to hybridize with cRNA of the human gene RGS1
ctggcttgtgaagactataagaaaacagagtctgatcttttgccctgtaa

SEQ ID NO: 410 - DNA, 50 bp, Artificial Sequence
  Probe SG410 Designed to hybridize with cRNA of the human gene HMMR
tttgccctgaagaccccattaaaagaaggcaatacaaactgttaccgagc

SEQ ID NO: 411 - DNA, 50 bp, Artificial Sequence
  Probe SG411 Designed to hybridize with cRNA of the human gene STAT5B
tccttgacaccgatggggacttcgatctggaggacacaatggacgtagcg

SEQ ID NO: 412 - DNA, 50 bp, Artificial Sequence
  Probe SG412 Designed to hybridize with cRNA of the human gene BIRC5
cgctttcctttctgtcaagaagcagtttgaagaattaacccttggtgaat

SEQ ID NO: 413 - DNA, 50 bp, Artificial Sequence
  Probe SG413 Designed to hybridize with cRNA of the human gene TAL1
ttccccctatgagatggagattactgatggtccccacaccaaagttgtgc

SEQ ID NO: 414 - DNA, 50 bp, Artificial Sequence
  Probe SG414 Designed to hybridize with cRNA of the human gene NXF1
tatggtgaaggaaaccgtaggtctggaagaggcggttctggtattcggtc

SEQ ID NO: 415 - DNA, 50 bp, Artificial Sequence
  Probe SG415 Designed to hybridize with cRNA of the human gene TCL1A
ttctggtggagagaagtgagaataggcagcccccaaataaaaaatattca

SEQ ID NO: 416 - DNA, 50 bp, Artificial Sequence
  Probe SG416 Designed to hybridize with cRNA of the human gene TRA@
attattccagaagacaccttcttccccagcccagaaagttcctgtgatgt

SEQ ID NO: 417 - DNA, 50 bp, Artificial Sequence
  Probe SG417 Designed to hybridize with cRNA of the human gene TCR beta
cagccgtgtatctccgtgccagcagcttcaaaggtgcccaggccggttag

SEQ ID NO: 418 - DNA, 50 bp, Artificial Sequence
  Probe SG418 Designed to hybridize with cRNA of the human gene DNTT
ctgtatttggagtggggctgaagacttctgagaagtggttcaggatgggt

SEQ ID NO: 419 - DNA, 50 bp, Artificial Sequence
  Probe SG419 Designed to hybridize with cRNA of the human gene TERT
attcctgctcaagctgactcgacaccgtgtcacctacgtgccactcctgg

SEQ ID NO: 420 - DNA, 50 bp, Artificial Sequence
  Probe SG420 Designed to hybridize with cRNA of the human gene ERBB4
ttgacaaccctgactactggaaccacagcctgccacctcggagcaccctt

SEQ ID NO: 421 - DNA, 50 bp, Artificial Sequence
  Probe SG421 Designed to hybridize with cRNA of the human gene ERBB2
cacccagcaccttcaaagggacacctacggcagagaacccagagtacctg

SEQ ID NO: 422 - DNA, 50 bp, Artificial Sequence
  Probe SG422 Designed to hybridize with cRNA of the human gene EGFR
cccagaaaggcagccaccaaattagcctggacaaccctgactaccagcag

SEQ ID NO: 423 - DNA, 50 bp, Artificial Sequence
  Probe SG423 Designed to hybridize with cRNA of the human gene THPO
cttctaaacacatcctacacccactcccagaatctgtctcaggaagggta

SEQ ID NO: 424 - DNA, 50 bp, Artificial Sequence
  Probe SG424 Designed to hybridize with cRNA of the human gene TIAM1
aggaagtcatttgggttaggcgtgaagactttgccccctccaggaaactg

SEQ ID NO: 425 - DNA, 50 bp, Artificial Sequence
  Probe SG425 Designed to hybridize with cRNA of the human gene TK1
ggaagctctttgccccacagcagattctgcaatgcagccctgccaactga

SEQ ID NO: 426 - DNA, 50 bp, Artificial Sequence
  Probe SG426 Designed to hybridize with cRNA of the human gene TNFRSF1A
agaagtgggaggacagcgcccacaagccacagagcctagacactgatgac

SEQ ID NO: 427 - DNA, 50 bp, Artificial Sequence
  Probe SG427 Designed to hybridize with cRNA of the human gene TOP2A
gcctgatcctgccaaaaccaagaatcgccgcaaaaggaagccatccactt

SEQ ID NO: 428 - DNA, 50 bp, Artificial Sequence
  Probe SG428 Designed to hybridize with cRNA of the human gene FAIM3
accagcctgccgccatgatggaggacagtgattcagatgactacatcaat

SEQ ID NO: 429 - DNA, 50 bp, Artificial Sequence
  Probe SG429 Designed to hybridize with cRNA of the human gene PLS3
gtggcaatctaacagaagatgacaagcacaataatgccaagtatgcagtg

SEQ ID NO: 430 - DNA, 50 bp, Artificial Sequence
  Probe SG430 Designed to hybridize with cRNA of the human gene TRADD
cgccacctgcccagacttttctgttccagggtcagcctgtagtgaatcgg

SEQ ID NO: 431 - DNA, 50 bp, Artificial Sequence
  Probe SG431 Designed to hybridize with cRNA of the human gene TNFSF10
gtaacaaatgagcacttgatagacatggaccatgaagccagttttttcgg

SEQ ID NO: 432 - DNA, 50 bp, Artificial Sequence
  Probe SG432 Designed to hybridize with cRNA of the human gene TRAP 1
aagtttgaggacaggtccccagccgccgagtgcctatcagagaaggagac

SEQ ID NO: 433 - DNA, 50 bp, Artificial Sequence
  Probe SG433 Designed to hybridize with cRNA of the human gene THBS1
tagagtggtgatgtatgaagggaagaaaatcatggctgactcaggaccca

SEQ ID NO: 434 - DNA, 50 bp, Artificial Sequence
  Probe SG434 Designed to hybridize with cRNA of the human gene SUMO1
atgtgattgaagtttatcaggaacaaacggggggtcattcaacagtttag

SEQ ID NO: 435 - DNA, 50 bp, Artificial Sequence
  Probe SG435 Designed to hybridize with cRNA of the human gene UVRAG
gaagaaatcatcgggctggaagcacaggtttcgcctcaggtgatcagcta

SEQ ID NO: 436 - DNA, 50 bp, Artificial Sequence
  Probe SG436 Designed to hybridize with cRNA of the human gene VPREB1
ctgtcctgctcatgctgtttgtctactgcacaggttgtggtcctcagccg

SEQ ID NO: 437 - DNA, 50 bp, Artificial Sequence
  Probe SG437 Designed to hybridize with cRNA of the human gene WNT16
tggtgctgctatgtccgttgcaggaggtgtgaaagcatgactgatgtcca

SEQ ID NO: 438 - DNA, 50 bp, Artificial Sequence
  Probe SG438 Designed to hybridize with cRNA of the human gene WT1
accacagcacagggtacgagagcgataaccacacaacgcccatcctctgc

SEQ ID NO: 439 - DNA, 50 bp, Artificial Sequence
  Probe SG439 Designed to hybridize with cRNA of the human gene XRCC3
gacaccttgttggagtgtgtgaataagaaggtccccgtactgctgtctcg

SEQ ID NO: 440 - DNA, 50 bp, Artificial Sequence
  Probe SG440 Designed to hybridize with cRNA of the human gene CCND3
ttatacctttgccatgtacccgccatccatgatcgccacgggcagcattg

SEQ ID NO: 441 - DNA, 50 bp, Artificial Sequence
  Probe SG441 Designed to hybridize with cRNA of the human gene CDK2
accctttcttccaggatgtgaccaagccagtaccccatcttcgactctga

SEQ ID NO: 442 - DNA, 50 bp, Artificial Sequence
  Probe SG442 Designed to hybridize with cRNA of the human gene p14ARF
aataaaataaggggaataggggagcggggacgcgagcagcaccagaatcc

SEQ ID NO: 443 - DNA, 50 bp, Artificial Sequence
  Probe SG443 Designed to hybridize with cRNA of the human gene HELLS
gaccgagaaagagctgttgtggaagtgaatatccctgtagaatctgaagt

SEQ ID NO: 444 - DNA, 50 bp, Artificial Sequence
  Probe SG444 Designed to hybridize with cRNA of the human gene GATA2
tttcggcttcccacccacgccacccaaagaagtgtctcctgaccctagca

SEQ ID NO: 445 - DNA, 50 bp, Artificial Sequence
  Probe SG445 Designed to hybridize with cRNA of the human gene GATA1
atccggcccaagaagcgcctgattgtcagtaaacgggcaggtactcagtg

SEQ ID NO: 446 - DNA, 50 bp, Artificial Sequence
  Probe SG446 Designed to hybridize with cRNA of the human gene MLLT10
ttaaattctcaacagctcacaccagtacacaggcacccccacttcacaca

SEQ ID NO: 447 - DNA, 50 bp, Artificial Sequence
  Probe SG447 Designed to hybridize with cRNA of the human gene PICALM
cggaaatggaaccactaagaatgatgtaaattggagtcaaccaggtgaaa

SEQ ID NO: 448 - DNA, 50 bp, Artificial Sequence
  Probe SG448 Designed to hybridize with cRNA of the human gene KIT
atgtatgacataatgaagacttgctgggatgcagatcccctaaaaagacc

SEQ ID NO: 449 - DNA, 50 bp, Artificial Sequence
  Probe SG449 Designed to hybridize with cRNA of the human gene IL3RA
gcctggaggagtgtctggtgactgaagtacaggtcgtgcagaaaacttga

SEQ ID NO: 450 - DNA, 50 bp, Artificial Sequence
  Probe SG450 Designed to hybridize with cRNA of the human gene CEBPA
aacgtggagacgcagcagaaggtgctggagctgaccagtgacaatgaccg

SEQ ID NO: 451 - DNA, 50 bp, Artificial Sequence
  Probe SG451 Designed to hybridize with cRNA of the human gene ITGA6
agatccatgctcagccatctgataaagagaggcttacttctgatgcatag

SEQ ID NO: 452 - DNA, 50 bp, Artificial Sequence
  Probe SG452 Designed to hybridize with cRNA of the human gene CML66
caggttgctaagcagcaagtagcaagcctagaaaccaatgatcctatttt

SEQ ID NO: 453 - DNA, 50 bp, Artificial Sequence
  Probe SG453 Designed to hybridize with cRNA of the human gene TRAF3
actggagagatgaatatcgcctctggctgcccagtctttgtggcccaaac

SEQ ID NO: 454 - DNA, 50 bp, Artificial Sequence
  Probe SG454 Designed to hybridize with cRNA of the human gene DAPK1
ccttttgctgaaggcatcctctgtgttcaaaatcaacctggatggcaatg

SEQ ID NO: 455 - DNA, 50 bp, Artificial Sequence
  Probe SG455 Designed to hybridize with cRNA of the human gene MAP3K12
cttcccggcctgcaacccccaaaatggattctgcgaggatgacaatgttt

SEQ ID NO: 456 - DNA, 50 bp, Artificial Sequence
  Probe SG456 Designed to hybridize with cRNA of the human gene PRKDC
ttaggattaattgagtggcttgaaaatactgttaccttgaaggaccttct

SEQ ID NO: 457 - DNA, 50 bp, Artificial Sequence
  Probe SG457 Designed to hybridize with cRNA of the human gene DNMT3B
ggcggaagcccatgcaacgatctctcaaatgtgaatccagccaggaaagg

SEQ ID NO: 458 - DNA, 50 bp, Artificial Sequence
  Probe SG458 Designed to hybridize with cRNA of the human gene GSTP1
cctcccctgagtacgtgaacctccccatcaatggcaacgggaaacagtga

SEQ ID NO: 459 - DNA, 50 bp, Artificial Sequence
  Probe SG459 Designed to hybridize with cRNA of the human gene HLA-DRA
agccctgtggaactgagagagcccaacgtcctcatctgtttcatcgacaa

SEQ ID NO: 460 - DNA, 50 bp, Artificial Sequence
  Probe SG460 Designed to hybridize with cRNA of the human gene HOXA10
tcccttcgccaaattatcccacaacaatgtcatcgtcggagagccccgcc

SEQ ID NO: 461 - DNA, 50 bp, Artificial Sequence
  Probe SG461 Designed to hybridize with cRNA of the human gene IRF8
ggatatggcccccttgcgctccaaactcattctcgtgcagattgagcagc

SEQ ID NO: 462 - DNA, 50 bp, Artificial Sequence
  Probe SG462 Designed to hybridize with cRNA of the human gene ID2
aattcccttctgagttaatgtcaaatgacagcaaagcactgtgtggctga

SEQ ID NO: 463 - DNA, 50 bp, Artificial Sequence
  Probe SG463 Designed to hybridize with cRNA of the human gene ACTB
tgatgatatcgccgcgctcgtcgtcgacaacggctccggcatgtgcaagg

SEQ ID NO: 464 - DNA, 50 bp, Artificial Sequence
  Probe SG464 Designed to hybridize with cRNA of the human gene ACTB
acgttgctatccaggctgtgctatccctgtacgcctctggccgtaccact

SEQ ID NO: 465 - DNA, 50 bp, Artificial Sequence
  Probe SG465 Designed to hybridize with cRNA of the human gene GRK4
atggtgaaaccccgtctctactaaaaatacaaaaattagccgggcgtggt

SEQ ID NO: 466 - DNA, 50 bp, Artificial Sequence
  Probe SG466 Designed to hybridize with cRNA of the human gene GAPD
tgcttttaactctggtaaagtggatattgttgccatcaatgaccccttca

SEQ ID NO: 467 - DNA, 50 bp, Artificial Sequence
  Probe SG467 Designed to hybridize with cRNA of the human gene GAPD
gtctcctctgacttcaacagcgacacccactcctccacctttgacgctgg

SEQ ID NO: 468 - DNA, 50 bp, Artificial Sequence
  Probe SG468 Designed to hybridize with cRNA of the human gene STAT1
atccaaatattgacaaagaccatgcctttggaaagtattactccaggcca

SEQ ID NO: 469 - DNA, 50 bp, Artificial Sequence
  Probe SG469 Designed to hybridize with cRNA of the human gene 18S rRNA
ttgactatctagaggaagtaaaagtcgtaacaaggtttccgtaggtgaac

SEQ ID NO: 470 - DNA, 50 bp, Artificial Sequence
  Probe SG470 Designed to hybridize with cRNA of the human gene TFRC
ccagctttactggagaacttgaaactgcgtaaacaaaataacggtgcttt

SEQ ID NO: 471 - DNA, 50 bp, Artificial Sequence
  Probe SG471 Designed to hybridize with cRNA of the human gene 28S rRNA
tctccccttccttggtgccttctcggctcttgacacttagccgctgtctc

SEQ ID NO: 472 - DNA, 50 bp, Artificial Sequence
  Probe SG472 Designed to hybridize with cRNA of the human gene RPL37A
tcacggtaaagtccgccatcagaagactgaaggagttgaaagaccagtag

SEQ ID NO: 473 - DNA, 50 bp, Artificial Sequence
  Probe SG473 Designed to hybridize with cRNA of the human gene RPL41
tggaggaagaagcgaatgcgcaggctgaagcgcaaaagaagaaagatgag

SEQ ID NO: 474 - DNA, 50 bp, Artificial Sequence
  Probe SG474 Designed to hybridize with cRNA of the human gene HNRPL
acccttacactctgaagttgtgtttctccactgctcagcacgcctcctaa

SEQ ID NO: 475 - DNA, 50 bp, Artificial Sequence
  Probe SG475 Designed to hybridize with cRNA of the human gene ILF2
ctatcttgcttctgaaatatctacctgggatggagtgatagtaacacctt

SEQ ID NO: 476 - DNA, 50 bp, Artificial Sequence
  Probe SG476 Designed to hybridize with cRNA of the human gene TAGLN2
gttccctaagaaatccaaggagaatcctcggaacttctcagataaccagc

SEQ ID NO: 477 - DNA, 50 bp, Artificial Sequence
  Probe SG477 Designed to hybridize with cRNA of the human gene CANP2
aaggcattagaagaagcaggtttcaagatgccctgtcaactccaccaagt

SEQ ID NO: 478 - DNA, 50 bp, Artificial Sequence
  Probe SG478 Designed to hybridize with cRNA of the human gene PSMA5
accacaagtctatgactttgaaagaagccatcaagtcttcactcatcatc

SEQ ID NO: 479 - DNA, 50 bp, Artificial Sequence
  Probe SG479 Designed to hybridize with cRNA of the human gene PMM1
atccacttctttgggaacgagactagccctggtgggaacgactttgagat

SEQ ID NO: 480 - DNA, 50 bp, Artificial Sequence
  Probe SG480 Designed to hybridize with cRNA of the human gene MLF2
aatacgggtgatggtgcccccaaggtctaccaagagacatcagagatgcg

SEQ ID NO: 481 - DNA, 50 bp, Artificial Sequence
  Probe SG481 Designed to hybridize with cRNA of the human gene PPP1CC
gagacctgtaacgcctccaaggggtatgatcacaaagcaagcaaagaaat

SEQ ID NO: 482 - DNA, 50 bp, Artificial Sequence
  Probe SG482 Designed to hybridize with cRNA of the human gene CASC3
atcacaggtatatggaggagtgacctactataaccccgcccagcagcagg

SEQ ID NO: 483 - DNA, 50 bp, Artificial Sequence
  Probe SG483 Designed to hybridize with cRNA of the human gene KIAA0864
tgccttaccatctctgccacctgtggaatcgctgagagattgccagaagc

SEQ ID NO: 484 - DNA, 50 bp, Artificial Sequence
  Probe SG484 Designed to hybridize with cRNA of the human gene TXNRD1
gccattggaatggacgattccgtcaagagataacaacaaatgttatgcaa

SEQ ID NO: 485 - DNA, 50 bp, Artificial Sequence
  Probe SG485 Designed to hybridize with cRNA of the human gene PSMD7
tgcacaacctcatcaacaacaagattgccaaccgggatgcagagaagaaa

SEQ ID NO: 486 - DNA, 50 bp, Artificial Sequence
  Probe SG486 Designed to hybridize with cRNA of the human gene EIF2B2
caccttcctacatctaccgcctgatgagtgaactctaccatcctgatgat

SEQ ID NO: 487 - DNA, 50 bp, Artificial Sequence
  Probe SG487 Designed to hybridize with cRNA of the human gene HLA-A
tagaaaaggagggagctactctcaggctgcaagcagtgacagtgcccagg

SEQ ID NO: 488 - DNA, 50 bp, Artificial Sequence
  Probe SG488 Designed to hybridize with cRNA of the human gene MCM7
ggcagcttcaatcgccccagaaatatacgggcatgaagatgtgaagaagg

SEQ ID NO: 489 - DNA, 50 bp, Artificial Sequence
  DProbe SG489 esigned to hybridize with cRNA of the human gene TNFRSF25
ctgacctacacataccgccactgctggcctcacaagcccctggttactgc

SEQ ID NO: 490 - DNA, 50 bp, Artificial Sequence
  Probe SG490 Designed to hybridize with cRNA of the human gene LGALS3
agtacaatcatcgggttaaaaaactcaatgaaatcagcaaactgggaatt

SEQ ID NO: 491 - DNA, 50 bp, Artificial Sequence
  Probe SG491 Designed to hybridize with cRNA of the human gene HLA-DPA1
gaacagattacagcttccacaagttccattacctgacctttgtgccctca

SEQ ID NO: 492 - DNA, 50 bp, Artificial Sequence
  Probe SG492 Designed to hybridize with cRNA of the human gene PLAU
ctcgtctgttccctccaaggccgcatgactttgactggaattgtgagctg

SEQ ID NO: 493 - DNA, 50 bp, Artificial Sequence
  Probe SG493 Designed to hybridize with cRNA of the human gene COL3A1
tcgaggattccctggtaatccaggtgccccaggttctccaggccctgctg

SEQ ID NO: 494 - DNA, 50 bp, Artificial Sequence
  Probe SG494 Designed to hybridize with cRNA of the human gene ANK2
ctcagttcctgaggacatctttgacacaaggcccatttgggatgagtcta

SEQ ID NO: 495 - DNA, 50 bp, Artificial Sequence
  Probe SG495 Designed to hybridize with cRNA of the human gene SERPINA9
gcacctctattgcagaattacaataacacattcaataaaactaaaatatg

SEQ ID NO: 496 - DNA, 50 bp, Artificial Sequence
  Probe SG496 Designed to hybridize with cRNA of the human gene NPM3
ggcaccagattgttacgatgagcaatgatgtttctgaggaggagagcgag

SEQ ID NO: 497 - DNA, 50 bp, Artificial Sequence
  Probe SG497 Designed to hybridize with cRNA of the human gene CCR6
gcagaccagtgagaccgcagataacgacaatgcgtcgtccttcactatgt

SEQ ID NO: 498 - DNA, 50 bp, Artificial Sequence
  Probe SG498 Designed to hybridize with cRNA of the human gene HCK
ccgagctgccaacatcttggtctctgcatccctggtgtgtaagattgctg

SEQ ID NO: 499 - DNA, 50 bp, Artificial Sequence
  Probe SG499 Designed to hybridize with cRNA of the human gene GNL3
aggacatacatgaagaattgccaaaacggaaagaaaggaagcaggaggag

SEQ ID NO: 500 - DNA, 50 bp, Artificial Sequence
  Probe SG500 Designed to hybridize with cRNA of the human gene GRB2
ccggcatgtttccccgcaattatgtcacccccgtgaaccggaacgtctaa

SEQ ID NO: 501 - DNA, 50 bp, Artificial Sequence
  Probe SG501 Designed to hybridize with cRNA of the human gene BCL2A1
ggagttcataatgaataacacaggagaatggataaggcaaaacggaggct

SEQ ID NO: 502 - DNA, 50 bp, Artificial Sequence
  Probe SG502 Designed to hybridize with cRNA of the human gene ELF1
gtttagaggtctaatgctatgttttcatattacagagtgaatttgtattt

SEQ ID NO: 503 - DNA, 50 bp, Artificial Sequence
  Probe SG503 Designed to hybridize with cRNA of the human gene CTSB
tgctctacaagtcaggagtgtaccaacacgtcaccggagagatgatgggt

SEQ ID NO: 504 - DNA, 50 bp, Artificial Sequence
  Probe SG504 Designed to hybridize with cRNA of the human gene GCET2
atcctggttctctctcttcctccatctgtgactttgaacatgctacttgg

SEQ ID NO: 505 - DNA, 50 bp, Artificial Sequence
  Probe SG505 Designed to hybridize with cRNA of the human gene FN1
tcgtgctttgacccctacacagtttcccattatgccgttggagatgagtg

SEQ ID NO: 506 - DNA, 50 bp, Artificial Sequence
  Probe SG506 Designed to hybridize with cRNA of the human gene PDCD1
ttcctagcggaatgggcacctcatcccccgcccgcaggggctcagccgac

SEQ ID NO: 507 - DNA, 50 bp, Artificial Sequence
  Probe SG507 Designed to hybridize with cRNA of the human gene HLA-DRB3
gttcatctacttcaggaatcagaaaggacactctggacttcagccaacag

SEQ ID NO: 508 - DNA, 50 bp, Artificial Sequence
  Probe SG508 Designed to hybridize with cRNA of the human gene HLA-DQA1
gcagtcagtcacagaagatgtttctgagaccagcttcctctccaagagtg

SEQ ID NO: 509 - DNA, 50 bp, Artificial Sequence
  Probe SG509 Designed to hybridize with cRNA of the human gene GCET2
ggtgaaggctccacaaacctgtgacacatgcaagctcaagccattgcatt

SEQ ID NO: 510 - DNA, 50 bp, Artificial Sequence
  Probe SG510 Designed to hybridize with cRNA of the human gene SERPNINA9
ggagccccaaggaatgaaatatggtaacccagcaacatcccatgaagcta

SEQ ID NO: 511 - DNA, 50 bp, Artificial Sequence
  Probe SG511 Designed to hybridize with cRNA of the human gene CDKN3
ctgcacatctatcatcaagagattcacaatcaagatctgtatcaagataa

SEQ ID NO: 512 - DNA, 50 bp, Artificial Sequence
  Probe SG512 Designed to hybridize with cRNA of the human gene ELF1
cttctctcaagcttcttggccttcaagtattctttctctatcgtcttttg

SEQ ID NO: 513 - DNA, 50 bp, Artificial Sequence
  Probe SG513 Designed to hybridize with cRNA of the human gene CATSD
gtgccgctgattcagggcgagtacatgatcccctgtgagaaggtgtccac

SEQ ID NO: 514 - DNA, 50 bp, Artificial Sequence
  Probe SG514 Designed to hybridize with cRNA of the human gene HSPB1
atggctacatctcccggtgcttcacgcggaaatacacgctgccccccggt

SEQ ID NO: 515 - DNA, 50 bp, Artificial Sequence
  Probe SG515 Designed to hybridize with cRNA of the human gene ACTN1
cccctacacaaccatcacgcctcaggagatcaatggcaaatgggaccacg

SEQ ID NO: 516 - DNA, 50 bp, Artificial Sequence
  Probe SG516 Designed to hybridize with cRNA of the human gene BMP6
gaaccccgagtatgtccccaaaccgtgctgtgcgccaactaagctaaatg

SEQ ID NO: 517 - DNA, 50 bp, Artificial Sequence
  Probe SG517 Designed to hybridize with cRNA of the human gene FAM38A
gtctgtggtcatccccaatctcttccccaagtacatccgtgcccccaacg

SEQ ID NO: 518 - DNA, 50 bp, Artificial Sequence
  Probe SG518 Designed to hybridize with cRNA of the human gene CD48
ttatacttgccaagtcagcaattctgtgagcagcaagaatggcaccgtct

SEQ ID NO: 519 - DNA, 50 bp, Artificial Sequence
  Probe SG519 Designed to hybridize with cRNA of the human gene IL4R
tcacagtgggagaagcggtcccgaggccaggaaccagccaagtgcccaca

SEQ ID NO: 520 - DNA, 50 bp, Artificial Sequence
  Probe SG520 Designed to hybridize with cRNA of the human gene DRP2
ccagtgtgcgaagttctgatgtgactgccaacaccctgctggcctcttga

SEQ ID NO: 521 - DNA, 50 bp, Artificial Sequence
  Probe SG521 Designed to hybridize with cRNA of the human gene JUNB
ggaggacaaggtgaagacgctcaaggccgagaacgcggggctgtcgagta

SEQ ID NO: 522 - DNA, 50 bp, Artificial Sequence
  Probe SG522 Designed to hybridize with cRNA of the human gene S100A8
aattgctagagaccgagtgtcctcagtatatcaggaaaaagggtgcagac

SEQ ID NO: 523 - DNA, 50 bp, Artificial Sequence
  Probe SG523 Designed to hybridize with cRNA of the human gene ZNFN1A1
agttggaggcattcgacttcctaacggaaaactaaagtgtgatatctgtg

SEQ ID NO: 524 - DNA, 50 bp, Artificial Sequence
  Probe SG524 Designed to hybridize with cRNA of the human gene MERTK
tcattgcccgatgaacttttgtttgctgacgactcctcagaaggctcaga

SEQ ID NO: 525 - DNA, 50 bp, Artificial Sequence
  Probe SG525 Designed to hybridize with cRNA of the human gene KLF13
tgcctagtgctgaggcctctggggctggaaagcctcagcagaaaggaggc

SEQ ID NO: 526 - DNA, 50 bp, Artificial Sequence
  Probe SG526 Designed to hybridize with cRNA of the human gene CBFB
aatttcccctgagggaatcgctttttaagtgatccttacagtggtgtttt

SEQ ID NO: 527 - DNA, 50 bp, Artificial Sequence
  Probe SG527 Designed to hybridize with cRNA of the human gene CEBPB
tcctctccgacctcttctccgacgactacgggggcaagaactgcaagaag

SEQ ID NO: 528 - DNA, 50 bp, Artificial Sequence
  Probe SG528 Designed to hybridize with cRNA of the human gene CEBPD
cggggcgggagagactcagcaacgacccatacctcagacccgacggcccg

SEQ ID NO: 529 - DNA, 50 bp, Artificial Sequence
  Probe SG529 Designed to hybridize with cRNA of the human gene TFCP2
ctgcagttgagggagcagcaacaacagcagcagcaacagcagcagaagca

SEQ ID NO: 530 - DNA, 50 bp, Artificial Sequence
  Probe SG530 Designed to hybridize with cRNA of the human gene CREB1
cgcctgtaatcccagcactttgggaggccaaggtgggtggatcacctgtg

SEQ ID NO: 531 - DNA, 50 bp, Artificial Sequence
  Probe SG531 Designed to hybridize with cRNA of the human gene NFIC
gcggcgattactacacttcgcccagctcgcccacgagtagcagccgcaac

SEQ ID NO: 532 - DNA, 50 bp, Artificial Sequence
  Probe SG532 Designed to hybridize with cRNA of the human gene GABPB2
tgctccattgtccaattcttcagaaactccagtagtggccacagaagaag

SEQ ID NO: 533 - DNA, 50 bp, Artificial Sequence
  Probe SG533 Designed to hybridize with cRNA of the human gene EGR1
gatggcttgacatgtgcaattgtgagggacatgctcacctctagccttaa

SEQ ID NO: 534 - DNA, 50 bp, Artificial Sequence
  Probe SG534 Designed to hybridize with cRNA of the human gene KLF1
gccactaccggaaacacacggggcagcgccccttccgctgccagctctgc

SEQ ID NO: 535 - DNA, 50 bp, Artificial Sequence
  Probe SG535 Designed to hybridize with cRNA of the human gene ELF1
tatggctgaccctgtttcagaagcaggatagtataaaagcatcagcctaa

SEQ ID NO: 536 - DNA, 50 bp, Artificial Sequence
  Probe SG536 Designed to hybridize with cRNA of the human gene ETS1
ctcgagctggccccagactttgttggggacatcttatgggaacatctaga

SEQ ID NO: 537 - DNA, 50 bp, Artificial Sequence
  Probe SG537 Designed to hybridize with cRNA of the human gene ETS2
tatttgaaataagaattcagacatctgaggttttatttcatttttcaata

SEQ ID NO: 538 - DNA, 50 bp, Artificial Sequence
  Probe SG538 Designed to hybridize with cRNA of the human gene FLI1
attcgcaagtgctgtgcgcttgtcagaccatcagaccagggccaaccaat

SEQ ID NO: 539 - DNA, 50 bp, Artificial Sequence
  Probe SG539 Designed to hybridize with cRNA of the human gene GATA3
gagccctgctcgatgctcacagggcccccagcgagagtccctgcagtccc

SEQ ID NO: 540 - DNA, 50 bp, Artificial Sequence
  Probe SG540 Designed to hybridize with cRNA of the human gene CBFA2T1
tgttggacaaatgtgaagatgcattgtagtttaaccatatgcccacattt

SEQ ID NO: 541 - DNA, 50 bp, Artificial Sequence
  Probe SG541 Designed to hybridize with cRNA of the human gene HIF1A
atgcattcttagcaaaattgcctagtatgttaatttgctcaaaatacaat

SEQ ID NO: 542 - DNA, 50 bp, Artificial Sequence
  Probe SG542 Designed to hybridize with cRNA of the human gene TCF1
cccgagcagctgagcagggccggggaactggccaagctgaggtgcccagg

SEQ ID NO: 543 - DNA, 50 bp, Artificial Sequence
  Probe SG543 Designed to hybridize with cRNA of the human gene HOXD8
caaatttcccgtttcccggcaggaggtgaaggacggggaaacgaaaaagg

SEQ ID NO: 544 - DNA, 50 bp, Artificial Sequence
  Probe SG544 Designed to hybridize with cRNA of the human gene HOXD9
cctcagcttgcagcgaccacccgatcccaggctgttcgctgaaggaggag

SEQ ID NO: 545 - DNA, 50 bp, Artificial Sequence
  Probe SG545 Designed to hybridize with cRNA of the human gene MAFB
agcgctcctagggtgagaggcttagccatccctgaccctggcagtgcact

SEQ ID NO: 546 - DNA, 50 bp, Artificial Sequence
  Probe SG546 Designed to hybridize with cRNA of the human gene MAFK
gcggcgggcaggcgggtgggggcacacccctcgtacctgtcactgggatg

SEQ ID NO: 547 - DNA, 50 bp, Artificial Sequence
  Probe SG547 Designed to hybridize with cRNA of the human gene EDF1
ttggccgccagttccgttctcctcacgggccgaacggaacaaggggtcca

SEQ ID NO: 548 - DNA, 50 bp, Artificial Sequence
  Probe SG548 Designed to hybridize with cRNA of the human gene ELF4
ctagatggggtccacccccattcctgctcaagcatgggcacctaccacat

SEQ ID NO: 549 - DNA, 50 bp, Artificial Sequence
  Probe SG549 Designed to hybridize with cRNA of the human gene ZNF42
tccgcgtacacacgggcgagaaaccctttgcctgccccgagtgtggccag

SEQ ID NO: 550 - DNA, 50 bp, Artificial Sequence
  Probe SG550 Designed to hybridize with cRNA of the human gene NF1
ggaattgatgaagaaaccagtgaagaatccctcctgactcccacatctcc

SEQ ID NO: 551 - DNA, 50 bp, Artificial Sequence
  Probe SG551 Designed to hybridize with cRNA of the human gene NFATC1
ttctcgtctcacgcagttcgaggaggaccctagaaagccaggagctgtga

SEQ ID NO: 552 - DNA, 50 bp, Artificial Sequence
  Probe SG552 Designed to hybridize with cRNA of the human gene PAX6
ggtgtgtcagttccagttcaagttcccggaagtgaacctgatatgtctca

SEQ ID NO: 553 - DNA, 50 bp, Artificial Sequence
  Probe SG553 Designed to hybridize with cRNA of the human gene PAX8
gcaccaccccaggtcctcctgcagtgcggcatccccttggcagctgccgt

SEQ ID NO: 554 - DNA, 50 bp, Artificial Sequence
  Probe SG554 Designed to hybridize with cRNA of the human gene PLP
gccagtgatggtgggtgggggctgggccttccccgccacctccacccctg

SEQ ID NO: 555 - DNA, 50 bp, Artificial Sequence
  Probe SG555 Designed to hybridize with cRNA of the human gene PLZF
gtgctccctctcactggtggggcgtggcttcctcacagagagggacgaca

SEQ ID NO: 556 - DNA, 50 bp, Artificial Sequence
  Probe SG556 Designed to hybridize with cRNA of the human gene RBP4
gtacaggctgatcgtccacaacggttactgcgatggcagatcagaaagaa

SEQ ID NO: 557 - DNA, 50 bp, Artificial Sequence
  Probe SG557 Designed to hybridize with cRNA of the human gene RORA
cagacctggagcgccacacactgcacatcttttggtgatcggggtcaggc

SEQ ID NO: 558 - DNA, 50 bp, Artificial Sequence
  Probe SG558 Designed to hybridize with cRNA of the human gene SP1
cctgcttccccttcctaagtctgtcatcctctggaagggatgggtggtgc

SEQ ID NO: 559 - DNA, 50 bp, Artificial Sequence
  Probe SG559 Designed to hybridize with cRNA of the human gene STAT1
atatcaatagaaggatgtacatttccaaattcacaagttgtgtttgatat

SEQ ID NO: 560 - DNA, 50 bp, Artificial Sequence
  Probe SG560 Designed to hybridize with cRNA of the human gene TBP
tgcagggtgtggcaccaggtgatgcccttctgtaagtgcccaccgcggga

SEQ ID NO: 561 - DNA, 50 bp, Artificial Sequence
  Probe SG561 Designed to hybridize with cRNA of the human gene TCF7
aagggctgccgggccaggcgcggtggctcacgcctgtaatcccagcactt

SEQ ID NO: 562 - DNA, 50 bp, Artificial Sequence
  Probe SG562 Designed to hybridize with cRNA of the human gene ETV7
cagccattgcttgggaaggctgggaggcctcccatccaggacactggggg

SEQ ID NO: 563 - DNA, 50 bp, Artificial Sequence
  Probe SG563 Designed to hybridize with cRNA of the human gene ZAP70
cgcggaaggagcagggcacatacgccctgtccctcatctatgggaagacg

SEQ ID NO: 564 - DNA, 55 bp, Artificial Sequence
  Negative control
ggggaaagaaagatcgatgagaaagaggaacggggattgacaggaatgaagaagg

SEQ ID NO: 565 - DNA, 50 bp, Artificial Sequence
  Negative control
cgtctcggtgaactcaagtgaatcaagggaagagaattcctaactgcacg

SEQ ID NO: 566 - DNA, 55 bp, Artificial Sequence
  Negative control
tcagcttcctttctccctttcctccttccttccttctcaccatcctgtccttttt

SEQ ID NO: 567 - DNA, 55 bp, Artificial Sequence
  Negative control
gaggaaacaggaaagaagaggaaggaaggaagacagggaagaaaagagggaggga

SEQ ID NO: 568 - DNA, 54 bp, Artificial Sequence
  Negative control
tctgtggggaggggttgttttggggttgtttttgttttttcttgccaggtagat

SEQ ID NO: 569 - DNA, 55 bp, Artificial Sequence
  Negative control
ctctctctctctctctctctctctctgtctctccccacctctctcacatcccttg

SEQ ID NO: 570 - DNA, 50 bp, Artificial Sequence
  Negative control
tgtgtgtgtgtgtgtgtgtgtgtgtgtgtgtggtgttgtgtgtgtttgtg

SEQ ID NO: 571 - DNA, 25 bp, Artificial Sequence
  Negative control
gtcgtcaagatgctaccgttcagga

SEQ ID NO: 572 - DNA, 50 bp, Artificial Sequence
  Positive hybridization control
aggccgtgaagatatattcatcagctaccatgcgaaagacgatgtccaaa

SEQ ID NO: 573 - DNA, 50 bp, Artificial Sequence
  Positive hybridization control
cgagccaggcaaagcggagtaactcagttcgaagtaaggggaaaatgtcg

SEQ ID NO: 574 - DNA, 50 bp, Artificial Sequence
  Gives rise to non-specific hybridization
gcttttcttcgtttatcatcaccatcaccatcatcatcatcatcatcatc

SEQ ID NO: 575 - DNA, 50 bp, Artificial Sequence
  Negative control
gccaggatctcaaacccaccaaaccgttccagaatcatggaccgctttcg

SEQ ID NO: 576 - DNA, 50 bp, Artificial Sequence
  Positive hybridization control
gtccaagcgagaagatacttaacaatatcaacattaccattaccagcaga

SEQ ID NO: 577 - DNA, 50 bp, Artificial Sequence
  Negative control
atcccgatgcaaaaggcggacgacaaggactacggtctagaagctctgga

SEQ ID NO: 578 - DNA, 50 bp, Artificial Sequence
  Positive hybridization control
ccacatcaccttcaccatttttcttcttcgttgattctctgtttgtgttt

SEQ ID NO: 579 - DNA, 50 bp, Artificial Sequence
  Gives rise to non-specific hybridization
tcctctgttttgctcttgccttatccgctgcttctctctccctctctttc

SEQ ID NO: 580 - DNA, 50 bp, Artificial Sequence
  Negative control
tgtgacacgacctacaacaatggctgcaacggtggtctcatggactatgc

SEQ ID NO: 581 - DNA, 50 bp, Artificial Sequence
  Positive hybridization control
agaataataaatgaagtgatgatgattgatgattgatgattgatgattat

SEQ ID NO: 582 - DNA, 50 bp, Artificial Sequence
  Positive hybridization control
agtgacaagtttcgctgattatagtggttaatgctggtcttatcttcgtc

SEQ ID NO: 583 - DNA, 50 bp, Artificial Sequence
  Positive hybridization control
atgggcggtgcggggttgatgttgacgacggtgtggctgcggttgggtat

SEQ ID NO: 584 - DNA, 50 bp, Artificial Sequence
  Acts as positive internal process control spiked control
cctcaacgcctacaaaagccagtttatcccgtcaaaggattccgtttcta

SEQ ID NO: 585 - DNA, 50 bp, Artificial Sequence
  Acts as positive internal process control spiked control
cgttatacggaagatgatgaaccgcttcatgccactgaatacgttgaaaa

SEQ ID NO: 586 - DNA, 50 bp, Artificial Sequence
  Acts as positive internal process control spiked control
ctaccaagtggctaaacggaccgcagatttgtacggaaaagaaatgcctc

SEQ ID NO: 587 - DNA, 50 bp, Artificial Sequence
  Acts as positive internal process control spiked control
aacacaagcggtttccaccatgtccacaagaaaggggaggtaacaccagg

SEQ ID NO: 588 - DNA, 50 bp, Artificial Sequence
  Acts as positive internal process control spiked control
gccagcaacaactcaaccagcaacaaaaccagtttcacaggtgtcaggac

SEQ ID NO: 589 - DNA, 50 bp, Artificial Sequence
  Acts as positive internal process control spiked control
aagcagattcagtagcagatgccgttcaaaaggtcgatttaagtagaagt

SEQ ID NO: 590 - DNA, 50 bp, Artificial Sequence
  Acts as positive internal process control spiked control
gacttcctgatattgttgatacatgcggaacagggggagacggtatttcc

SEQ ID NO: 591 - DNA, 70 bp, Artificial Sequence
  Antisense oligonucleotide C2 complementary to SCN2. Biotin residue at 5′
acgctataaacatccactaccgacattttccccttactttgaactgagttactccgctttgcctggctcg

SEQ ID NO: 592 - DNA, 70 bp, Artificial Sequence
  Antisense oligonucleotide C3 complementary to SCN3.Biotin residue at 5′
gagcgaaccggcgtaagttgtttggacatcgtctttcgcatggtagctgatgaatatatcttcacggcct

SEQ ID NO: 593 - DNA, 70 bp, Artificial Sequence
  Antisense oligonucleotide C6 complementary to SCN6. Biotin residue at n 5′
atcatacgccacttcatgtttctgctggtaatggtaatgttgatattgttaagtatcttctcgcttggac

SEQ ID NO: 594 - DNA, 70 bp, Artificial Sequence
  Antisense oligonucleotide C8 complementary to SCN8. Residue of biotin at 5′
actgaaaacagaacaaacacaaacacaaacagagaatcaacgaagaagaaaaatggtgaaggtgatgtgg

SEQ ID NO: 595 - DNA, 70 bp, Artificial Sequence
  Antisense oligonucleotide C11 complementary to SCN11. Residue of biotin at 5′
aacattcaagcatgatgatgataatcatcaatcatcaatcatcaatcatcatcacttcatttattattct

SEQ ID NO: 596 - DNA, 70 bp, Artificial Sequence
  Antisense oligonucleotide C12 complementary to SCN12. Residue of biotin at 5′
ttaggattctaaatagtagcgacgaagataagaccagcattaaccactataatcagcgaaacttgtcact

SEQ ID NO: 597 - DNA, 70 bp, Artificial Sequence
  Antisense oligonucleotide C13 complementary to SCN13. Residue of biotin at 5′
tctgaaggcttgcttgatccatacccaaccgcagccacaccgtcgtcaacatcaaccccgcaccgcccat

SEQ ID NO: 598 - DNA, 24 bp, Artificial Sequence
  Primer PSMB4_Forward of Gene PSMB4
ttctgggagatggacacagctata

SEQ ID NO: 599 - DNA, 21 bp, Artificial Sequence
  Primer PSMB4_Reverse of Gene PSMB4
ccacaaagggttcatcttcga

SEQ ID NO: 600 - DNA, 21 bp, Artificial Sequence
  Primer CD23A_Forward of Gene CD23A
tgccctgaaaagtggatcaat

SEQ ID NO: 601 - DNA, 21 bp, Artificial Sequence
  Primer CD23A_Reverse of Gene CD23A
ccatgtcgtcacaggcatacc

SEQ ID NO: 602 - DNA, 21 bp, Artificial Sequence
  Primer LCP1_Forward of Gene LCP1
ccaggtacccttctcgctttt

SEQ ID NO: 603 - DNA, 20 bp, Artificial Sequence
  Primer LCP1_Reverse of Gene LCP1
ctcctggccctcatcttgaa

SEQ ID NO: 604 - DNA, 24 bp, Artificial Sequence
  Primer ABCC5_Forward of Gene ABCC5
ccctcaaagtctgcaactttaagc

SEQ ID NO: 605 - DNA, 21 bp, Artificial Sequence
  Primer ABCC5_Reverse of Gene ABCC5
acacaccaaaccacacagcaa

SEQ ID NO: 606 - DNA, 21 bp, Artificial Sequence
  Primer POU2F2_Forward of Gene POU2F2
gaggaccagcatcgagacaaa

SEQ ID NO: 607 - DNA, 20 bp, Artificial Sequence
  Primer POU2F2_Reverse of Gene POU2F2
aaccagacgcggatcacttc